(19) United States
(12) Patent Application Publication
(10) Pub. No.: US 2024/0390471 A1
(43) Pub. Date: Nov. 28, 2024

(54) COMPOUNDS AND ADJUVANT FORMULATIONS USEFUL IN PNEUMOCOCCAL VACCINES

(71) Applicant: Merck Sharp & Dohme LLC, Rahway, NJ (US)

(72) Inventors: Patrick L. Ahl, Yardley, PA (US); Peter J. Manley, Harleysville, PA (US); Izzat T. Raheem, Doylestown, PA (US); W. Michael Seganish, Castro Valley, CA (US); William J. Smith, Harleysville, PA (US)

(73) Assignee: Merck Sharp & Dohme LLC, Rahway, NJ (US)

(21) Appl. No.: 18/665,887

(22) Filed: May 16, 2024

Related U.S. Application Data
(60) Provisional application No. 63/503,021, filed on May 18, 2023.

(51) Int. Cl. A61K 39/09; A61K 39/00; A61K 39/385; A61K 39/39; A61K 47/06; A61K 47/22; A61K 47/26; A61P 31/04

(52) U.S. Cl. CPC A61K 39/092; A61K 39/385; A61K 39/39; A61K 47/06; A61K 47/22; A61K 47/26; A61P 31/04; A61K 2039/55511; A61K 2039/575; A61K 2039/6037; A61K 2039/70

(57) ABSTRACT

The invention relates to novel compounds and formulations. Further, the invention relates to novel compounds and formulations useful in pneumococcal and pneumococcal conjugate vaccines. More specifically, the invention relates to compositions comprising pneumococcal conjugates and one or more compounds of Formula I, Ia, II, IIa, III, IIIa, IV, or IVa, or a pharmaceutically acceptable salt thereof, prepared as stable nanoemulsions (herein referred to as “SNE adjuvant compositions” or “SNEs”).

### CROSS-REFERENCE TO RELATED APPLICATIONS

This application claims the benefit of U.S. Provisional Patent Application Ser. No. 63/503,021 filed May 18, 2023, the entire contents of which are incorporated by reference herein.

### BACKGROUND OF THE INVENTION

Pneumococcal disease is caused by infection with the bacteria Streptococcus pneumoniae (pneumococcus). Different pneumococcal serotypes are known to cause different manifestations of the disease and infections can cause a range of symptoms from ear and sinus infections to pneumonia and bloodstream infections. Pneumococcal disease has a high associated morbidity and mortality worldwide, particularly among the elderly and young children. Currently 100 capsular polysaccharides (which, in part, are used to define or designate a particular serotype) have been identified (Ganaie, F. et al. (2020) Clinical Science and Epidemiology, Vol. 11, Issue 3, pages 1-15). These serotypes are distinguished by their chemical structure, serological response, and other related genetic mutations.

In 1983, PNEUMOVAX® (Merck & Co., Inc., Rahway, NJ, USA), a 23-valent pneumococcal vaccine (PV) was approved in the United States. This vaccine demonstrated reduced immunogenicity in infants due to T-cell independent responses. To address this issue, particularly in infants, pneumococcal conjugate vaccines (PCVs) were developed. By covalently coupling the polysaccharide to a carrier protein, the immunogenic response became T-cell dependent. In 2000, PREVNAR® (Pfizer Inc., Philadelphia, PA, USA), a 7-valent PCV was approved in the United States. In 2010, PREVNAR13® (Pfizer Inc.), a 13-valent PCV was approved in the United States. In 2021, a 15-valent PCV, VAXNEUVANCE® (Merck & Co., Inc., Rahway, NJ, USA) and a 20-valent PCV, PREVNAR20® (Pfizer Inc.) were approved in the United States.

Licensed PCVs currently utilize aluminum containing derivatives as adjuvants to increase immunogenicity. Even though aluminum adjuvants increase immunogenic responses from baseline, it is unknown whether the immunogenic response is sufficient for higher valency PCVs, particularly in infants. Therefore, there is a need to identify other adjuvants that can provide increased immunogenicity for multivalent PCVs over the current aluminum adjuvant standard.

### SUMMARY OF THE INVENTION

The invention relates to formulations and compositions comprising (i) one or more S. pneumoniae antigens, (ii) one or more compounds of Formula I, Ia, II, IIa, III, IIIa, IV or IVa, or pharmaceutically acceptable salt(s) thereof and (iii) a pharmaceutically acceptable carrier, wherein the one or more S. pneumoniae antigens are capable of inducing an immune response against one or more serotypes of S. pneumoniae.

The invention also relates to formulations and compositions comprising (i) one or more S. pneumoniae polysaccharide-carrier protein conjugates, wherein each of the conjugates comprises a polysaccharide of a particular S. pneumoniae serotype conjugated to a carrier protein, and the carrier protein is CRM197, (ii) one or more compounds of Formula I, Ia, II, IIa, III, IIIa, IV or IVa, or pharmaceutically acceptable salt(s) thereof and (iii) a pharmaceutically acceptable carrier. Further, the invention relates to the use of a formulation or composition comprising (i) one or more S. pneumoniae polysaccharide-carrier protein conjugates, wherein each of the conjugates comprises a polysaccharide of a particular S. pneumoniae serotype conjugated to a carrier protein, and the carrier protein is CRM197, (ii) one or more compounds of Formula I, Ia, II, IIa, III, IIIa, IV or IVa, or pharmaceutically acceptable salt(s) thereof, and (iii) a pharmaceutically acceptable carrier, for the prevention of pneumococcal disease or for the prevention of or reduction of the likelihood of infection with Streptococcus pneumoniae.

The invention also relates to formulations and compositions comprising (i) one or more S. pneumoniae antigens; (ii) one or more compounds of Formula I, Ia, II, IIa, III, IIIa, IV or IVa, or a pharmaceutically acceptable salt thereof; (iii) one or more sorbitan-based surfactants; and iv) one or more terpenes. In some embodiments, the one or more S. pneumoniae antigens are S. pneumoniae polysaccharide-carrier protein conjugates, wherein each of the conjugates comprises a polysaccharide of a particular S. pneumoniae serotype conjugated to a carrier protein. In some embodiments, the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B, and the carrier protein is CRM197.

The invention also relates to formulations and compositions comprising (i) one or more S. pneumoniae antigens, (ii) one or more compounds of Formula I, Ia, II, IIa, III, IIIa, IV or IVa, or a pharmaceutically acceptable salt thereof, (iii) sorbitan trioleate (SPAN-85); (iv) polysorbate-20 (PS-20) or polysorbate-80 (PS-80), and (v) squalene. In some embodiments, the one or more S. pneumoniae antigens are S. pneumoniae polysaccharide-carrier protein conjugates, wherein each of the conjugates comprises a polysaccharide of a particular S. pneumoniae serotype conjugated to a carrier protein. In some embodiments, the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and the carrier protein is CRM197. In some embodiments, the S. pneumoniae antigens are polysaccharide-carrier protein conjugates wherein the polysaccharide consists of serotypes 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B, and the carrier protein is CRM197.

The invention also relates to compositions comprising pneumococcal conjugates and a stable nanoemulsion “SNE” disclosed herein. As an example, a particular SNE comprises N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide, otherwise referred to as “Compound A-1”. This particular SNE is referred to as Compound A-1-SNE and comprises i) Compound A-1, ii) sorbitan trioleate (SPAN-85); (iii) polysorbate-20 (PS-20) or polysorbate-80 (PS-80), and (iv) squalene. As another example, a particular SNE comprises N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide, otherwise referred to as “Compound B-1”. This particular SNE is referred to as Compound B-1-SNE and comprises i) Compound B-1, ii) SPAN-85; (iii) PS-20 or PS-80, and (iv) squalene. As another example, a particular SNE comprises N-(4-((4-((5-amino-7-(butylamino)-2H-pyrazolo[4,3-d]pyrimidin-2-yl)methyl)-3,5-dimethoxybenzyl)(methyl)amino)butyl)stearamide, otherwise referred to as “Compound C-4”. This particular SNE is referred to as Compound C-4-SNE and comprises i) Compound C-4, ii) SPAN-85; (iii) PS-20 or PS-80, and (iv) squalene.

The invention also relates to immunogenic compositions comprising pneumococcal polysaccharides or pneumococcal conjugates and an SNE disclosed herein.

The invention also relates to methods of treating or preventing a pneumococcal disease in a patient in need thereof by administering an immunogenic composition of the invention to the patient.

The invention also relates to methods of preventing or reducing the likelihood of infection with one or more serotypes of S. pneumoniae comprising administering an immunogenic composition of the invention to a patient in need thereof.

### BRIEF DESCRIPTION OF THE DRAWINGS

FIG. 1 (Example 10) shows the OD640 readout following incubation with different concentrations of Compound B-1-SNE with human TLR7 (top panel) or TLR8 (bottom panel) expressing HEK-Blue™ 293 cells (InvivoGen). The Y-axis represents total Compound B-1-SNE concentration.

FIG. 2 (Example 11) shows pre-immune (day 0, pooled sera), post-dose 1 (day 21), post-dose 2 (day 42) and post-dose 3 (day 70) anti-6B IgG titers after immunization of mice with formulations described in Table 7. Error bars are geometric means with 95% confidence intervals. Transformed data analyzed by one-way ANOVA with Dunnett post-test, *p<0.05.

FIG. 3 (Example 11) shows pre-immune (day 0), post-dose 1 (day 21), post-dose 2 (day 42) post-dose 3 (day 70) anti-6B OPA titers from pooled sera after immunization of mice with formulations described in Table 7.

FIG. 4 (Example 12) shows the ratio of serotype specific IgG titers in infant rhesus macaques following immunization of PCV24 with Compound B-1-SNE (150 μg dose of Compound B-1), PCV24 with Compound B-1-SNE (50 μg Compound B-1), PCV24 with Compound B-1-SNE (15 μg Compound B-1) compared to an immunization of PCV24 formulated without adjuvant at post dose 3. Serotype 6C and 15B data are included to evaluate cross reactivity.

FIG. 5 (Example 12) shows the ratio of serotype specific IgG titers in infant rhesus macaques following immunization with PCV24 with Compound B-1-SNE (150 μg dose of Compound B-1), PCV24 with Compound B-1-SNE (50 μg Compound B-1), PCV24 with Compound B-1-SNE (15 μg Compound B-1) compared to an immunization of PCV24 formulated with APA at post dose 3. Serotype 6C and 15B data are included to evaluate cross reactivity.

FIG. 6 (Example 12) shows the ratio of serotype specific IgG titers in infant rhesus macaques following immunization of PCV24 with Compound B-1-SNE (150 μg dose of Compound B-1), PCV24 with Compound B-1-SNE (50 μg Compound B-1), PCV24 with Compound B-1-SNE (15 μg Compound B-1) compared to an immunization of PCV20 at post dose 3.

FIG. 7 (Example 13) shows the ratio of serotype specific IgG titers in infant rhesus macaques following immunization of PCV24 with Compound B-1-SNE (100 μg dose of Compound B-1), PCV24 with Compound B-1-SNE (10 μg Compound B-1), PCV24 with Compound B-1-SNE (1 μg Compound B-1), PCV24 with Compound B-1-SNE (0.1 μg Compound B-1) compared to an immunization of PCV20 at post dose 3.

FIG. 8 (Example 14) shows the ratio of serotype specific IgG titers in mice following immunization of PCV26 with Compound B-1-SNE (10 μg dose of Compound B-1) compared to an immunization of PCV26 formulated with APA at post dose 2. Serotype 6C and 15B data are included to evaluate cross reactivity.

FIG. 9 (Example 14) shows the IgG2a/IgG1 titer ratio of serotype specific antibodies in mice following immunization of PCV26 with Compound B-1-SNE (10 μg dose of Compound B-1) or PCV26 formulated with APA at post dose 3. Responses from three representative serotypes 18C, 19F and 24F are shown (left to right).

FIG. 10A (Example 14) shows mice immunized with PCV26 with Compound B-1-SNE (10 μg dose of Compound B-1) or PCV26 formulated with APA are protected from S. pneumoniae 24F intra tracheal challenge. Bacteremia was quantified at 24 hours post-infection with serotype 24F bacteria and presented as log CFU/mL.

FIG. 10B (Example 14) shows mice immunized with PCV26 with Compound B-1-SNE (10 μg dose of Compound B-1) or PCV26 formulated with APA are protected from S. pneumoniae 24F intra tracheal challenge. Survival was evaluated for approximately 240 hours post-infection with serotype 24F bacteria.

### DETAILED DESCRIPTION OF THE INVENTION

The invention provides a composition comprising one or more S. pneumoniae antigens and a compound having the structure set forth in Formula I:

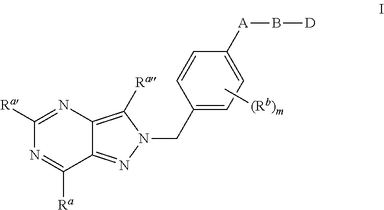

wherein:

R^{a} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and —NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

R^{a′} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and —NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

R^{a″} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and —NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

R′ and R″ are independently selected from H, (C₁-C₆)alkyl, (C₁-C₆)alkenyl, and (C₁-C₆)alkynyl, wherein said (C₁-C₆)alkyl, (C₁-C₆)alkenyl and (C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine, or R′ and R″, together with the nitrogen to which they are attached, join together to form a (C₃-C₆)heterocycloalkyl, wherein said (C₃-C₆)heterocycloalkyl is optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

each occurrence of R^{b} is independently selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, or NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

A is a carbon- or nitrogen-linked spacer selected from (C₁-C₆)alkyl, (C₃-C₆)heterocycloalkyl, heterocycloalkyl-C(O)—R^{z}—, (C₁-C₄)alkyl-N(R^{z})—R^{z}—, aryl, and heteroaryl, wherein said (C₁-C₆)alkyl, (C₃-C₆)heterocycloalkyl, aryl and heteroaryl are optionally substituted with one to six substituents, independently selected from the group consisting of —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine;

each occurrence of R^{z} is independently H or (C₁-C₆)alkyl;

B is a functional group selected from

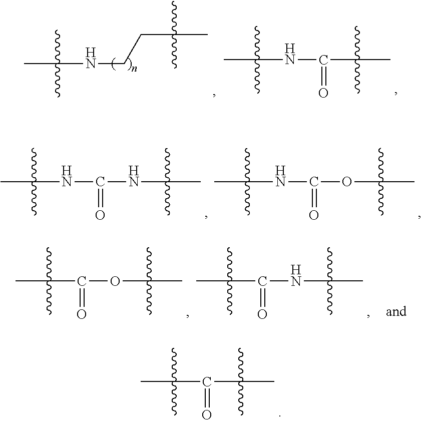

D is a lipid selected from (C₆-C₂₀)alkyl, (C₆-C₂₀)alkenyl and (C₆-C₂₀)alkynyl, wherein said (C₆-C₂₀)alkyl, (C₆-C₂₀)alkenyl, and (C₆-C₂₀)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine; or D is

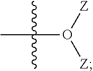

each occurrence of Z is independently selected from (C₆-C₂₀)alkyl, (C₆-C₂₀)alkenyl, and (C₆-C₂₀)alkynyl, wherein said (C₆-C₂₀)alkyl, (C₆-C₂₀)alkenyl, and (C₆-C₂₀)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine;

m is 0, 1, 2, 3, 4 or 5; and

n is 0, 1, 2, 3, 4 or 5;

or a pharmaceutically acceptable salt thereof.

The invention provides a composition comprising one or more S. pneumoniae antigens and a compound having the structure set forth in Formula Ia:

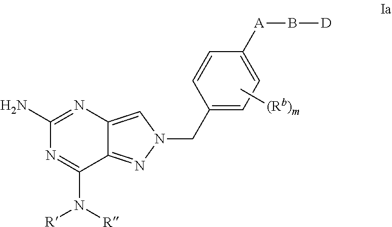

wherein:

R′ and R″ are independently selected from H, (C₁-C₆)alkyl, (C₁-C₆)alkenyl, and (C₁-C₆)alkynyl, wherein said (C₁-C₆)alkyl, (C₁-C₆)alkenyl and (C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine, or R′ and R″, together with the nitrogen to which they are attached, join together to form a (C₃-C₆)heterocycloalkyl, wherein said (C₃-C₆)heterocycloalkyl is optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

each occurrence of R^{b} is —O(C₁-C₄)alkyl, wherein said —O(C₁-C₄)alkyl is optionally substituted with one or two substituents, independently selected from the group consisting of —OH, —O(C₁-C₄)alkyl, —O(C₁-C₄)alkenyl, —O(C₁-C₄)alkynyl, chlorine and fluorine;

A is selected from

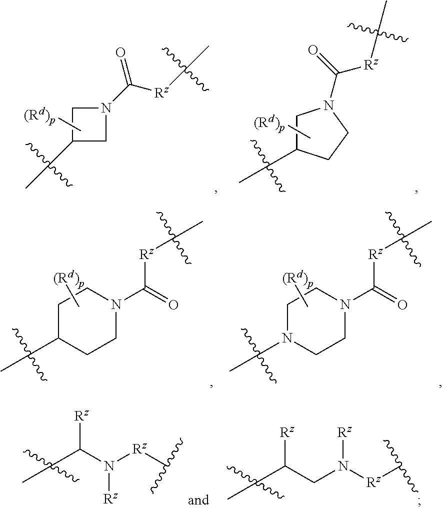

each occurrence of R^{z} is independently H or (C₁-C₆)alkyl;

each occurrence of R^{d} is independently selected from —OH, (C₁-C₄)alkyl, —O(C₁-C₄)alkyl, chlorine and fluorine;

B is

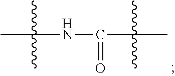 [SMILES: *NC(*)=O]

D is a lipid chain selected from:

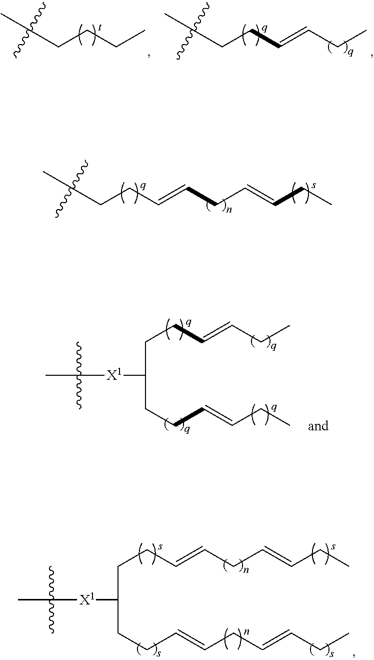

wherein any carbon on the lipid chain is optionally substituted with —OH, —O(C₁-C₄)alkyl, —O(C₁-C₄)alkenyl, —O(C₁-C₄)alkynyl, chlorine or fluorine,

wherein [symbol] is cis or trans stereochemistry,

X¹ is —O—, —C(R)²—, or —NR—, and

each occurrence of R is independently selected from H, (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine;

m is 0, 1 or 2;

n is 0, 1, 2 or 3;

p is 0, 1 or 2;

q is 0, 1, 2, 3, 4, 5, 6, 7, 8 or 9;

s is, 1, 2, 3, 4, 5, 6, 7 or 8; and

t is 6, 7, 8, 9, 10, 11, 12, 13, 14, 15, 16, 17 or 18;

or a pharmaceutically acceptable salt thereof.

The invention provides a composition comprising one or more S. pneumoniae antigens and a compound having the structure set forth in Formula II:

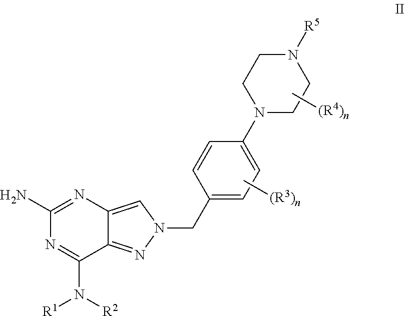

wherein:

R¹ is (C₁-C₆)alkyl, wherein said (C₁-C₆)alkyl is optionally substituted with one to four substituents independently selected from —OH and —O(CH₃);

R² is H, methyl or —O(CH₃);

each occurrence of R³ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);

each occurrence of R⁴ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);

R⁵ is

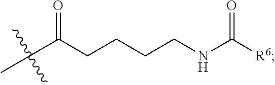

R⁶ is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl, and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine and fluorine; and

each occurrence of n is 4;

or a pharmaceutically acceptable salt thereof.

The invention provides a composition comprising one or more S. pneumoniae antigens and a compound having the structure set forth in Formula IIa:

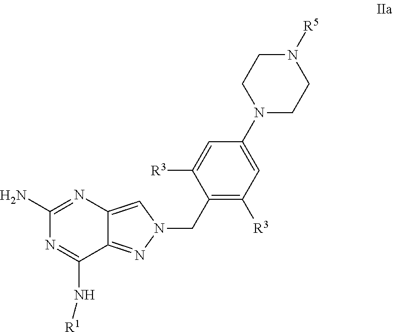

wherein:

R¹ is butyl, wherein said butyl is optionally substituted with one or two —OH;

each occurrence of R³ is independently H or —O(CH₃); and

R⁵ is

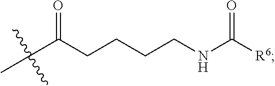

and

R⁶ is selected from (C₁₀-C₂₀)alkyl, (C₁₀-C₂₀)alkenyl and (C₁₀-C₂₀)alkynyl;

or a pharmaceutically acceptable salt thereof.

The invention provides a composition comprising one or more S. pneumoniae antigens and a compound having the structure set forth in Formula III:

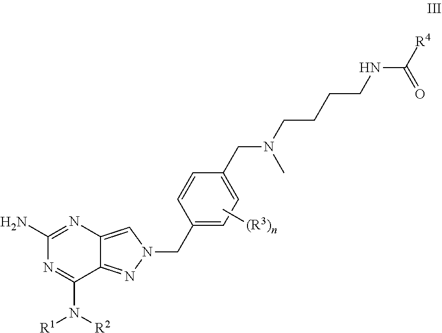

wherein:

R¹ is (C₁-C₆)alkyl, wherein said (C₁-C₆)alkyl is optionally substituted with one to four substituents independently selected from —OH and —O(CH₃);

R² is H, methyl or —O(CH₃);

each occurrence of R³ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);

R⁴ is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine and fluorine; and

n is 4;

or a pharmaceutically acceptable salt thereof.

The invention provides a composition comprising one or more S. pneumoniae antigens and a compound having the structure set forth in Formula IIIa:

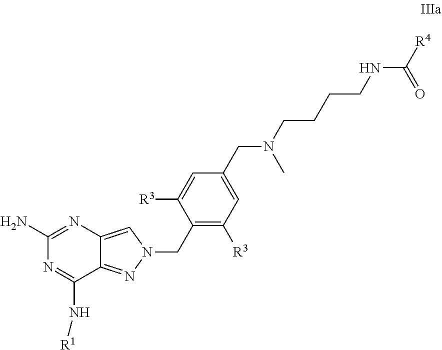

wherein:

R¹ is butyl, wherein said butyl is optionally substituted with one or two —OH;

each occurrence of R³ is independently H or —O(CH₃); and

R⁴ is selected from (C₁₀-C₂₀)alkyl, (C₁₀-C₂₀)alkenyl and (C₁₀-C₂₀)alkynyl;

or a pharmaceutically acceptable salt thereof.

The invention provides a composition comprising one or more S. pneumoniae antigens and a compound having the structure set forth in Formula IV:

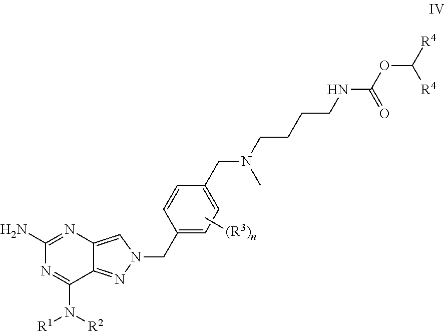

wherein:

R¹ is (C₁-C₆)alkyl, wherein said (C₁-C₆)alkyl is optionally substituted with one to four substituents independently selected from —OH and —O(CH₃);

R² is H, methyl or —O(CH₃);

each occurrence of R³ is independently H, (C₁-C₄)alkyl, (C₁-C₄)alkenyl, (C₁-C₄)alkynyl, or —O(C₁-C₄)alkyl wherein said (C₁-C₄)alkyl, (C₁-C₄)alkenyl, (C₁-C₄)alkynyl, and —O(C₁-C₄)alkyl are optionally substituted with one or two substituents independently selected from —OH and —O(CH₃);

each occurrence of R⁴ is independently selected from (C₆-C₂₀)alkyl, (C₆-C₂₀)alkenyl and (C₆-C₂₀)alkynyl, wherein said (C₆-C₂₀)alkyl, (C₆-C₂₀)alkenyl and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine or fluorine; and

n is 4;

or a pharmaceutically acceptable salt thereof.

The invention provides a composition comprising one or more S. pneumoniae antigens and a compound having the structure set forth in Formula IVa:

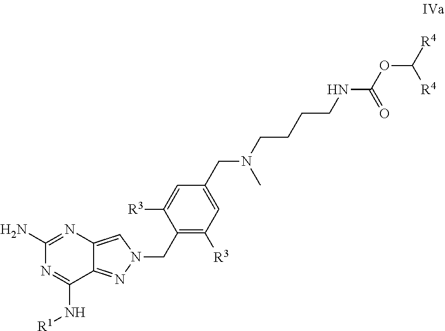

wherein:

R¹ is butyl, wherein said butyl is optionally substituted with one or two —OH;

each occurrence of R³ is independently H or —O(CH₃); and

each occurrence of R⁴ is independently selected from (C₁₀-C₂₀)alkyl, (C₁₀-C₂₀)alkenyl and (C₁₀-C₂₀)alkynyl;

or a pharmaceutically acceptable salt thereof.

The invention provides a composition comprising one or more S. pneumoniae antigens and one or more of the following compounds:
(N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide, also referred to as Compound A-1;
(S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide, also referred to as Compound A-2;
(S)-1-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)octadecan-1-one, also referred to as Compound A-3;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide, also referred to as Compound B-1;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)tetradecanamide, also referred to as Compound B-2;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)oleamide, also referred to as Compound B-3;
(9Z,12Z)—N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)octadeca-9,12-dienamide, also referred to as Compound B-4;
N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide, also referred to as Compound B-5;
N-(5-(4-(4-((5-amino-7-(butylamino)-2H-pyrazolo[4,3-d]pyrimidin-2-yl)methyl)-3-methoxyphenyl)piperidin-1-yl)-5-oxopentyl)stearamide, also referred to as Compound B-6;
N-(5-(3-(4-((5-amino-7-(butylamino)-2H-pyrazolo[4,3-d]pyrimidin-2-yl)methyl)-3-methoxyphenyl)azetidin-1-yl)-5-oxopentyl)stearamide, also referred to as Compound B-7;
1-(4-((5-amino-7-(butylamino)-2H-pyrazolo[4,3-d]pyrimidin-2-yl)methyl)-3-methoxyphenyl)-N-(3-stearamidopropyl)piperidine-4-carboxamide, also referred to as Compound B-8;
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide, also referred to as Compound B-9;
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide, also referred to as Compound B-10;
N-(3-(4-(4-((5-amino-7-(butylamino)-2H-pyrazolo[4,3-d]pyrimidin-2-yl)methyl)-3-methoxyphenyl)piperazin-1-yl)-3-oxopropyl)stearamide, also referred to as Compound B-11;
N-(7-(4-(4-((5-amino-7-(butylamino)-2H-pyrazolo[4,3-d]pyrimidin-2-yl)methyl)-3-methoxyphenyl)piperazin-1-yl)-7-oxoheptyl)stearamide, also referred to as Compound B-12;
N-(3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)cyclobutyl)stearamide, also referred to as Compound B-13;
N-(5-(4-(4-((5-amino-7-(butylamino)-2H-pyrazolo[4,3-d]pyrimidin-2-yl)methyl)-3-methoxyphenyl)piperazin-1-yl)-4,4-dimethyl-5-oxopentyl)stearamide, also referred to as Compound B-14;
N-(6-(4-(4-((5-amino-7-(butylamino)-2H-pyrazolo[4,3-d]pyrimidin-2-yl)methyl)-3-methoxyphenyl)piperazin-1-yl)-2-methyl-6-oxohexan-2-yl)stearamide, also referred to as Compound B-15;
1-(4-(4-((5-amino-7-(butylamino)-2H-pyrazolo[4,3-d]pyrimidin-2-yl)methyl)-3-methoxyphenyl)piperazin-1-yl)-5-(octadecyloxy)pentan-1-one, also referred to as Compound B-16;
1-(4-(4-((5-amino-7-(butylamino)-2H-pyrazolo[4,3-d]pyrimidin-2-yl)methyl)-3-methoxyphenyl)piperazin-1-yl)-5-(octadecylamino)pentan-1-one, also referred to as Compound B-17;
N-(5-(4-(4-((5-amino-7-(butylamino)-3-methyl-2H-pyrazolo[4,3-d]pyrimidin-2-yl)methyl)-3-methoxyphenyl)piperazin-1-yl)-5-oxopentyl)stearamide, also referred to as Compound B-18;
(9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide, also referred to as Compound C-1;
N-(4-((4-((5-amino-7-(butylamino)-2H-pyrazolo[4,3-d]pyrimidin-2-yl)methyl)-3,5-dimethoxybenzyl)(methyl)amino)butyl)tetradecanamide, also referred to as Compound C-2;
N-(4-((4-((5-amino-7-(butylamino)-2H-pyrazolo[4,3-d]pyrimidin-2-yl)methyl)-3,5-dimethoxybenzyl)(methyl)amino)butyl)oleamide, also referred to as Compound C-3;
N-(4-((4-((5-amino-7-(butylamino)-2H-pyrazolo[4,3-d]pyrimidin-2-yl)methyl)-3,5-dimethoxybenzyl)(methyl)amino)butyl)stearamide, also referred to as Compound C-4;
N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide, also referred to as Compound C-5;
(6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)amino)butyl)carbamate,
also referred to as Compound D-1;
4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide, also referred to as Compound D-2;
3-stearamidopropyl 4-(4-((5-amino-7-(butylamino)-2H-pyrazolo[4,3-d]pyrimidin-2-yl)methyl)-3-methoxyphenyl)piperazine-1-carboxylate, also referred to as Compound D-3;
N-(5-(4-(4-((5-amino-7-(butylamino)-2H-pyrazolo[4,3-d]pyrimidin-2-yl)methyl)-3-methoxyphenyl)piperazin-1-yl)-6,6,6-trifluorohexyl)stearamide, also referred to as Compound D-4;
N-(4-((4-((7-(butylamino)-5-hydroxy-2H-pyrazolo[4,3-d]pyrimidin-2-yl)methyl)-3,5-dimethoxybenzyl)(methyl)amino)butyl)stearamide, also referred to as Compound D-5; and
(6Z,9Z,28Z,31Z)-heptatriaconta-6,9,28,31-tetraen-19-yl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate, also referred to as Compound D-6;
   or a pharmaceutically acceptable salt thereof.

The invention also provides a composition comprising one or more S. pneumoniae antigens and a stable nanoemulsion, the stable nanoemulsion comprising: (i) one or more compounds having the structure set forth in Formula I, Ia, II, IIa, III, IIIa, IV or IVa or pharmaceutically acceptable salt(s) thereof; ii) one or more emulsifiers; and iii) one or more terpenes.

The invention also provides a composition comprising one or more S. pneumoniae antigens and a stable nanoemulsion, the stable nanoemulsion comprising: (i) one or more compounds having the structure set forth in Formula I, Ia, II, IIa, III, IIIa, IV or IVa or pharmaceutically acceptable salt(s) thereof; ii) one or more sorbitan-based surfactants; and iii) one or more terpenes.

The invention also provides a composition comprising one or more S. pneumoniae antigens and a stable nanoemulsion, the stable nanoemulsion comprising: (i) a compound having the structure set forth in Formula I, Ia, II, IIa, III, IIIa, IV or IVa or a pharmaceutically acceptable salt thereof; (ii) SPAN-85; (iii) PS-20 or PS-80; and (iv) squalene.

The invention also provides a composition comprising one or more S. pneumoniae antigens and a stable nanoemulsion, the stable nanoemulsion comprising: (i) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (ii) SPAN-85; (iii) PS-20 or PS-80; and (iv) squalene.

The invention also provides a pharmaceutical composition comprising: (i) at least one Streptococcus pneumoniae (S. pneumoniae) polysaccharide; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a pharmaceutical composition comprising: (i) at least one Streptococcus pneumoniae (S. pneumoniae) polysaccharide; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier.

The invention also provides an immunogenic composition comprising: (i) at least one S. pneumoniae polysaccharide; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides an immunogenic composition comprising: (i) at least one S. pneumoniae polysaccharide; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier.

The invention also provides a single-dose vaccine composition comprising: (i) at least one S. pneumoniae polysaccharide; (ii) a compound having the structure set forth in Formula I, Ia, II, IIa, III, IIIa, IV or IVa or a pharmaceutically acceptable salt thereof; (iii) SPAN-85; (iv) PS-20 or PS-80; wherein a single dose of the vaccine composition is sufficient to elicit a desired immune response against the at least one S. pneumoniae polysaccharide.

The invention also provides a single-dose vaccine composition comprising: (i) at least one S. pneumoniae polysaccharide; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier; wherein a single dose of the vaccine composition is sufficient to elicit a desired immune response against the at least one S. pneumoniae polysaccharide.

The invention also provides a method of treating or preventing pneumococcal diseases in a human patient comprising administrating to the patient a pharmaceutical composition comprising: (i) at least one S. pneumoniae polysaccharide; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a method of treating or preventing pneumococcal diseases in a human patient comprising administrating to the patient a pharmaceutical composition comprising: (i) at least one S. pneumoniae polysaccharide; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier.

The invention also provides a pharmaceutical composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides a pharmaceutical composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; and (v) squalene.

The invention also provides a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier.

The invention also provides an immunogenic composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides an immunogenic composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides an immunogenic composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides an immunogenic composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides an immunogenic composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, and e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; and (v) squalene.

The invention also provides an immunogenic composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier.

The invention also provides a single-dose vaccine composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene, wherein a single dose of the vaccine composition is sufficient to elicit a desired immune response against the at least one polysaccharide-carrier protein conjugate.

The invention also provides a single-dose vaccine composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier. The invention also provides a single-dose vaccine composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene; wherein a single dose of the vaccine composition is sufficient to elicit a desired immune response against the polysaccharide-carrier protein conjugates.

The invention also provides a single-dose vaccine composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier; wherein a single dose of the vaccine composition is sufficient to elicit a desired immune response against the polysaccharide-carrier protein conjugates.

The invention also provides a single-dose vaccine composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, and e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; and (v) squalene; wherein a single dose of the vaccine composition is sufficient to elicit a desired immune response against the polysaccharide-carrier protein conjugates.

The invention also provides a single-dose vaccine composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier; wherein a single dose of the vaccine composition is sufficient to elicit a desired immune response against the polysaccharide-carrier protein conjugates.

The invention also provides a method of treating or preventing pneumococcal diseases in a human patient comprising administrating to the patient a pharmaceutical composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides a method of treating or preventing pneumococcal diseases in a human patient comprising administrating to the patient a pharmaceutical composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a method of treating or preventing pneumococcal diseases in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides a method of treating or preventing pneumococcal diseases in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a method of treating or preventing pneumococcal diseases in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; and (v) squalene.

The invention also provides a method of treating or preventing pneumococcal diseases in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier.

The invention also provides a method of preventing or reducing the likelihood of infection with S. pneumoniae in a human patient comprising administrating to the patient a pharmaceutical composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides a method of preventing or reducing the likelihood of infection with S. pneumoniae in a human patient comprising administrating to the patient a pharmaceutical composition comprising: (i) at least one S. pneumoniae polysaccharide-carrier protein conjugate; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a method of preventing or reducing the likelihood of infection with S. pneumoniae in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; and (v) squalene.

The invention also provides a method of preventing or reducing the likelihood of infection with S. pneumoniae in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) a compound having the structure set forth in Formula I, Ia, II, IIa, III, IIIa, IV or IVa or a pharmaceutically acceptable salt thereof; (iii) SPAN-85; (iv) PS-20 or PS-80; (v) squalene; and (vi) a pharmaceutically acceptable carrier.

The invention also provides a method of preventing or reducing the likelihood of infection with S. pneumoniae in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; and (v) squalene.

The invention also provides a method of preventing or reducing the likelihood of infection with S. pneumoniae in a human patient comprising administrating to the patient a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharide consists of a group of serotypes selected from a) 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, b) 1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B, c) 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, d) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, e) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, and f) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B; wherein the carrier protein is CRM197; (ii) N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide; or a pharmaceutically acceptable salt thereof; (iii) SPAN-85; (iv) PS-20 or PS-80; (v) squalene; and (vi) a pharmaceutically acceptable carrier.

In specific embodiments of the i) pharmaceutical compositions, ii) immunogenic compositions, and iii) single-dose vaccine compositions above, the compositions comprise more than twenty (20) different S. pneumoniae polysaccharide-carrier protein conjugates. For example, the invention provides a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharides comprise of a group of serotypes selected from more than twenty (20) different S. pneumoniae polysaccharides.

In the specific embodiment above the group of serotypes is a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

In the specific embodiment above the group of serotypes is a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B, and the carrier protein is CRM197.

In specific embodiments of the i) pharmaceutical compositions, ii) immunogenic compositions, and iii) single-dose vaccine compositions above, the compositions comprise more than twenty-five (25) different S. pneumoniae polysaccharide-carrier protein conjugates. For example, the invention provides a pharmaceutical composition comprising: (i) S. pneumoniae polysaccharide-carrier protein conjugates, wherein each of the conjugates comprises a polysaccharide of a particular S. pneumoniae serotype conjugated to a carrier protein, wherein the S. pneumoniae polysaccharides comprise of a group of serotypes selected from more than twenty-five (25) different S. pneumoniae polysaccharides.

In the specific embodiment above the group of serotypes is a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

In the specific embodiment above the group of serotypes is a) 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B, or b) 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B, or c) 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B, and the carrier protein is CRM197.

In some embodiments of the i) pharmaceutical compositions, ii) immunogenic compositions, and iii) single-dose vaccine compositions above, the concentration of compound is 0.01 μg/mL to 1000 μg/mL or 0.1 μg/mL to 100 μg/mL or 80 μg/mL or 16 μg/mL or 4 μg/mL.

In some embodiments of the i) pharmaceutical compositions, ii) immunogenic compositions, and iii) single-dose vaccine compositions above, the concentration of SPAN-85 is 0.001 mg/mL to 100 mg/mL or 0.01 mg/mL to 50 mg/mL or 0.1 mg/mL to 10 mg/mL.

In some embodiments of the i) pharmaceutical compositions, ii) immunogenic compositions, and iii) single-dose vaccine compositions above, the concentration of PS-20 or PS-80 is 0.001 mg/mL to 100 mg/mL or 0.01 mg/mL to 50 mg/mL or 0.1 mg/mL to 10 mg/mL.

In some embodiments of the i) pharmaceutical compositions, ii) immunogenic compositions, and iii) single-dose vaccine compositions above, the concentration of squalene is 0.01 mg/mL to 100 mg/mL or 0.02 mg/mL to 20 mg/mL or 1 mg/mL to 20 mg/mL.

In some embodiments of the i) pharmaceutical compositions, ii) immunogenic compositions, and ii) single-dose vaccine compositions above, the concentration of compound is 80 μg/mL, the concentration of SPAN-85 is 4.8 mg/mL, the concentration of PS-20 or PS-80 is 4.8 mg/mL, and the concentration of squalene is 16 mg/mL.

In some embodiments of the i) pharmaceutical compositions, ii) immunogenic compositions, and ii) single-dose vaccine compositions above, and iv) single-dose vaccine compositions above, the concentration of compound is 16 μg/mL, the concentration of SPAN-85 is 4.8 mg/mL, the concentration of PS-20 or PS-80 is 4.8 mg/mL, and the concentration of squalene is 16 mg/mL.

In some embodiments of the i) pharmaceutical compositions, ii) immunogenic compositions, and ii) single-dose vaccine compositions above, and iv) single-dose vaccine compositions above, the concentration of compound is 4 μg/mL, the concentration of SPAN-85 is 4.8 mg/mL, the concentration of PS-20 or PS-80 is 4.8 mg/mL, and the concentration of squalene is 16 mg/mL.

In some embodiments of the i) pharmaceutical compositions, ii) immunogenic compositions, and iii) single-dose vaccine compositions above, the compositions further comprise L-met and EDTA.

### Abbreviations and Definitions

The following abbreviations are used herein:
[Ni(dtbbpy)(H₂O)₄]Cl₂ tetraaquo[4,4′-bis(1,1-dimethylethyl)-2,2′-bipyridine]nickel(II) dichloride
AcOH acetic acid
Anh Anhydrous
APA aluminum phosphate adjuvant
Aq Aqueous
Bn Benzyl
Boc or BOC tert-butoxycarbonyl
BOP (benzotriazol-1-yloxytris(dimethylamino)phosphonium hexafluorophosphate)
Bs broad singlet
BSI bloodstream infection
BTMG 2-tert-butyl-1,1,3,3-tetramethylguanidine
Bz Benzoyl
calc'd Calculated
Cbz Benzyloxycarbonyl
CD₃OD deuterated methanol
CDCl₃ deuterated chloroform
CDI carbonyl diimidazole
Celite diatomaceous earth
CH₃CN Acetonitrile
CMBP (cyanomethylene)tributylphosphorane
CRAB carbapenem-resistant Acinetobacter baumannii
Cs₂CO₃ cesium carbonate
D Doublet
Dba Dibenzylidineacetone
DBAB di-tert-butyl azodicarboxylate
DBU 1,8-diazabicyclo(5.4.0)undec-7-ene
DCE Dichloroethane
DCM Dichloromethane
DIEA or DIPEA N,N-diisopropylethylamine
DMAP 4-dimethylaminopyridine
DMF N,N-dimethylformamide
DMP Dess-Martin periodinane
DMSO dimethyl sulfoxide
DNA deoxyribonucleic acid
Dppf 1,1′-bis(diphenylphosphino)ferrocene
EDC 1-ethyl-3-(3-dimethylaminopropyl)carbodiimide
ESI electrospray ionization
Et Ethyl
Et₂O diethyl ether
Et₃N Triethylamine
EtOAc ethyl acetate
EtOH Ethanol
FL Compound
G Gram
H Hour
HAP hospital-acquired pneumonia
HATU 1-[bis(dimethylamino)methylene]-1H-1,2,3-triazolo[4,5-b]pyridinium 3-oxide hexafluorophosphate
HCl hydrochloric acid
Hex Hexanes
HMDS hexamethyldisilazane
HPLC high-performance liquid chromatography
HPSEC high performance size exclusion chromatography
IBX 2-iodoxybenzoic acid
IM inner membrane
Int Intermediate
IPA Isopropanol
iPr Isopropyl
Ir[df(CF₃)ppy]₂(dtbbpy)PF₆ [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate
K₂CO₃ potassium carbonate
LC liquid chromatography
LC/MS liquid chromatography mass spectrometry
LED light emitting diode
LOS Lipooligosaccharide
LPS Lipopolysaccharide
M Multiplet
MDR multi-drug resistant
Me Methyl
Me₂S dimethyl sulfide
MeCN Acetonitrile
MeOH Methanol
Mg Milligrams
MgSO₄ magnesium sulfate
Min Minutes
mL Milliliters
Mmol Millimoles
MP-(OAc)₃BH MP-triacetoxyborohydride
MS mass spectrometry
Mw molecular weight
Na₂SO₄ sodium sulfate
NaBH(OAc)₃ sodium triacetoxyborohydride
NaBH₄ sodium borohydride
NaN₃ sodium azide
NaOH sodium hydroxide
NaOMe sodium methoxide
NaOtBu sodium tert-butoxide
n-Bu n-butyl
NCS N-chlorosuccinimide
NH₄OH ammonium hydroxide
NMI N-methylimidazole
NMR nuclear magnetic resonance spectroscopy
NMWCO nominal molecular weight cut off
OAc Acetate
OM outer membrane
PCV pneumococcal conjugate vaccine
Pd Palladium
PD1 post dose 1
PD2 post dose 2
PD3 post dose 3
Pet. Petroleum
Ph Phenyl
PMe₃ Trimethylphosphine
PnPs pneumococcal polysaccharide
PPh₃ Triphenylphosphine
Ppm parts per million
prep-TLC preparative thin layer chromatography
Ps Polysaccharide
PS-20 polysorbate-20
PS-80 polysorbate-80
Psi pounds per square inch
Py Pyridine
Q Quartet
RT or rt room temperature
RuPhos 2-dicyclohexylphosphino-2′,6′-diisopropoxybiphenyl
RuPhos Pd G2 chloro(2-dicyclohexylphosphino-2′,6′-diisopropoxy-1,1′-biphenyl) [2-(2′-amino-1,1′-biphenyl)]palladium(II)
S Singlet
Sat Saturated
SFC supercritical fluid chromatography
SNE stable nanoemulsion
SPAN-85 sorbitan-trioleate
ST Serotype
T Triplet
TBAF tert-butyl ammonium fluoride
TBDPS tert-butyldiphenylsilyl
TBDPSCl tert-butyldiphenylsilyl chloride
TBS or TBDMS tert-butyldimethylsilyl
TBSCl tert-butyldimethylsilyl chloride
t-Bu tert-butyl
TCFH chloro-N,N,N′,N′-tetramethylformamidinium hexafluorophosphate
TEA Triethylamine
TFA trifluoroacetic acid
THF Tetrahydrofuran
TLC thin layer chromatography
TMS Trimethylsilyl
TMSBr trimethylsilyl bromide
TMSCl Chlorotrimethylsilane
TMSN₃ trimethylsilyl azide
Tris tris(hydroxymethyl)aminomethane
UPLC ultra-performance liquid chromatography
w/v weight per volume
μL Microliters

As used throughout the specification and in the appended claims, the singular forms “a,” “an,” and “the” include the plural reference unless the context clearly dictates otherwise.

As used herein, the term “about,” when used herein in reference to a value, refers to a value that is the same as or, in context, is similar to the referenced value. In general, those skilled in the art, familiar with the context, will appreciate the absolute amount and/or relative degree of difference encompassed by “about” in that context. For example, in some embodiments, the term “about” can encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

An “adjuvant,” as defined herein, refers to a compound or compound formulation or composition that serves to enhance the immunogenicity of a composition of the invention. An adjuvant may i) enhance an immune response to an antigen (for example, a pneumococcal polysaccharide) that is weakly immunogenic when administered alone, e.g., inducing no or weak antibody titers or cell-mediated immune response, ii) increase antibody titers to the antigen, and/or iii) lowers the dose of the antigen effective to achieve an immune response in the individual.

As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscular (IM), intradermal (ID) etc.) and the like.

As used herein, an “agent” refers to a particle, compound, molecule, or entity of any chemical class.

As used herein, the term “alkyl” refers to a straight chain, cyclic or branched saturated aliphatic hydrocarbon having the specified number of carbon atoms. A numerical range, which refers to the chain length in total, may be given. For example, C₁-C₆ alkyl has a chain length of 1 to 6 atoms. An alkyl group can be optionally substituted with one or more “substituents” which may be the same or different and are as defined herein below. Unless otherwise indicated, an alkyl group is unsubstituted.

As used herein, the term “alkenyl” means a straight chain, cyclic or branched unsaturated aliphatic hydrocarbon having the specified number of carbon atoms including but not limited to diene, triene and tetraene unsaturated aliphatic hydrocarbons. An alkenyl group can be optionally substituted with one or more “substituents” which may be the same or different and are as defined herein below. Unless otherwise indicated, an alkenyl group is unsubstituted.

As used herein, the term “alkynyl” means a straight chain, cyclic or branched unsaturated aliphatic hydrocarbon having the specified number of carbon atoms including but not limited to diene, triene and tetraene unsaturated aliphatic hydrocarbons. An alkynyl group can be optionally substituted with one or more “substituents” which may be the same or different and are as defined herein below. Unless otherwise indicated, an alkynyl group is unsubstituted.

As used herein, the term “antigen” refers to any antigen that can generate one or more immune responses. The antigen may be a protein, peptide or polypeptide. In certain embodiments, the antigen is a lipid or a carbohydrate. In certain embodiments, the antigen is a polysaccharide. In certain embodiments the antigen is a pneumococcal polysaccharide. In certain embodiments the polysaccharide is an S. pneumoniae polysaccharide. The antigen may be one that generates a humoral and/or CTL immune response.

As used herein, the term “aryl” refers to a carbocycle aromatic monocyclic or bicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different and are as defined herein below. Non-limiting examples of aryl groups include phenyl and naphthyl. In one embodiment, an aryl group is phenyl. Unless otherwise indicated, an aryl group is unsubstituted.

As used herein, the term “composition” refers to a formulation containing an active pharmaceutical or biological ingredient (for example, a pneumococcal polysaccharide-carrier protein conjugate and a compound), along with one or more additional components. The term “composition” is used interchangeably with “pharmaceutical composition” and “formulation”. The compositions can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. Compositions are nontoxic to recipients at the dosages and concentrations employed.

As used herein the term “comprises” when used with the composition of the invention refers to the inclusion of any other components, such as adjuvants and excipients, or the addition of one or more polysaccharide-carrier protein conjugates that are not specifically enumerated.

As used herein, the term “consisting of” and variations such as “consist of” when used with a multivalent polysaccharide mixture or a multivalent polysaccharide-carrier protein conjugate mixture refers to a mixture having those particular S. pneumoniae polysaccharides or S. pneumoniae polysaccharide-carrier protein conjugates and no other S. pneumoniae polysaccharides or S. pneumoniae polysaccharide-carrier protein conjugates from a different serotype.

As used herein, the term “consists essentially of” and variations such as “consist essentially of” or “consisting essentially of,” indicate the inclusion of any recited elements or group of elements, and the optional inclusion of other elements, of similar or different nature than the recited elements, which do not materially change the basic or novel properties of the specified dosage regimen, method, or composition.

As used herein, the term “de-O-acetylated-15B” or “de-O-acetyl-15B” or “de-O—Ac-15B” refers to a de-O-acetylated serotype 15B wherein the O-acetyl content is less than 10% per repeating unit. In another embodiment the O-acetyl content is less than 5% per repeating unit. In another embodiment the O-acetyl content is less than about 1% per repeating unit. In another embodiment the O-acetyl content is less than 1% per repeating unit. In another embodiment the O-acetyl content is less than 0.5% per repeating unit. In another embodiment the O-acetyl content is less than 0.1% per repeating unit. In another embodiment the O-acetyl content is 0% per repeating unit. Processes for de-O-acetylation are known in the art, for example as described in Rajam et al., Clinical and Vaccine Immunology, 2007, 14(9):1223-1227.

As used herein, the term “dose” means a quantity of an agent, API (active pharmaceutical ingredient), formulation, composition, pharmaceutical composition or immunogenic composition taken or recommended to be taken at a particular time.

As used herein, the term “heteroaryl” refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different and are as defined herein below. A heteroaryl group is joined via a ring carbon atom, and any nitrogen atom of a heteroaryl can be optionally oxidized to the corresponding N-oxide. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

As used herein, the term “heterocycloalkyl” means a saturated or partly unsaturated non-aromatic monocyclic, bicyclic (including spirocyclic) or bridged carbocyclic ring or ring system comprising 3 to about 11 ring atoms, containing at least one ring heteroatom selected from N, S and O and the remainder of the ring atoms are carbon atoms. A heterocycloalkyl group can be joined via a ring carbon, or ring nitrogen atom, unless specified otherwise. The heterocycloalkyl ring may be substituted on the ring carbons and/or the ring nitrogen(s). In one embodiment, a heterocycloalkyl group is monocyclic and has from about 3 to about 7 ring atoms. In another embodiment, a heterocycloalkyl group is monocyclic has from about 4 to about 7 ring atoms. In other embodiments, the heterocycloalkyl group is bicyclic and has 7-10 ring atoms, 8-10 ring atoms, or 9 or 10 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. The heterocycloalkyl groups may be substituted. In some embodiments, the heterocycloalkyl groups have 1-2 heteroatoms selected from nitrogen, sulfur and oxygen atoms in the ring. In some embodiments, the heterocycloalkyl groups have 1 heteroatom selected from nitrogen, sulfur and oxygen atoms in the ring. In some embodiments, the heteroatoms are selected from O, S, S(O), S(O)₂, and NH—, N(alkyl)-. Non-limiting examples include ethers, thioethers, amines, hydroxymethyl, 3-hydroxypropyl, 1,2-dihydroxyethyl, 2-methoxyethyl, 2-aminoethyl, 2-dimethylaminoethyl, and the like an aliphatic group containing a heteroatom.

As used herein, the term “immunogenic” or “immunogenicity” refers to the ability of an antigen (for example, an S. pneumoniae polysaccharide) to provoke an immune response in a subject. The term “immunogenic composition” refers to the ability of an agent, API, formulation, composition or pharmaceutical composition to provoke an immune response in a subject.

As used herein, the phrase patients or subjects “in need of treatment” include those previously exposed to or infected with S. pneumoniae, those who were previously vaccinated against S. pneumoniae, as well as those prone to have an infection or any person in which a reduction in the likelihood of infection is desired, e.g., the immunocompromised, the elderly, children, adults, or healthy individuals.

As used herein, the phrase “indicated for the prevention of pneumococcal disease” means that a vaccine or composition is approved by one or more regulatory authorities, such as the US Food and Drug Administration, for the prophylaxis of one or more diseases caused by any serotype of S. pneumoniae, including, but not limited to: pneumococcal disease generally, invasive pneumococcal disease (IPD), pneumococcal pneumonia (PP), pneumococcal meningitis, pneumococcal bacteremia, invasive disease caused by S. pneumoniae, and otitis media caused by S. pneumoniae.

As used herein, the term “multiple-dose” refers to a vaccine composition, or pharmaceutical composition, or an immunogenic composition that requires more than one dose or administration or injection of the components therein in a clinical regimen to induce a durable immune response and provide protection from a disease or to decrease the likelihood of infection with an infectious agent. One of skill in the art would understand how to determine a durable immune response, e.g., by measuring antibody titers over a specified period of time.

As used herein, a “patient” (alternatively referred to herein as a “subject”) refers to a mammal capable of being infected with S. pneumoniae. In preferred embodiments, the patient is a human. A patient can be treated prophylactically or therapeutically. Prophylactic treatment provides sufficient protective immunity to reduce the likelihood or severity of a pneumococcal infection or the effects thereof, e.g., pneumococcal pneumonia. Therapeutic treatment can be performed to reduce the severity or prevent recurrence of a S. pneumoniae infection or the clinical effects thereof. Prophylactic treatment can be performed using a pneumococcal conjugate composition or vaccine or immunogenic composition of the invention, as described herein. The pneumococcal conjugate compositions or vaccines or immunogenic compositions of the invention can be administered to the general population or to those persons at an increased risk of pneumococcal infection, e.g., the elderly, or those who live with or care for the elderly.

As used herein, the term “PCV1” refers to a 1-valent pneumococcal conjugate vaccine or composition comprising one S. pneumoniae polysaccharide-carrier protein conjugate, comprising capsular polysaccharide from a S. pneumoniae serotype conjugated to a carrier protein. In specific embodiments, the carrier protein is CRM197.

As used herein, the term “PCV24” refers to a 24-valent pneumococcal conjugate vaccine or composition comprising twenty-three S. pneumoniae polysaccharide-carrier protein conjugates, each comprising capsular polysaccharide from a S. pneumoniae serotype conjugated to a carrier protein, wherein the serotypes of S. pneumoniae are: 1, 3, 4, 5, 6A, 6B, 7F, 8, 9V, 10A, 11A, 12F, 14, 15A, 18C, 19A, 19F, 22F, 23B, 24F, 31, 33F and 35B, and at least one of the following serogroup 15 serotypes: 15B, 15C or de-O-acetylated-15B. In a particular embodiment, the serogroup 15 serotype is serotype 15C or de-O-acetylated 15B. In another embodiment, the serogroup 15 serotype is serotype de-O-acetylated 15B. In specific embodiments, the carrier protein of one or more of the S. pneumoniae polysaccharide-carrier protein conjugates is CRM197. In further embodiments, the carrier protein of each of the S. pneumoniae polysaccharide-carrier protein conjugates is CRM197.

As used herein, the term “PCV26” refers to a 26-valent pneumococcal conjugate vaccine or composition comprising twenty-five S. pneumoniae polysaccharide-carrier protein conjugates, each comprising capsular polysaccharide from a S. pneumoniae serotype conjugated to a carrier protein, wherein the serotypes of S. pneumoniae are: 1, 3, 4, 5, 6A, 6B, 7F, 8, 9V, 10A, 11A, 12F, 14, 15A, 16F, 18C, 19A, 19F, 22F, 23A, 23B, 24F, 31, 33F and 35B, and at least one of the following serogroup 15 serotypes: 15B, 15C or de-O-acetylated-15B. In a particular embodiment, the serogroup 15 serotype is serotype 15C or de-O-acetylated 15B. In another embodiment, the serogroup 15 serotype is serotype de-O-acetylated 15B. In specific embodiments, the carrier protein of one or more of the S. pneumoniae polysaccharide-carrier protein conjugates is CRM197. In further embodiments, the carrier protein of each of the S. pneumoniae polysaccharide-carrier protein conjugates is CRM197.

As used herein, the term “pharmaceutically acceptable”, with respect to a carrier, diluent or excipient of a pharmaceutical composition, indicates that a carrier, diluent, or excipient must be compatible with the other ingredients of the composition and not deleterious to the recipient thereof.

As used herein, the term “pharmaceutical composition” refers to a composition containing an active pharmaceutical or biological ingredient, along with one or more additional components, e.g., a composition in which an active agent is formulated together with one or more pharmaceutically acceptable carriers. As used herein, the terms “pharmaceutical formulation” and “formulation” are used interchangeably with “pharmaceutical composition.” In some embodiments, the active agent is present in a unit dose amount appropriate for administration in a therapeutic regimen that shows a statistically significant probability of achieving a predetermined therapeutic effect when administered to a relevant population. The pharmaceutical compositions or formulations can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. The pharmaceutical compositions or formulations are nontoxic to recipients at the dosages and concentrations employed. In some embodiments, a pharmaceutical composition can be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, the term formulation refers to a single-dose of vaccine, which can be included in any volume suitable for injection.

As used herein, the terms “pneumococcal conjugate” or “pneumococcal polysaccharide-carrier protein conjugate” refer to an S. pneumoniae polysaccharide-carrier protein conjugate.

As used herein, the term “pneumococcal conjugate vaccine” (or “PCV”) is a pharmaceutical preparation or composition comprising pneumococcal polysaccharide-carrier protein conjugate(s) that provide active immunity to disease or pathological conditions caused by serotype(s) of S. pneumoniae.

As used herein, the term, “ring system substituent” refers to a substituent group attached to an aromatic or non-aromatic ring system which, for example, replaces an available hydrogen on the ring system. Ring system substituents may be the same, or different, and are each independently selected. Examples of ring system substituents include alkyl, alkenyl, alkynyl, aryl, heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)— heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, and —O—C(O)-cycloalkyl. A further example of ring system substituents include (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine.

As used herein, the term “single-dose” refers to a vaccine composition that only requires one administration or injection in a clinical regimen to induce a durable immune response and provide protection from a disease (for example, a pneumococcal disease). One of skill in the art would understand how to determine a durable immune response, e.g., by measuring antibody titers over a specified period of time.

As used herein, the term, “substituent” or “optional substituent” or “optionally substituted” refers to a substituent group attached to an alkyl group, or an alkenyl group, or an alkynyl group which, for example, replaces an available hydrogen on the group. Substituents may be the same, or different, and are each independently selected. Examples of substituents include alkyl, alkenyl, alkynyl, aryl, heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, and —O—C(O)-cycloalkyl. A further example of substituents include (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine.

An “SNE” or “stable nanoemulsion”, as used herein, refers to a composition comprising a compound of Formula I, Ia, II, IIa, III, IIIa, IV or IVa or a pharmaceutically acceptable salt thereof. In some embodiments, a SNE comprise a compound of Formula I, Ia, II, IIa, III, IIIa, IV or IVa or a pharmaceutically acceptable salt thereof, s trioleate (SPAN-85), polysorbate-20 (PS-20) or polysorbate-80 (PS-80) and squalene, wherein the composition is in the form of a nanoemulsion. In some embodiments, an SNE comprises a compound of Formula I, Ia, II, IIa, III, IIIa, IV or IVa or a pharmaceutically acceptable salt thereof, SPAN-85, PS-20 and squalene, In some embodiments, an SNE comprises 0.001 mg/mL to 10 mg/mL SPAN-85, 0.001 mg/mL to 10 mg/mL PS-20 and 0.01 mg/mL to 100 mg/mL squalene. In further embodiments, an SNE comprises 0.05 mg/mL to 5 mg/mL SPAN-85, 0.05 mg/mL to 5 mg/mL PS-20 and 0.05 mg/mL to 50 mg/mL squalene. In still further embodiments, an SNE comprises 0.1 mg/mL to 1 mg/mL SPAN-85, 0.1 mg/mL to 1 mg/mL PS-20 and 1 mg/mL to 10 mg/mL squalene. As used herein, SNE is used interchangeably with “squalene emulsion”.

A “nanoemulsion”, also known as a nanometric-sized emulsion, is a fine oil-in-water (o/w) dispersion of two immiscible fluids. Nanoemulsions are a colloidal particulate system in the submicron size range acting as carriers of drug molecules. Their size varies from 10 to 1,000 nm. These carriers are solid spheres and their surface is amorphous and lipophilic.

As used herein, the term “therapeutically effective amount” refers to an amount of an active ingredient (antigen) sufficient to produce the desired therapeutic effect in a human or animal, e.g., the amount necessary to elicit an immune response, treat, cure, prevent, or inhibit development and progression of a disease or the symptoms thereof and/or the amount necessary to ameliorate symptoms or cause regression of a disease. Therapeutically effective amount may vary depending on the structure and potency of the active ingredient and the contemplated mode of administration. One of skill in the art can readily determine a therapeutically effective amount of a given active ingredient in a vaccine.

As used herein, the term “valent” refers to the presence of a specified number of polysaccharides or polysaccharide-carrier protein conjugates in a composition.

As used herein, the term “vaccine” or “vaccine composition” refers to a biological preparation used to stimulate the production of antibodies and provide immunity against an infectious disease.

As used herein, the term “carbon- or nitrogen-linked spacer” refers to any chemistry that links a carbon (—C) or a nitrogen (—N) to the benzyl group of the Formulas of the invention (see attachment of variable “A” in Formulas I and Ia). Examples of carbon or nitrogen linked spacers are C₁-C₆ alkyl, heterocycloalkyl, aryl, and heteroaryl, wherein said alkyl, heterocycloalkyl, aryl and heteroaryl are optionally substituted.

Unless otherwise noted, when describing a linker throughout this disclosure, the first available bond on a linker group connects to the portion of the compound flanking the linker group to the left and the terminal available bond of the linker group connects to the terminal portion of the compound flanking the linker group to the right. For example, if the linker (L) is —CH₂—CF₂—, the definition of L includes only A-CH₂—CF₂— B and does not include A-CF₂—CH₂—B.

As used herein, the term “functional group” refers to any chemistry that links the carbon or nitrogen linked spacer to the lipid group. An example of a functional group is:

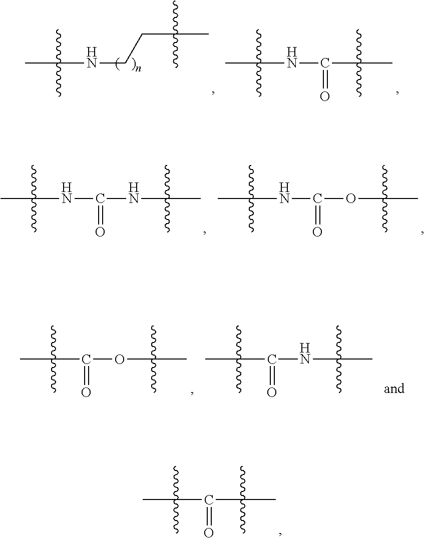

wherein n is 0, 1, 2, 3, 4, or 5.

A further example of a functional group is

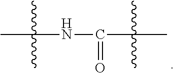 [SMILES: *NC(*)=O]

As used herein, the term “lipid” refers to any chemistry that is insoluble in water but soluble in organic solvents. Examples of lipids are represented by variable D (see Formulas I and Ia) and are selected from (C₆-C₂₀)alkyl, (C₆-C₂₀)alkenyl and (C₆-C₂₀)alkynyl, wherein said alkyl, alkenyl, and alkynyl are optionally substituted.

A further example of a lipid is

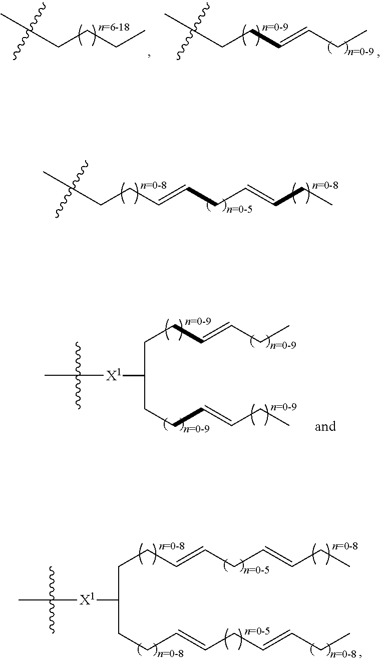

wherein any carbon on the lipid chain is optionally substituted, [symbol]
is cis or trans stereochemistry,

### X is O, C(R)², or NR, and

R is independently selected from H, (C₁-C₄)alkyl, (C₁-C₄)alkenyl, (C₁-C₄)alkynyl, OH, O(C₁-C₄)alkyl, O(C₁-C₄)alkenyl, O(C₁-C₄)alkynyl, chlorine and fluorine.

A further example of a lipid is represented by variable D (see Formulas I and Ia) wherein D is

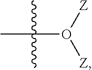

wherein Z is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl and (C₆-C₂₀)alkynyl are optionally substituted.

A compound of the invention that has one or more asymmetric centers can occur as a mixture of the stereoisomers or a substantially pure individual diastereomer or enantiomer unless expressly depicted otherwise. The invention encompasses all stereoisomeric forms of the compounds of Formula I, Ia, II, IIa, III, IIIa, IV and IVa. Unless a specific stereochemistry is indicated, the invention is meant to include all such isomeric forms of these compounds. Centers of asymmetry that are present in the compounds of Formula I, Ia, II, IIa, III, IIIa, IV and IVa can all independently of one another have (R) configuration or (S) configuration. When bonds to the chiral carbon are depicted as straight lines in the structural Formulas of the invention, it is understood that both the (R) and (S) configurations of the chiral carbon, and hence both enantiomers and mixtures thereof, are embraced within the Formula. Similarly, when a compound name is recited without a chiral designation for a chiral carbon, it is understood that both the (R) and (S) configurations of the chiral carbon, and hence individual enantiomers, diastereomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

When any variable (e.g., n, R^{b}, etc.) occurs more than one time in any constituent or in Formula I, Ia, II, IIa, III, IIIa, IV, and IVa, its definition on each occurrence is independent of its definition at every other occurrence. Also, combinations of substituents and/or variables are permissible only if such combinations result in stable compounds.

A “stable” compound is a compound which can be prepared and isolated and whose structure and properties remain or can be caused to remain essentially unchanged for a period of time sufficient to allow use of the compound for the purposes described herein (e.g., use as an adjuvant in an immunogenic composition to be administered to a subject). The compounds of the present invention are limited to stable compounds embraced by Formulas I, Ia II, IIa, III, IIIa, IV, and IVa.

A wavy line [symbol] , as used herein, indicates a point of attachment to the rest of the compound. Lines drawn into a ring system, for example:

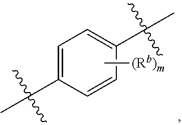

indicate that the bond may be attached to any of the substitutable ring atoms.

### Compounds

The compounds described herein are useful in the adjuvant formulations of the invention, which are useful to boost the immunological response of a pneumococcal composition or a pneumococcal conjugate composition or pneumococcal vaccine (PV) or pneumococcal conjugate vaccine (PCV) described herein.

In some embodiments, a composition of the invention comprises one or more S. pneumoniae antigens and a compound represented by the structure set forth in Formula I:

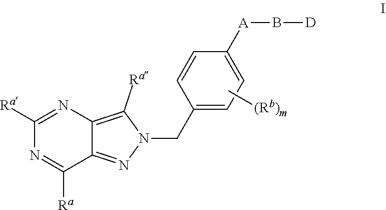

wherein:

R^{a} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

R^{a′} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and —NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

R^{a″} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and —NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

R′ and R″ are independently selected from H, (C₁-C₆)alkyl, (C₁-C₆)alkenyl, and (C₁-C₆)alkynyl, wherein said (C₁-C₆)alkyl, (C₁-C₆)alkenyl and (C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine, or R′ and R″, together with the nitrogen to which they are attached, join together to form a (C₃-C₆)heterocycloalkyl, wherein said (C₃-C₆)heterocycloalkyl is optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

each occurrence of R^{b} is independently selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, or NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

A is a carbon- or nitrogen-linked spacer selected from (C₁-C₆)alkyl, heterocycloalkyl, heterocycloalkyl-C(O)—R^{z}—, (C₁-C₄)alkyl-N(R^{z})—R^{z}—, aryl, and heteroaryl, wherein said (C₁-C₆)alkyl, heterocycloalkyl, aryl and heteroaryl are optionally substituted with one to six substituents, independently selected from the group consisting of —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine;

each occurrence of R^{z} is independently H or (C₁-C₆)alkyl;

B is a functional group selected from

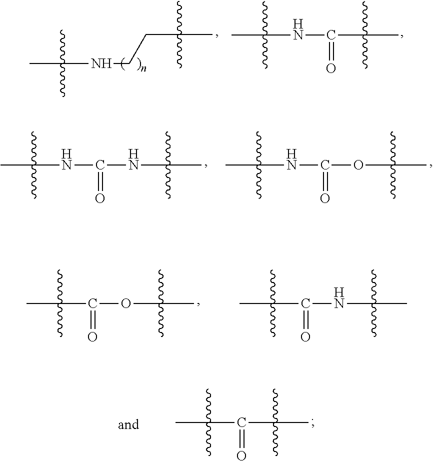

D is a lipid selected from (C₆-C₂₀)alkyl, (C₆-C₂₀)alkenyl and (C₆-C₂₀)alkynyl, wherein said (C₆-C₂₀)alkyl, (C₆-C₂₀)alkenyl, and (C₆-C₂₀)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine; or D is

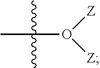

each occurrence of Z is independently selected from (C₆-C₂₀)alkyl, (C₆-C₂₀)alkenyl, and (C₆-C₂₀)alkynyl, wherein said (C₆-C₂₀)alkyl, (C₆-C₂₀)alkenyl, and (C₆-C₂₀)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine;

m is 0, 1, 2, 3, 4 or 5; and

n is 0, 1, 2, 3, 4 or 5.

In some embodiments, a composition of the invention comprises one or more S. pneumoniae antigens and a compound represented by the structure set forth in Formula I, wherein m is 0, 1, 2, 3, 4 or 5.

In some embodiments, a composition of the invention comprises one or more S. pneumoniae antigens and a compound represented by the structure set forth in Formula I, wherein m is 0, 1, 2, 3 or 4.

In some embodiments, a composition of the invention comprises one or more S. pneumoniae antigens and a compound represented by the structure set forth in Formula I, wherein m is 0, 1, 2 or 3.

In some embodiments, a composition of the invention comprises one or more S. pneumoniae antigens and a compound represented by the structure set forth in Formula I, wherein m is 0, 1 or 2.

In some embodiments, a composition of the invention comprises one or more S. pneumoniae antigens and a compound represented by the structure set forth in Formula I, wherein m is 2.

In some embodiments, a composition of the invention comprises one or more S. pneumoniae antigens and a compound represented by the structure set forth in Formula I, wherein m is 1.

In some embodiments, a composition of the invention comprises one or more S. pneumoniae antigens and a compound represented by the structure set forth in Formula I, wherein m is 0.

In some embodiments, a composition of the invention comprises one or more S. pneumoniae antigens and a compound represented by the structure set forth in Formula I, wherein n is 0, 1, 2, 3, 4 or 5.

In some embodiments, a composition of the invention comprises one or more S. pneumoniae antigens and a compound represented by the structure set forth in Formula I, wherein n is 0, 1, 2, 3 or 4.

In some embodiments, a composition of the invention comprises one or more S. pneumoniae antigens and a compound represented by the structure set forth in Formula I, wherein n is 0, 1, 2 or 3.

In some embodiments, a composition of the invention comprises one or more S. pneumoniae antigens and a compound represented by the structure set forth in Formula I, wherein n is 0, 1 or 2.

In some embodiments, a composition of the invention comprises one or more S. pneumoniae antigens and a compound represented by the structure set forth in Formula I, wherein n is 2.

In some embodiments, a composition of the invention comprises one or more S. pneumoniae antigens and a compound represented by the structure set forth in Formula I, wherein n is 1.

In some embodiments, a composition of the invention comprises one or more S. pneumoniae antigens and a compound represented by the structure set forth in Formula I, wherein n is 0.

In some embodiments, a composition of the invention comprises one or more S. pneumoniae antigens and a compound represented by the structure set forth in Formula I, wherein R′ and R″ are independently H, (C₁-C₆)alkyl, (C₁-C₆)alkenyl, or (C₁-C₆)alkynyl, wherein said (C₁-C₆)alkyl, (C₁-C₆)alkenyl and (C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R′ and R″ are independently H, (C₁-C₆)alkyl, (C₁-C₆)alkenyl, or (C₁-C₆)alkynyl, wherein said (C₁-C₆)alkyl, (C₁-C₆)alkenyl and (C₁-C₆)alkynyl are optionally substituted with one to four substituents independently selected from —OH and —O(CH₃).

In some embodiments, a composition of the invention comprises one or more S. pneumoniae antigens and a compound represented by the structure set forth in Formula I, wherein R′ and R″ are independently H, (C₁-C₄)alkyl, (C₁-C₄)alkenyl, or (C₁-C₄)alkynyl, wherein said (C₁-C₄)alkyl, (C₁-C₄)alkenyl and (C₁-C₄)alkynyl are optionally substituted with one or two substituents independently selected from —OH and —O(CH₃).

In some embodiments, a composition of the invention comprises one or more S. pneumoniae antigens and a compound represented by the structure set forth in Formula I, wherein R′ and R″ are independently H and (C₁-C₄)alkyl, wherein said (C₁-C₄)alkyl is optionally substituted with one or two substituents independently selected from —OH and —O(CH₃).

In some embodiments, a composition of the invention comprises one or more S. pneumoniae antigens and a compound represented by the structure set forth in Formula I, wherein R′ and R″ are independently H and (C₁-C₄)alkyl.

In some embodiments, a composition of the invention comprises one or more S. pneumoniae antigens and a compound represented by the structure set forth in Formula I, wherein R′ and R″, together with the nitrogen to which they are attached, join together to form a (C₃-C₆)heterocycloalkyl, wherein said (C₃-C₆)heterocycloalkyl is optionally substituted with one to four substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a} is H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, or NR′R″, wherein said (C₁-C₆)alkyl, (C₁—C₆)alkenyl, (C₁-C₆)alkynyl, —O(C₁-C₆)alkyl, —O(C₁-C₆)alkenyl and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″, wherein said (C₁-C₄)alkyl, (C₁-C₄)alkenyl, (C₁-C₄)alkynyl, —O(C₁-C₄)alkyl, —O(C₁-C₄)alkenyl and —O(C₁-C₄)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″, wherein said (C₁-C₄)alkyl, (C₁-C₄)alkenyl, (C₁-C₄)alkynyl, —O(C₁-C₄)alkyl, —O(C₁-C₄)alkenyl and —O(C₁-C₄)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″.

In some embodiments, a compound of the invention is represented by the structure set forth in Formula I, wherein R^{a} is NR′R″.

In some embodiments, a composition of the invention comprises one or more S. pneumoniae antigens and a compound represented by the structure set forth in Formula I, wherein R^{a} is NR′R″.

In some embodiments, a composition of the invention comprises one or more S. pneumoniae antigens and a compound represented by the structure set forth in Formula I, wherein R′ is H and R″ is pentyl.

In some embodiments, a composition of the invention comprises one or more S. pneumoniae antigens and a compound represented by the structure set forth in Formula I, wherein R′ is H and R″ is butyl.

In some embodiments, a composition of the invention comprises one or more S. pneumoniae antigens and a compound represented by the structure set forth in Formula I, wherein R′ is H and R″ is propyl.

In some embodiments, a composition of the invention comprises one or more S. pneumoniae antigens and a compound represented by the structure set forth in Formula I, wherein R′ is H and R″ is ethyl.

In some embodiments, a composition of the invention comprises one or more S. pneumoniae antigens and a compound represented by the structure set forth in Formula I, wherein R′ is H and R″ is methyl.

In some embodiments, a composition of the invention comprises one or more S. pneumoniae antigens and a compound represented by the structure set forth in Formula I, wherein R′ is H and R″ is H.

In some embodiments, a composition of the invention comprises one or more S. pneumoniae antigens and a compound represented by the structure set forth in Formula I, wherein R^{a′} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″, wherein said (C₁-C₄)alkyl, (C₁-C₄)alkenyl, (C₁-C₄)alkynyl, —O(C₁-C₄)alkyl, —O(C₁-C₄)alkenyl and —O(C₁-C₄)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a′} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″, wherein said (C₁-C₄)alkyl, (C₁-C₄)alkenyl, (C₁-C₄)alkynyl, —O(C₁-C₄)alkyl, —O(C₁-C₄)alkenyl and —O(C₁-C₄)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a′} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″.

In some embodiments, a composition of the invention comprises one or more S. pneumoniae antigens and a compound represented by the structure set forth in Formula I, wherein R^{a′} is NR′R″.

In some embodiments, a composition of the invention comprises one or more S. pneumoniae antigens and a compound represented by the structure set forth in Formula I, wherein R′ is H and R″ is pentyl.

In some embodiments, a composition of the invention comprises one or more S. pneumoniae antigens and a compound represented by the structure set forth in Formula I, wherein R′ is H and R″ is butyl.

In some embodiments, a composition of the invention comprises one or more S. pneumoniae antigens and a compound represented by the structure set forth in Formula I, wherein R′ is H and R″ is propyl.

In some embodiments, a composition of the invention comprises one or more S. pneumoniae antigens and a compound represented by the structure set forth in Formula I, wherein R′ is H and R″ is ethyl.

In some embodiments, a composition of the invention comprises one or more S. pneumoniae antigens and a compound represented by the structure set forth in Formula I, wherein R′ is H and R″ is methyl.

In some embodiments, a composition of the invention comprises one or more S. pneumoniae antigens and a compound represented by the structure set forth in Formula I, wherein R′ is H and R″ is H.

In some embodiments, a composition of the invention comprises one or more S. pneumoniae antigens and a compound represented by the structure set forth in Formula I, wherein R^{a″} is H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, or NR′R″, wherein said (C₁-C₆)alkyl, (C₁-C₆)alkenyl, (C₁-C₆)alkynyl, —O(C₁-C₆)alkyl, —O(C₁-C₆)alkenyl and —O(C₁-C₆)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a″} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″, wherein said (C₁-C₄)alkyl, (C₁-C₄)alkenyl, (C₁-C₄)alkynyl, —O(C₁-C₄)alkyl, —O(C₁-C₄)alkenyl and —O(C₁-C₄)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{a″} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″.

In some embodiments, a composition of the invention comprises one or more S. pneumoniae antigens and a compound represented by the structure set forth in Formula I, wherein R^{a″} is NR′R″.

In some embodiments, a composition of the invention comprises one or more S. pneumoniae antigens and a compound represented by the structure set forth in Formula I, wherein R′ is H and R″ is pentyl.

In some embodiments, a composition of the invention comprises one or more S. pneumoniae antigens and a compound represented by the structure set forth in Formula I, wherein R′ is H and R″ is butyl.

In some embodiments, a composition of the invention comprises one or more S. pneumoniae antigens and a compound represented by the structure set forth in Formula I, wherein R′ is H and R″ is propyl.

In some embodiments, a composition of the invention comprises one or more S. pneumoniae antigens and a compound represented by the structure set forth in Formula I, wherein R′ is H and R″ is ethyl.

In some embodiments, a composition of the invention comprises one or more S. pneumoniae antigens and a compound represented by the structure set forth in Formula I, wherein R′ is H and R″ is methyl.

In some embodiments, a composition of the invention comprises one or more S. pneumoniae antigens and a compound represented by the structure set forth in Formula I, wherein R′ is H and R″ is H.

In some embodiments, a composition of the invention comprises one or more S. pneumoniae antigens and a compound represented by the structure set forth in Formula I, wherein R^{a″} is H.

In some embodiments, a composition of the invention comprises one or more S. pneumoniae antigens and a compound represented by the structure set forth in Formula I, wherein R^{b} is H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, or NR′R″, wherein said (C₁-C₆)alkyl, (C₁-C₆)alkenyl, (C₁-C₆)alkynyl, —O(C₁-C₆)alkyl, —O(C₁-C₆)alkenyl and —O(C₁-C₆)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{b} is H, —OH, (C₁-C₄)alkyl, (C₁-C₄)alkenyl, (C₁-C₄)alkynyl, —O(C₁-C₄)alkyl, —O(C₁-C₄)alkenyl, —O(C₁-C₄)alkynyl, chlorine, fluorine, or NR′R″, wherein said (C₁-C₄)alkyl, (C₁-C₄)alkenyl, (C₁-C₄)alkynyl, —O(C₁-C₄)alkyl, —O(C₁-C₄)alkenyl and —O(C₁-C₄)alkynyl are optionally substituted with one or two substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein R^{b} is independently H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, or NR′R″.

In some embodiments, a composition of the invention comprises one or more S. pneumoniae antigens and a compound represented by the structure set forth in Formula I, wherein R^{b} is independently H, —OH, (C₁-C₄)alkyl, —O(C₁-C₄)alkyl, chlorine, fluorine, or NH₂.

In some embodiments, a composition of the invention comprises one or more S. pneumoniae antigens and a compound represented by the structure set forth in Formula I, wherein R^{b} is independently H and O(CH₃).

In some embodiments, a composition of the invention comprises one or more S. pneumoniae antigens and a compound represented by the structure set forth in Formula I, wherein A is a (C₁-C₆)alkyl or heterocycloalkyl, wherein said alkyl or heterocycloalkyl are optionally substituted with one to four substituents, independently selected from the group consisting of —OH, oxo, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein A is a (C₁-C₄)alkyl or heterocycloalkyl, wherein said alkyl or heterocycloalkyl are optionally substituted with one to four substituents, independently selected from the group consisting of —OH, oxo, (C₁-C₄)alkyl, (C₁-C₄)alkenyl, (C₁-C₄)alkynyl, —O(C₁-C₄)alkyl, —O(C₁—C₄)alkenyl, —O(C₁-C₄)alkynyl, chlorine, fluorine, and NR′R″, wherein said (C₁-C₄)alkyl, (C₁-C₄)alkenyl, (C₁-C₄)alkynyl, —O(C₁-C₄)alkyl, —O(C₁-C₄)alkenyl, and —O(C₁-C₄)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein A is heterocycloalkyl-C(O)—R^{z}— or (C₁-C₄)alkyl-N(R^{z})²—; wherein each occurrence of R^{z} is independently H or (C₁-C₆)alkyl.

In some embodiments, a composition of the invention comprises one or more S. pneumoniae antigens and a compound represented by the structure set forth in Formula I, wherein A is selected from

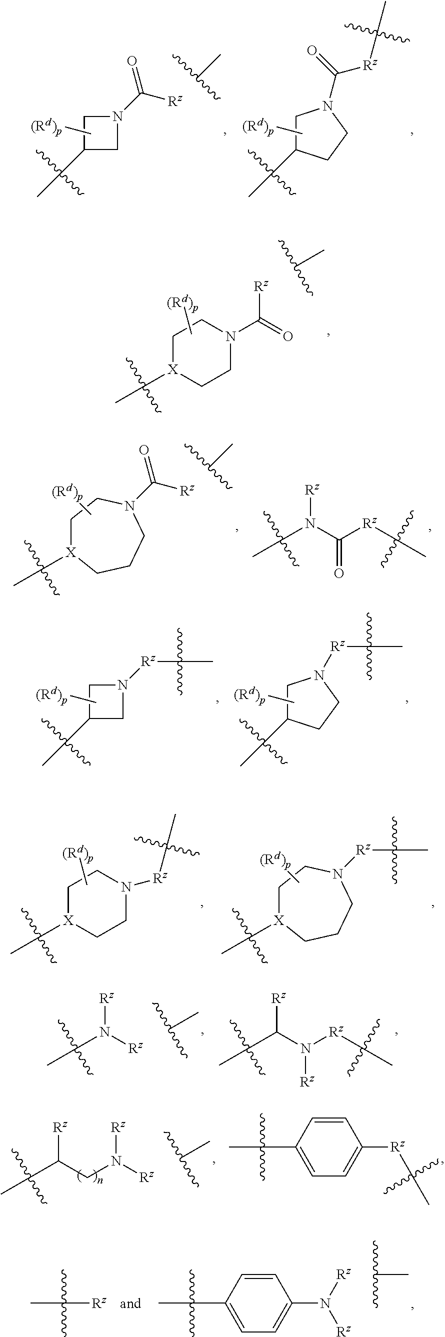

wherein R^{z} is independently selected from H, (C₁-C₆)alkyl, (C₁-C₆)alkenyl, (C₁-C₆)alkynyl, —O(C₁-C₆)alkyl, —O(C₁-C₆)alkenyl and —O(C₁-C₆)alkynyl,

X is CH or N,

R^{d} is independently selected from H, —OH, oxo, (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine,
n is 0, 1, 2, 3 or 4, and
p is 0, 1, 2, 3 or 4.

In some embodiments, a composition of the invention comprises one or more S. pneumoniae antigens and a compound represented by the structure set forth in Formula I, wherein A is selected from

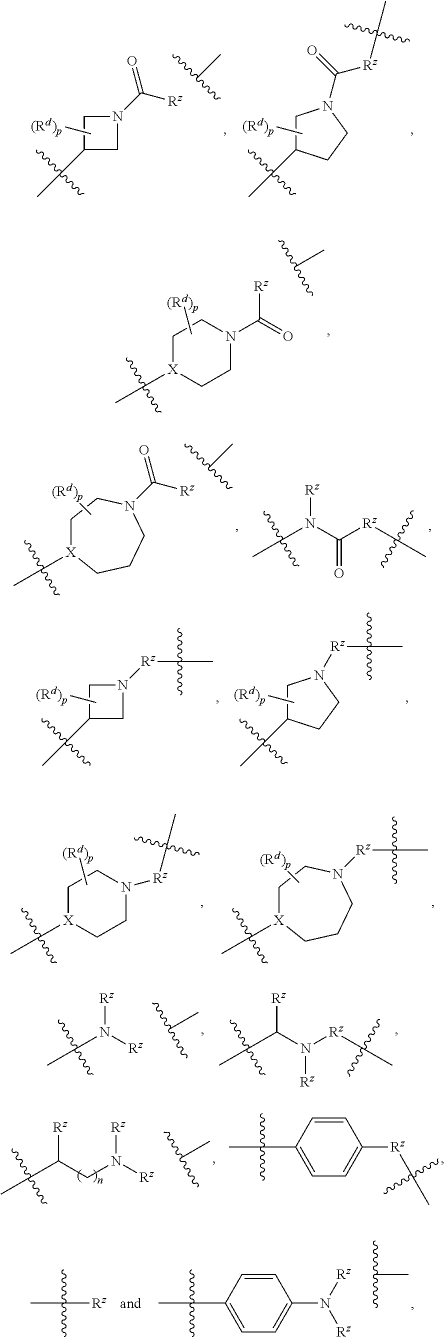

wherein R^{z} is independently selected from H, (C₁-C₆)alkyl, (C₁-C₆)alkenyl and (C₁-C₆)alkynyl, X is CH or N,
R^{d} is independently selected from —OH, (C₁-C₄)alkyl, —O(C₁-C₄)alkyl, chlorine and fluorine,
n is 0, 1, 2 or 3, and
p is 0, 1 or 2.

In some embodiments, a composition of the invention comprises one or more S. pneumoniae antigens and a compound represented by the structure set forth in Formula I, wherein A is selected from

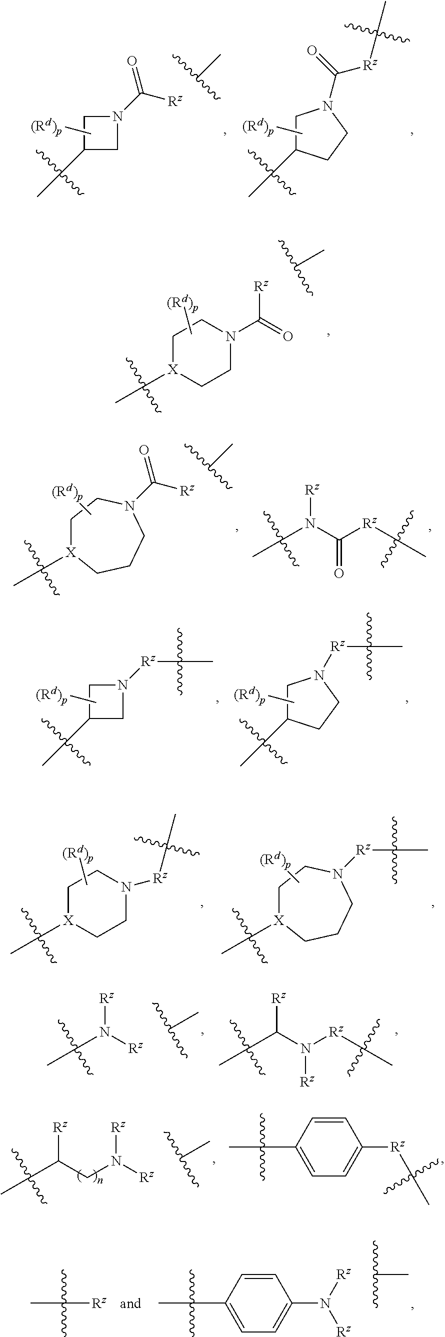

wherein R^{z} is independently H or (C₁-C₆)alkyl,

### X is CH or N,

R^{d} is independently selected from —OH, (C₁-C₄)alkyl, —O(C₁-C₄)alkyl, chlorine and fluorine,
n is 0, 1, 2 or 3, and
p is 0, 1 or 2.

In some embodiments, a composition of the invention comprises one or more S. pneumoniae antigens and a compound represented by the structure set forth in Formula I, wherein A is selected from

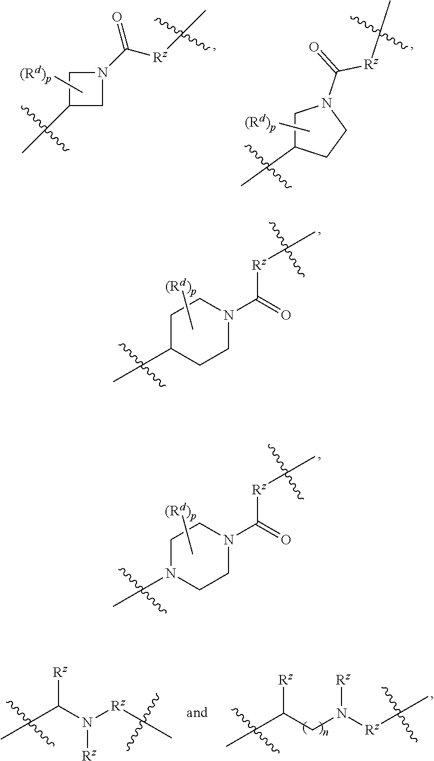

wherein R^{z} is independently H or (C₁-C₆)alkyl,
R^{d} is independently selected from —OH, (C₁-C₄)alkyl, —O(C₁-C₄)alkyl, chlorine and fluorine,
wherein n is 0, 1, 2 or 3, and
wherein p is 0, 1 or 2.

In some embodiments, a composition of the invention comprises one or more S. pneumoniae antigens and a compound represented by the structure set forth in Formula I,

wherein A is selected from

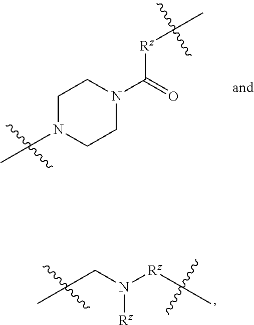

wherein R^{z} is independently H or (C₁-C₆)alkyl.

In some embodiments, a composition of the invention comprises one or more S. pneumoniae antigens and a compound represented by the structure set forth in Formula I, wherein B is selected from

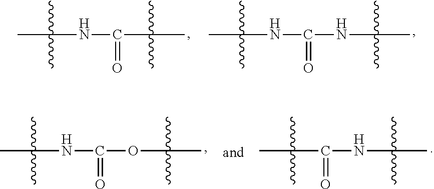 [SMILES: *NC(*)=O ; *NC(=O)N* ; *NC(=O)O* ; *NC(*)=O]

In some embodiments, a composition of the invention comprises one or more S. pneumoniae antigens and a compound represented by the structure set forth in Formula I, wherein B is

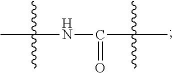 [SMILES: *NC(*)=O]

In some embodiments, a composition of the invention comprises one or more S. pneumoniae antigens and a compound represented by the structure set forth in Formula I, wherein D is (C₆-C₂₀)alkyl, (C₆-C₂₀)alkenyl, or (C₆-C₂₀)alkynyl, wherein said (C₆-C₂₀)alkyl, (C₆-C₂₀)alkenyl, or (C₆-C₂₀)alkynyl are optionally substituted with one to six substituents independently selected from —OH, —O(CH₃), chlorine and fluorine, or

### D is

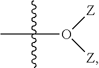

wherein Z is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl, and (C₆-C₂₀)alkynyl are optionally substituted with one to six substituents independently selected from —OH, —O(CH₃), chlorine and fluorine.

In some embodiments, a composition of the invention comprises one or more S. pneumoniae antigens and a compound represented by the structure set forth in Formula I, wherein D is (C₆-C₂₀)alkyl, (C₆-C₂₀)alkenyl, or (C₆-C₂₀)alkynyl, or

### D is

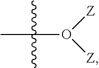

wherein Z is selected from (C₆-C₂₀)alkyl, (C₆-C₂₀)alkenyl and (C₆-C₂₀)alkynyl.

In some embodiments, a composition of the invention comprises one or more S. pneumoniae antigens and a compound represented by the structure set forth in Formula I, wherein D is selected from

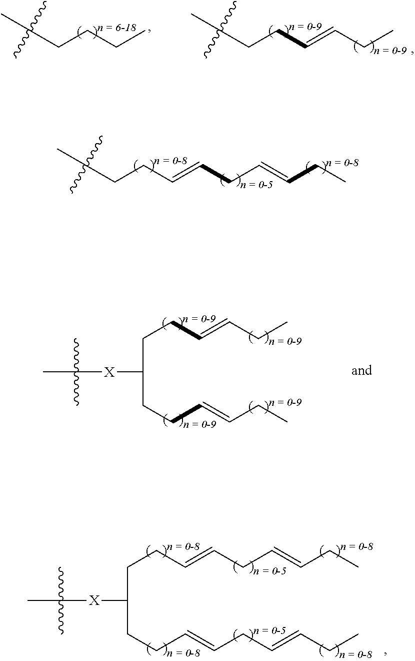

wherein any carbon on the lipid chain is optionally substituted with —OH, —O(CH₃), chlorine or fluorine, [symbol]
is cis or trans stereochemistry,

### X is O, C(R)², or NR, and

R is independently selected from H, (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine.

In some embodiments, a composition of the invention comprises one or more S. pneumoniae antigens and a compound represented by the structure set forth in Formula I, wherein D is selected from

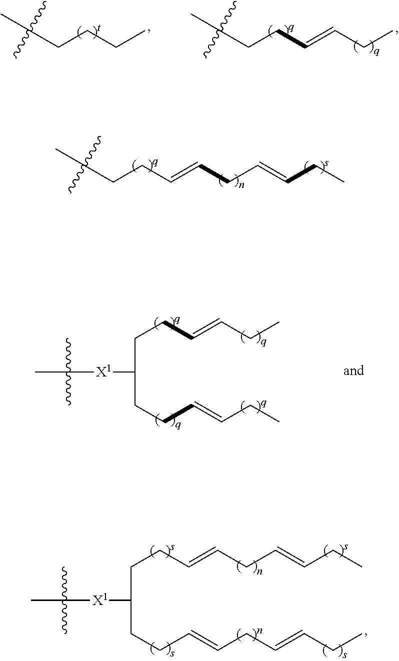

wherein any carbon on the lipid chain is optionally substituted with —OH, —O(CH₃), chlorine or fluorine, [symbol]
is cis or trans stereochemistry,
X¹ is —O—, —C(R)²—, or —NR—, and
each occurrence of R is independently selected from H, (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine;
n is 0, 1, 2, 3, 4 or 5;
q is 0, 1, 2, 3, 4, 5, 6, 7, 8 or 9;
s is 0, 1, 2, 3, 4, 5, 6, 7 or 8; and
t is 6, 7, 8, 9, 10, 11, 12, 13, 14, 15, 16, 17 or 18.

In some embodiments, a composition of the invention comprises one or more S. pneumoniae antigens and a compound represented by the structure set forth in Formula Ia:

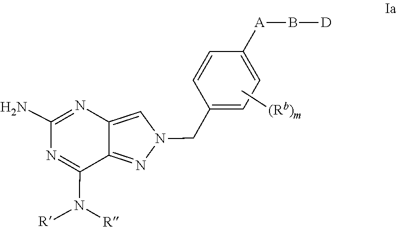

wherein:

R′ and R″ are independently selected from H, (C₁-C₆)alkyl, (C₁-C₆)alkenyl, and (C₁-C₆)alkynyl, wherein said (C₁-C₆)alkyl, (C₁-C₆)alkenyl and (C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine, or R′ and R″, together with the nitrogen to which they are attached, join together to form a (C₃-C₆)heterocycloalkyl, wherein said (C₃-C₆)heterocycloalkyl is optionally substituted with one to four substituents, independently selected from the group consisting of —OH, —O(C₁-C₄)alkyl, —O(C₁-C₄)alkenyl, —O(C₁-C₄)alkynyl, chlorine and fluorine;

each occurrence of R^{b} is —O(C₁-C₄)alkyl, wherein said —O(C₁-C₄)alkyl is optionally substituted with one or two substituents, independently selected from the group consisting of —OH, —O(C₁-C₄)alkyl, —O(C₁-C₄)alkenyl, —O(C₁-C₄)alkynyl, chlorine and fluorine;

A is selected from

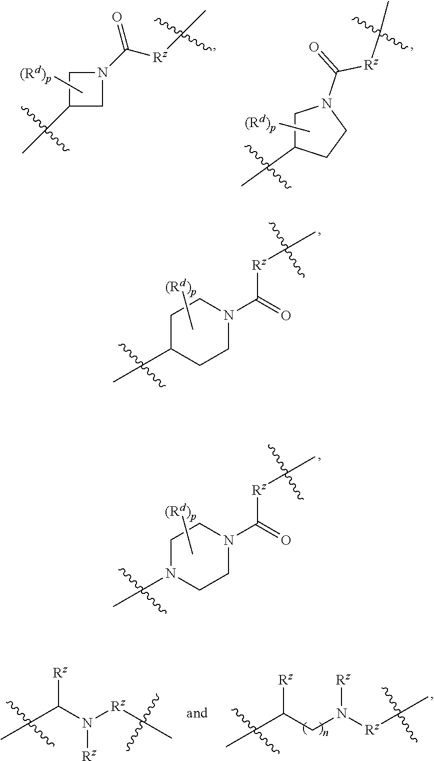

each occurrence of R^{z} is independently H or (C₁-C₆)alkyl;

each occurrence of R^{d} is independently selected from —OH, (C₁-C₄)alkyl, —O(C₁-C₄)alkyl, chlorine and fluorine;

B is

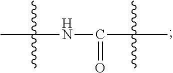 [SMILES: *NC(*)=O]

D is a lipid chain selected from:

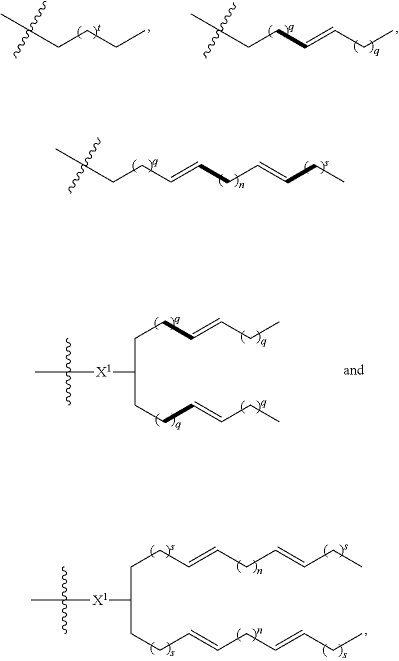

wherein any carbon on the lipid chain is optionally substituted with —OH, —O(C₁-C₄)alkyl, —O(C₁-C₄)alkenyl, —O(C₁-C₄)alkynyl, chlorine or fluorine,
wherein [symbol] is cis or trans stereochemistry,

X¹ is —O—, —C(R)²—, or —NR—, and

each occurrence of R is independently selected from H, (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine;

m is 0, 1 or 2;

n is 0, 1, 2 or 3;

p is 0, 1 or 2;

q is 0, 1, 2, 3, 4, 5, 6, 7, 8 or 9;

s is, 1, 2, 3, 4, 5, 6, 7 or 8; and

t is 6, 7, 8, 9, 10, 11, 12, 13, 14, 15, 16, 17 or 18.

In some embodiments, a composition of the invention comprises one or more S. pneumoniae antigens and a compound represented by the structure set forth in Formula Ia, wherein m is 2

In some embodiments, a composition of the invention comprises one or more S. pneumoniae antigens and a compound represented by the structure set forth in Formula Ia, wherein m is 1.

In some embodiments, a composition of the invention comprises one or more S. pneumoniae antigens and a compound represented by the structure set forth in Formula Ia, wherein m is 0.

In some embodiments, a composition of the invention comprises one or more S. pneumoniae antigens and a compound represented by the structure set forth in Formula Ia, wherein R′ and R″ are independently selected from H and (C₁-C₆)alkyl, wherein said alkyl is optionally substituted with one to four —OH and/or —O(CH₃).

In some embodiments, a composition of the invention comprises one or more S. pneumoniae antigens and a compound represented by the structure set forth in Formula Ia, wherein R′ and R″ are independently selected from H and (C₁-C₄)alkyl, wherein said alkyl is optionally substituted with one to four substituents independently selected from —OH and —O(CH₃).

In some embodiments, a composition of the invention comprises one or more S. pneumoniae antigens and a compound represented by the structure set forth in Formula Ia, wherein R′ is H and R″ is pentyl.

In some embodiments, a composition of the invention comprises one or more S. pneumoniae antigens and a compound represented by the structure set forth in Formula Ia, wherein R′ is H and R″ is butyl.

In some embodiments, a composition of the invention comprises one or more S. pneumoniae antigens and a compound represented by the structure set forth in Formula Ia, wherein R′ is H and R″ is propyl.

In some embodiments, a composition of the invention comprises one or more S. pneumoniae antigens and a compound represented by the structure set forth in Formula Ia, wherein R′ is H and R″ is ethyl.

In some embodiments, a composition of the invention comprises one or more S. pneumoniae antigens and a compound represented by the structure set forth in Formula Ia, wherein R′ is H and R″ is methyl.

In some embodiments, a composition of the invention comprises one or more S. pneumoniae antigens and a compound represented by the structure set forth in Formula Ia, wherein R′ is H and R″ is H.

In some embodiments, a composition of the invention comprises one or more S. pneumoniae antigens and a compound represented by the structure set forth in Formula Ia, wherein A is selected from

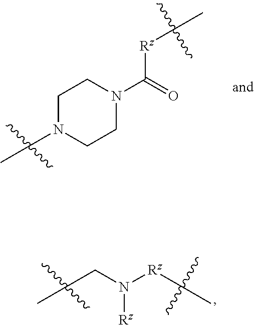

wherein R^{z} is independently H or (C₁-C₆)alkyl.

In some embodiments, a composition of the invention comprises one or more S. pneumoniae antigens and a compound represented by the structure set forth in Formula II:

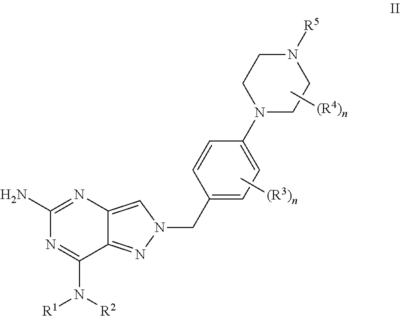

wherein:

R¹ is (C₁-C₆)alkyl, wherein said (C₁-C₆)alkyl is optionally substituted with one to four substituents independently selected from —OH and —O(CH₃);

R² is H, methyl or —O(CH₃);

each occurrence of R³ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);

each occurrence of R⁴ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);

R⁵ is H

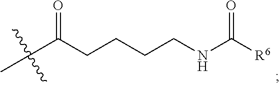

R⁶ is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl, and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine and fluorine; and

each occurrence of n is 4.

In some embodiments, a composition of the invention comprises one or more S. pneumoniae antigens and a compound represented by the structure set forth in Formula II, wherein each occurrence of n is independently 0, 1, 2 or 3.

In some embodiments, a composition of the invention comprises one or more S. pneumoniae antigens and a compound represented by the structure set forth in Formula II, wherein each occurrence of n is independently 0, 1 or 2.

In some embodiments, a composition of the invention comprises one or more S. pneumoniae antigens and a compound represented by the structure set forth in Formula II, wherein each occurrence of n is independently 0 or 1.

In some embodiments, a composition of the invention comprises one or more S. pneumoniae antigens and a compound represented by the structure set forth in Formula II, wherein n is absent.

In some embodiments, a composition of the invention comprises one or more S. pneumoniae antigens and a compound represented by the structure set forth in Formula II, wherein R¹ is ethyl, butyl (branched or a straight chain) or pentyl (branched or a straight chain), wherein said ethyl, butyl or pentyl is optionally substituted with one to four substituents independently selected from —OH, —O(CH₃), chlorine and fluorine.

In some embodiments, a composition of the invention comprises one or more S. pneumoniae antigens and a compound represented by the structure set forth in Formula II, wherein R¹ is butyl (branched or a straight chain), wherein said butyl is optionally substituted with one to four substituents independently selected from —OH, —O(CH₃), chlorine and fluorine.

In some embodiments, a composition of the invention comprises one or more S. pneumoniae antigens and a compound represented by the structure set forth in Formula II, wherein R¹ is ethyl, butyl (branched or a straight chain) or pentyl (branched or a straight chain), wherein said ethyl, butyl or pentyl is optionally substituted with one or two substituents independently selected from —OH and —O(CH₃).

In some embodiments, a composition of the invention comprises one or more S. pneumoniae antigens and a compound represented by the structure set forth in Formula II, wherein R¹ is butyl (branched or a straight chain), wherein said butyl is optionally substituted with one or two substituents independently selected from —OH and —O(CH₃).

In some embodiments, a composition of the invention comprises one or more S. pneumoniae antigens and a compound represented by the structure set forth in Formula II, wherein R² is H or methyl.

In some embodiments, a compound of the invention is represented by the structure set forth in Formula II, wherein R² is H.

In some embodiments, a composition of the invention comprises one or more S. pneumoniae antigens and a compound represented by the structure set forth in Formula II, wherein each occurrence of R³ is independently (C₁-C₄)alkyl, (C₁-C₄)alkenyl or —O(C₁-C₄)alkyl, wherein said (C₁-C₄)alkyl, (C₁-C₄)alkenyl or —O(C₁-C₄)alkyl, is optionally substituted with one to four substituents independently selected from —OH or —O(CH₃).

In some embodiments, a composition of the invention comprises one or more S. pneumoniae antigens and a compound represented by the structure set forth in Formula II, wherein each occurrence of R³ is independently (C₁-C₄)alkyl or —O(C₁-C₄)alkyl, wherein said (C₁-C₄)alkyl or —O(C₁-C₄)alkyl is optionally substituted with one to four substituents independently selected from —OH or —O(CH₃).

In some embodiments, a composition of the invention comprises one or more S. pneumoniae antigens and a compound represented by the structure set forth in Formula II, wherein each occurrence of R³ is independently (C₁-C₄)alkyl or —O(C₁-C₄)alkyl, wherein said (C₁-C₄)alkyl or —O(C₁-C₄)alkyl is optionally substituted with one or two substituents independently selected from —OH or —O(CH₃).

In some embodiments, a composition of the invention comprises one or more S. pneumoniae antigens and a compound represented by the structure set forth in Formula II, wherein each occurrence of R³ is independently methyl or —O(CH₃).

In some embodiments, a composition of the invention comprises one or more S. pneumoniae antigens and a compound represented by the structure set forth in Formula II, wherein each occurrence of R³ is —O(CH₃).

In some embodiments, a composition of the invention comprises one or more S. pneumoniae antigens and a compound represented by the structure set forth in Formula II, wherein each occurrence of R⁴ is independently (C₁-C₄)alkyl or —O(C₁-C₄)alkyl, wherein said (C₁-C₄)alkyl or —O(C₁-C₄)alkyl are optionally substituted with one to four substituents independently selected from —OH, —O(CH₃), chlorine and fluorine.

In some embodiments, a composition of the invention comprises one or more S. pneumoniae antigens and a compound represented by the structure set forth in Formula II, wherein each occurrence of R⁴ is independently (C₁-C₄)alkyl or —O(C₁-C₄)alkyl.

In some embodiments, a composition of the invention comprises one or more S. pneumoniae antigens and a compound represented by the structure set forth in Formula II, wherein R⁶ is selected from (C₆-C₂₀)alkyl and (C₆-C₂₀)alkenyl wherein said (C₆-C₂₀)alkyl and (C₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula II, wherein R⁶ is selected from (C₈-C₂₀)alkyl and (C₈-C₂₀)alkenyl wherein said (C₈-C₂₀)alkyl and (C₈-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula II, wherein R⁶ is selected from (C₁₀-C₂₀)alkyl and (C₁₀-C₂₀)alkenyl wherein said (C₁₀-C₂₀)alkyl and (C₁₀-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula II, wherein R⁶ is selected from (C₁₂-C₂₀)alkyl and (C₁₂-C₂₀)alkenyl wherein said (C₁₂-C₂₀)alkyl and (C₁₂-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula II, wherein R⁶ is selected from (C₁₄-C₂₀)alkyl and (C₁₄-C₂₀)alkenyl wherein said (C₁₄-C₂₀)alkyl and (C₁₄-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula II, wherein R⁶ is selected from (C₁₆-C₂₀)alkyl and (C₁₆-C₂₀)alkenyl wherein said (C₁₆-C₂₀)alkyl and (C₁₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula II, wherein R⁶ is selected from (C₆-C₂₀)alkyl and (C₆-C₂₀)alkenyl.

In some embodiments, a composition of the invention comprises one or more S. pneumoniae antigens and a compound represented by the structure set forth in Formula II, wherein R⁶ is selected from (C₈-C₂₀)alkyl and (C₈-C₂₀)alkenyl.

In some embodiments, a composition of the invention comprises one or more S. pneumoniae antigens and a compound represented by the structure set forth in Formula II, wherein R⁶ is selected from (C₁₀-C₂₀)alkyl and (C₁₀-C₂₀)alkenyl.

In some embodiments, a composition of the invention comprises one or more S. pneumoniae antigens and a compound represented by the structure set forth in Formula II, wherein R⁶ is selected from (C₁₂-C₂₀)alkyl and (C₁₂-C₂₀)alkenyl.

In some embodiments, a composition of the invention comprises one or more S. pneumoniae antigens and a compound represented by the structure set forth in Formula II, wherein R⁶ is selected from (C₁₄-C₂₀)alkyl and (C₁₄-C₂₀)alkenyl.

In some embodiments, a composition of the invention comprises one or more S. pneumoniae antigens and a compound represented by the structure set forth in Formula II, wherein R⁶ is selected from (C₁₆-C₂₀)alkyl and (C₁₆-C₂₀)alkenyl.

The invention further provides a composition comprising a compound having the structure set forth in Formula IIa:

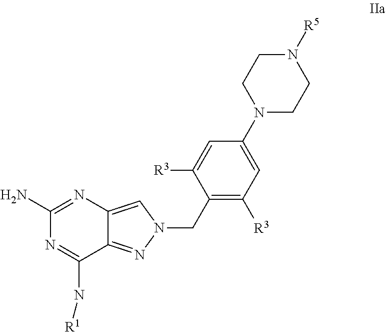

wherein:

R¹ is butyl, wherein said butyl is optionally substituted with one or two —OH; each occurrence of R³ is independently H or —O(CH₃); and

R⁵ is

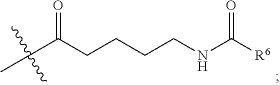

and

R⁶ is selected from (C₁₀-C₂₀)alkyl, (C₁₀-C₂₀)alkenyl and (C₁₀-C₂₀)alkynyl.

In some embodiments, a compound of the invention is represented by the structure set forth in Formula IIa, wherein R⁶ is selected from (C₁₀-C₂₀)alkyl and (C₁₀-C₂₀)alkenyl wherein said (C₁₀-C₂₀)alkyl and (C₁₀-C₂₀)alkenyl are optionally substituted with one or two substituents independently selected from —OH and —O(CH₃).

In some embodiments, a composition of the invention comprises one or more S. pneumoniae antigens and a compound represented by the structure set forth in Formula IIa, wherein R⁶ is selected from (C₁₂-C₂₀)alkyl and (C₁₂-C₂₀)alkenyl wherein said (C₁₂-C₂₀)alkyl and (C₁₂-C₂₀)alkenyl are optionally substituted with one or two substituents independently selected from —OH and —O(CH₃).

In some embodiments, a composition of the invention comprises one or more S. pneumoniae antigens and a compound represented by the structure set forth in Formula IIa, wherein R⁶ is selected from (C₁₄-C₂₀)alkyl and (C₁₄-C₂₀)alkenyl wherein said (C₁₄-C₂₀)alkyl and (C₁₄-C₂₀)alkenyl are optionally substituted with one or two substituents independently selected from —OH and —O(CH₃).

In some embodiments, a composition of the invention comprises one or more S. pneumoniae antigens and a compound represented by the structure set forth in Formula IIa, wherein R⁶ is selected from (C₁₆-C₂₀)alkyl and (C₁₆-C₂₀)alkenyl wherein said (C₁₆-C₂₀)alkyl and (C₁₆-C₂₀)alkenyl are optionally substituted with one or two substituents independently selected from —OH and —O(CH₃).

In some embodiments, a composition of the invention comprises one or more S. pneumoniae antigens and a compound represented by the structure set forth in Formula IIa, wherein R⁶ is selected from (C₁₀-C₂₀)alkyl and (C₁₀-C₂₀)alkenyl.

In some embodiments, a composition of the invention comprises one or more S. pneumoniae antigens and a compound represented by the structure set forth in Formula IIa, wherein R⁶ is selected from (C₁₂-C₂₀)alkyl and (C₁₂-C₂₀)alkenyl.

In some embodiments, a composition of the invention comprises one or more S. pneumoniae antigens and a compound represented by the structure set forth in Formula IIa, wherein R⁶ is selected from (C₁₄-C₂₀)alkyl and (C₁₄-C₂₀)alkenyl.

In some embodiments, a composition of the invention comprises one or more S. pneumoniae antigens and a compound represented by the structure set forth in Formula IIa, wherein R⁶ is selected from (C₁₆-C₂₀)alkyl and (C₁₆-C₂₀)alkenyl.

The invention provides a composition comprising a compound having the structure set forth in Formula III:

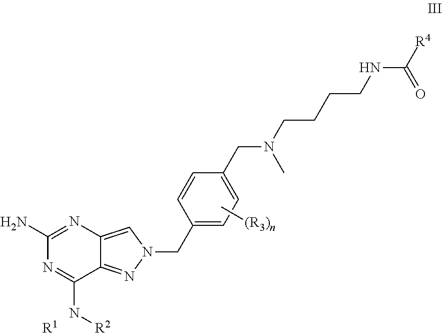

wherein:

R¹ is (C₁-C₆)alkyl, wherein said (C₁-C₆)alkyl is optionally substituted with one to four substituents independently selected from —OH and —O(CH₃);

R² is H, methyl or —O(CH₃);

each occurrence of R³ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);

R⁴ is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine and fluorine; and

n is 4.

In some embodiments, a composition of the invention comprises one or more S. pneumoniae antigens and a compound represented by the structure set forth in Formula III, wherein n is 0, 1, 2 or 3.

In some embodiments, a composition of the invention comprises one or more S. pneumoniae antigens and a compound represented by the structure set forth in Formula III, wherein n is 0, 1 or 2.

In some embodiments, a composition of the invention comprises one or more S. pneumoniae antigens and a compound represented by the structure set forth in Formula III, wherein n is 0 or 1.

In some embodiments, a composition of the invention comprises one or more S. pneumoniae antigens and a compound represented by the structure set forth in Formula III, wherein n is absent.

In some embodiments, a composition of the invention comprises one or more S. pneumoniae antigens and a compound represented by the structure set forth in Formula III, wherein R¹ is ethyl, butyl (branched or a straight chain) or pentyl (branched or a straight chain), wherein said ethyl, butyl or pentyl is optionally substituted with one to four substituents independently selected from —OH, —O(CH₃), chlorine and fluorine.

In some embodiments, a composition of the invention comprises one or more S. pneumoniae antigens and a compound represented by the structure set forth in Formula III, wherein R¹ is butyl (branched or a straight chain), wherein said butyl is optionally substituted with one to four substituents independently selected from —OH, —O(CH₃), chlorine and fluorine.

In some embodiments, a composition of the invention comprises one or more S. pneumoniae antigens and a compound represented by the structure set forth in Formula III, wherein R¹ is ethyl, butyl (branched or a straight chain) or pentyl (branched or a straight chain), wherein said ethyl, butyl or pentyl is optionally substituted with one or two substituents independently selected from —OH and —O(CH₃).

In some embodiments, a composition of the invention comprises one or more S. pneumoniae antigens and a compound represented by the structure set forth in Formula III, wherein R¹ is butyl (branched or a straight chain), wherein said butyl is optionally substituted with one or two substituents independently selected from —OH and —O(CH₃).

In some embodiments, a composition of the invention comprises one or more S. pneumoniae antigens and a compound represented by the structure set forth in Formula III, wherein R² is H or methyl.

In some embodiments, a composition of the invention comprises one or more S. pneumoniae antigens and a compound represented by the structure set forth in Formula III, wherein R² is H.

In some embodiments, a composition of the invention comprises one or more S. pneumoniae antigens and a compound represented by the structure set forth in Formula III, wherein each occurrence of R³ is independently (C₁-C₄)alkyl, (C₁-C₄)alkenyl or —O(C₁-C₄)alkyl, wherein said (C₁-C₄)alkyl, (C₁-C₄)alkenyl or —O(C₁-C₄)alkyl, is optionally substituted with one to four substituents independently selected from —OH or —O(CH₃).

In some embodiments, a composition of the invention comprises one or more S. pneumoniae antigens and a compound represented by the structure set forth in Formula III, wherein each occurrence of R³ is independently (C₁-C₄)alkyl or —O(C₁-C₄)alkyl, wherein said (C₁-C₄)alkyl or —O(C₁-C₄)alkyl is optionally substituted with one to four substituents independently selected from —OH or —O(CH₃).

In some embodiments, a composition of the invention comprises one or more S. pneumoniae antigens and a compound represented by the structure set forth in Formula III, wherein each occurrence of R³ is independently (C₁-C₄)alkyl or —O(C₁-C₄)alkyl, wherein said (C₁-C₄)alkyl or —O(C₁-C₄)alkyl is optionally substituted with one or two substituents independently selected from —OH or —O(CH₃).

In some embodiments, a composition of the invention comprises one or more S. pneumoniae antigens and a compound represented by the structure set forth in Formula III, wherein each occurrence of R³ is independently methyl or —O(CH₃).

In some embodiments, a composition of the invention comprises one or more S. pneumoniae antigens and a compound represented by the structure set forth in Formula III, wherein each occurrence of R³ is —O(CH₃).

In some embodiments, a composition of the invention comprises one or more S. pneumoniae antigens and a compound represented by the structure set forth in Formula III, wherein R⁴ is selected from (C₆-C₂₀)alkyl and (C₆-C₂₀)alkenyl wherein said (C₆-C₂₀)alkyl and (C₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula III, wherein R⁴ is selected from (C₈-C₂₀)alkyl and (C₈-C₂₀)alkenyl wherein said (C₈-C₂₀)alkyl and (C₈-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula III, wherein R⁴ is selected from (C₁₀-C₂₀)alkyl and (C₁₀-C₂₀)alkenyl wherein said (C₁₀-C₂₀)alkyl and (C₁₀-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula III, wherein R⁴ is selected from (C₁₂-C₂₀)alkyl and (C₁₂-C₂₀)alkenyl wherein said (C₁₂-C₂₀)alkyl and (C₁₂-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula III, wherein R⁴ is selected from (C₁₄-C₂₀)alkyl and (C₁₄-C₂₀)alkenyl wherein said (C₁₄-C₂₀)alkyl and (C₁₄-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula III, wherein R⁴ is selected from (C₁₆-C₂₀)alkyl and (C₁₆-C₂₀)alkenyl wherein said (C₁₆-C₂₀)alkyl and (C₁₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula III, wherein R⁴ is selected from (C₆-C₂₀)alkyl and (C₆-C₂₀)alkenyl.

In some embodiments, a composition of the invention comprises one or more S. pneumoniae antigens and a compound represented by the structure set forth in Formula III, wherein R⁴ is selected from (C₈-C₂₀)alkyl and (C₈-C₂₀)alkenyl.

In some embodiments, a composition of the invention comprises one or more S. pneumoniae antigens and a compound represented by the structure set forth in Formula III, wherein R⁴ is selected from (C₁₀-C₂₀)alkyl and (C₁₀-C₂₀)alkenyl.

In some embodiments, a composition of the invention comprises one or more S. pneumoniae antigens and a compound represented by the structure set forth in Formula III, wherein R⁴ is selected from (C₁₂-C₂₀)alkyl and (C₁₂-C₂₀)alkenyl.

In some embodiments, a composition of the invention comprises one or more S. pneumoniae antigens and a compound represented by the structure set forth in Formula III, wherein R⁴ is selected from (C₁₄-C₂₀)alkyl and (C₁₄-C₂₀)alkenyl.

In some embodiments, a composition of the invention comprises one or more S. pneumoniae antigens and a compound represented by the structure set forth in Formula III, wherein R⁴ is selected from (C₁₆-C₂₀)alkyl and (C₁₆-C₂₀)alkenyl.

The invention also provides a composition comprising a compound having the structure set forth in Formula IIIa:

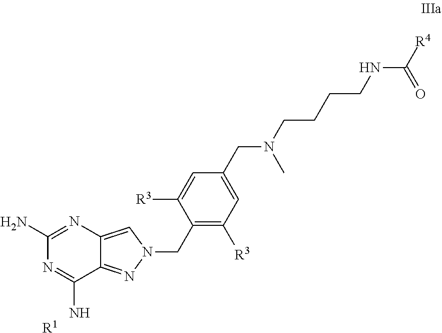

wherein:

R¹ is butyl, wherein said butyl is optionally substituted with one or two —OH;

each occurrence of R³ is independently H or —O(CH₃); and

R⁴ is selected from (C₁₀-C₂₀)alkyl, (C₁₀-C₂₀)alkenyl and (C₁₀-C₂₀)alkynyl.

In some embodiments, a composition of the invention comprises one or more S. pneumoniae antigens and a compound represented by the structure set forth in Formula IIIa, wherein R⁴ is selected from (C₆-C₂₀)alkyl and (C₆-C₂₀)alkenyl wherein said (C₆-C₂₀)alkyl and (C₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IIIa, wherein R⁴ is selected from (C₈-C₂₀)alkyl and (C₈-C₂₀)alkenyl wherein said (C₈-C₂₀)alkyl and (C₈-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IIIa, wherein R⁴ is selected from (C₁₀-C₂₀)alkyl and (C₁₀-C₂₀)alkenyl wherein said (C₁₀-C₂₀)alkyl and (C₁₀-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IIIa, wherein R⁴ is selected from (C₁₂-C₂₀)alkyl and (C₁₂-C₂₀)alkenyl wherein said (C₁₂-C₂₀)alkyl and (C₁₂-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IIIa, wherein R⁴ is selected from (C₁₄-C₂₀)alkyl and (C₁₄-C₂₀)alkenyl wherein said (C₁₄-C₂₀)alkyl and (C₁₄-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IIIa, wherein R⁴ is selected from (C₁₆-C₂₀)alkyl and (C₁₆-C₂₀)alkenyl wherein said (C₁₆-C₂₀)alkyl and (C₁₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IIIa, wherein R⁴ is selected from (C₆-C₂₀)alkyl and (C₆-C₂₀)alkenyl.

In some embodiments, a composition of the invention comprises one or more S. pneumoniae antigens and a compound represented by the structure set forth in Formula IIIa, wherein R⁴ is selected from (C₈-C₂₀)alkyl and (C₈-C₂₀)alkenyl.

In some embodiments, a composition of the invention comprises one or more S. pneumoniae antigens and a compound represented by the structure set forth in Formula IIIa, wherein R⁴ is selected from (C₁₀-C₂₀)alkyl and (C₁₀-C₂₀)alkenyl.

In some embodiments, a composition of the invention comprises one or more S. pneumoniae antigens and a compound represented by the structure set forth in Formula IIIa, wherein R⁴ is selected from (C₁₂-C₂₀)alkyl and (C₁₂-C₂₀)alkenyl.

In some embodiments, a composition of the invention comprises one or more S. pneumoniae antigens and a compound represented by the structure set forth in Formula IIIa, wherein R⁴ is selected from (C₁₄-C₂₀)alkyl and (C₁₄-C₂₀)alkenyl.

In some embodiments, a composition of the invention comprises one or more S. pneumoniae antigens and a compound represented by the structure set forth in Formula IIIa, wherein R⁴ is selected from (C₁₆-C₂₀)alkyl and (C₁₆-C₂₀)alkenyl.

The invention provides a composition comprising a compound having the structure set forth in Formula IV:

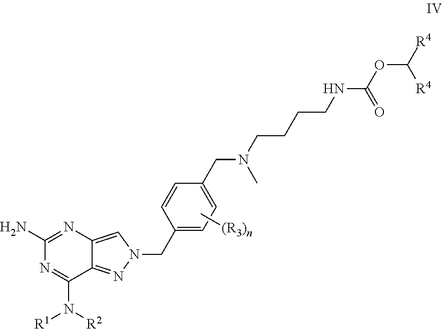

wherein:

R¹ is (C₁-C₆)alkyl, wherein said (C₁-C₆)alkyl is optionally substituted with one to four substituents independently selected from —OH and —O(CH₃);

R² is H, methyl or —O(CH₃);

each occurrence of R³ is independently H, (C₁-C₄)alkyl, (C₁-C₄)alkenyl, (C₁-C₄)alkynyl, or —O(C₁-C₄)alkyl wherein said (C₁-C₄)alkyl, (C₁-C₄)alkenyl, (C₁-C₄)alkynyl, and —O(C₁-C₄)alkyl are optionally substituted with one or two substituents independently selected from —OH and —O(CH₃);

each occurrence of R⁴ is independently selected from (C₆-C₂₀)alkyl, (C₆-C₂₀)alkenyl and (C₆-C₂₀)alkynyl, wherein said (C₆-C₂₀)alkyl, (C₆-C₂₀)alkenyl and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine or fluorine; and

n is 4.

In some embodiments, a composition of the invention comprises one or more S. pneumoniae antigens and a compound represented by the structure set forth in Formula IV, wherein n is 0, 1, 2 or 3.

In some embodiments, the a composition of the invention comprises one or more S. pneumoniae antigens and a compound represented by the structure set forth in Formula IV, wherein n is 0, 1 or 2.

In some embodiments, a composition of the invention comprises one or more S. pneumoniae antigens and a compound represented by the structure set forth in Formula IV, wherein n is 0 or 1.

In some embodiments, a composition of the invention comprises one or more S. pneumoniae antigens and a compound represented by the structure set forth in Formula IV, wherein n is absent.

In some embodiments, a composition of the invention comprises one or more S. pneumoniae antigens and a compound represented by the structure set forth in Formula IV, wherein R¹ is ethyl, butyl (branched or a straight chain) or pentyl (branched or a straight chain), wherein said ethyl, butyl or pentyl is optionally substituted with one to four substituents independently selected from —OH, —O(CH₃), chlorine and fluorine.

In some embodiments, a composition of the invention comprises one or more S. pneumoniae antigens and a compound represented by the structure set forth in Formula IV, wherein R¹ is butyl (branched or a straight chain), wherein said butyl is optionally substituted with one to four substituents independently selected from —OH, —O(CH₃), chlorine and fluorine.

In some embodiments, a composition of the invention comprises one or more S. pneumoniae antigens and a compound represented by the structure set forth in Formula IV, wherein R¹ is ethyl, butyl (branched or a straight chain) or pentyl (branched or a straight chain), wherein said ethyl, butyl or pentyl is optionally substituted with one or two substituents independently selected from —OH and —O(CH₃).

In some embodiments, a composition of the invention comprises one or more S. pneumoniae antigens and a compound represented by the structure set forth in Formula IV, wherein R¹ is butyl (branched or a straight chain), wherein said butyl is optionally substituted with one or two substituents independently selected from —OH and —O(CH₃).

In some embodiments, a composition of the invention comprises one or more S. pneumoniae antigens and a compound represented by the structure set forth in Formula IV, wherein R² is H or methyl.

In some embodiments, a composition of the invention comprises one or more S. pneumoniae antigens and a compound represented by the structure set forth in Formula IV, wherein R² is H.

In some embodiments, a composition of the invention comprises one or more S. pneumoniae antigens and a compound represented by the structure set forth in Formula IV, wherein R³ is independently (C₁-C₄)alkyl, (C₁-C₄)alkenyl or —O(C₁-C₄)alkyl, wherein said (C₁-C₄)alkyl, (C₁-C₄)alkenyl or —O(C₁-C₄)alkyl, is optionally substituted with one to four substituents independently selected from —OH or —O(CH₃).

In some embodiments, a composition of the invention comprises one or more S. pneumoniae antigens and a compound represented by the structure set forth in Formula IV, wherein each occurrence of R³ is independently (C₁-C₄)alkyl or —O(C₁-C₄)alkyl, wherein said (C₁-C₄)alkyl or —O(C₁-C₄)alkyl is optionally substituted with one to four substituents independently selected from —OH or —O(CH₃).

In some embodiments, a composition of the invention comprises one or more S. pneumoniae antigens and a compound represented by the structure set forth in Formula IV, wherein each occurrence of R³ is independently (C₁-C₄)alkyl or —O(C₁-C₄)alkyl, wherein said (C₁-C₄)alkyl or —O(C₁-C₄)alkyl is optionally substituted with one or two substituents independently selected from —OH or —O(CH₃).

In some embodiments, a composition of the invention comprises one or more S. pneumoniae antigens and a compound represented by the structure set forth in Formula IV, wherein each occurrence of R³ is independently methyl or —O(CH₃).

In some embodiments, a composition of the invention comprises one or more S. pneumoniae antigens and a compound represented by the structure set forth in Formula IV, wherein each occurrence of R³ is —O(CH₃).

In some embodiments, a composition of the invention comprises one or more S. pneumoniae antigens and a compound represented by the structure set forth in Formula IV, wherein each occurrence of R⁴ is independently selected from (C₆-C₂₀)alkyl and (C₆-C₂₀)alkenyl wherein said (C₆-C₂₀)alkyl and (C₆-C₂₀)alkenyl are optionally substituted with one to six —OH or —O(CH₃), independently selected.

In some embodiments, a composition of the invention comprises one or more S. pneumoniae antigens and a compound represented by the structure set forth in Formula IV, wherein each occurrence of R⁴ is independently selected from (C₈-C₂₀)alkyl and (C₈-C₂₀)alkenyl wherein said (C₈-C₂₀)alkyl and (C₈-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IV, wherein each occurrence of R⁴ is independently selected from (C₁₀-C₂₀)alkyl and (C₁₀-C₂₀)alkenyl wherein said (C₁₀-C₂₀)alkyl and (C₁₀-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IV, wherein each occurrence of R⁴ is independently selected from (C₁₂-C₂₀)alkyl and (C₁₂-C₂₀)alkenyl wherein said (C₁₂-C₂₀)alkyl and (C₁₂-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IV, wherein each occurrence of R⁴ is independently selected from (C₁₄-C₂₀)alkyl and (C₁₄-C₂₀)alkenyl wherein said (C₁₄-C₂₀)alkyl and (C₁₄-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IV, wherein each occurrence of R⁴ is independently selected from (C₁₆-C₂₀)alkyl and (C₁₆-C₂₀)alkenyl wherein said (C₁₆-C₂₀)alkyl and (C₁₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IV, wherein each occurrence of R⁴ is independently selected from (C₆-C₂₀)alkyl and (C₆-C₂₀)alkenyl.

In some embodiments, a composition of the invention comprises one or more S. pneumoniae antigens and a compound represented by the structure set forth in Formula IV, wherein each occurrence of R⁴ is independently selected from (C₈-C₂₀)alkyl and (C₈-C₂₀)alkenyl.

In some embodiments, a composition of the invention comprises one or more S. pneumoniae antigens and a compound represented by the structure set forth in Formula IV, wherein each occurrence of R⁴ is independently selected from (C₁₀-C₂₀)alkyl and (C₁₀-C₂₀)alkenyl.

In some embodiments, a composition of the invention comprises one or more S. pneumoniae antigens and a compound represented by the structure set forth in Formula IV, wherein each occurrence of R⁴ is independently selected from (C₁₂-C₂₀)alkyl and (C₁₂-C₂₀)alkenyl.

In some embodiments, a composition of the invention comprises one or more S. pneumoniae antigens and a compound represented by the structure set forth in Formula IV, wherein each occurrence of R⁴ is independently selected from (C₁₄-C₂₀)alkyl and (C₁₄-C₂₀)alkenyl.

In some embodiments, a composition of the invention comprises one or more S. pneumoniae antigens and a compound represented by the structure set forth in Formula IV, wherein each occurrence of R⁴ is independently selected from (C₁₆-C₂₀)alkyl and (C₁₆-C₂₀)alkenyl.

The invention provides a composition of the invention comprising a compound having the structure set forth in Formula IVa:

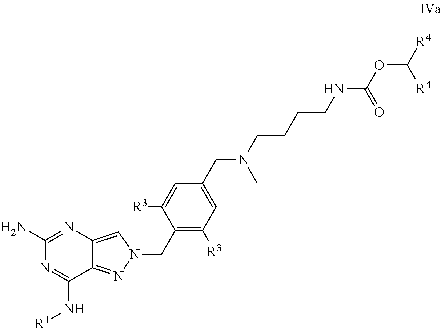

wherein:

R¹ is butyl, wherein said butyl is optionally substituted with one or two —OH;

each occurrence of R³ is independently H or —O(CH₃); and

each occurrence of R⁴ is independently selected from (C₁₀-C₂₀)alkyl, (C₁₀-C₂₀)alkenyl and (C₁₀-C₂₀)alkynyl.

In some embodiments, a composition of the invention comprises one or more S. pneumoniae antigens and a compound represented by the structure set forth in Formula IVa, wherein each occurrence of R⁴ is independently selected from (C₆-C₂₀)alkyl and (C₆-C₂₀)alkenyl wherein said (C₆-C₂₀)alkyl and (C₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IVa, wherein each occurrence of R⁴ is independently selected from (C₈-C₂₀)alkyl and (C₈-C₂₀)alkenyl wherein said (C₈-C₂₀)alkyl and (C₈-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IVa, wherein each occurrence of R⁴ is independently selected from (C₁₀-C₂₀)alkyl and (C₁₀-C₂₀)alkenyl wherein said (C₁₀-C₂₀)alkyl and (C₁₀-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IVa, wherein each occurrence of R⁴ is independently selected from (C₁₂-C₂₀)alkyl and (C₁₂-C₂₀)alkenyl wherein said (C₁₂-C₂₀)alkyl and (C₁₂-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IVa, wherein each occurrence of R⁴ is independently selected from (C₁₄-C₂₀)alkyl and (C₁₄-C₂₀)alkenyl wherein said (C₁₄-C₂₀)alkyl and (C₁₄-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IVa, wherein each occurrence of R⁴ is independently selected from (C₁₆-C₂₀)alkyl and (C₁₆-C₂₀)alkenyl wherein said (C₁₆-C₂₀)alkyl and (C₁₆-C₂₀)alkenyl are optionally substituted with one to six substituents independently selected from —OH and —O(CH₃).

In some embodiments, a composition of the invention comprises one or more S. pneumoniae antigens and a compound represented by the structure set forth in Formula IVa, wherein each occurrence of R⁴ is independently selected from (C₆-C₂₀)alkyl and (C₆-C₂₀)alkenyl.

In some embodiments, a composition of the invention comprises one or more S. pneumoniae antigens and a compound represented by the structure set forth in Formula IVa, wherein each occurrence of R⁴ is independently selected from (C₈-C₂₀)alkyl and (C₈-C₂₀)alkenyl.

In some embodiments, a composition of the invention comprises one or more S. pneumoniae antigens and a compound represented by the structure set forth in Formula IVa, wherein each occurrence of R⁴ is independently selected from (C₁₀-C₂₀)alkyl and (C₁₀-C₂₀)alkenyl.

In some embodiments, a composition of the invention comprises one or more S. pneumoniae antigens and a compound represented by the structure set forth in Formula IVa, wherein each occurrence of R⁴ is independently selected from (C₁₂-C₂₀)alkyl and (C₁₂-C₂₀)alkenyl.

In some embodiments, a composition of the invention comprises one or more S. pneumoniae antigens and a compound represented by the structure set forth in Formula IVa, wherein each occurrence of R⁴ is independently selected from (C₁₄-C₂₀)alkyl and (C₁₄-C₂₀)alkenyl.

In some embodiments, a composition of the invention comprises one or more S. pneumoniae antigens and a compound represented by the structure set forth in Formula IVa, wherein each occurrence of R⁴ is independently selected from (C₁₆-C₂₀)alkyl and (C₁₆-C₂₀)alkenyl.

In some embodiments, the invention provides a composition comprising one or more S. pneumoniae antigens and a compound selected from the group consisting of:
(N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide;
(S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide;
(S)-1-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)octadecan-1-one;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)tetradecanamide;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)oleamide;
(9Z,12Z)—N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)octadeca-9,12-dienamide;
N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide;
N-(5-(4-(4-((5-amino-7-(butylamino)-2H-pyrazolo[4,3-d]pyrimidin-2-yl)methyl)-3-methoxyphenyl)piperidin-1-yl)-5-oxopentyl)stearamide;
N-(5-(3-(4-((5-amino-7-(butylamino)-2H-pyrazolo[4,3-d]pyrimidin-2-yl)methyl)-3-methoxyphenyl)azetidin-1-yl)-5-oxopentyl)stearamide;
1-(4-((5-amino-7-(butylamino)-2H-pyrazolo[4,3-d]pyrimidin-2-yl)methyl)-3-methoxyphenyl)-N-(3-stearamidopropyl)piperidine-4-carboxamide;
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide;
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide;
N-(3-(4-(4-((5-amino-7-(butylamino)-2H-pyrazolo[4,3-d]pyrimidin-2-yl)methyl)-3-methoxyphenyl)piperazin-1-yl)-3-oxopropyl)stearamide;
N-(7-(4-(4-((5-amino-7-(butylamino)-2H-pyrazolo[4,3-d]pyrimidin-2-yl)methyl)-3-methoxyphenyl)piperazin-1-yl)-7-oxoheptyl)stearamide;
N-(3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)cyclobutyl)stearamide;
N-(5-(4-(4-((5-amino-7-(butylamino)-2H-pyrazolo[4,3-d]pyrimidin-2-yl)methyl)-3-methoxyphenyl)piperazin-1-yl)-4,4-dimethyl-5-oxopentyl)stearamide;
N-(6-(4-(4-((5-amino-7-(butylamino)-2H-pyrazolo[4,3-d]pyrimidin-2-yl)methyl)-3-methoxyphenyl)piperazin-1-yl)-2-methyl-6-oxohexan-2-yl)stearamide;
1-(4-(4-((5-amino-7-(butylamino)-2H-pyrazolo[4,3-d]pyrimidin-2-yl)methyl)-3-methoxyphenyl)piperazin-1-yl)-5-(octadecyloxy)pentan-1-one;
1-(4-(4-((5-amino-7-(butylamino)-2H-pyrazolo[4,3-d]pyrimidin-2-yl)methyl)-3-methoxyphenyl)piperazin-1-yl)-5-(octadecylamino)pentan-1-one;
N-(5-(4-(4-((5-amino-7-(butylamino)-3-methyl-2H-pyrazolo[4,3-d]pyrimidin-2-yl)methyl)-3-methoxyphenyl)piperazin-1-yl)-5-oxopentyl)stearamide;
(9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide;
N-(4-((4-((5-amino-7-(butylamino)-2H-pyrazolo[4,3-d]pyrimidin-2-yl)methyl)-3,5-dimethoxybenzyl)(methyl)amino)butyl)tetradecanamide;
N-(4-((4-((5-amino-7-(butylamino)-2H-pyrazolo[4,3-d]pyrimidin-2-yl)methyl)-3,5-dimethoxybenzyl)(methyl)amino)butyl)oleamide;
N-(4-((4-((5-amino-7-(butylamino)-2H-pyrazolo[4,3-d]pyrimidin-2-yl)methyl)-3,5-dimethoxybenzyl)(methyl)amino)butyl)stearamide;
N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide;
(6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)amino)butyl)carbamate;
4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide;
3-stearamidopropyl 4-(4-((5-amino-7-(butylamino)-2H-pyrazolo[4,3-d]pyrimidin-2-yl)methyl)-3-methoxyphenyl)piperazine-1-carboxylate;
N-(5-(4-(4-((5-amino-7-(butylamino)-2H-pyrazolo[4,3-d]pyrimidin-2-yl)methyl)-3-methoxyphenyl)piperazin-1-yl)-6,6,6-trifluorohexyl)stearamide;
N-(4-((4-((7-(butylamino)-5-hydroxy-2H-pyrazolo[4,3-d]pyrimidin-2-yl)methyl)-3,5-dimethoxybenzyl)(methyl)amino)butyl)stearamide; and
(6Z,9Z,28Z,31Z)-heptatriaconta-6,9,28,31-tetraen-19-yl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate;
   or a pharmaceutically acceptable salt thereof.

In some embodiments, the invention provides a composition comprising one or more S. pneumoniae antigens and (N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide, or a pharmaceutically acceptable salt thereof.

In some embodiments, the invention provides a composition comprising one or more S. pneumoniae antigens and (N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide, or a pharmaceutically acceptable salt thereof.

In some embodiments, the invention provides a composition comprising one or more S. pneumoniae antigens and (S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide, or a pharmaceutically acceptable salt thereof.

In some embodiments, the invention provides a composition comprising one or more S. pneumoniae antigens and (S)-1-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)octadecan-1-one, or a pharmaceutically acceptable salt thereof.

In some embodiments, the invention provides a composition comprising one or more S. pneumoniae antigens and N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide, or a pharmaceutically acceptable salt thereof.

In some embodiments, the invention provides a composition comprising one or more S. pneumoniae antigens and N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)tetradecanamide, or a pharmaceutically acceptable salt thereof.

In some embodiments, the invention provides a composition comprising one or more S. pneumoniae antigens and N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)oleamide, or a pharmaceutically acceptable salt thereof.

In some embodiments, the invention provides a composition comprising one or more S. pneumoniae antigens and (9Z,12Z)—N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)octadeca-9,12-dienamide, or a pharmaceutically acceptable salt thereof.

In some embodiments, the invention provides a composition comprising one or more S. pneumoniae antigens and N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide, or a pharmaceutically acceptable salt thereof.

In some embodiments, the invention provides a composition comprising one or more S. pneumoniae antigens and N-(5-(4-(4-((5-amino-7-(butylamino)-2H-pyrazolo[4,3-d]pyrimidin-2-yl)methyl)-3-methoxyphenyl)piperidin-1-yl)-5-oxopentyl)stearamide, or a pharmaceutically acceptable salt thereof.

In some embodiments, the invention provides a composition comprising one or more S. pneumoniae antigens and N-(5-(3-(4-((5-amino-7-(butylamino)-2H-pyrazolo[4,3-d]pyrimidin-2-yl)methyl)-3-methoxyphenyl)azetidin-1-yl)-5-oxopentyl)stearamide, or a pharmaceutically acceptable salt thereof.

In some embodiments, the invention provides a composition comprising one or more S. pneumoniae antigens and 1-(4-((5-amino-7-(butylamino)-2H-pyrazolo[4,3-d]pyrimidin-2-yl)methyl)-3-methoxyphenyl)-N-(3-stearamidopropyl)piperidine-4-carboxamide, or a pharmaceutically acceptable salt thereof.

In some embodiments, the invention provides a composition comprising one or more S. pneumoniae antigens and (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide, or a pharmaceutically acceptable salt thereof.

In some embodiments, the invention provides a composition comprising one or more S. pneumoniae antigens and (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide, or a pharmaceutically acceptable salt thereof.

In some embodiments, the invention provides a composition comprising one or more S. pneumoniae antigens and N-(3-(4-(4-((5-amino-7-(butylamino)-2H-pyrazolo[4,3-d]pyrimidin-2-yl)methyl)-3-methoxyphenyl)piperazin-1-yl)-3-oxopropyl)stearamide, or a pharmaceutically acceptable salt thereof.

In some embodiments, the invention provides a composition comprising one or more S. pneumoniae antigens and N-(7-(4-(4-((5-amino-7-(butylamino)-2H-pyrazolo[4,3-d]pyrimidin-2-yl)methyl)-3-methoxyphenyl)piperazin-1-yl)-7-oxoheptyl)stearamide, or a pharmaceutically acceptable salt thereof.

In some embodiments, the invention provides a composition comprising one or more S. pneumoniae antigens and N-(3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)cyclobutyl)stearamide, or a pharmaceutically acceptable salt thereof.

In some embodiments, the invention provides a composition comprising one or more S. pneumoniae antigens and N-(5-(4-(4-((5-amino-7-(butylamino)-2H-pyrazolo[4,3-d]pyrimidin-2-yl)methyl)-3-methoxyphenyl)piperazin-1-yl)-4,4-dimethyl-5-oxopentyl)stearamide, or a pharmaceutically acceptable salt thereof.

In some embodiments, the invention provides a composition comprising one or more S. pneumoniae antigens and N-(6-(4-(4-((5-amino-7-(butylamino)-2H-pyrazolo[4,3-d]pyrimidin-2-yl)methyl)-3-methoxyphenyl)piperazin-1-yl)-2-methyl-6-oxohexan-2-yl)stearamide, or a pharmaceutically acceptable salt thereof.

In some embodiments, the invention provides a composition comprising one or more S. pneumoniae antigens and 1-(4-(4-((5-amino-7-(butylamino)-2H-pyrazolo[4,3-d]pyrimidin-2-yl)methyl)-3-methoxyphenyl)piperazin-1-yl)-5-(octadecyloxy)pentan-1-one, or a pharmaceutically acceptable salt thereof.

In some embodiments, the invention provides a composition comprising one or more S. pneumoniae antigens and 1-(4-(4-((5-amino-7-(butylamino)-2H-pyrazolo[4,3-d]pyrimidin-2-yl)methyl)-3-methoxyphenyl)piperazin-1-yl)-5-(octadecylamino)pentan-1-one, or a pharmaceutically acceptable salt thereof.

In some embodiments, the invention provides a composition comprising one or more S. pneumoniae antigens and N-(5-(4-(4-((5-amino-7-(butylamino)-3-methyl-2H-pyrazolo[4,3-d]pyrimidin-2-yl)methyl)-3-methoxyphenyl)piperazin-1-yl)-5-oxopentyl)stearamide, or a pharmaceutically acceptable salt thereof.

In some embodiments, the invention provides a composition comprising one or more S. pneumoniae antigens and (9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide, or a pharmaceutically acceptable salt thereof.

In some embodiments, the invention provides a composition comprising one or more S. pneumoniae antigens and N-(4-((4-((5-amino-7-(butylamino)-2H-pyrazolo[4,3-d]pyrimidin-2-yl)methyl)-3,5-dimethoxybenzyl)(methyl)amino)butyl) tetradecanamide, or a pharmaceutically acceptable salt thereof.

In some embodiments, the invention provides a composition comprising one or more S. pneumoniae antigens and N-(4-((4-((5-amino-7-(butylamino)-2H-pyrazolo[4,3-d]pyrimidin-2-yl)methyl)-3,5-dimethoxybenzyl)(methyl)amino)butyl)oleamide, or a pharmaceutically acceptable salt thereof.

In some embodiments, the invention provides a composition comprising one or more S. pneumoniae antigens and N-(4-((4-((5-amino-7-(butylamino)-2H-pyrazolo[4,3-d]pyrimidin-2-yl)methyl)-3,5-dimethoxybenzyl)(methyl)amino)butyl)stearamide, or a pharmaceutically acceptable salt thereof.

In some embodiments, the invention provides a composition comprising one or more S. pneumoniae antigens and N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide, or a pharmaceutically acceptable salt thereof.

In some embodiments, the invention provides a composition comprising one or more S. pneumoniae antigens and (6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)amino)butyl)carbamate, or a pharmaceutically acceptable salt thereof.

In some embodiments, the invention provides a composition comprising one or more S. pneumoniae antigens and 4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide, or a pharmaceutically acceptable salt thereof.

In some embodiments, the invention provides a composition comprising one or more S. pneumoniae antigens and 3-stearamidopropyl 4-(4-((5-amino-7-(butylamino)-2H-pyrazolo[4,3-d]pyrimidin-2-yl)methyl)-3-methoxyphenyl)piperazine-1-carboxylate, or a pharmaceutically acceptable salt thereof.

In some embodiments, the invention provides a composition comprising one or more S. pneumoniae antigens and N-(5-(4-(4-((5-amino-7-(butylamino)-2H-pyrazolo[4,3-d]pyrimidin-2-yl)methyl)-3-methoxyphenyl)piperazin-1-yl)-6,6,6-trifluorohexyl)stearamide, or a pharmaceutically acceptable salt thereof.

In some embodiments, the invention provides a composition comprising one or more S. pneumoniae antigens and N-(4-((4-((7-(butylamino)-5-hydroxy-2H-pyrazolo[4,3-d]pyrimidin-2-yl)methyl)-3,5-dimethoxybenzyl)(methyl)amino)butyl)stearamide, or a pharmaceutically acceptable salt thereof.

In some embodiments, the invention provides a composition comprising one or more S. pneumoniae antigens and (6Z,9Z,28Z,31Z)-heptatriaconta-6,9,28,31-tetraen-19-yl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate, or a pharmaceutically acceptable salt thereof.

### Adjuvants

Adjuvant-based approaches are being investigated to enhance vaccine immunogenicity and help address residual unmet medical need, particularly for populations with increased susceptibility to infectious disease (i.e., immunocompromised, elderly, or pediatric populations).

For example, while invasive pneumococcal disease (IPD) incidence has dramatically declined in both children and adults following the introduction of pneumococcal conjugate vaccines, residual disease caused by persistent vaccine-type serotypes remains. Further, serotype-specific immune responses vary and the introduction of expanded valency PCVs has resulted in an overall decrease in serotype-specific immune responses. Adjuvant-based strategies to enhance immunogenicity of PCVs may increase vaccine efficacy for challenging serotypes, provide more durable immune responses, or allow for reduction in dosing schedule.

Preclinical data indicates that a TLR7/8 agonist can increase the immunogenicity of PCVs. Dowling et al. have reported both accelerated and enhanced serotype specific antibody responses, including both binding antibody titers and functional opsonophagocytic killing, in neonatal and infant rhesus monkeys immunized with PCV13 plus the TLR7/8 agonist 3M-052 compared to PCV13 alone (PCV13 contains alum adjuvant). The addition of the TLR7/8 agonist to PCV13 also induced Th1-polarized CRM197-specific CD4 T cells and early life S. pneumonia antigen-specific B cells in infant rhesus monkeys and enhanced type II IFN and Th1 polarizing cytokine production after in vitro stimulation of human neonatal cord blood (JCI Insight 2017: e91020).

### Adjuvant Formulations

The present disclosure provides an adjuvant formulation that comprises 1) one or more compounds of the invention or pharmaceutically acceptable salt(s) thereof; 2) SPAN-85; 3) PS-20, and 4) squalene.

The present disclosure provides an adjuvant formulation that comprises 1) a compound of the invention or a pharmaceutically acceptable salt thereof; 2) SPAN-85; 3) PS-20 or PS-80, and 4) squalene.

The present disclosure provides an adjuvant formulation that comprises 1) a compound of the invention or a pharmaceutically acceptable salt thereof; 2) SPAN-85; 3) PS-20, and 4) squalene.

### General Methods of Making SNE Formulations

Generally, SNEs may be formed, for example, by initially combining and mixing components together. Once mixed and blended, an aqueous buffer is added and mixed with initial compound components to form a blended emulsion mixture. In some embodiments, the blended emulsion components are first subjected to course homogenization followed by fine homogenization. Then, the resulting formulation is subjected to a final filtration step and stored at 4° C. The solution may include one or more compounds; one or more sorbitan based surfactants (e.g., PS-20; PS-80; SPAN-85); and one or more terpenes (e.g., squalene) at specific molar ratios.

Alternatively, a process of preparing an SNE of the invention consists of 4 primary steps: 1) solution preparation of a component mixture including functional and non-functional ingredients and an aqueous buffer; 2) SNE formation by means of split stream mixing; 3) ultra-filtration; and 4) filtration.

Generally, components are dissolved in ethanol before being sterile filtered to form a mixture. Several aqueous buffers are also prepared. The mixture and buffer streams are then combined using a T-tube or Y mixer and then, immediately after exit, are diluted and mixed with an aqueous buffer to form an SNE intermediate. An SNE intermediate is then subjected to ultra-filtration or dialysis to both concentrate the material as well as exchange the material against a suitable buffer to remove residual ethanol. After the diafiltration, there is a final concentration step performed in order to achieve a final target concentration. SNE bulk is then filtered with a sterilizing filter.

### SNE Formulations

In some embodiments, a formulation is provided wherein the SNE comprises: 1) about 0-75 mol % of a compound of Formula I, Ia, II, IIa, III, IIIa, IV and IVa or a pharmaceutically acceptable salt thereof; 2) about 0-30 mol % of SPAN-85; 3) about 0-30 mol % of PS-20 or PS-80; and 4) 25-85 mol % of squalene.

In some embodiments, a formulation is provided wherein the SNE comprises: 1) about 0-50 mol % of a compound of Formula I, Ia, II, IIa, III, IIIa, IV and IVa or a pharmaceutically acceptable salt thereof; 2) about 0-10 mol % of SPAN-85; 3) about 0-10 mol % PS-20 or PS-80; and 4) about 50-80 mol % of squalene.

In some embodiments, a formulation is provided wherein the SNE comprises: 1) about 0-24 mol % of a compound of Formula I, Ia, II, IIa, III, IIIa, IV and IVa or a pharmaceutically acceptable salt thereof; 2) about 1-8 mol % of SPAN-85; 3) about 1-8 mol % of PS-20 or PS-80; and 4) about 60-75 mol % of squalene.

In some embodiments, a formulation is provided wherein the SNE comprises: 1) about 10-14 mol % of a compound of Formula I, Ia, II, IIa, III, IIIa, IV and IVa or a pharmaceutically acceptable salt thereof; 2) about 1-4 mol % of SPAN-85; 3) about 1-4 mol % of PS-20 or PS-80; and 4) about 50-80 mol % of squalene.

In some embodiments, a formulation is provided wherein the SNE comprises: 1) about 30-65 mol % of a compound of Formula I, Ia, II, IIa, III, IIIa, IV and IVa or a pharmaceutically acceptable salt thereof; 2) about 5-30 mol % of SPAN-85; 3) about 0.5-4 mol % of PS-20 or PS-80; and 4) about 10-40 mol % of squalene.

In some embodiments, a formulation is provided wherein the SNE comprises: 1) about 55-65 mol % of a compound of Formula I, Ia, II, IIa, III, IIIa, IV and IVa or a pharmaceutically acceptable salt thereof; 2) about 5-15 mol % SPAN-85; 3) about 1-2.5 mol % of PS-20 or PS-80; and 4) about 25-35 mol % of squalene.

In some embodiments, a formulation is provided wherein the SNE comprises: 1) about 13-45 mol % of a compound of Formula I, Ia, II, IIa, III, IIIa, IV and IVa or a pharmaceutically acceptable salt thereof; 2) about 2-4 mol % SPAN-85; 3) about 1.5-3 mol % of PS-20 or PS-80; and 4) about 50-82 mol % of squalene.

In some embodiments, a formulation is provided wherein the SNE comprises: 1) about 13-14 mol % of a compound of Formula I, Ia, II, IIa, III, IIIa, IV and IVa or a pharmaceutically acceptable salt thereof; 2) about 1-2 mol % SPAN-85; 3) about 1-2 mol % PS-20 or PS-80; and 4) about 79-81 mol % of squalene.

In some embodiments, a formulation is provided wherein the SNE comprises: about 1-60 mol % of a compound of Formula I, Ia, II, IIa, III, IIIa, IV and IVa or a pharmaceutically acceptable salt thereof; 2) about 1-4 mol % SPAN-85; 3) about 1-4 mol % of PS-20 or PS-80; and 4) about 32-97 mol % of squalene.

In some embodiments, a formulation is provided wherein the SNE comprises: 1) about 0-45 mol % of a compound of Formula I, Ia, II, IIa, III, IIIa, IV and IVa or a pharmaceutically acceptable salt thereof; 2) about 1-10 mol % of a non-ionic surfactant; and 3) about 50-85 mol % of squalene. In one aspect of this embodiment, the non-ionic surfactant comprises a mixture of SPAN-85 and PS-20 or PS-80.

In some embodiments, a formulation is provided wherein the SNE comprises: 1) about 0-75 mol % of a compound of Formula I, Ia, II, IIa, III, IIIa, IV and IVa or a pharmaceutically acceptable salt thereof; 2) about 0-30 mol % of SPAN-85; 3) about 0-30 mol % of PS-20; and 4) 25-85 mol % of squalene.

In some embodiments, a formulation is provided wherein the SNE comprises: 1) about 0-50 mol % of a compound of Formula I, Ia, II, IIa, III, IIIa, IV and IVa or a pharmaceutically acceptable salt thereof; 2) about 0-10 mol % of SPAN-85; 3) about 0-10 mol % PS-20; and 4) about 50-80 mol % of squalene.

In some embodiments, a formulation is provided wherein the SNE comprises: 1) about 0-24 mol % of a compound of Formula I, Ia, II, IIa, III, IIIa, IV and IVa or a pharmaceutically acceptable salt thereof; 2) about 1-8 mol % of SPAN-85; 3) about 1-8 mol % of PS-20; and 4) about 60-75 mol % of squalene.

In some embodiments, a formulation is provided wherein the SNE comprises: 1) about 10-14 mol % of a compound of Formula I, Ia, II, IIa, III, IIIa, IV and IVa or a pharmaceutically acceptable salt thereof; 2) about 1-4 mol % of SPAN-85; 3) about 1-4 mol % of PS-20; and 4) about 50-80 mol % of squalene.

In some embodiments, a formulation is provided wherein the SNE comprises: 1) about 30-65 mol % of a compound of Formula I, Ia, II, IIa, III, IIIa, IV and IVa or a pharmaceutically acceptable salt thereof; 2) about 5-30 mol % of SPAN-85; 3) about 0.5-4 mol % of PS-20; and 4) about 10-40 mol % of squalene.

In some embodiments, a formulation is provided wherein the SNE comprises: 1) about 55-65 mol % of a compound of Formula I, Ia, II, IIa, III, IIIa, IV and IVa or a pharmaceutically acceptable salt thereof; 2) about 5-15 mol % SPAN-85; 3) about 1-2.5 mol % of PS-20; and 4) about 25-35 mol % of squalene.

In some embodiments, a formulation is provided wherein the SNE comprises: 1) about 13-45 mol % of a compound of Formula I, Ia, II, IIa, III, IIIa, IV and IVa or a pharmaceutically acceptable salt thereof; 2) about 2-4 mol % SPAN-85; 3) about 1.5-3 mol % of PS-20; and 4) about 50-82 mol % of squalene.

In some embodiments, a formulation is provided wherein the SNE comprises: 1) about 13-14 mol % of a compound of Formula I, Ia, II, IIa, III, IIIa, IV and IVa or a pharmaceutically acceptable salt thereof; 2) about 1-2 mol % SPAN-85; 3) about 1-2 mol % PS-20; and 4) about 79-81 mol % of squalene.

In some embodiments, a formulation is provided wherein the SNE comprises: about 1-60 mol % of a compound of Formula I, Ia, II, IIa, III, IIIa, IV and IVa or a pharmaceutically acceptable salt thereof; 2) about 1-4 mol % SPAN-85; 3) about 1-4 mol % of PS-20; and 4) about 32-97 mol % of squalene.

In some embodiments, a formulation is provided wherein the SNE comprises: 1) about 0-45 mol % of a compound of Formula I, Ia, II, IIa, III, IIIa, IV and IVa or a pharmaceutically acceptable salt thereof; 2) about 1-10 mol % of a non-ionic surfactant; and 3) about 50-85 mol % of squalene. In one aspect of this embodiment, the non-ionic surfactant comprises a mixture of SPAN-85 and PS-20.

In some embodiments, the formulations provided above comprise a particular compound selected from:
(N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide (Compound A-1);
(S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide (Compound A-2);
(S)-1-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)octadecan-1-one (Compound A-3);
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide (Compound B-1);
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)tetradecanamide (Compound B-2);
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)oleamide (Compound B-3);
(9Z,12Z)—N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)octadeca-9,12-dienamide (Compound B-4);
N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide (Compound B-5);
N-(5-(4-(4-((5-amino-7-(butylamino)-2H-pyrazolo[4,3-d]pyrimidin-2-yl)methyl)-3-methoxyphenyl)piperidin-1-yl)-5-oxopentyl)stearamide (Compound B-6);
N-(5-(3-(4-((5-amino-7-(butylamino)-2H-pyrazolo[4,3-d]pyrimidin-2-yl)methyl)-3-methoxyphenyl)azetidin-1-yl)-5-oxopentyl)stearamide (Compound B-7);
1-(4-((5-amino-7-(butylamino)-2H-pyrazolo[4,3-d]pyrimidin-2-yl)methyl)-3-methoxyphenyl)-N-(3-stearamidopropyl)piperidine-4-carboxamide (Compound B-8);
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide (Compound B-9);
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide (Compound B-10);
N-(3-(4-(4-((5-amino-7-(butylamino)-2H-pyrazolo[4,3-d]pyrimidin-2-yl)methyl)-3-methoxyphenyl)piperazin-1-yl)-3-oxopropyl)stearamide (Compound B-11);
N-(7-(4-(4-((5-amino-7-(butylamino)-2H-pyrazolo[4,3-d]pyrimidin-2-yl)methyl)-3-methoxyphenyl)piperazin-1-yl)-7-oxoheptyl)stearamide (Compound B-12);
N-(3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)cyclobutyl)stearamide (Compound B-13);
N-(5-(4-(4-((5-amino-7-(butylamino)-2H-pyrazolo[4,3-d]pyrimidin-2-yl)methyl)-3-methoxyphenyl)piperazin-1-yl)-4,4-dimethyl-5-oxopentyl)stearamide (Compound B-14);
N-(6-(4-(4-((5-amino-7-(butylamino)-2H-pyrazolo[4,3-d]pyrimidin-2-yl)methyl)-3-methoxyphenyl)piperazin-1-yl)-2-methyl-6-oxohexan-2-yl)stearamide (Compound B-15);
1-(4-(4-((5-amino-7-(butylamino)-2H-pyrazolo[4,3-d]pyrimidin-2-yl)methyl)-3-methoxyphenyl)piperazin-1-yl)-5-(octadecyloxy)pentan-1-one (Compound B-16);
1-(4-(4-((5-amino-7-(butylamino)-2H-pyrazolo[4,3-d]pyrimidin-2-yl)methyl)-3-methoxyphenyl)piperazin-1-yl)-5-(octadecylamino)pentan-1-one (Compound B-17);
N-(5-(4-(4-((5-amino-7-(butylamino)-3-methyl-2H-pyrazolo[4,3-d]pyrimidin-2-yl)methyl)-3-methoxyphenyl)piperazin-1-yl)-5-oxopentyl)stearamide (Compound B-18);
(9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide (Compound C-1);
N-(4-((4-((5-amino-7-(butylamino)-2H-pyrazolo[4,3-d]pyrimidin-2-yl)methyl)-3,5-dimethoxybenzyl)(methyl)amino)butyl)tetradecanamide (Compound C-2);
N-(4-((4-((5-amino-7-(butylamino)-2H-pyrazolo[4,3-d]pyrimidin-2-yl)methyl)-3,5-dimethoxybenzyl)(methyl)amino)butyl)oleamide (Compound C-3);
N-(4-((4-((5-amino-7-(butylamino)-2H-pyrazolo[4,3-d]pyrimidin-2-yl)methyl)-3,5-dimethoxybenzyl)(methyl)amino)butyl)stearamide (Compound C-4);
N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide (Compound C-5);
(6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)amino)butyl)carbamate (Compound D-1);
4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide (Compound D-2);
3-stearamidopropyl 4-(4-((5-amino-7-(butylamino)-2H-pyrazolo[4,3-d]pyrimidin-2-yl)methyl)-3-methoxyphenyl)piperazine-1-carboxylate (Compound) D-3);
N-(5-(4-(4-((5-amino-7-(butylamino)-2H-pyrazolo[4,3-d]pyrimidin-2-yl)methyl)-3-methoxyphenyl)piperazin-1-yl)-6,6,6-trifluorohexyl)stearamide (Compound D-4);
N-(4-((4-((7-(butylamino)-5-hydroxy-2H-pyrazolo[4,3-d]pyrimidin-2-yl)methyl)-3,5-dimethoxybenzyl)(methyl)amino)butyl)stearamide (Compound D-5); and
(6Z,9Z,28Z,31Z)-heptatriaconta-6,9,28,31-tetraen-19-yl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate (Compound D-6);
   or a pharmaceutically acceptable salt thereof.

In some embodiments of the invention, a formulation is provided wherein the SNE further comprises one or more additional components which are selected from a surfactant, a mixture of surfactants, a phospholipid, a terpene, a terpenoid, a triterpene or a combination thereof.

In some embodiments of the invention, the surfactant includes, but is not limited to: the polyoxyethylene sorbitan esters surfactants (commonly referred to as the Tweens), especially PS-20 and PS-80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span-85, Tween-85 or [2-[(2R,3S,4R)-4-hydroxy-3-[(Z)-octadec-9-enoyl]oxyoxolan-2-yl]-2-[(Z)-octadec-9-enoyl]oxyethyl] (Z)-octadec-9-enoate) and sorbitan monolaurate.

In some embodiments of the invention, mixtures of surfactants are used, e.g., PS-20/Span 85 mixtures. A combination of a polyoxyethylene sorbitan ester such as polyoxyethylene sorbitan monooleate (PS-80) and an octoxynol such as t-octylphenoxypolyethoxyethanol (Triton X-100) are also suitable. Another useful combination comprises laureth 9 plus a polyoxyethylene sorbitan ester and/or an octoxynol.

In some embodiments of the invention, the amounts of surfactants or emulsifiers are: polyoxyethylene sorbitan esters (such as PS-20) 0.01 to 10 mole %, in particular about 1 to 4 mol %; octyl- or nonylphenoxy polyoxyethanols (such as Triton X-100, or other detergents in the Triton series) 0.001 to 10 mol %, in particular about 1 to 4 mol %; w/v, in particular 0.01 to 0.1% w/v; polyoxyethylene ethers (such as laureth 9) 0.1 to 20 mol %, preferably 0.5 to 10 mol % and in particular 1 to 4 mol % or about 10% by mass.

In some embodiments of the invention, the phospholipid is selected from, a natural phospholipid including phosphatidylcholine (PC), phosphatidylethanolamine (PE), and phosphatidylglycerol (PG), phosphatidylserine (PS), phosphatidylinositol (PI), phosphatidic acid (phosphatidate) (PA), dipalmitoylphosphatidylcholine, monoacyl-phosphatidylcholine (lyso PC), 1-palmitoyl-2-oleoyl-sn-glycero-3-phosphocholine (POPC), N-Acyl-PE, a phosphoinositide, and a phosphosphingolipid. Phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS). Fatty acids include C14:0, palmitic acid (C16:0), stearic acid (C18:0), oleic acid (C18:1), linoleic acid (C18:2), linolenic acid (C18:3), and arachidonic acid (C20:4), C20:0, C22:0 and lethicin. In certain embodiments of the invention, the phospholipid is phosphatidylserine, 1,2-Distearoyl-sn-glycero-3-phosphocholine (DSPC), 1,2-dipalmitoleoyl-sn-glycero-3-phosphocholine, 1,2-dimyristoyl-sn-glycero-3-phosphocholine (DMPC), dilauroylphosphatidylcholine (DLPC), 1,2-dieicosenoyl-sn-glycero-3-phosphocholine, or 1,2-dioleoyl-sn-glycero-3-phosphocholine (DOPC).

In some embodiments of the invention, the terpene is selected from a monoterpene, such as geraniol, terpeneol, limonene, myrcene, linalool or pinene. In some embodiments, the formulation comprises a sesquiterpene consisting of humulene, a farnesene, or farnesol; a diterpene such as cafestol, kahweol, cembrene or taxadiene; A triterpene such as squalene or squalante; a tetraterpene such as acyclic lycopene, monocyclic gamma-carotene, or bicyclic alpha- and beta-carotenes; a polyterpene or a norisoprenoids.

### Pneumococcal Vaccine Compositions

Pneumococcal vaccines or compositions are well known (for example PNEUMOVAX® (Merck & Co., Inc., Rahway, NJ, USA)). Pneumococcal conjugate vaccines or compositions have been previously disclosed. See WO2011/100151, WO2019/139692 and WO2020/131763.

Example bacterial capsular polysaccharides from S. pneumoniae are serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38, among others.

### General Method for Making Capsular Polysaccharides

Bacterial capsular polysaccharides, particularly those that have been used as antigens, are suitable for use in the invention and can readily be identified by methods for identifying immunogenic and/or antigenic polysaccharides. Example bacterial capsular polysaccharides from S. pneumoniae are serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38, among others.

As used herein, de-O-acetylated serotype 15B (DeOAc15B) pneumococcal polysaccharide is substantially equivalent to serotype 15C pneumococcal polysaccharide and has a substantially identical NMR spectra (data not shown). As used herein, de-O-acetylated serotype 15B pneumococcal polysaccharide and serotype 15C pneumococcal polysaccharide may each have an O-Acetyl content per repeating unit in the range of 0-5%, or in the range of 0-4%, or in the range of 0-3%, or in the range of 0-2%, or in the range of 0-1%, or in the range of 0-0.5%, or in the range of 0-0.1%, or no O-acetyl content. In a report by Spencer B. L., et al., pneumococcal polysaccharide 15C may be slightly O-acetylated (Spencer, B. L. et al., Clin. Vac. Immuno. (2017) 24(8): 1-13). Thus, in any of the embodiments of the multivalent immunogenic compositions described herein, de-O-acetylated serotype 15B (DeOAc15B) can be used in place of serotype 15C. Processes for de-O-acetylation are known in the art, for example as described in Rajam et al., Clinical and Vaccine Immunology, 2007, 14(9):1223-1227.

Polysaccharides can be purified by known techniques. The invention is not limited to polysaccharides purified from natural sources, however, and the polysaccharides may be obtained by other methods, such as total or partial synthesis. Capsular polysaccharides from S. pneumoniae can be prepared by standard techniques known to those skilled in the art. For example, polysaccharides can be isolated from bacteria and may be sized to some degree by known methods (see, e.g., European Patent Nos. EP497524 and EP497525); and preferably by microfluidization accomplished using a homogenizer or by chemical hydrolysis. S. pneumoniae strains corresponding to each polysaccharide serotype may be grown in a soy-based medium. The individual polysaccharides may then be purified through standard steps including centrifugation, precipitation, and ultrafiltration. See, e.g., U.S. Patent Application Publication No. 2008/0286838 and U.S. Pat. No. 5,847,112. Polysaccharides can be sized in order to reduce viscosity and/or to improve filterability and the lot-to-lot consistency of subsequent conjugated products.

Purified polysaccharides can be chemically activated to introduce functionalities capable of reacting with a carrier protein using standard techniques. The chemical activation of polysaccharides and subsequent conjugation to a carrier protein are achieved by means described in U.S. Pat. Nos. 4,365,170, 4,673,574 and 4,902,506. Briefly, the pneumococcal polysaccharide is reacted with a periodate-based oxidizing agent such as sodium periodate, potassium periodate, or periodic acid resulting in oxidative cleavage of vicinal hydroxyl groups to generate reactive aldehyde groups. Suitable molar equivalents of periodate (e.g., sodium periodate, sodium metaperiodate and the like) include 0.05 to 0.5 molar equivalents (molar ratio of periodate to polysaccharide repeat unit) or 0.1 to 0.5 molar equivalents. The periodate reaction can be varied from 30 minutes to 24 hours depending on the diol conformation (e.g., acyclic diols, cis diols, trans diols), which controls accessibility of the reactive hydroxyl groups to the sodium periodate.

The term “periodate” includes both periodate and periodic acid; the term also includes both metaperiodate (IO⁴⁻) and orthoperiodate (IO⁶⁻) and includes the various salts of periodate (e.g., sodium periodate and potassium periodate). Capsular polysaccharide may be oxidized in the presence of metaperiodate, or in the presence of sodium periodate (NaIO₄). Further, capsular polysaccharide may be oxidized in the presence of orthoperiodate, or in the presence of periodic acid.

Purified polysaccharides can also be connected to a linker. Once activated or connected to a linker, each capsular polysaccharide may be separately conjugated to a carrier protein to form a glycoconjugate. The polysaccharide conjugates may be prepared by known coupling techniques.

Polysaccharide can be coupled to a linker to form a polysaccharide-linker intermediate in which the free terminus of the linker is an ester group. The linker is therefore one in which at least one terminus is an ester group. The other terminus is selected so that it can react with the polysaccharide to form the polysaccharide-linker intermediate.

Polysaccharide can be coupled to a linker using a primary amine group in the polysaccharide. In this case, the linker typically has an ester group at both termini. This allows the coupling to take place by reacting one of the ester groups with the primary amine group in the polysaccharide by nucleophilic acyl substitution. The reaction results in a polysaccharide-linker intermediate in which the polysaccharide is coupled to the linker via an amide linkage. The linker is therefore a bifunctional linker that provides a first ester group for reacting with the primary amine group in the polysaccharide and a second ester group for reacting with the primary amine group in the carrier molecule. A typical linker is adipic acid N-hydroxysuccinimide diester (SIDEA).

The coupling can also take place indirectly, i.e., with an additional linker that is used to derivatize the polysaccharide prior to coupling to the linker.

Polysaccharide can be coupled to the additional linker using a carbonyl group at the reducing terminus of the polysaccharide. This coupling comprises two steps: (al) reacting the carbonyl group with the additional linker; and (a2) reacting the free terminus of the additional linker with the linker. In these embodiments, the additional linker typically has a primary amine group at both termini, thereby allowing step (a1) to take place by reacting one of the primary amine groups with the carbonyl group in the polysaccharide by reductive amination. A primary amine group is used that is reactive with the carbonyl group in the polysaccharide. Hydrazide or hydroxylamino groups are suitable. The same primary amine group is typically present at both termini of the additional linker which allows for the possibility of polysaccharide (Ps)-Ps coupling. The reaction results in a polysaccharide-additional linker intermediate in which the polysaccharide is coupled to the additional linker via a C—N linkage.

Polysaccharide can be coupled to the additional linker using a different group in the polysaccharide, particularly a carboxyl group. This coupling comprises two steps: (a1) reacting the group with the additional linker; and (a2) reacting the free terminus of the additional linker with the linker. In this case, the additional linker typically has a primary amine group at both termini, thereby allowing step (a1) to take place by reacting one of the primary amine groups with the carboxyl group in the polysaccharide by EDAC activation. A primary amine group is used that is reactive with the EDAC-activated carboxyl group in the polysaccharide. A hydrazide group is suitable. The same primary amine group is typically present at both termini of the additional linker. The reaction results in a polysaccharide-additional linker intermediate in which the polysaccharide is coupled to the additional linker via an amide linkage.

### Carrier Protein

In a particular embodiment of the present invention, CRM197 is used as the carrier protein. CRM197 is a non-toxic variant (i.e., toxoid) of diphtheria toxin. CRM197 may be isolated from cultures of Corynebacterium diphtheria strain C7 (β197) grown in casamino acids and yeast extract-based medium. Further, CRM197 may be prepared recombinantly in accordance with the methods described in U.S. Pat. No. 5,614,382. Typically, CRM₁₉₇ is purified through a combination of ultrafiltration, ammonium sulfate precipitation, and ion-exchange chromatography. In some embodiments, CRM197 is prepared in Pseudomonas fluorescens using Pfenex Expression Technology™ (Pfenex Inc., San Diego, CA).

Other suitable carrier proteins include additional inactivated bacterial toxins such as DT (Diphtheria toxoid), TT (tetanus toxoid) or fragment C of TT, pertussis toxoid, cholera toxoid (e.g., as described in International Patent Application Publication No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal surface adhesin protein (PsaA), C5a peptidase from Group A or Group B streptococcus, or Haemophilus influenzae protein D, pneumococcal pneumolysin (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et al., 1998, Immunol Lett 64:9) can also be used as carrier proteins.

Where multivalent vaccines are used, a second carrier can be used for one or more of the antigens in a multivalent vaccine. The second carrier protein is preferably a protein that is non-toxic and non-reactogenic and obtainable in sufficient amount and purity. The second carrier protein is also conjugated or joined with an antigen, e.g., a S. pneumoniae polysaccharide to enhance immunogenicity of the antigen. Carrier proteins should be amenable to standard conjugation procedures. Each capsular polysaccharide not conjugated to a first carrier protein may be conjugated to the same second carrier protein (e.g., each capsular polysaccharide molecule being conjugated to a single carrier protein). Capsular polysaccharides not conjugated to a first carrier protein may be conjugated to two or more carrier proteins (each capsular polysaccharide molecule being conjugated to a single carrier protein). In such embodiments, each capsular polysaccharide of the same serotype is typically conjugated to the same carrier protein. Other DT mutants can be used as the second carrier protein, such as CRM176, CRM228, CRM45 (Uchida et al., 1973, J Biol Chem 218:3838-3844); CRM9, CRM45 CRM102, CRM103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711.

### Conjugation by Reductive Amination

Covalent coupling of polysaccharide to carrier protein can be performed via reductive amination in which an amine-reactive moiety on the polysaccharide is directly coupled to primary amine groups (mainly lysine residues) of the protein. As is well known, a reductive amination reaction proceeds via a two-step mechanism. First, a Schiff base intermediate, of formula R—CH═N—R′, is formed by reaction of an aldehyde group on molecule 1 (R—CHO) with a primary amine group (R′—NH2) on molecule 2. In the second step, the Schiff base is reduced to form an amino compound of formula R—CH2-NH—R′. While many reducing agents are capable of being utilized, most often a highly selective reducing agent such as sodium cyanoborohydride (NaCNBH₃) is employed since such reagents will specifically reduce only the imine function of the Schiff base.

Since all the polysaccharides have an aldehyde function at the end of the chain (terminal aldehyde function), the conjugation methods comprising a reductive amination of the polysaccharide can be applied very generally and, when there is no other aldehyde function in the repeating unit (intrachain aldehyde function), such methods make it possible to obtain conjugates in which a polysaccharide molecule is coupled to a single molecule of carrier protein.

A typical reducing agent is cyanoborohydride salt such as sodium cyanoborohydride. The imine-selective reducing agent typically employed is sodium cyanoborohydride, although other cyanoborohydride salts can be used including potassium cyanoborohydride. Differences in starting cyanide levels in sodium cyanoborohydride reagent lots and residual cyanide in the conjugation reaction can lead to inconsistent conjugation performance, resulting in variable product attributes, such as conjugate size and conjugate Ps-to-CRM₁₉₇ ratio. By controlling and/or reducing the free cyanide levels in the final reaction product, conjugation variability can be reduced.

Residual unreacted aldehydes on the polysaccharide are optionally reduced with the addition of a strong reducing agent, such as sodium borohydride. Generally, use of a strong reducing agent is preferred. However, for some polysaccharides, it is preferred to avoid this step. For example, S. pneumoniae serotype 5 contains a ketone group that may react readily with a strong reductant. In this case, it is preferable to bypass the reduction step to protect the antigenic structure of the polysaccharide.

Following conjugation, the polysaccharide-protein conjugates are purified to remove excess conjugation reagents as well as residual free protein and free polysaccharide by one or more of any techniques well known to the skilled artisan, including concentration/diafiltration operations, ultrafiltration, precipitation/elution, column chromatography, and depth filtration. See, e.g., U.S. Pat. No. 6,146,902. In one embodiment, the purifying step is by ultrafiltration. Pneumococcal Conjugate Compositions

The present invention provides pneumococcal conjugate compositions comprising, consisting essentially of, or alternatively, consisting of any of the polysaccharide serotype-conjugate combinations described herein and/or known in the art together with a pharmaceutically acceptable carrier and the SNEs described above. The compositions may comprise, consist essentially of, or consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 or more distinct polysaccharide-carrier protein conjugates, wherein each of the conjugates contains a different capsular polysaccharide conjugated to a carrier protein (the carrier protein may be one particular carrier protein or 2 or more different carrier proteins). In an embodiment, the capsular polysaccharides are selected from at least one of serotypes 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A or 20B), 22F, 23A, 23B, 23F, 24F, 33F, 35B, 35F, or 38, or more of S. pneumoniae. In another embodiment the serotypes are conjugated to the carrier protein CRM197.

In some embodiments the polysaccharides, contained in the polysaccharide-carrier protein conjugates, are selected from polysaccharides that are selected from a group of serotypes which consist of serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and/or 20B), 22F, 23A, 23B, 23F, 24F, 33F, 35B, 35F, or 38. In other embodiments, the group of serotypes consist of 4, 6B, 9V, 14, 18C, 19F and 23F. In other embodiments, the group of serotypes consist of 1, 3, 4, 5, 6A, 6B, 7F, 9V, 14, 18C, 19A, 19F and 23F. In other embodiments, the group of serotypes consist of 1, 3, 4, 5, 6A, 6B, 7F, 9V, 14, 18C, 19A, 19F, 22F, 23F and 33F. In other embodiments, the group of serotypes consist of 1, 3, 4, 5, 6A, 6B, 7F, 8, 9V, 10A, 11A, 12F, 14, 15B, 18C, 19A, 19F, 22F, 23F and 33F. In other embodiments, the group of serotypes consist of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In other embodiments, the group of serotypes consist of 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B. In other embodiments, the group of serotypes consist of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In other embodiments, the group of serotypes consist of 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B. In other embodiments, the group of serotypes consist of 1, 3, 4, 5, 6A, 6B, 7F, 8, 9V, 10A, 11A, 12F, 14, 15A, de-O-acetylated-15B, 16F, 18C, 19A, 19F, 22F, 23A, 23B, 23F, 24F, 33F and 35B. In other embodiments, the group of serotypes consist of 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

In some embodiments, the carrier protein, contained in the polysaccharide-carrier protein conjugates, are selected from OMPC, PhtD, pLys, DT (Diphtheria toxoid), TT (tetanus toxoid), fragment C of TT, pertussis toxoid, cholera toxoid and CRM197. In other embodiments, the carrier protein is CRM197.

### Administration/Dosage

The compositions and formulations of the invention can be used to protect or treat a human susceptible to infection, e.g., a pneumococcal infection, by means of administering the composition to a patient in need thereof, formulation or vaccine via a systemic or mucosal route. In one embodiment, the invention provides a method of inducing an immune response to a S. pneumoniae capsular polysaccharide conjugate, comprising administering to a human an immunologically effective amount of an immunogenic composition of the invention. In another embodiment, the invention provides a method of vaccinating a human against a pneumococcal infection, comprising the step of administering to the human an immunologically effective amount of an immunogenic composition, formulation or vaccine of the present invention.

Optimal amounts of components for a particular composition, formulation or vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

The methods of the invention can be used for the prevention and/or reduction of primary clinical syndromes caused S. pneumonia, including both invasive infections (meningitis, pneumonia, and bacteremia), and noninvasive infections (acute otitis media, and sinusitis).

Administration of the compositions, formulations or vaccines of the invention can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage).

The amount of conjugate in each vaccine (or composition or formulation) dose may be selected as an amount that induces an immunoprotective response without significant, adverse effects. Such amount can vary depending upon the pneumococcal serotype. Generally, for polysaccharide-based conjugates, each dose will comprise 0.1 to 100 μg of each polysaccharide, particularly 0.1 to 10 μg, and more particularly 1 to 5 μg. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 μg.

According to any of the methods of the invention and in one embodiment, the subject is human. In certain embodiments, the human patient is an infant (less than 1 year of age), toddler (approximately 12 to 24 months), or young child (approximately 2 to 5 years). In other embodiments, the human patient is an elderly patient (>65 years). The compositions of this invention are also suitable for use with older children, adolescents and adults (e.g., aged 18 to 45 years or 18 to 65 years).

In one embodiment of the methods of the invention, a composition, formulation or vaccine of the present invention is administered as a single inoculation. In another embodiment, the composition, formulation or vaccine is administered twice, three times or four times or more, adequately spaced apart. For example, the composition, formulation or vaccine may be administered at 1-, 2-, 3-, 4-, 5-, or 6-month intervals or any combination thereof. The immunization schedule can follow that designated for pneumococcal vaccines. For example, the routine schedule for infants and toddlers against invasive disease caused by S. pneumoniae is 2-, 4-, 6- and 12-15-months of age. Thus, in a preferred embodiment, the composition, formulation or vaccine is administered as a 4-dose series at 2-, 4-, 6-, and 12-15-months of age.

The compositions, formulations or vaccines of this invention may also include one or more proteins from S. pneumoniae. Examples of S. pneumoniae proteins suitable for inclusion include those identified in International Patent Application Publication Nos. WO 02/083855 and WO 02/053761.

### Formulations

In the embodiments of the formulations below, a composition refers to a pharmaceutical composition and/or an immunogenic composition and/or a single dose vaccine composition. In some embodiments, a composition is provided that includes one or more compounds as described herein and one or more S. pneumoniae polysaccharides or one or more S. pneumoniae polysaccharide-carrier protein conjugates. In some embodiments, a composition is provided that includes a compound as described herein and S. pneumoniae polysaccharides or S. pneumoniae polysaccharide-carrier protein conjugates containing at least 7, or at least 10, or at least 13 or at least 15, or at least 20, or at least 24, or at least 27, or at least 30 or more S. pneumoniae serotypes. In some embodiments, a composition is provided that includes a compound, as described herein, and S. pneumoniae polysaccharides or S. pneumoniae polysaccharide-carrier protein conjugates containing 20, 21, 22, 23, 24, 25, 26, 27, 28, 29, 30, 31, 32, 33, 34, 35, 36, 37, 38, 39 or 40 or more S. pneumoniae serotypes. In some embodiments, a composition is provided that includes a compound, as described herein, and S. pneumoniae polysaccharide-carrier protein conjugates containing S. pneumoniae serotypes consisting of serotypes 4, 6B, 9V, 14, 18C, 19F and 23F. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 9V, 14, 18C, 19A, 19F and 23F. In some embodiments, a composition is provided that includes a compound, a described herein, and S. pneumoniae polysaccharide-carrier protein conjugates containing S. pneumoniae serotypes consisting of serotypes 1, 3, 4, 5, 6A, 6B, 7F, 9V, 14, 18C, 19A, 19F, 22F, 23F and 33F. In some embodiments, a composition is provided that includes a compound, as described herein and S. pneumoniae polysaccharide-carrier protein conjugates containing S. pneumoniae serotypes consisting of serotypes 1, 3, 4, 5, 6A, 6B, 7F, 8, 9V, 10A, 11A, 12F, 14, 15B, 18C, 19A, 19F, 22F, 23F and 33F. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 9V, 10A, 12F, 14, 15A, 15B, 18C, 19A, 19F, 22F, 23B, 23F, 24F, 33F and 35B. In some embodiments, a composition is provided that includes a compound, as described herein and S. pneumoniae polysaccharide-carrier protein conjugates containing S. pneumoniae serotypes consisting of serotypes 1, 3, 4, 5, 6A, 6B, 7F, 9V, 10A, 12F, 14, 15A, de-O-acetyl-15B, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 9V, 10A, 12F, 14, 15A, 15C, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 9V, 10A, 12F, 14, 15A, 15B, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 9V, 10A, 12F, 14, 15A, de-O-acetyl-15B, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 9V, 10A, 12F, 14, 15A, 15C, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 8, 9V, 10A, 11A, 12F, 14, 15A, de-O-acetyl-15B, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 8, 9V, 10A, 11A, 12F, 14, 15A, 15B, 16F, 18C, 19A, 19F, 22F, 23A, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 8, 9V, 10A, 11A, 12F, 14, 15A, de-O-acetyl-15B, 16F, 18C, 19A, 19F, 22F, 23A, 23B, 23F, 24F, 33F and 35B. In some embodiments, a composition is provided that includes a compound, as described herein, and S. pneumoniae polysaccharide-carrier protein conjugates containing S. pneumoniae serotypes consisting of serotypes 1, 3, 4, 5, 6A, 6B, 7F, 8, 9V, 10A, 11A, 12F, 14, 15A, 15C, 16F, 18C, 19A, 19F, 22F, 23A, 23B, 23F, 24F, 33F and 35B.

In some embodiments, the compositions described above contain an additional 1, 2, 3, 4 or 5 S. pneumoniae polysaccharide-carrier protein conjugates containing other S. pneumoniae serotypes known in the art.

In some embodiments, the compositions described above contain an additional 1, 2, 3, 4 or 5 S. pneumoniae polysaccharide-carrier protein conjugates containing other S. pneumoniae serotypes selected from the group 7C, 35F, 21, 34, 16F, 9N, 31, 17F, 23A, and 27.

In some embodiments, the compositions described above contain an additional 1 or 2 S. pneumoniae polysaccharide-carrier protein conjugates containing other S. pneumoniae serotypes selected from the group 7C, 35F, 21 and 34.

In some embodiments, the compositions described above contain an additional 1 or 2 S. pneumoniae polysaccharide-carrier protein conjugates containing other S. pneumoniae serotypes selected from the group 16F, 9N, 31 and 17F.

In some embodiments, the compositions described above contain an additional 1 or 2 S. pneumoniae polysaccharide-carrier protein conjugates containing other S. pneumoniae serotypes selected from the group 23A and 27.

In some embodiments, the compositions described above comprise polysaccharide-carrier protein conjugates wherein the protein is the carrier protein CRM197.

In some embodiments, a composition is provided that includes about 1 μg to about 200 mg of a compound of the invention, or a pharmaceutically acceptable salt thereof, and at least one S. pneumoniae polysaccharide-carrier protein conjugate, wherein each of the conjugates is present in a concentration of about 0.01 μg to about 100 μg per 0.5 mL of the vaccine formulation.

In some embodiments, a composition is provided that includes about 0.02 μg to about 40 mg of a compound of the invention, or a pharmaceutically acceptable salt thereof, and at least one S. pneumoniae polysaccharide-carrier protein conjugate, wherein each of the conjugates is present in a concentration of about 0.002 μg to about 20 μg per 0.1 mL of the vaccine formulation.

In some embodiments, a composition is provided that includes about 50 μg to about 2.1 mg of a compound of the invention, or a pharmaceutically acceptable salt thereof, and at least one S. pneumoniae polysaccharide-carrier protein conjugate, wherein each of the conjugates is present in a concentration of about 0.002 μg to about 20 μg per 0.5 mL of the vaccine formulation.

In some embodiments, a composition is provided that includes about 50 μg to about 10 mg of a compound of the invention, or a pharmaceutically acceptable salt thereof, and at least one S. pneumoniae polysaccharide-carrier protein conjugate, wherein each of the conjugates is present in a concentration of about 0.002 μg to about 20 μg per 0.5 mL of the vaccine formulation.

In some embodiments, a composition is provided that includes about 50 μg to about 10 mg of a compound of the invention, or a pharmaceutically acceptable salt thereof, and at least one S. pneumoniae polysaccharide-carrier protein conjugate, wherein each of the conjugates is present in a concentration of about 0.002 μg to about 20 μg per 0.5 mL of the vaccine formulation.

In some embodiments, a composition is provided that includes about 1 μg to about 200 mg of a compound of the invention, or a pharmaceutically acceptable salt thereof, and at least one S. pneumoniae polysaccharide-carrier protein conjugate, wherein each of the conjugates is present in a concentration of about 0.01 μg to about 100 μg per 0.5 mL of the vaccine formulation prepared as a co-lyophilized formulation.

In some embodiments, a composition is provided that includes about 1 μg to about 200 mg of a compound of the invention, or a pharmaceutically acceptable salt thereof, 1 μg to about 1 mg of Aluminum in the form of APA and at least one S. pneumoniae polysaccharide-carrier protein conjugate, wherein each of the conjugates is present in a concentration of about 0.01 μg to about 100 μg per 0.5 mL of the vaccine formulation prepared as a co-lyophilized formulation.

In some embodiments, a composition, as highlighted in the various embodiments above, is provided that includes about 0.05 μg to about 200 mg of a compound of the invention, or a pharmaceutically acceptable salt thereof, and further includes SPAN-85, PS-80 or PS-20 and squalene. In some embodiments, a composition, as highlighted in the various embodiments above, is provided that includes about 0.05 μg to about 200 mg of a compound of the invention, or a pharmaceutically acceptable salt thereof, and further includes SPAN-85, PS-20 and squalene. In another embodiment, the compound is Compound A-1, or A-2, or A-3. In another embodiment, the compound is Compound B-1, or B-2, or B-3, or B-4, or B-5, or B-6, or B-7, or B-8, or B-9, or B-10, or B-11, or B-12, or B-13, or B-14, or B-15, or B-16, or B-17, or B-18. In another embodiment, the compound is Compound C-1, or C-2, or C-3, or C-4, or C-5. In another embodiment, the compound is Compound D-1, or D-2, or D-3, or D-4, or D-5, or D-6.

Compositions of the invention may be administered subcutaneously, topically, orally, on the mucosa, intravenously, or intramuscularly. The compositions are administered in an amount sufficient to elicit a protective response. Compositions can be administered by various routes, for example, orally, parenterally, subcutaneously, on the mucosa, or intramuscularly. The dose administered may vary depending on the general condition, sex, weight and age of the patient, and the route of administration.

Compositions of the invention, as highlighted in the various embodiments above, may be referred to as immunogenic compositions.

Compositions of the invention, as highlighted in the various embodiments above may be referred to as vaccines or vaccine compositions.

In each of the embodiments described above the composition further comprises Streptococcus pneumoniae polysaccharide-carrier protein conjugates.

The invention provides methods of treating or preventing pneumococcal diseases by administration of the compositions described above.

The invention provides the use of the compositions described above for treating or preventing pneumococcal diseases.

All publications mentioned herein are incorporated by-reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be used by one skilled in the art without departing from the scope or spirit of the invention as defined in the appended claims.

The following examples illustrate, but do not limit the invention.

### Example 1

### General Methods for Making the Compounds of Formula I, Ia, II, IIa, III, IIIa, IV and IVa

The compounds may be prepared from known or readily prepared starting materials, following methods known to one skilled in the art of organic synthesis. Representative methods useful for making the compounds are set forth in the Examples below. Alternative synthetic pathways and analogous structures will be apparent to those skilled in the art of organic synthesis.

One skilled in the art of organic synthesis will recognize that the synthesis of multicyclic and/or heterocyclic cores contained in compounds of the invention may require protection of certain functional groups (i.e., derivatization for the purpose of chemical compatibility with a particular reaction condition). Suitable protecting groups for the various functional groups of these compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the multicyclic heterocycle cores of the compounds of the invention may be more desirable depending on the choice of appendage substituents.

Additionally, one skilled in the art will recognize that in some cases, the order of reactions may differ from that presented herein to avoid functional group incompatibilities and thus adjust the synthetic route accordingly.

The preparation of multicyclic intermediates useful for making the multicyclic and/or heterocyclic cores of the compounds of the invention have been described in the literature and in compendia such as “Comprehensive Heterocyclic Chemistry” editions I, II and III, published by Elsevier and edited by A. R. Katritzky & R. JK Taylor. Manipulation of the required substitution patterns have also been described in the available chemical literature as summarized in compendia such as “Comprehensive Organic Chemistry” published by Elsevier and edited by DH R. Barton and W. D. Ollis; “Comprehensive Organic Functional Group Transformations” edited by edited by A. R. Katritzky & R. JK Taylor and “Comprehensive Organic Transformation” published by Wiley-CVH and edited by R. C. Larock.

The starting materials used and the intermediates prepared using the methods set forth in the Examples below may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analytical Development for Low-Dose Oral Drug Products,” Wiley, 2009, ISBN.

### Preparation of Compounds and Intermediates

The invention is illustrated by the following Examples. For all of the Examples, standard work-up and purification methods known to those skilled in the art can be utilized. Unless otherwise indicated, all temperatures are expressed in ° C. (degrees Celsius). All reactions are conducted at room temperature unless otherwise noted. Synthetic methodologies illustrated herein are intended to exemplify the applicable chemistry through the use of specific examples and are not indicative of the scope of the disclosure.

### General Methods

Solvents, reagents, and intermediates that are commercially available were used as received. Intermediates that are not commercially available were prepared in the manner as described below. ¹H NMR spectra are reported as ppm downfield from Me₄Si with number of protons, multiplicities, and coupling constants in Hertz indicated parenthetically. Where LC/MS data are presented, the observed parent ion is given. Flash column chromatography was performed using pre-packed normal phase silica or bulk silica.

### Example 2

### Methods for Making the Intermediates of Compounds of the Invention

### Intermediate 1-1

### Preparation of Compound Int. 1-1

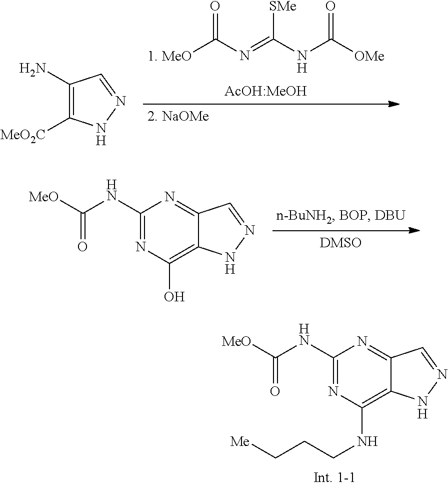

### methyl (7-(butylamino)-1H-pyrazolo[4,3-d]pyrimidin-5-yl)carbamate

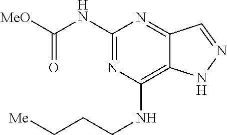 [SMILES: CCCCNc1nc(NC(=O)OC)nc2cn[nH]c12]

### Step 1: methyl (7-hydroxy-1H-pyrazolo[4,3-d]pyrimidin-5-yl)carbamate

To a stirred mixture of methyl 4-amino-1H-pyrazole-5-carboxylate (2 g, 14.17 mmol) and 1,3-bis(methoxycarbonyl)-2-methyl-2-thiopseudourea (2.92 g, 14.17 mmol) in MeOH (20 mL) was added AcOH (4.87 mL, 85 mmol) at ambient temperature. The resulting mixture was stirred for 16 h then NaOMe (25.5 g, 142 mmol) in MeOH was added dropwise. After the addition was complete, the reaction mixture was acidified to pH 1-2 with AcOH. The resulting solid was collected by filtration then washed with CH₃CN (200 mL) then dried in vacuo to give the title compound. MS m/z (M+H)+: calculated 210.1, observed 210.1. ¹H-NMR (400 MHz, DMSO-d6) δ 7.83 (s, 1H), 3.67 (s, 3H).

### Step 2: methyl (7-(butylamino)-1H-pyrazolo[4,3-d]pyrimidin-5-yl)carbamate

To a stirred mixture of methyl (7-hydroxy-1H-pyrazolo[4,3-d]pyrimidin-5-yl)carbamate (5 g, 23.90 mmol), butan-1-amine (4.73 ml, 47.8 mmol) and DBU (3.92 mL, 26.3 mmol) in DMSO (100 mL) was added BOP (12.69 g, 28.7 mmol) at ambient temperature. The resulting mixture was warmed to 60° C. for 3 h. The resulting mixture was cooled to ambient temperature, then filtered, then purified directly by reverse-phase chromatography (0-50% CH₃CN/water with 0.1% formic acid modifier) to give the title compound. MS m/z (M+H)⁺: calculated 265.1, observed 265.1. ¹H-NMR (400 MHz, DMSO-d6) δ 12.94 (br., 1H), 9.89 (s, 1H), 8.14-7.99 (m, 2H), 3.67 (s, 3H), 3.56-3.51 (m, 2H), 1.66-1.58 (m, 2H), 1.42-1.33 (m, 2H), 0.92 (t, J=7.2 Hz, 3H).

### Intermediate 2-1

### Preparation of Compound Int. 2-1

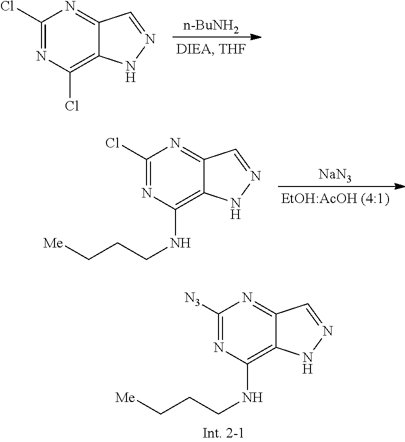

### 5-azido-N-butyl-1H-pyrazolo[4,3-d]pyrimidin-7-amine

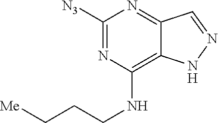 [SMILES: CCCCNc1nc(N=[N+]=[N-])nc2cn[nH]c12]

### Step 1: N-butyl-5-chloro-1H-pyrazolo[4,3-d]pyrimidin-7-amine

To a mixture of 5,7-dichloro-1H-pyrazolo[4,3-d]pyrimidine (3 g, 15.87 mmol) in THF (30 mL) at 0° C. was added DIEA (3.08 g, 23.81 mmol) and butan-1-amine (1.741 g, 23.81 mmol). After the addition was complete, the mixture was warmed to ambient temperature and stirred for 2 h. The resulting mixture was diluted with water (200 mL), then extracted with EtOAc (3×300 mL). The combined organic extracts were washed with brine (2×300 mL), then dried (Na₂SO₄), then filtered. The filtrate was concentrated to give the title compound, which was used directly in the next step without purification. MS m/z (M+H)⁺: calculated 226.1, observed 226.2.

### Step 2: 5-azido-N-butyl-1H-pyrazolo[4,3-d]pyrimidin-7-amine

To a mixture of N-butyl-5-chloro-1H-pyrazolo[4,3-d]pyrimidin-7-amine (3.5 g, 15.51 mmol) in AcOH (6 mL) and EtOH (24 mL) at ambient temperature was added sodium azide (1.512 g, 23.26 mmol), then the mixture was heated to 100° C. for 3 h. The resulting mixture was cooled to ambient temperature, then diluted with water (200 mL), then extracted with EtOAc (3×300 mL). The combined organic extracts were washed with brine (2×300 mL), then dried (Na₂SO₄), then filtered, then the filtrate was concentrated. The crude product was subjected to silica gel chromatography (0-20% EtOAc/petroleum ether) to give the title compound. MS m/z (M+H)⁺: calculated 233.1, observed 233.1.

Utilizing the procedures described for Intermediate 2-1, the following compounds were prepared substituting the appropriate reagents for butan-1-amine.

| Cmpd/ | | | Calc'd | Observed | |
|---|---|---|---|---|---|
| Int. # | Structure | Name | [M + H]⁺ | [M + H]⁺ | Comments |
| Int. 2-2 | 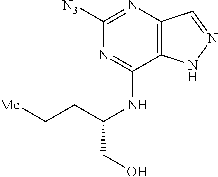 [SMILES: CCC[C@@H](CO)Nc1nc(N=[N+]=[N-])nc2cn[nH]c12] | (S)-2-((5-azido-1H- pyrazolo[4,3- d]pyrimidin-7-yl) amino)pentan-1-ol | 263.3 | 263.3 | |

### Intermediate 2-3

### Preparation of Compound Int. 2-3

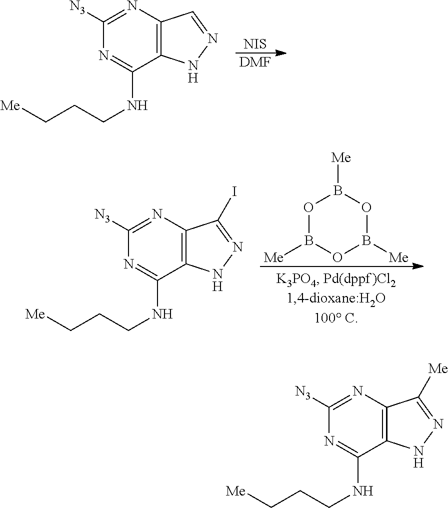

### 5-azido-N-butyl-3-methyl-1H-pyrazolo[4,3-d]pyrimidin-7-amine

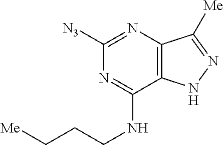 [SMILES: CCCCNc1nc(N=[N+]=[N-])nc2c(C)n[nH]c12]

### Step 1: 5-azido-N-butyl-3-iodo-1H-pyrazolo[4,3-d]pyrimidin-7-amine

To a solution of 5-azido-N-butyl-2H-pyrazolo[4,3-d]pyrimidin-7-amine (1 g, 4.31 mmol) in DMF (30 mL) was added N-iodosuccinimide (1.937 g, 8.61 mmol) then the mixture was heated to 40° C. After heating overnight the mixture was cooled to rt then diluted 10% Na₂SO₃ (40 mL). The resulting solid was collected by filtration then washed with water (10 mL×3) then dried under vacuum to give the title compound. MS m/z (M+H)⁺: calculated 359.0, observed 359.0.

### Step 2: 5-azido-N-butyl-3-methyl-1H-pyrazolo[4,3-d]pyrimidin-7-amine

To a solution of 5-azido-N-butyl-3-iodo-2H-pyrazolo[4,3-d]pyrimidin-7-amine (1 g, 2.79 mmol) in 1,4-dioxane (10 mL) and H₂O (1 mL) was added Pd(dppf)Cl₂ (0.204 g, 0.279 mmol), K₃PO₄ (1.185 g, 5.58 mmol) and 2,4,6-trimethyl-1,3,5,2,4,6-trioxatriborinane (3.51 g, 13.96 mmol) under N₂ then the mixture was heated to 100° C. overnight. The resulting mixture was cooled to rt then diluted with EtOAc (30 mL) then dried (Na₂SO₄) then filtered then the filtrate was concentrated. The crude product was subjected to silica gel chromatography (30-70% EtOAc/pet. ether) to give the title compound. MS m/z (M+H)⁺: calculated 247.1, observed 247.4.

### Intermediate 3-1

### Preparation of Compound Int. 3-1

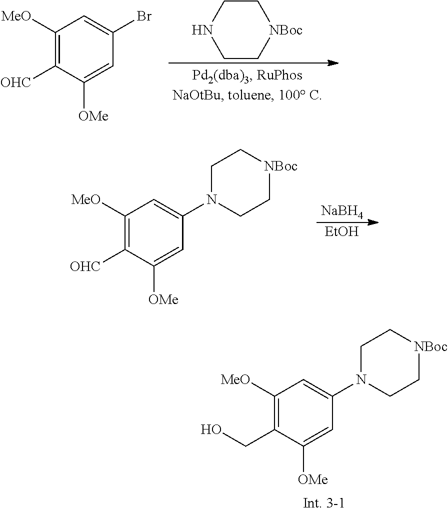 [SMILES: "EtOH" ; COc1cc(Br)cc(OC)c1C=O ; CC(C)(C)OC(=O)N1CCNCC1 ; COc1cc(N2CCN(C(=O)OC(C)(C)C)CC2)cc(OC)c1C=O ; "NaBH4" ; COc1cc(N2CCN(C(=O)OC(C)(C)C)CC2)cc(OC)c1CO]

### tert-butyl 4-(4-(hydroxymethyl)-3,5-dimethoxyphenyl)piperazine-1-carboxylate

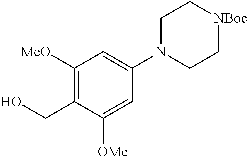 [SMILES: COc1cc(N2CCN(C(=O)OC(C)(C)C)CC2)cc(OC)c1CO]

### Step 1: tert-butyl 4-(4-formyl-3,5-dimethoxyphenyl)piperazine-1-carboxylate

To a stirred mixture of 4-bromo-2,6-dimethoxybenzaldehyde (5 g, 20.40 mmol) in toluene (20 mL) was added tert-butyl piperazine-1-carboxylate (5.70 g, 30.6 mmol), RuPhos (1.904 g, 4.08 mmol), Pd₂(dba)₃ (1.868 g, 2.040 mmol), and NaOtBu (5.88 g, 61.2 mmol) under an argon atmosphere, then the mixture was heated to 100° C. After 6 h the mixture was cooled to ambient temperature, then diluted with water (50 mL), then extracted with EtOAc (3×80 mL). The combined organic extracts were washed with brine (3×50 mL), then dried (Na₂SO₄), then filtered, then the filtrate was concentrated. The crude product was subjected to silica gel chromatography (0-100% EtOAc/DCM) to give the title compound. MS m/z (M+H)⁺: calculated 351.2, observed 351.3.

### Step 2: tert-butyl 4-(4-(hydroxymethyl)-3,5-dimethoxyphenyl)piperazine-1-carboxylate

To a stirred mixture of tert-butyl 4-(4-formyl-3,5-dimethoxyphenyl)piperazine-1-carboxylate (3 g, 8.56 mmol) in EtOH (10 mL) was added NaBH₄ (0.389 g, 10.27 mmol) at ambient temperature. After 15 minutes the mixture was diluted with water (15 mL) then extracted with Et₂O (3×50 mL). The combined organic extracts were washed with brine (3×50 mL), then dried (Na₂SO₄), then filtered. The filtrate was then concentrated give the title compound which was used directly in the next step without purification. MS m/z (M+H)⁺: calculated 353.2, observed 353.3.

### Intermediate 4-1

### Preparation of Compound Int. 4-1

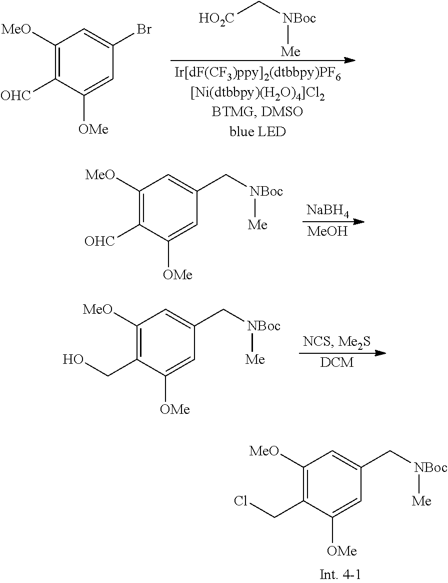

### tert-butyl (4-(chloromethyl)-3,5-dimethoxybenzyl)(methyl)carbamate

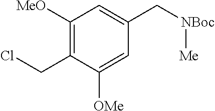 [SMILES: COc1cc(CN(C)C(=O)OC(C)(C)C)cc(OC)c1CCl]

### Step 1: tert-butyl (4-formyl-3,5-dimethoxybenzyl)(methyl)carbamate

4-bromo-2,6-dimethoxybenzaldehyde (6 g, 24.48 mmol), N-(tert-butoxycarbonyl)-N-methylglycine (9.26 g, 49.0 mmol), Ir[dF(CF₃)ppy]₂(dtbbpy)PF₆ (0.275 g, 0.245 mmol), and [Ni(dtbbpy)(H₂O)₄]Cl₂ (0.575 g, 1.224 mmol) were combined in DMSO (120 mL). To this was added BTMG (10 mL, 49.0 mmol). The mixture was sonicated until all solids dissolved. The resulting mixture was divided evenly into 4 screw cap vials with stir bars. N₂ was bubbled through each mixture for 5 minutes. The vials were capped then the mixtures were irradiated in a PennOC Photoreactor® (wavelength: 420 nm; LED intensity: 100%; fan speed: 5000 rpm; stir: 1200 rpm) overnight. The separated mixtures were combined then diluted with H₂O then extracted with EtOAc (3×). The combined organic extracts were washed with H₂O and brine then dried (MgSO₄) then filtered then the filtrate was concentrated. The crude product was subjected to silica gel chromatography (0-50% 3:1 EtOAc:EtOH/heptane) to give the title compound. ¹H-NMR (400 MHz, CDCl₃) δ 10.47 (s, 1H), 6.44 (bs, 2H), 4.41 (s, 2H), 3.88 (s, 6H), 2.87 (bs, 3H), 1.49 (bs, 9H).

### Step 2: tert-butyl (4-(hydroxymethyl)-3,5-dimethoxybenzyl)(methyl)carbamate

A solution of tert-butyl (4-formyl-3,5-dimethoxybenzyl)(methyl)carbamate (6.98 g, 22.56 mmol) in MeOH (100 mL) was cooled to 0° C. To this was added NaBH₄ (1.03 g, 27.2 mmol) in portions. After 1 h gas evolution had ceased. The cooling bath was removed and the mixture warmed to ambient temperature. After 90 minutes the mixture was concentrated. The residue was taken up in DCM, filtered through a pad of Celite® washing with DCM, then the filtrate was concentrated. The crude product was subjected to silica gel chromatography (0-15% 3:1 EtOAc:EtOH/heptane) to give the title compound. ¹H-NMR (400 MHz, CDCl₃) δ 6.45 (bs, 2H), 4.76 (d, J=6.6 Hz, 2H), 4.39 (s, 2H), 3.83 (s, 6H), 2.86 (bs, 3H), 1.49 (s, 9H).

### Step 3: tert-butyl (4-(chloromethyl)-3,5-dimethoxybenzyl)(methyl)carbamate

A solution of N-chlorosuccinimide (0.472 g, 3.53 mmol) in DCM (1 mL) was cooled to 0° C. To this was added dimethylsulfide (0.239 g, 3.85 mmol) slowly resulting in a precipitate. The reaction mixture was cooled to −20° C. then a solution of tert-butyl (4-(hydroxymethyl)-3,5-dimethoxybenzyl)(methyl)carbamate (1 g, 3.21 mmol) in DCM was added dropwise. The mixture was stirred for 2 h allowing the temperature to reach 0° C. during which time all the solid precipitate had dissolved giving a clear solution. The solution was poured over cold brine then extracted with Et2O (2×). The combined organic extracts were washed with cold brine then dried (Na₂SO₄) then filtered then the filtrate was concentrated give the title compound which was used directly in the next step without purification. MS m/z (M+H)⁺: calculated 330.1, observed 329.2.

### Intermediate 5-1

### Preparation of Compound Int. 5-1

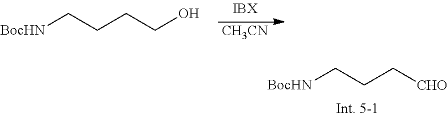

### tert-butyl (4-oxobutyl)carbamate

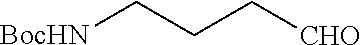 [SMILES: CC(C)(C)OC(=O)NCCCC=O]

To a mixture of tert-butyl (4-hydroxybutyl)carbamate (1 g, 5.28 mmol) in CH₃CN (30 mL) at ambient temperature was added IBX (1.231 g, 6.34 mmol) then the mixture was heated to 80° C. After 1 h the mixture was cooled to ambient temperature then diluted with water (100 mL) then extracted with EtOAc (3×200 mL). The combined organic extracts were washed with brine (2×200 mL) then dried (Na₂SO₄) then filtered then the filtrate was concentrated. The crude product was subjected to silica gel chromatography (0-70% EtOAc/petroleum ether) to give the title compound. ¹H-NMR (400 MHz, CDCl₃): δ 3.76-3.73 (m, 2H), 2.53-2.49 (t, 2H), 2.03-1.97 (m, 2H), 1.53-1.45 (s, 9H).

### Intermediate 6-1

### Preparation of Compound Int. 6-1

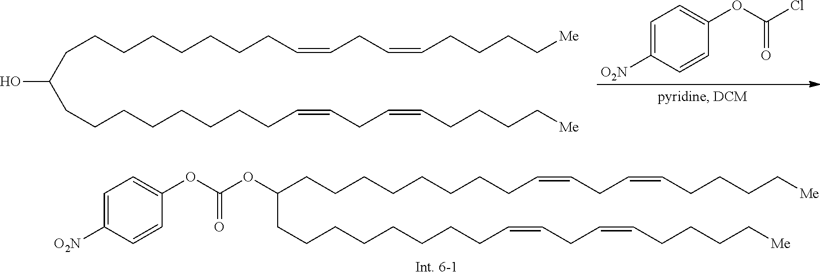 [SMILES: CCCCC/C=C\C/C=C\CCCCCCCCC(O)CCCCCCCC/C=C\C/C=C\CCCCC ; O=C(Cl)Oc1ccc([N+](=O)[O-])cc1 ; CCCCC/C=C\C/C=C\CCCCCCCCC(CCCCCCCC/C=C\C/C=C\CCCCC)OC(=O)Oc1ccc([N+](=O)[O-])cc1]

### (6Z,9Z,28Z,31Z)-heptatriaconta-6,9,28,31-tetraen-19-yl (4-nitrophenyl) carbonate

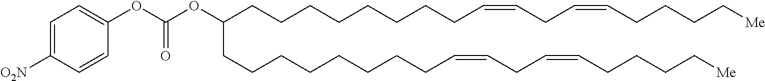 [SMILES: CCCCC/C=C\C/C=C\CCCCCCCCC(CCCCCCCC/C=C\C/C=C\CCCCC)OC(=O)Oc1ccc([N+](=O)[O-])cc1]

A mixture of (6Z,9Z,28Z,31Z)-heptatriaconta-6,9,28,31-tetraen-19-ol (300 mg, 0.567 mmol) and hypochlorous 4-nitrobenzoic anhydride (229 mg, 1.134 mmol) in DCM (4.5 mL) and pyridine (1.5 mL) was stirred at ambient temperature for 5 h. The resulting mixture was concentrated then purified by prep-TLC (petroleum ether:DCM=1:1) to give the title compound.

Utilizing the procedures described for Intermediate 6-1, the following compounds were prepared substituting the appropriate reagents for (6Z,9Z,28Z,31Z)-heptatriaconta-6,9,28,31-tetraen-19-ol.

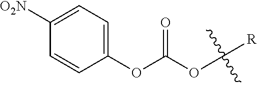

| Cmpd/ | | | Calc'd | Observed | |
|---|---|---|---|---|---|
| Int. # | R | Name | [M + H]⁺ | [M + H]⁺ | Comments |
| Int. 6-2 | 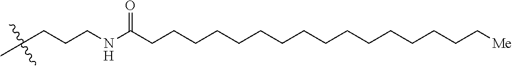 [SMILES: *CCCNC(=O)CCCCCCCCCCCCCCCCC] | 4- nitrophenyl (3- stearamido- propyl) carbonate | 507.3 | 507.4 | from Int. 8-2; substitute DIEA for pyridine as base |

### Intermediate 7-1

### Preparation of Compound Int. 7-1

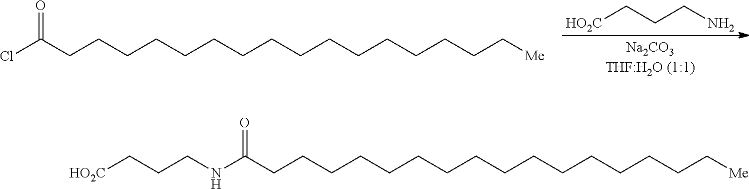 [SMILES: CCCCCCCCCCCCCCCCCC(=O)Cl ; NCCCC(=O)O ; CCCCCCCCCCCCCCCCCC(=O)NCCCC(=O)O]

### 4-Stearamidobutanoic Acid

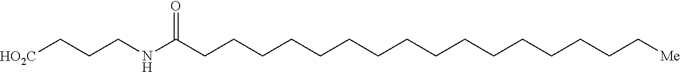 [SMILES: CCCCCCCCCCCCCCCCCC(=O)NCCCC(=O)O]

A mixture of 4-aminobutanoic acid (1 g, 9.70 mmol) and Na₂CO₃ (1.542 g, 14.55 mmol) in THF (30 mL) and water (30 mL) was cooled to 0° C. To this was added stearoyl chloride (2.94 g, 9.70 mmol). After the addition was complete the mixture was stirred for 16 h at 25° C. The resulting mixture was quenched with saturated KHSO₄ then extracted with CHCl₃ (500 mL). The combined organic layers were washed with brine (3×100 mL) then dried (Na₂SO₄) then filtered then the filtrate was concentrated to give the title compound. MS m/z (M−H)⁻: calculated 368.6, observed 368.3.

Utilizing the procedures described for Intermediate 7-1, the following compounds were prepared substituting the appropriate reagents for 4-aminobutanoic acid.

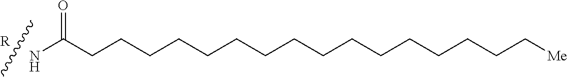

| Cmpd/ | | | Calc'd | Observed | |
|---|---|---|---|---|---|
| Int. # | R | Name | [M − H]⁻ | [M − H]⁻ | Comments |
| Int. 7-2 | 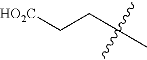 [SMILES: *CCC(=O)O] | 3- stearamidopropanoic acid | 354.4 | 354.3 | |
| Int. 7-3 | 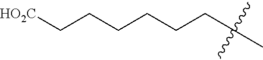 [SMILES: *CCCCCCC(=O)O] | 7- stearamidoheptanoic acid | 410.7 | 410.4 | methyl 7- aminoheptanoate hydrochloride was used as the amine; ester hydrolysis was achieved with NaOH in THF:H₂O (5:1) |
| Int. 7-4 | 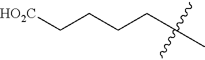 [SMILES: *CCCCC(=O)O] | 5- stearamidopentanoic acid | 382.6 | 382.3 | |
| Int. 7-5 | 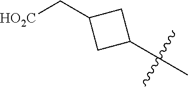 [SMILES: *C1CC(CC(=O)O)C1] | 2-(3- stearamidocyclobutyl) acetic acid | 396.6 | 396.6 | MS [M + H]⁺ |

### Intermediate 8-1

### Preparation of Compound Int. 8-1

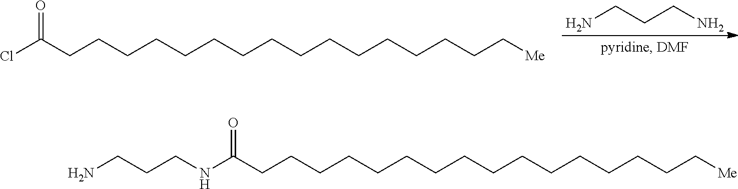 [SMILES: CCCCCCCCCCCCCCCCCC(=O)Cl ; NCCCN ; CCCCCCCCCCCCCCCCCC(=O)NCCCN]

### N-(3-aminopropyl)stearamide

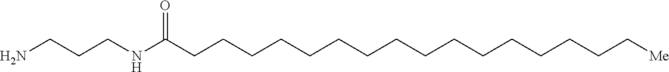 [SMILES: CCCCCCCCCCCCCCCCCC(=O)NCCCN]

A mixture of propane-1,3-diamine (1 g, 13.49 mmol) and pyridine (1.091 mL, 13.49 mmol) in DMF (15 mL) was cooled to 0° C. To this was added stearoyl chloride (4.09 g, 13.49 mmol). After the addition was complete the mixture was stirred for 16 h at 25° C. The resulting mixture was diluted with ice water resulting in a solid. The solid was collected by filtration then washed with water (3×100 mL) then dried to give the title compound. MS m/z (M+H)⁺: calculated 341.6, observed 341.3.

Utilizing the procedures described in Intermediate 8-1 the following compounds were prepared substituting the appropriate reagents for propane-1,3-diamine.

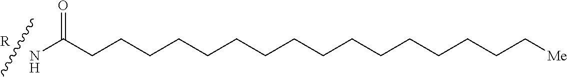

| Cmpd/ | | | Calc'd | Observed | |
|---|---|---|---|---|---|
| Int. # | R | Name | [M + H]⁺ | [M + H]⁺ | Comments |
| Int. 8-2 | 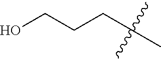 [SMILES: *CCCO] | N-(3- hydroxypropyl) stearamide | 342.3 | 342.3 | |
| Int. 8-3 | 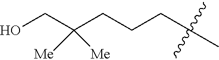 [SMILES: *CCCC(C)(C)CO] | N-(5-hydroxy-4,4- dimethylpentyl) stearamide | 398.4 | 398.7 | |

### Intermediate 9-1

### Preparation of Compound Int. 9-1

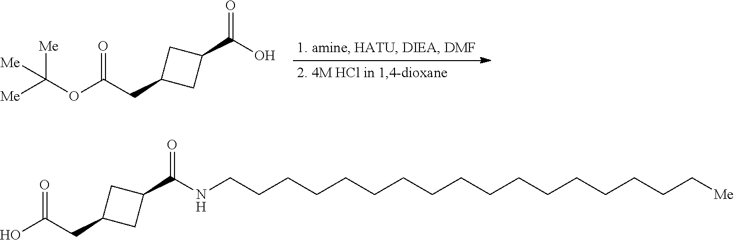 [SMILES: CC(C)(C)OC(=O)C[C@H]1C[C@@H](C(=O)O)C1 ; CCCCCCCCCCCCCCCCCCNC(=O)[C@H]1C[C@@H](CC(=O)O)C1]

### 2-((1s,3s)-3-(octadecylcarbamoyl)cyclobutyl)acetic Acid

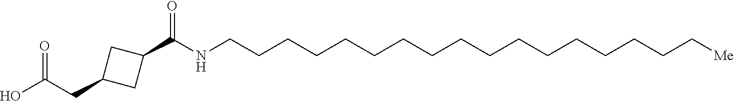 [SMILES: CCCCCCCCCCCCCCCCCCNC(=O)[C@H]1C[C@@H](CC(=O)O)C1]

### Step 1: tert-butyl 2-((1s,3s)-3-(octadecylcarbamoyl)cyclobutyl)acetate

To a stirred mixture of (1s,3s)-3-(2-(tert-butoxy)-2-oxoethyl)cyclobutane-1-carboxylic acid (0.5 g, 2.334 mmol), HATU (1.78 g, 4.67 mmol), and DIEA (1.22 mL, 7.00 mmol) in DMF (10 mL) was added a solution of octadecan-1-amine (0.818 g, 3.03 mmol) in DMF (10 mL) at 25° C. After 12 hr the mixture was diluted with water (10 mL) then extracted with EtOAc (3×10 mL). The combined organic layers were washed with brine (3×10 mL) then dried (Na₂SO₄) then filtered then the filtrate was concentrated to give the title compound as an 80:20 mixture with tert-butyl 2-((1s,3s)-3-(hexadecylcarbamoyl)cyclobutyl)acetate. MS m/z (M+H)⁺: calculated 466.4, observed 466.2.

### Step 2: 2-((1s,3s)-3-(octadecylcarbamoyl)cyclobutyl)acetic acid

To a stirred mixture of tert-butyl 2-(3-(octadecylcarbamoyl)cyclobutyl)acetate (0.932 g, 2 mmol) in 1,4-dioxane (16 mL) was added 4M HCl (4 mL) in 1,4-dioxane at 25° C. After 6 hr the mixture was diluted with EtOAc then washed with brine (3×30 mL). The organic layer was dried (Na₂SO₄) then filtered then the filtrate was concentrated to give the title compound as an 80:20 mixture with 2-((1s,3s)-3-(hexadecylcarbamoyl)cyclobutyl)acetic acid. MS m/z (M+H)⁺: calculated 410.4, observed 410.4.

### Intermediate 10-1

### Preparation of Compound Int. 10-1

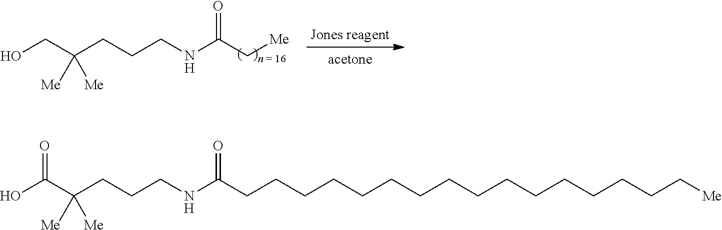

### 2,2-dimethyl-5-stearamidopentanoic Acid

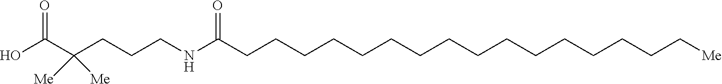 [SMILES: CCCCCCCCCCCCCCCCCC(=O)NCCCC(C)(C)C(=O)O]

To a mixture of N-(5-hydroxy-4,4-dimethylpentyl)stearamide (Int. 8-3, 500 mg, 1.257 mmol) in acetone (10 mL) was added chromium trioxide solution in sulfuric acid (0.691 mL, 1.383 mmol) at 0° C. After 6 hr the mixture was diluted with water. The resulting solid was collected by filtration then washed with water then air dried. MS m/z (M+H)⁺: calculated 412.4, observed 412.1.

### Intermediate 11-1

### Preparation of Compound Int. 11-1

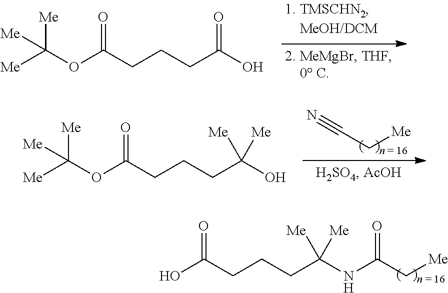

### 5-methyl-5-stearamidohexanoic Acid

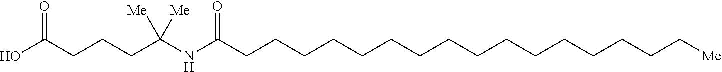 [SMILES: CCCCCCCCCCCCCCCCCC(=O)NC(C)(C)CCCC(=O)O]

### Step 1: tert-butyl methyl glutarate

A solution of 5-(tert-butoxy)-5-oxopentanoic acid (5 g, 26.6 mmol) in DCM (40 mL) and MeOH (40 mL) was cooled to 0° C. To this was added (diazomethyl)trimethylsilane (39.8 mL, 80 mmol, 2M in hexane). After the addition was complete the mixture was warmed to rt. After 3 hr the mixture was concentrated. The crude product was subjected to silica gel chromatography (0-5% EtOAc/pet. ether) to give the title compound. ¹H NMR (400 MHz, CDCl₃) δ 3.68 (s, 3H), 2.37 (t, J=7.2 Hz, 2H), 2.28 (t, J=7.6 Hz, 2H), 1.91 (quin, J=7.2 Hz, 2H), 1.44 (s, 9H).

### Step 2: tert-butyl 5-hydroxy-5-methylhexanoate

To a solution of tert-butyl methyl glutarate (1 g, 4.94 mmol) in THF (16 mL) was added MeMgBr (4.12 mL, 12.36 mmol, 3M in THF) at 0° C. After 2 hr the mixture was quenched with saturated NH₄Cl (25 mL) then warmed to rt then extracted with EtOAc (10 mL×3). The combined organic layers were washed with brine (30 mL) then dried (Na₂SO₄) then filtered then the filtrate concentrated. The crude product was subjected to silica gel chromatography (0-20% EtOAc/pet. ether) to give the title compound. ¹H NMR (400 MHz, CDCl₃) δ 2.25 (t, J=7.2 Hz, 2H), 1.69-1.64 (m, 2H), 1.50-1.48 (m, 2H), 1.45 (s, 9H), 1.23 (s, 6H).

### Step 3: 5-methyl-5-stearamidohexanoic Acid

To a solution of tert-butyl 5-hydroxy-5-methylhexanoate (300 mg, 1.483 mmol) in AcOH (2 mL) was added stearonitrile (787 mg, 2.97 mmol) at rt then the mixture was cooled to 0° C. H₂SO₄ (0.5 mL) was added. After the addition was complete the cooling bath was removed and the mixture warmed to rt. After stirring overnight the mixture was quenched with saturated NaHCO₃ to pH=5. The resulting mixture was extracted with EtOAc (2 mL×3). The combined organic layers were dried (Na₂SO₄) then filtered then the filtrate was concentrated. The residue was taken up in MeCN (5 mL). The resulting precipitated solid was collected then recrystallized from EtOAc to give the title compound. MS m/z (M+H)⁺: calculated 412.7, observed 412.3.

### Intermediate 12-1

### Preparation of Compound Int. 12-1

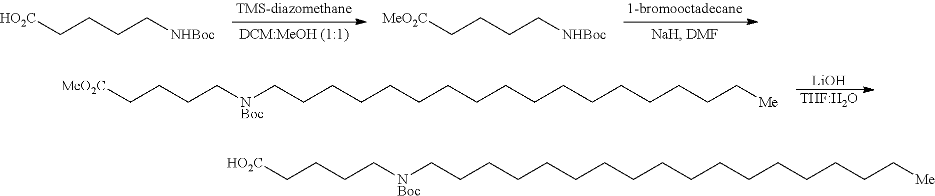

### 5-((tert-butoxycarbonyl)(octadecyl)amino)pentanoic Acid

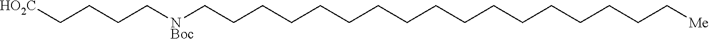 [SMILES: CCCCCCCCCCCCCCCCCCN(CCCCC(=O)O)C(=O)OC(C)(C)C]

### Step 1: methyl 5-((tert-butoxycarbonyl)amino)pentanoate

To a solution of 5-((tert-butoxycarbonyl)amino)pentanoic acid (2 g, 9.21 mmol) in MeOH (15 mL) and DCM (15 mL) was added 2M (trimethylsilyl)diazomethane (23.01 mL, 46.0 mmol) in hexane at rt. After 1 hr the mixture was concentrated to give the title compound which was used without purification. ¹H NMR (400 MHz, CDCl₃) δ 4.56 (s, 1H), 3.68 (s, 3H), 3.13 (q, J=6.4 Hz, 2H), 2.34 (t, J=7.2 Hz, 2H), 1.70-1.62 (m, 2H), 1.49-1.56 (m, 2H), 1.45 (s, 9H).

### Step 2: methyl 5-((tert-butoxycarbonyl)(octadecyl)amino)pentanoate

To a solution of methyl 5-((tert-butoxycarbonyl)amino)pentanoate (1 g, 4.32 mmol) in DMF (20 mL) was added NaH (0.259 g, 6.49 mmol, 60% in mineral oil) at 0° C. After the addition was complete the mixture was warmed to rt. After 30 minutes 1-bromooctadecane (4.32 g, 12.97 mmol) was added then the mixture was heated to 50° C. After 2 hr the mixture was cooled to rt then diluted with water (100 mL) then extracted with EtOAc (3×). The combined organic layers were washed with brine (50 mL) then dried (Na₂SO₄) then filtered then the filtrate was concentrated. The crude product was subjected to silica gel chromatography (0-20% EtOAc/pet. ether) to give the title compound. ¹H NMR (400 MHz, CD₃OD) δ 3.67-3.64 (m, 3H), 3.23-3.15 (m, 4H), 2.39-2.33 (m, 2H), 1.57 (d, J=4.4 Hz, 6H), 1.46 (s, 9H), 1.29 (s, 30H), 0.92-0.88 (m, 3H).

### Step 3: 5-((tert-butoxycarbonyl)(octadecyl)amino)pentanoic Acid

To a stirred solution of methyl 5-((tert-butoxycarbonyl)(octadecyl)amino)pentanoate (240 mg, 0.496 mmol) in THF (6 mL) and H₂O (2 mL) was added LiOH monohydrate (62.5 mg, 1.488 mmol) at rt then the mixture was warmed to 40° C. After stirring overnight the mixture was cooled to rt then the pH was adjusted to 7 with 1N HCl. The resulting mixture was extracted with EtOAc (3×5 mL). The combined organic layers were washed with brine (3×5 mL) then dried (Na₂SO₄) then filtered then the filtrate was concentrated to give the title compound which was used in the next step without purification. ¹H NMR (400 MHz, CD₃OD) δ 3.23-3.14 (m, 4H), 2.35-2.28 (m, 2H), 1.65-1.52 (m, 6H), 1.46 (s, 9H), 1.29 (s, 30H), 0.92-0.88 ppm (m, 3H).

### Intermediate 13-1

### Preparation of Compound Int. 13-1

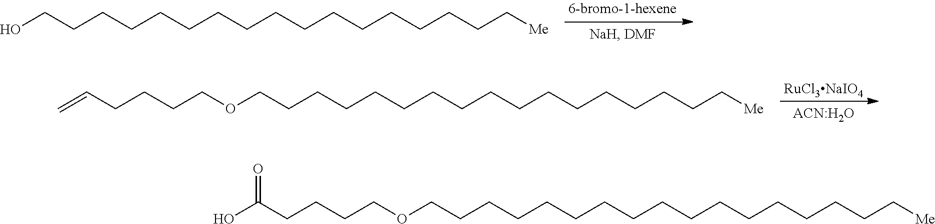

### 5-(octadecyloxy)pentanoic Acid

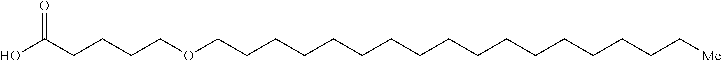 [SMILES: CCCCCCCCCCCCCCCCCCOCCCCC(=O)O]

### Step 1: 1-(hex-5-en-1-yloxy)octadecane

To a solution of octadecan-1-ol (5 g, 18.48 mmol) in DMF (100 mL) was added NaH (3.70 g, 92 mmol) (60% in mineral oil) in portions at rt. After stirring for 30 minutes 6-bromo-1-hexene (24.70 mL, 185 mmol) was added then the mixture was heated to 80° C. After heating overnight the mixture was cooled to rt then diluted with water (200 mL) then extracted with EtOAc (100 mL×3). The combined organic layers were dried (Na₂SO₄) then filtered then the filtrated was concentrated. The crude product was subjected to silica gel chromatography (0-100% EtOAc/pet. ether) to give the title compound. ¹H NMR (400 MHz, CDCl₃) δ 5.82 (ddt, J=17.2, 10.4, 6.8, 6.8 Hz, 1H), 5.01 (dq, J=17.6, 1.2 Hz, 1H), 4.95 (d, J=10.0 Hz, 1H), 3.45-3.36 (m, 4H), 2.08 (d, J=7.2 Hz, 2H), 1.65-1.55 (m, 5H), 1.49-1.44 (m, 2H), 1.39-1.1.17 (m, 29H), 0.85-0.92 (m, 3H).

### Step 2: 5-(octadecyloxy)pentanoic acid

To s solution of 1-(hex-5-en-1-yloxy) octadecane (1 g, 2.84 mmol) in MeCN (10 mL) was added a solution of RuCl₃ (0.059 g, 0.284 mmol) and NaIO₄ (2.426 g, 11.34 mmol) in H₂O (10 mL) at rt. After 2 hr the mixture was diluted with Na₂SO₃ (25 mL) then extracted with EtOAc (25 mL×3). The combined organic layers were washed with 1N HCl (20 mL), saturated NaHCO₃ (20 mL), and brine (20 mL) then dried (Na₂SO₄) then filtered then the filtrate was concentrated to give the title compound which was used in the next step without purification. ¹H NMR (400 MHz, CDCl₃) δ ppm 3.39-3.44 (m, 4H), 2.40 (t, J=7.27 Hz, 2H), 1.70-1.75 (m, 2H), 1.65 (br d, J=7.51 Hz, 2H), 1.55-1.58 (m, 2H), 1.26 (s, 30H), 0.87-0.89 (m, 3H).

### Intermediate 14-1

### Preparation of Compound Int. 14-1

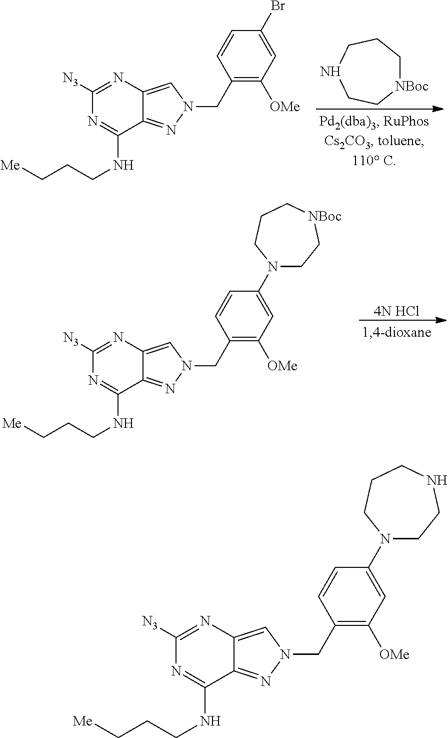

### 2-(4-(1,4-diazepan-1-yl)-2-methoxybenzyl)-5-azido-N-butyl-2H-pyrazolo[4,3-d]pyrimidin-7-amine

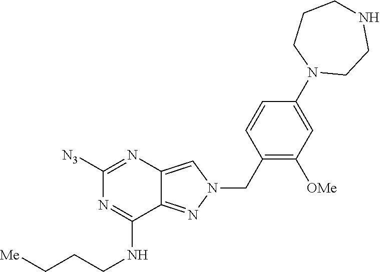 [SMILES: CCCCNc1nc(N=[N+]=[N-])nc2cn(Cc3ccc(N4CCCNCC4)cc3OC)nc12]

### Step 1: Tert-butyl 1-(4-((5-azido-7-(butylamino)-2H-pyrazolo[4,3-d]pyrimidin-2-yl)methyl)-3-methoxyphenyl)piperidine-4-carboxylate

To a mixture of 5-azido-2-(4-bromo-2-methoxybenzyl)-N-butyl-2H-pyrazolo[4,3-d]pyrimidin-7-amine (from Compound B-1 step 1, 500 mg, 1.159 mmol) and tert-butyl 1,4-diazepane-1-carboxylate (302 mg, 1.507 mmol) in toluene (10 mL) was added RuPhos (216 mg, 0.464 mmol), Pd₂(dba)₃ (212 mg, 0.232 mmol) and Cs₂CO₃ (1133 mg, 3.48 mmol) under N₂ then the mixture was heated to 110° C. After heating overnight the mixture was cooled to rt then diluted with water (100 mL) then the resulting mixture was extracted with EtOAc (3×100 mL). The combined organic layers were washed with brine (3×300 mL) then dried (Na₂SO₄) then filtered then the filtrate was concentrated. The crude product was subjected to silica gel chromatography (0-52% 3:1 EtOAc/pet. ether) to give the title compound. MS m/z (M+H)⁺: calculated 551.7, observed 551.1.

### Step 2: 2-(4-(1,4-diazepan-1-yl)-2-methoxybenzyl)-5-azido-N-butyl-2H-pyrazolo[4,3-d]pyrimidin-7-amine

A solution of tert-butyl 4-(4-((5-azido-7-(butylamino)-2H-pyrazolo[4,3-d]pyrimidin-2-yl)methyl)-3-methoxyphenyl)-1,4-diazepane-1-carboxylate (350 mg, 0.636 mmol) in DCE (2.00 mL) was cooled to 0° C. To this was added 4M HCl in 1,4-dioxane (2 mL) at 0° C. After 1 hr the mixture was warmed to rt then concentrated to give the title compound as the HCl salt which was used without purification. MS m/z (M+H)⁺: calculated 451.3, observed 451.3.

Utilizing the procedures described for Intermediate 14-1, the following compounds were prepared substituting the appropriate reagents for tert-butyl 1,4-diazepane-1-carboxylate.

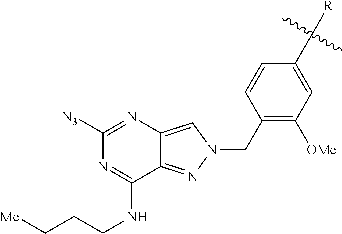

| Cmpd/ | | | Calc'd | Observed | |
|---|---|---|---|---|---|
| Int. # | R | Name | [M + H]⁺ | [M + H]⁺ | Comments |
| Int. 14-2 | 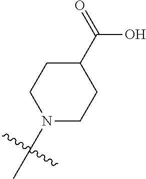 [SMILES: *N1CCC(C(=O)O)CC1] | 1-(4-((5-azido-7-(butylamino)- 2H-pyrazolo[4,3-d]pyrimidin-2- yl)methyl)-3- methoxyphenyl)piperidine-4- carboxylic acid | 480.2 | 480.2 | |

### Intermediate 15-1

### Preparation of Compound Int. 15-1

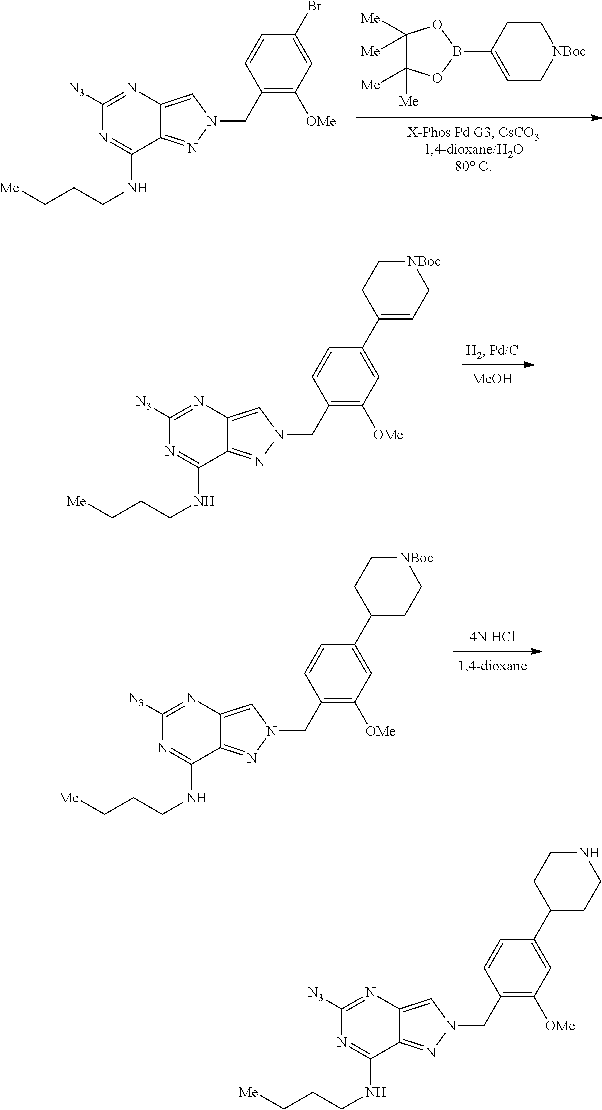

### 5-azido-N-butyl-2-(2-methoxy-4-(piperidin-4-yl)benzyl)-2H-pyrazolo[4,3-d]pyrimidin-7-amine

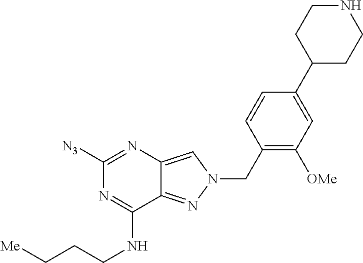 [SMILES: CCCCNc1nc(N=[N+]=[N-])nc2cn(Cc3ccc(C4CCNCC4)cc3OC)nc12]

### Step 1: Tert-butyl 4-(4-((5-azido-7-(butylamino)-2H-pyrazolo[4,3-d]pyrimidin-2-yl)methyl)-3-methoxyphenyl)-3,6-dihydropyridine-1(2H)-carboxylate

To a mixture of 5-azido-2-(4-bromo-2-methoxybenzyl)-N-butyl-2H-pyrazolo[4,3-d]pyrimidin-7-amine (from Compound B-1 step 1, 400 mg, 0.927 mmol) and tert-butyl 4-(4,4,5,5-tetramethyl-1,3,2-dioxaborolan-2-yl)-3,6-dihydropyridine-1(2H)-carboxylate (430 mg, 1.391 mmol) in 1,4-dioxane (4 mL)/H₂O (0.4 mL) was added Cs₂CO₃ (604 mg, 1.855 mmol) and X-Phos Pd G3 (39.3 mg, 0.046 mmol) under N₂ then the mixture was heated to 80° C. After 2 hr the mixture was cooled to rt then diluted with EtOAc (200 mL). The resulting mixture was washed with brine (3×100 mL) then dried (Na₂SO₄) then filtered then the filtrate was concentrated. The crude product was subjected to silica gel chromatography (0-50% EtOAc/pet. ether) to give the title compound. MS m/z (M+H)⁺: calculated 534.3, observed 534.4.

### Step 2: tert-butyl 4-(4-((5-azido-7-(butylamino)-2H-pyrazolo[4,3-d]pyrimidin-2-yl)methyl)-3-methoxyphenyl)piperidine-1-carboxylate

To a solution of tert-butyl 4-(4-((5-azido-7-(butylamino)-2H-pyrazolo[4,3-d]pyrimidin-2-yl)methyl)-3-methoxyphenyl)-3,6-dihydropyridine-1(2H)-carboxylate (290 mg, 0.543 mmol) in MeOH (10 mL) was added Pd/C (150 mg, 1.410 mmol) under N₂. The resulting mixture was placed under and atmosphere of H₂ (3×vacuum/H₂) and stirred at rt. After 16 hr the mixture was degassed (3×vacuum/N₂) then filtered washing with MeOH then the filtrated was concentrated to give the title compound. MS m/z (M+H)⁺: calculated 536.3, observed 536.3.

### Step 3: 5-azido-N-butyl-2-(2-methoxy-4-(piperidin-4-yl)benzyl)-2H-pyrazolo[4,3-d]pyrimidin-7-amine

To a solution of tert-butyl 4-(4-((5-azido-7-(butylamino)-2H-pyrazolo[4,3-d]pyrimidin-2-yl)methyl)-3-methoxyphenyl)piperidine-1-carboxylate (250 mg, 0.467 mmol) in DCM (2 mL) was added 4M HCl in 1,4-dioxane (2 mL) at rt. After 1 hr the mixture was concentrated to give the title compound as the HCl salt which was used without purification. MS m/z (M+H)⁺: calculated 436.3, observed 436.6.

### Intermediate 16-1

### Preparation of Compound Int. 16-1

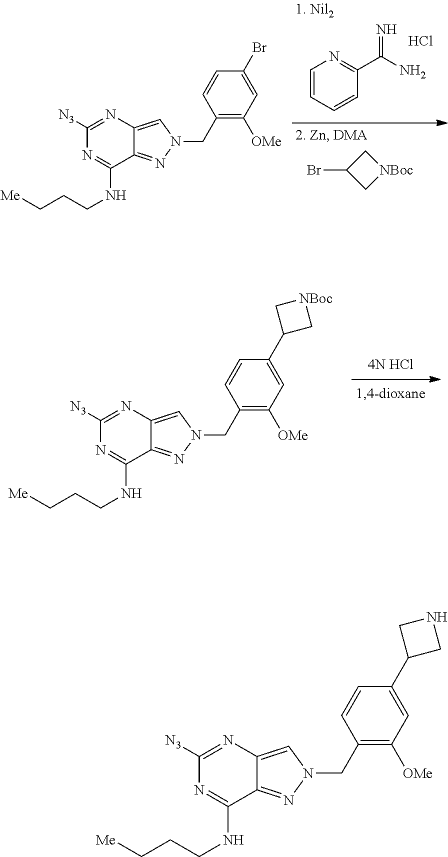

### 2-(4-(azetidin-3-yl)-2-methoxybenzyl)-5-azido-N-butyl-2H-pyrazolo[4,3-d]pyrimidin-7-amine

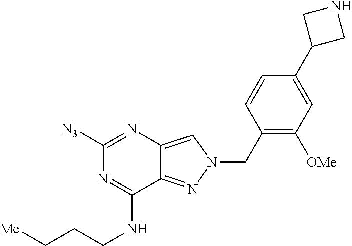 [SMILES: CCCCNc1nc(N=[N+]=[N-])nc2cn(Cc3ccc(C4CNC4)cc3OC)nc12]

### Step 1: Tert-butyl 3-(4-((5-azido-7-(butylamino)-2H-pyrazolo[4,3-d]pyrimidin-2-yl)methyl)-3-methoxyphenyl)azetidine-1-carboxylate

To a solution of NiI₂ (36.2 mg, 0.116 mmol) in DMA (1 mL) was added picolinimidamide hydrochloride (36.5 mg, 0.232 mmol) then the mixture was heated to 50° C. After 30 minutes zinc powder (152 mg, 2.319 mmol) was added then a solution of 5-azido-2-(4-bromo-2-methoxybenzyl)-N-butyl-2H-pyrazolo[4,3-d]pyrimidin-7-amine (from Compound B-1 step 1, 500 mg, 1.159 mmol) and tert-butyl 3-bromoazetidine-1-carboxylate (411 mg, 1.739 mmol) in DMA (10 mL). After stirring overnight at rt the mixture was purified directly by reverse-phase chromatography (CH₃CN/water with 0.1% TFA modifier) to give the title compound. MS m/z (M+H)⁺: calculated 508.3, observed 508.4.

### Step 2: 2-(4-(azetidin-3-yl)-2-methoxybenzyl)-5-azido-N-butyl-2H-pyrazolo[4,3-d]pyrimidin-7-amine

To a solution of tert-butyl 3-(4-((5-azido-7-(butylamino)-2H-pyrazolo[4,3-d]pyrimidin-2-yl)methyl)-3-methoxyphenyl)azetidine-1-carboxylate (60 mg, 0.118 mmol) in DCM (0.5 mL) was added 4M HCl in 1,4-dioxane (0.5 mL) at rt. After 1 hr the mixture was concentrated to give the title compound as the HCl salt which was used without purification. MS m/z (M+H)⁺: calculated 408.2, observed 408.3.

### Intermediate 17-1

### Preparation of Compound Int. 17-1

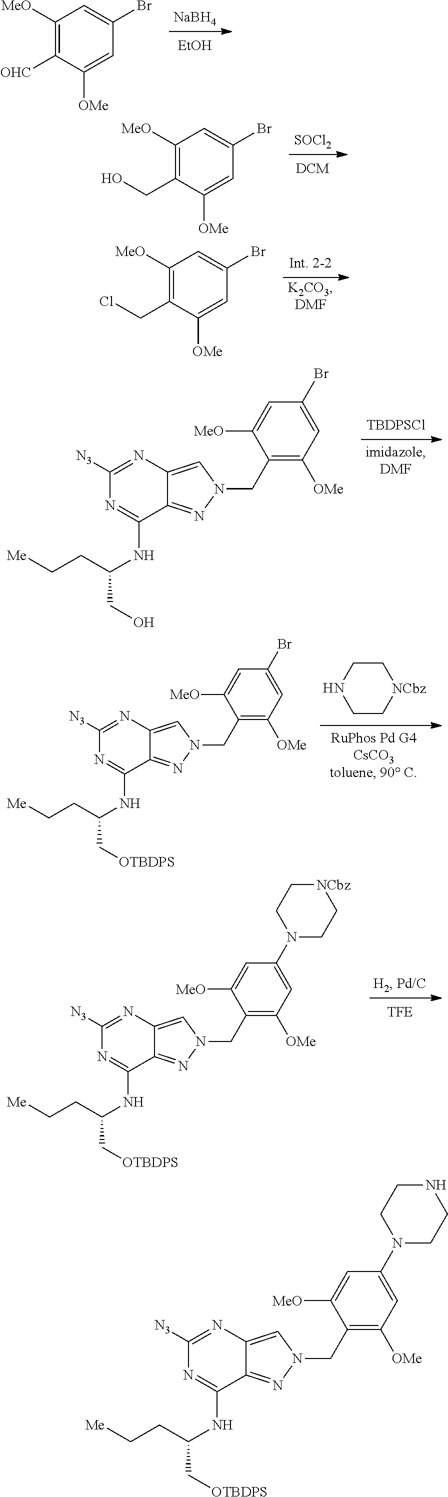

### (S)-5-azido-N-(1-((tert-butyldiphenylsilyl)oxy)pentan-2-yl)-2-(2,6-dimethoxy-4-(piperazin-1-yl)benzyl)-2H-pyrazolo[4,3-d]pyrimidin-7-amine

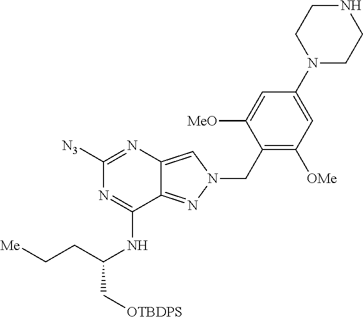

### Step 1: (4-bromo-2,6-dimethoxyphenyl)methanol

To a solution of 4-bromo-2,6-dimethoxybenzaldehyde (5 g, 20.40 mmol) in EtOH (60 mL) was added NaBH₄ (0.926 g, 24.48 mmol) at rt. After 1 hr the mixture was diluted with water (50 mL) then extracted with EtOAc (30 mL×2). The combined organic layers were washed with brine (50 mL) then dried (Na₂SO₄) then filtered then the filtrate was concentrated. The crude product was subjected to silica gel chromatography (15-50% EtOAc/pet. ether) to give the title compound. ¹H NMR (400 MHz, CDCl₃) δ 6.72 (s, 2H), 4.72 (d, J=6.8 Hz, 2H), 3.84 (s, 6H), 2.32 (t, J=6.8 Hz, 1H).

### Step 2: 5-bromo-2-(chloromethyl)-1,3-dimethoxybenzene

To a solution of (4-bromo-2,6-dimethoxyphenyl)methanol (1 g, 4.05 mmol) in DCM (10 mL) was added SOCl₂ (0.591 mL, 8.09 mmol) at 0° C. After the addition was complete the mixture was warmed to rt and stirred overnight. The resulting mixture was concentrated to give the title compound which was used in the next step without purification. ¹H NMR (400 MHz, CDCl₃) δ 6.72 (s, 2H), 4.70 (s, 2H), 3.87 (s, 6H).

### Step 3: (S)-2-((5-azido-2-(4-bromo-2,6-dimethoxybenzyl)-2H-pyrazolo[4,3-d]pyrimidin-7-yl)amino)pentan-1-ol

To a solution of (S)-2-((5-azido-2H-pyrazolo[4,3-d]pyrimidin-7-yl)amino)pentan-1-ol (Int. 2-2, 800 mg, 3.05 mmol) in DMF (15 mL) was added 5-bromo-2-(chloromethyl)-1,3-dimethoxybenzene (800 mg, 3.01 mmol) and K₂CO₃ (1.25 g, 9.04 mmol) at rt. After stirring overnight the mixture was diluted with water (10 mL). The resulting solid was collected by filtration then dried under vacuum to give the title compound. MS m/z (M+H)⁺: calculated 491.1, observed 491.2. ¹H NMR (400 MHz, CD₃OD) δ 8.53 (s, 1H), 6.93 (s, 2H), 5.64 (d, J=1.6 Hz, 2H), 4.50-4.63 (m, 1H), 3.88 (s, 6H), 3.68-3.76 (m, 2H), 1.61-1.86 (m, 2H), 1.38-1.54 (m, 2H), 0.97 (t, J=7.2 Hz, 3H).

### Step 4: (S)-5-azido-2-(4-bromo-2,6-dimethoxybenzyl)-N-(1-((tert-butyldiphenylsilyl)oxy)pentan-2-yl)-2H-pyrazolo[4,3-d]pyrimidin-7-amine

To a solution of (S)-2-((5-azido-2-(4-bromo-2,6-dimethoxybenzyl)-2H-pyrazolo[4,3-d]pyrimidin-7-yl)amino)pentan-1-ol (900 mg, 1.832 mmol) in DMF (15 mL) was added imidazole (374 mg, 5.50 mmol) and TBDPSCl (0.565 mL, 2.198 mmol) at rt. After stirring overnight the mixture was diluted with water (15 mL) then extracted with EtOAc (15 mL×2). The combined organic layers were washed with brine (20 mL) then dried (Na₂SO₄) then filtered then the filtrate was concentrated. The crude product was subjected to silica gel chromatography (0-10% MeOH/DCM) to give the title compound. MS m/z (M+H)⁺: calculated 731.2, observed 731.2.

### Step 5: benzyl (S)-4-(4-((5-azido-7-((1-((tert-butyldiphenylsilyl)oxy)pentan-2-yl)amino)-2H-pyrazolo[4,3-d]pyrimidin-2-yl)methyl)-3,5-dimethoxyphenyl)piperazine-1-carboxylate

To a solution of (S)-5-azido-2-(4-bromo-2,6-dimethoxybenzyl)-N-(1-((tert-butyldiphenylsilyl)oxy)pentan-2-yl)-2H-pyrazolo[4,3-d]pyrimidin-7-amine (500 mg, 0.685 mmol) in toluene (10 mL) was added benzyl piperazine-1-carboxylate (226 mg, 1.028 mmol), RuPhos Pd G4 (58.3 mg, 0.069 mmol), and Cs₂CO₃ (670 mg, 2.055 mmol). The mixture was purged with N₂ then heated to 90° C. After heating overnight the mixture was cooled to rt then diluted with water (10 mL) then extracted with DCM (15 mL×2). The combined organic layers were dried (Na₂SO₄) then filtered then the filtrate was concentrated. The crude product was subjected to silica gel chromatography (0-5% MeOH/DCM) to give the title compound. MS m/z (M+H)⁺: calculated 869.4, observed 869.4.

### Step 6: (S)-5-azido-N-(1-((tert-butyldiphenylsilyl)oxy)pentan-2-yl)-2-(2,6-dimethoxy-4-(piperazin-1-yl)benzyl)-2H-pyrazolo[4,3-d]pyrimidin-7-amine

To a solution of benzyl (S)-4-(4-((5-amino-7-((1-((tert-butyldiphenylsilyl)oxy)pentan-2-yl)amino)-2H-pyrazolo[4,3-d]pyrimidin-2-yl)methyl)-3,5-dimethoxyphenyl)piperazine-1-carboxylate (370 mg, 0.439 mmol) in TFE (8 mL) was added Pd/C (47 mg). The mixture was degassed (3×vacuum/N₂) then placed under an atmosphere of H₂ (balloon). After 2 hr the mixture was filtered then the filtrate was concentrated to give the title compound which was used in the next step without purification. MS m/z (M+H)⁺: calculated 735.4, observed 735.4.

### Example 3

### Preparation of Compound A-1

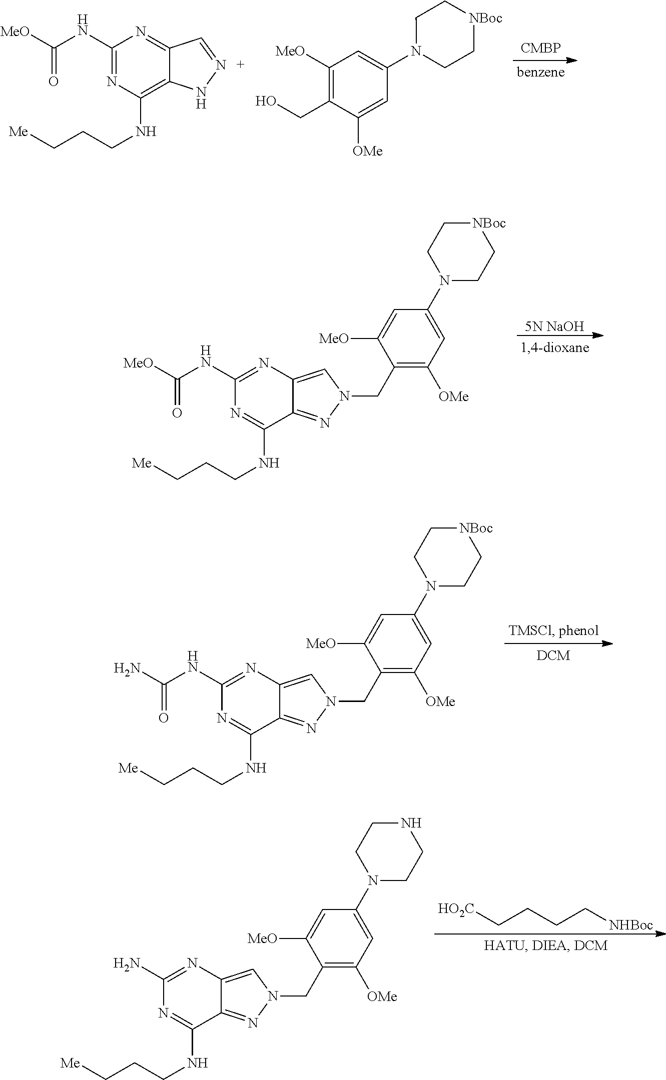

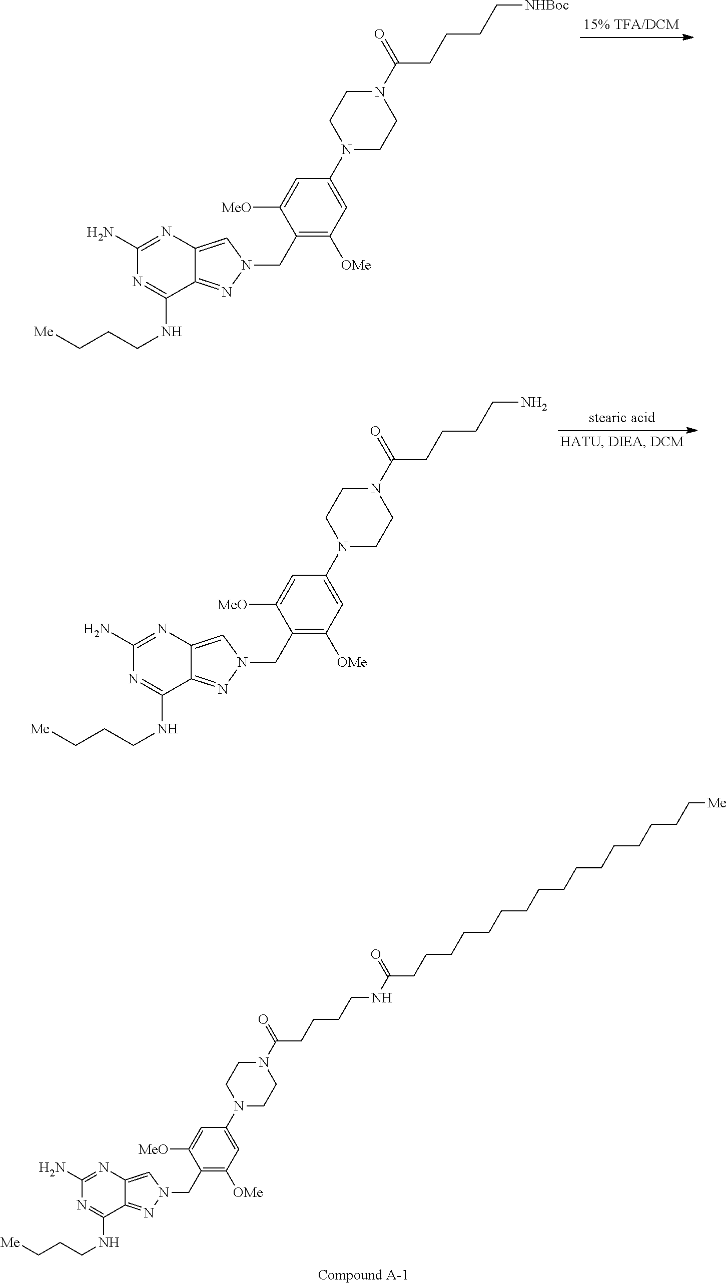 [SMILES: CCCCNc1nc(N)nc2cn(Cc3c(OC)cc(N4CCN(C(=O)CCCCNC(=O)OC(C)(C)C)CC4)cc3OC)nc12 ; CCCCNc1nc(N)nc2cn(Cc3c(OC)cc(N4CCN(C(=O)CCCCN)CC4)cc3OC)nc12 ; CCCCCCCCCCCCCCCCCC(=O)NCCCCC(=O)N1CCN(c2cc(OC)c(Cn3cc4nc(N)nc(NCCCC)c4n3)c(OC)c2)CC1]

### N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide (Compound A-1)

### Step 1: tert-butyl 4-(4-((7-(butylamino)-5-((methoxycarbonyl)amino)-2H-pyrazolo[4,3-d]pyrimidin-2-yl)methyl)-3,5-dimethoxyphenyl)piperazine-1-carboxylate

To a stirred mixture of tert-butyl 4-(4-(hydroxymethyl)-3,5-dimethoxyphenyl)piperazine-1-carboxylate (Int. 3-1, 13.87 g, 39.4 mmol) in benzene (80 mL) was added methyl (7-(butylamino)-1H-pyrazolo[4,3-d]pyrimidin-5-yl)carbamate (Int. 1-1, 8 g, 30.3 mmol) and cyanomethylenetributylphosphorane (10.96 g, 45.4 mmol) under an argon atmosphere, then the mixture was heated to 80° C. After 3 h, the mixture was cooled to ambient temperature, then diluted with water (10 mL), then extracted with EtOAc (3×20 mL). The combined organic extracts were washed with brine (3×10 mL) then dried (Na₂SO₄) then filtered then the filtrate was concentrated. The crude product was subjected to silica gel chromatography (0-100% EtOAc/DCM) to give the title compound. MS m/z (M+H)⁺: calculated 599.3, observed 599.3.

### Step 2: tert-butyl 4-(4-((5-amino-7-(butylamino)-2H-pyrazolo[4,3-d]pyrimidin-2-yl)methyl)-3,5-dimethoxyphenyl)piperazine-1-carboxylate

To a stirred mixture of tert-butyl 4-(4-((7-(butylamino)-5-((methoxycarbonyl)amino)-2H-pyrazolo[4,3-d]pyrimidin-2-yl)methyl)-3,5-dimethoxyphenyl)piperazine-1-carboxylate (2.6 g, 4.34 mmol) in 1,4-dioxane (5 mL) was added NaOH (10M, 20 mL, 200 mmol) then the mixture was heated to 60° C. After 12 h the mixture was cooled to ambient temperature then diluted with water (20 mL) then extracted with EtOAc (3×80 mL). The combined organic extracts were washed with brine (50 mL) then dried (Na₂SO₄) then filtered then the filtrate was concentrated. The crude product was subjected to silica gel chromatography (0-10% MeOH/DCM) to give the title compound. MS m/z (M+H)⁺: calculated 541.3, observed 541.4.

### Step 3: N⁷-butyl-2-(2,6-dimethoxy-4-(piperazin-1-yl)benzyl)-2H-pyrazolo[4,3-d]pyrimidine-5,7-diamine

To a stirred mixture of tert-butyl 4-(4-((5-amino-7-(butylamino)-2H-pyrazolo[4,3-d]pyrimidin-2-yl)methyl)-3,5-dimethoxyphenyl)piperazine-1-carboxylate (1.8 g, 3.33 mmol) in DCM (25 mL) was added a solution of phenol (9.40 g, 100 mmol) in DCM (5 mL) at ambient temperature. The mixture was cooled to 5° C. then a solution of chlorotrimethylsilane (0.24 ml, 1.878 mmol) in DCM (3 mL) was added dropwise. After the addition was complete the mixture was warmed to ambient temperature. After 25 minutes, the mixture was poured into ice-cold 2 N NaOH (30 mL), then extracted with ethyl EtOAc (5×150 mL). The combined organic extracts were dried (Na₂SO₄) then filtered then the filtrate was concentrated. The crude product was subjected to reverse-phase chromatography (0-60% MeCN/water with 0.5% NH₄OH modifier) to give the title compound. MS m/z (M+H)⁺: calculated 441.3, observed 441.2.

### Step 4: tert-butyl (5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)carbamate

To a stirred mixture of 5-((tert-butoxycarbonyl)amino)pentanoic acid (225 mg, 1.035 mmol) in DMF (2 mL) was added a solution of HATU (738 mg, 1.941 mmol) in DMF at ambient temperature. After 30 minutes N⁷-butyl-2-(2,6-dimethoxy-4-(piperazin-1-yl)benzyl)-2H-pyrazolo[4,3-d]pyrimidine-5,7-diamine (570 mg, 1.294 mmol) and DIEA (0.452 mL, 2.59 mmol) were added. After 5 h the mixture was cooled to ambient temperature then diluted with water (30 mL) then extracted with EtOAc (3×50 mL). The combined organic extracts were washed with brine (3×25 mL) then dried (Na₂SO₄) then filtered then the filtrate was concentrated. The crude product was subjected to silica gel chromatography (1-10% MeOH/DCM) to give the title compound. MS m/z (M+H)⁺: calculated 640.4, observed 640.5.

### Step 5: 5-amino-1-(4-(4-((5-amino-7-(butylamino)-2H-pyrazolo[4,3-d]pyrimidin-2-yl)methyl)-3,5-dimethoxyphenyl)piperazin-1-yl)pentan-1-one

A solution of tert-butyl (5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)carbamate (550 mg, 0.860 mmol) in 10% TFA/DCM (2 mL) was stirred at ambient temperature. After 30 minutes the mixture was diluted with toluene (30 mL) then concentrated. The crude product was subjected to reverse-phase chromatography (0-100% MeCN/water with 1% NH₄OH modifier) to give the title compound. MS m/z (M+H)⁺: calculated 540.3, observed 540.5.

### Step 6: N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide

To a stirred mixture of stearic acid (274 mg, 0.964 mmol) in DMF (7 mL) was added a solution of HATU (423 mg, 1.112 mmol) in DMF at ambient temperature. After 30 minutes 5-amino-1-(4-(4-((5-amino-7-(butylamino)-2H-pyrazolo[4,3-d]pyrimidin-2-yl)methyl)-3,5-dimethoxyphenyl)piperazin-1-yl)pentan-1-one (400 mg, 0.741 mmol) and DIEA (0.259 ml, 1.482 mmol) were added. After 2 h the mixture was cooled to ambient temperature then diluted with water (20 mL) then extracted with EtOAc (3×50 mL). The combined organic extracts were washed with brine (3×20 mL) then dried (Na₂SO₄) then filtered then the filtrate was concentrated. The crude product was subjected to silica gel chromatography (1-10% MeOH/DCM) to give the title compound. Compound A-1: MS m/z (M+H)⁺: calculated 806.6, observed 806.6. ¹H-NMR (400 MHz, CDCl₃) δ 12.41 (s, 1H), 7.41 (s, 1H), 6.71-6.68 (m, 1H), 6.11 (s, 2H), 5.78-5.76 (m, 1H), 5.38 (s, 2H), 3.85-3.82 (m, 9H), 3.65-3.60 (m, 5H), 3.28-3.25 (m, 7H), 2.41 (t, J=7.2 Hz, 2H), 2.16 (t, J=7.2 Hz, 2H), 1.70-1.56 (m, 18H), 1.51-1.49 (m, 4H), 1.25 (s, 9H), 1.00-0.90 (m, 4H), 0.89-0.80 (m, 6H).

### Preparation of Compound A-2

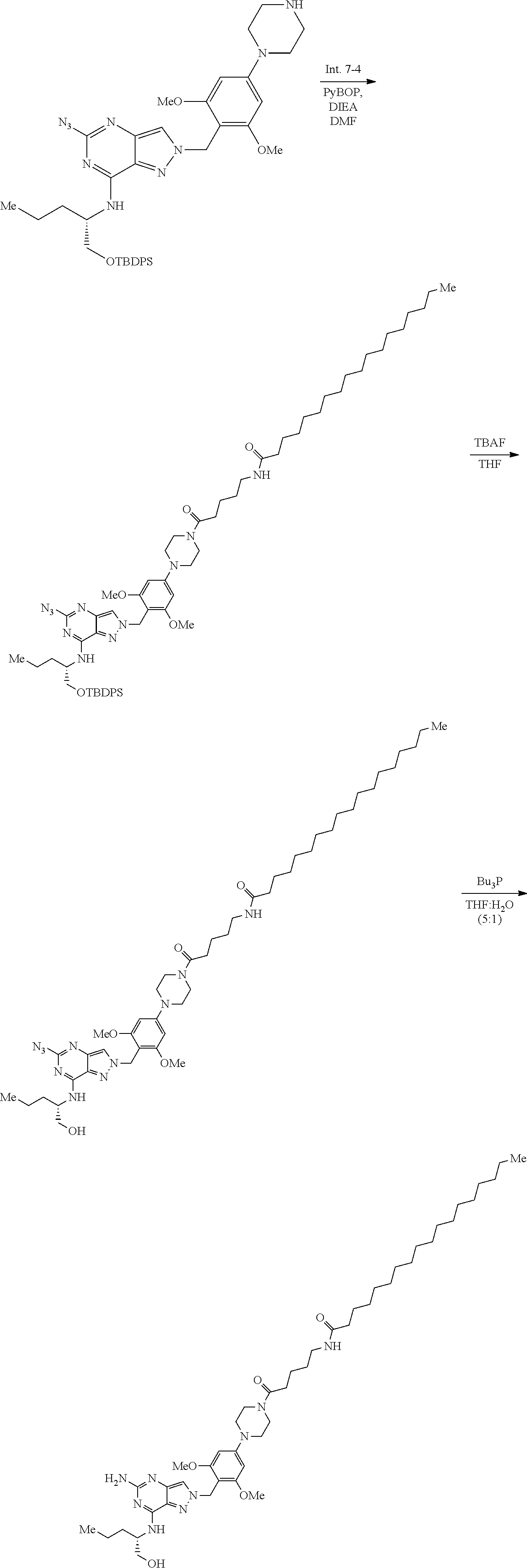

### (S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide (Compound A-2)

### Step 1: (S)—N-(5-(4-(4-((5-azido-7-((1-((tert-butyldiphenylsilyl)oxy)pentan-2-yl)amino)-2H-pyrazolo[4,3-d]pyrimidin-2-yl)methyl)-3,5-dimethoxyphenyl)piperazin-1-yl)-5-oxopentyl)stearamide

To a solution of 5-stearamidopentanoic acid (Int. 7-4, 129 mg, 0.337 mmol) in DMF (10 mL) was added DIEA (109 mg, 0.842 mmol) and PyBOP (175 mg, 0.337 mmol) at rt. After 10 minutes (S)-5-azido-N-(1-((tert-butyldiphenylsilyl)oxy)pentan-2-yl)-2-(2,6-dimethoxy-4-(piperazin-1-yl)benzyl)-2H-pyrazolo[4,3-d]pyrimidin-7-amine (Int. 17-1, 300 mg, 0.281 mmol) was added at rt. After 30 minutes the mixture was diluted with water (10 mL) then extracted with EtOAc (15 mL×2). The combined organic layers were dried (Na₂SO₄) then filtered then the filtrate was concentrated. The crude product was purified by prep-TLC (DCM/MeOH=10:1) to give the title compound and (S)-1-(4-(4-((5-azido-7-((1-((tert-butyldiphenylsilyl)oxy)pentan-2-yl)amino)-2H-pyrazolo[4,3-d]pyrimidin-2-yl)methyl)-3,5-dimethoxyphenyl)piperazin-1-yl)octadecan-1-one (Int. 18-1). Title Compound: MS m/z (M+H)⁺: calculated 1100.7, observed 1100.7. Int. 18-1: MS m/z (M+H)⁺: calculated 1001.6, observed 1001.7.

### Step 2: (S)—N-(5-(4-(4-((5-azido-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide

To a solution of (S)—N-(5-(4-(4-((5-azido-7-((1-((tert-butyldiphenylsilyl)oxy)pentan-2-yl)amino)-2H-pyrazolo[4,3-d]pyrimidin-2-yl)methyl)-3,5-dimethoxyphenyl)piperazin-1-yl)-5-oxopentyl)stearamide (150 mg, 0.075 mmol) in THE (2 mL) was added TBAF (0.112 mL, 0.112 mmol, 1M in THF) at rt. After 1 hr the mixture was diluted with water (10 mL) then extracted with EtOAc (15 mL×2). The combined organic layers were dried (Na₂SO₄) then filtered then the filtrate was concentrated to give the title compound which was used in the next step without purification. MS m/z (M+H)⁺: calculated 862.6, observed 862.5.

### Step 3: (S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide

To a solution of (S)—N-(5-(4-(4-((5-azido-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide (55 mg, 0.064 mmol) in THF (1.5 mL) and H₂O (0.3 mL) was added Bu₃P (0.079 mL, 0.319 mmol) then the mixture was heated to 50° C. After 1 hr the mixture was cooled to rt then concentrated. The crude product was purified by reverse-phase chromatography (CH₃CN/water with 0.2% FA modifier) to give the title compound. Compound A-2: MS m/z (M+H)⁺: calculated 836.6, observed 836.5. ¹H NMR (400 MHz, CD₃OD) δ 7.57 (s, 1H), 6.27 (s, 2H), 5.42 (s, 2H), 4.49-4.40 (m, 1H), 3.84 (s, 6H), 3.72 (dd, J=5.2, 10.0 Hz, 4H), 3.68-3.61 (m, 2H), 3.27-3.18 (m, 4H), 2.48 (t, J=7.2 Hz, 2H), 2.17 (t, J=7.2 Hz, 2H), 1.73-1.53 (m, 10H), 1.50-1.36 (m, 4H), 1.30-1.24 (m, 27H), 0.97 (t, J=7.2 Hz, 4H), 0.89 (t, J=6.8 Hz, 3H).

Utilizing the procedures described for Compound A-2, the following compounds were prepared substituting the appropriate reagents for 5-stearamidopentanoic acid in step 1.

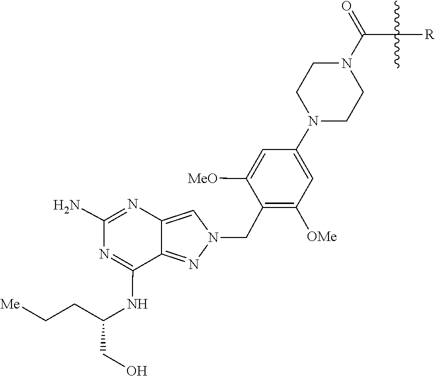

| Cmpd/ | | | Calc'd | Observed |
|---|---|---|---|---|
| Ex. # | R | Name | [M + H]⁺ | [M + H]⁺ |
| A-3 | 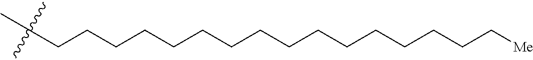 [SMILES: *CCCCCCCCCCCCCCCCC] | (S)-1-(4-(4-((5- amino-7-((1- hydroxypentan-2- yl)amino)-2H- pyrazolo[4,3- d]pyrimidin-2- yl)methyl)-3,5- dimethoxyphenyl) piperazin-1- yl)octadecan-1-one | 737.5 | 737.5 |

### Example 4

### Preparation of Compound B-1

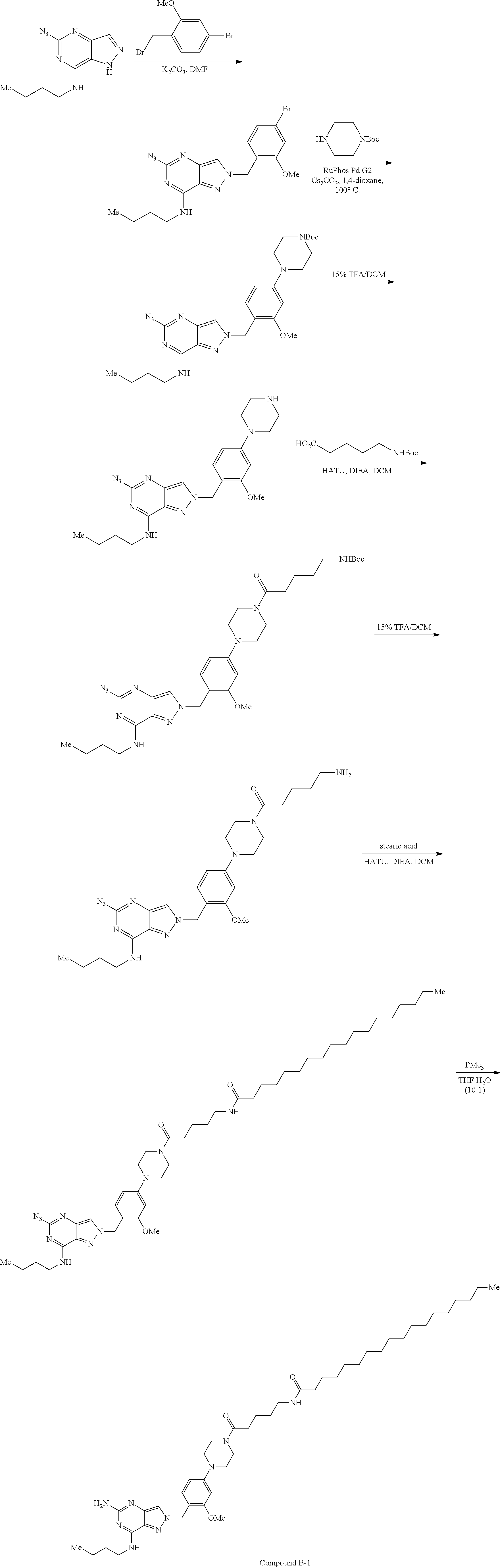

### N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide (Compound B-1)

### Step 1: 5-Azido-2-(4-bromo-2-methoxybenzyl)-N-butyl-2H-pyrazolo[4,3-d]pyrimidin-7-amine

5-Azido-N-butyl-1H-pyrazolo[4,3-d]pyrimidin-7-amine (Int. 2-1, 938 mg, 4.04 mmol), 4-bromo-1-(bromomethyl)-2-methoxybenzene (1.36 g, 4.86 mmol), and K₂CO₃ (1.2 g, 8.68 mmol) were combined in DMF (20 mL) at ambient temperature. After stirring overnight the mixture was diluted with H₂O then extracted with EtOAc (3×). The combined organic extracts were washed with brine then dried (Na₂SO₄) then filtered then the filtrate was concentrated. The crude product was subjected to silica gel chromatography (0-50% 3:1 EtOAc:EtOH/heptane) to give the title compound. MS m/z (M+H)⁺: calculated 431.3, observed 431.0.

### Step 2: tert-butyl 4-(4-((5-azido-7-(butylamino)-2H-pyrazolo[4,3-d]pyrimidin-2-yl)methyl)-3-methoxyphenyl)piperazine-1-carboxylate

5-Azido-2-(4-bromo-2-methoxybenzyl)-N-butyl-2H-pyrazolo[4,3-d]pyrimidin-7-amine (1.38 g, 3.20 mmol), tert-butyl piperazine-1-carboxylate (0.775 g, 4.16 mmol), and Cs₂CO₃ (3.13 g, 9.60 mmol) were combined in 1,4-dioxane (20 mL). The mixture was degassed (3×pump/N₂). RuPhos Pd G2 (0.25 g, 0.322 mmol) was added then the mixture was heated to 100° C. After stirring overnight at 100° C., the mixture was cooled to ambient temperature then diluted with EtOAc then filtered through a pad of Celite®, washing with EtOAc, then the filtrate was concentrated. The crude product was subjected to silica gel chromatography (0-50% 3:1 EtOAc:EtOH/heptane) to give the title compound. MS m/z (M+H)⁺: calculated 537.3, observed 537.1.

### Step 3: 5-azido-N-butyl-2-(2-methoxy-4-(piperazin-1-yl)benzyl)-2H-pyrazolo[4,3-d]pyrimidin-7-amine

To a solution of tert-butyl 4-(4-((5-azido-7-(butylamino)-2H-pyrazolo[4,3-d]pyrimidin-2-yl)methyl)-3-methoxyphenyl)piperazine-1-carboxylate (799 mg, 1.489 mmol) in DCM (8 mL) was added TFA (1.5 mL, 19.47 mmol) at ambient temperature. After 2 h the mixture was concentrated. The residue was taken up in 1:1 DCM:heptane then concentrated (2×) then dried under vacuum to give the TFA salt of the title compound which was used directly in the next step. MS m/z (M+H)⁺: calculated 437.2, observed 437.1.

### Step 4: tert-butyl (5-(4-(4-((5-azido-7-(butylamino)-2H-pyrazolo[4,3-d]pyrimidin-2-yl)methyl)-3-methoxyphenyl)piperazin-1-yl)-5-oxopentyl)carbamate

5-((tert-butoxycarbonyl)amino)pentanoic acid (421 mg, 1.936 mmol) and HATU (736 mg, 1.936 mmol) were combined in DCM (5 mL) then DIEA (1.300 ml, 7.45 mmol) was added at ambient temperature. After 1 h a solution of crude 5-azido-N-butyl-2-(2-methoxy-4-(piperazin-1-yl)benzyl)-2H-pyrazolo[4,3-d]pyrimidin-7-amine (TFA salt, 650 mg, 1.489 mmol) in DCM (10 mL) was added. After stirring overnight at ambient temperature the mixture was concentrated. The crude product was subjected to silica gel chromatography (0-100% 9:1 DCM:MeOH/DCM) to give impure product which was further purified by silica gel chromatography (0-100% 3:1 EtOAc:EtOH/heptane) to give the title compound. MS m/z (M+H)⁺: calculated 636.4, observed 636.2.

### Step 5: 5-amino-1-(4-(4-((5-azido-7-(butylamino)-2H-pyrazolo[4,3-d]pyrimidin-2-yl)methyl)-3-methoxyphenyl)piperazin-1-yl)pentan-1-one

Tert-butyl (5-(4-(4-((5-azido-7-(butylamino)-2H-pyrazolo[4,3-d]pyrimidin-2-yl)methyl)-3-methoxyphenyl)piperazin-1-yl)-5-oxopentyl)carbamate (848 mg, 1.334 mmol) was taken up in DCM (10 mL) then TFA (1.1 mL, 14.28 mmol) as added at ambient temperature. After 90 minutes the mixture was concentrated give the TFA salt of the title compound which was used directly in the next step. MS m/z (M+H)⁺: calculated 536.3, observed 536.3.

### Step 6: N-(5-(4-(4-((5-azido-7-(butylamino)-2H-pyrazolo[4,3-d]pyrimidin-2-yl)methyl)-3-methoxyphenyl)piperazin-1-yl)-5-oxopentyl)stearamide

Stearic acid (493 mg, 1.734 mmol) and HATU (659 mg, 1.734 mmol) were combined in DCM (5 mL) then DIEA (1.2 ml, 6.87 mmol) was added at ambient temperature. After 30 minutes the mixture was transferred to a flask containing crude 5-amino-1-(4-(4-((5-azido-7-(butylamino)-2H-pyrazolo[4,3-d]pyrimidin-2-yl)methyl)-3-methoxyphenyl)piperazin-1-yl)pentan-1-one (TFA salt, 715 mg, 1.334 mmol) and DCM (10 mL). After stirring overnight at ambient temperature the mixture was concentrated. The crude product was subjected to silica gel chromatography (0-100% 9:1 DCM:MeOH/DCM) to give impure product which was further purified by silica gel chromatography (0-100% 3:1 EtOAc:EtOH/heptane) to give the title compound. MS m/z (M+H)⁺: calculated 802.6, observed 802.9.

### Step 7: N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide

N-(5-(4-(4-((5-azido-7-(butylamino)-2H-pyrazolo[4,3-d]pyrimidin-2-yl)methyl)-3-methoxyphenyl)piperazin-1-yl)-5-oxopentyl)stearamide (641 mg, 0.799 mmol) was taken up in THF (8 mL):H₂O (0.800 mL) resulting in a thick suspension. The mixture was heated to 50° C. and as the mixture was heating the suspension became a solution. To this was added 1M trimethylphosphine (2.4 mL, 2.400 mmol) in THF and heating continued at 50° C. After 3 h the mixture was cooled to ambient temperature then quenched with MeOH then concentrated. The crude product was subjected to silica gel chromatography (0-15% MeOH:DCM) to give the title compound. Compound B-1: MS m/z (M+H)⁺: calculated 776.6, observed 776.5. ¹H-NMR (400 MHz, DMSO-d6) δ 11.5 (br., 1H), 8.11 (s, 1H), 7.72-7.65 (m, 2H), 7.06-6.80 (m, 1H), 6.60 (s, 1H), 6.49-6.47 (m, 1H), 6.32 (s, 1H), 5.32 (s, 2H), 3.81 (s, 3H), 3.57-3.42 (m, 6H), 3.32-3.02 (m, 4H), 2.36-2.32 (m, 2H), 2.07-1.99 (m, 4H), 1.47-1.34 (m, 8H), 1.29-1.18 (m, 30H), 0.91-0.83 (m, 6H).

Utilizing the procedures described for Compound B-1, the following compounds were prepared substituting the appropriate reagents for stearic acid.

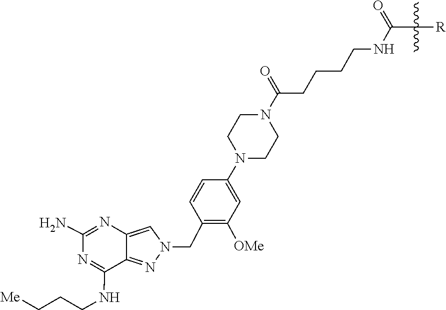

| | | | Calc'd | Observed |
|---|---|---|---|---|
| Cmpd. | R | Name | [M + H]⁺ | [M + H]⁺ |
| B-2 | 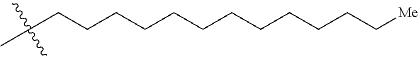 [SMILES: *CCCCCCCCCCCCC] | N-(5-(4-(4-((5- amino-7- (butylamino)-2H- pyrazolo[4,3- d]pyrimidin-2- yl)methyl)-3- methoxyphenyl) piperazin-1-yl)-5- oxopentyl)tetrade canamide | 720.5 | 720.5 |
| B-3 | 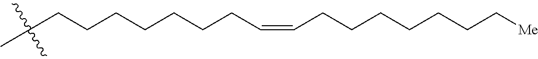 [SMILES: *CCCCCCC/C=C\CCCCCCCC] | N-(5-(4-(4-((5- amino-7- (butylamino)-2H- pyrazolo[4,3- d]pyrimidin-2- yl)methyl)-3- methoxyphenyl) piperazin-1-yl)-5- oxopentyl)oleamide | 774.6 | 774.6 |
| B-4 | 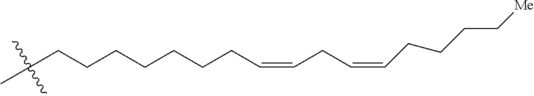 [SMILES: *CCCCCCC/C=C\C/C=C\CCCCC] | (9Z,12Z)-N-(5-(4- (4-((5-amino-7- (butylamino)-2H- pyrazolo[4,3- d]pyrimidin-2- yl)methyl)-3- methoxyphenyl) piperazin-1-yl)-5- oxopentyl) octadeca-9,12- dienamide | 772.6 | 772.5 |

### Preparation of Compound B-5

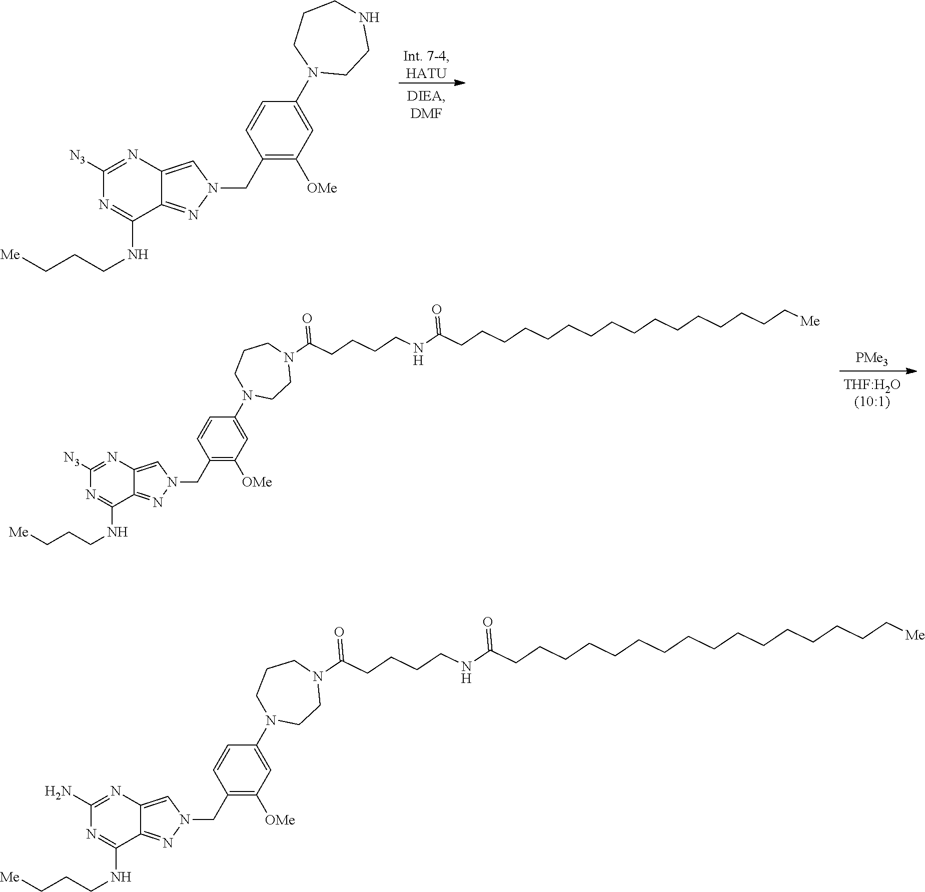 [SMILES: CCCCNc1nc(N=[N+]=[N-])nc2cn(Cc3ccc(N4CCCNCC4)cc3OC)nc12 ; "Int. 7-4,HATU" ; CCCCCCCCCCCCCCCCCC(=O)NCCCCC(=O)N1CCCN(c2ccc(Cn3cc4nc(N=[N+]=[N-])nc(NCCCC)c4n3)c(OC)c2)CC1 ; CCCCCCCCCCCCCCCCCC(=O)NCCCCC(=O)N1CCCN(c2ccc(Cn3cc4nc(N)nc(NCCCC)c4n3)c(OC)c2)CC1 ; "PMe3"]

### N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide

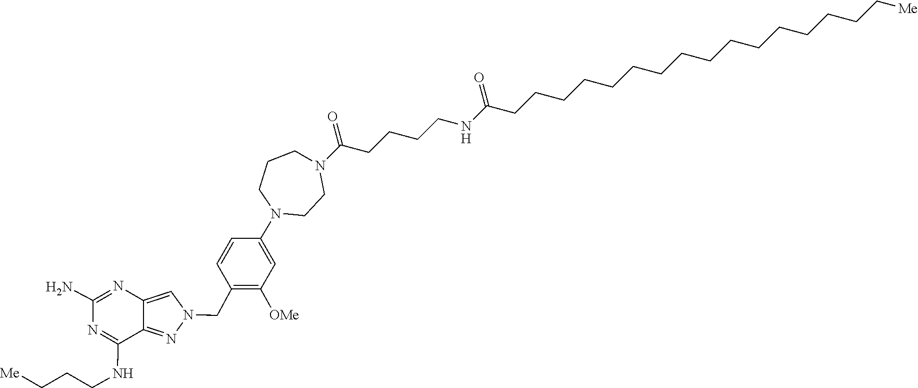 [SMILES: CCCCCCCCCCCCCCCCCC(=O)NCCCCC(=O)N1CCCN(c2ccc(Cn3cc4nc(N)nc(NCCCC)c4n3)c(OC)c2)CC1]

### Step 1: N-(5-(4-(4-((5-azido-7-(butylamino)-2H-pyrazolo[4,3-d]pyrimidin-2-yl)methyl)-3-methoxyphenyl)-1,4-diazepan-1-yl)-5-oxopentyl)stearamide

To a mixture of 2-(4-(1,4-diazepan-1-yl)-2-methoxybenzyl)-5-azido-N-butyl-2H-pyrazolo[4,3-d]pyrimidin-7-amine hydrochloride (Int. 14-1, 300 mg, 0.666 mmol) and 5-stearamidopentanoic acid (Int. 7-4, 383 mg, 0.999 mmol) in DMF (5 mL) was added HATU (506 mg, 1.332 mmol) and DIEA (0.349 mL, 1.998 mmol) at rt. After 2 hr the mixture was diluted with water (100 mL) then extracted with EtOAc (3×100 mL). The combined organic layers were washed with brine (3×100 mL) then dried (Na₂SO₄) then filtered then the filtrate was concentrated. The crude product was subjected to silica gel chromatography (0-6% MeOH/DCM) to give the title compound. MS m/z (M+H)⁺: calculated 816.6, observed 816.7.

### Step 2: N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide

To a solution of N-(5-(4-(4-((5-azido-7-(butylamino)-2H-pyrazolo[4,3-d]pyrimidin-2-yl)methyl)-3-methoxyphenyl)-1,4-diazepan-1-yl)-5-oxopentyl)stearamide (290 mg, 0.355 mmol) in THF (3 mL):H₂O (0.60 mL) was added PMe₃ (1.066 mL, 1.066 mmol) then the mixture was heated to 50° C. After heating overnight the mixture was cooled to rt then quenched with MeOH (5 mL) then the resulting mixture was concentrated. The crude product was subjected to silica gel chromatography (0-6% MeOH/DCM) to give the title compound. Compound B-5: MS m/z (M+H)⁺: calculated 790.6, observed 790.6. ¹H-NMR (400 MHz, DMSO-d6) δ 8.16 (s, 1H), 7.72 (d, J=7.2 Hz, 1H), 7.60-7.56 (m, 1H), 6.93 (d, J=8.0 Hz, 1H), 6.31-6.27 (m, 2H), 6.19 (s, 2H), 5.26 (s, 2H), 3.79 (s, 3H), 3.66-3.39 (m, 8H), 3.29-3.27 (m, 2H), 2.99-2.95 (m, 2H), 2.26 (t, J=7.2 Hz, 1H), 2.16 (t, J=7.2 Hz, 1H), 2.03-1.98 (m, 2H), 1.87-1.76 (m, 2H), 1.59-1.55 (m, 2H), 1.47-1.22 (m, 36H), 0.90 (t, J=7.2 Hz, 3H), 0.85 (t, J=7.2 Hz, 3H).

Utilizing the procedures described for Compound B-5, the following compounds were prepared substituting the appropriate reagents for 2-(4-(1,4-diazepan-1-yl)-2-methoxybenzyl)-5-azido-N-butyl-2H-pyrazolo[4,3-d]pyrimidin-7-amine hydrochloride and 5-stearamidopentanoic acid in step 1.

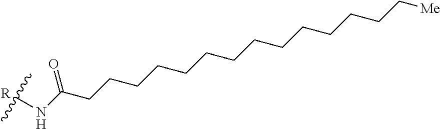

| Cmpd/ | | | Calc'd | Observed |
|---|---|---|---|---|
| Ex. # | R | Name | [M + H]⁺ | [M + H]⁺ |
| B-6 | 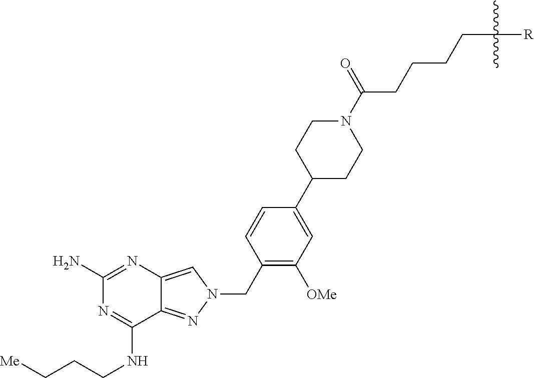 | N-(5-(4-(4-((5- amino-7- (butylamino)-2H- pyrazolo [4,3- d]pyrimidin-2- yl)methyl)-3- methoxyphenyl) piperidin-1-yl)-5- oxopentyl) stearamide | 775.6 | 775.6 |
| B-7 | 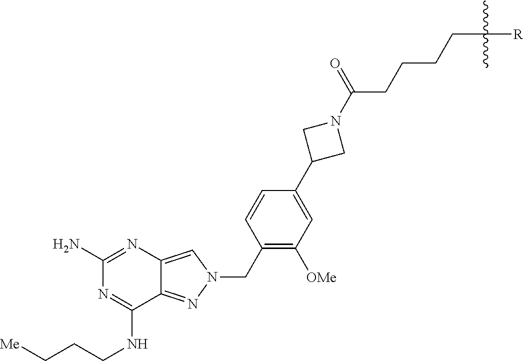 | N-(5-(3-(4-((5- amino-7- (butylamino)-2H- pyrazolo[4,3- d]pyrimidin-2- yl)methyl)-3- methoxyphenyl) azetidin-1-yl)-5- oxopentyl) stearamide | 747.6 | 747.6 |
| B-8 | 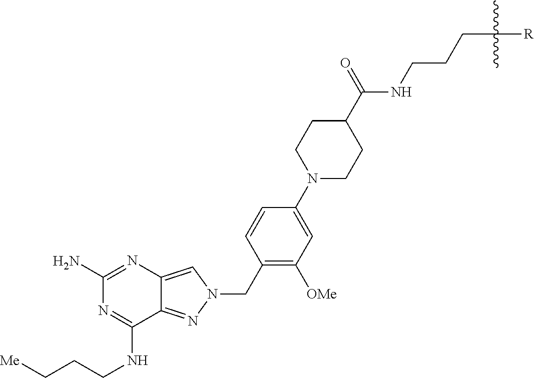 | 1-(4-((5-amino-7- (butylamino)-2H- pyrazolo[4,3- d]pyrimidin-2- yl)methyl)-3- methoxyphenyl)-N- (3- stearamidopropyl) piperidine-4- carboxamide | 776.6 | 776.6 |

### Preparation of Compound B-9 and B-10

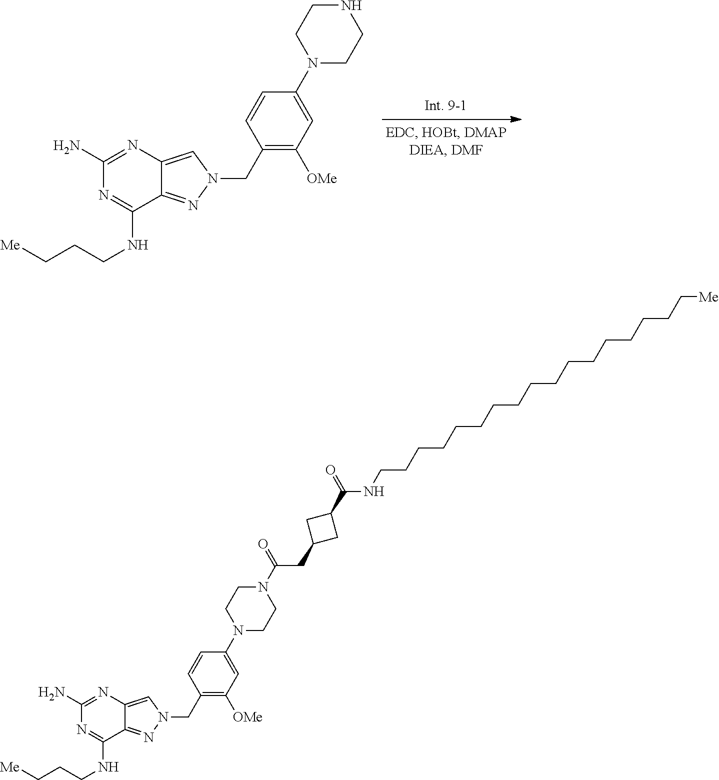 [SMILES: CCCCNc1nc(N)nc2cn(Cc3ccc(N4CCNCC4)cc3OC)nc12 ; "Int. 9-1" ; CCCCCCCCCCCCCCCCCCNC(=O)[C@H]1C[C@@H](CC(=O)N2CCN(c3ccc(Cn4cc5nc(N)nc(NCCCC)c5n4)c(OC)c3)CC2)C1]

### (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide (B-9)

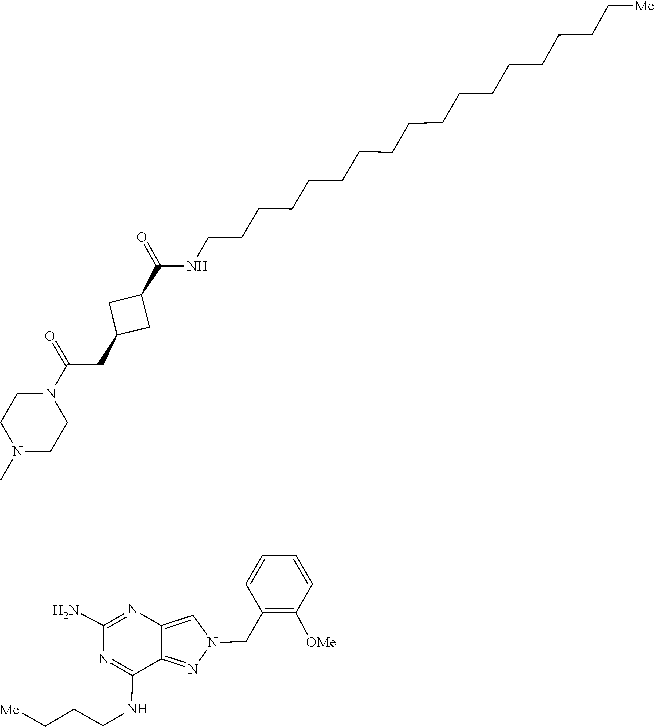 [SMILES: CCCCCCCCCCCCCCCCCCNC(=O)[C@H]1C[C@@H](CC(=O)N2CCN(C)CC2)C1 ; CCCCNc1nc(N)nc2cn(Cc3ccccc3OC)nc12]

### (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide (B-10)

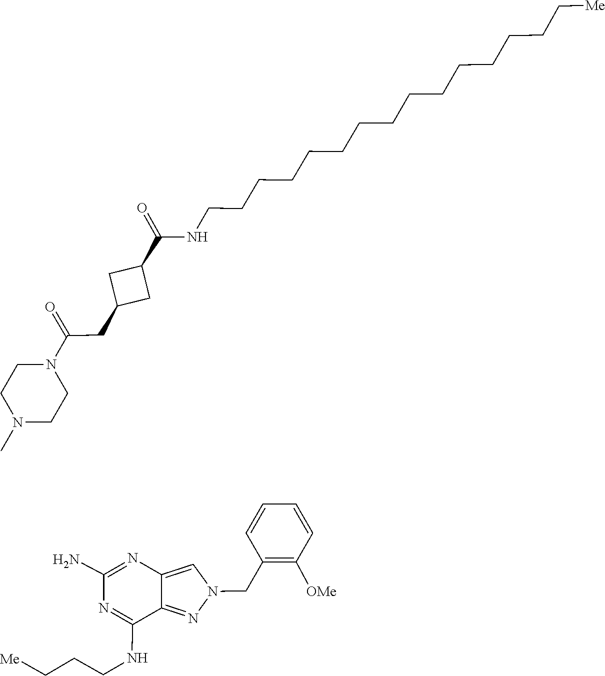 [SMILES: CCCCCCCCCCCCCCCCNC(=O)[C@H]1C[C@@H](CC(=O)N2CCN(C)CC2)C1 ; CCCCNc1nc(N)nc2cn(Cc3ccccc3OC)nc12]

### Step 1: (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide and (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide

To a stirred mixture of 2-(3-(octadecylcarbamoyl)cyclobutyl)acetic acid (Int. 9-1 as an 80:20 mixture with 2-((1s,3s)-3-(hexadecylcarbamoyl)cyclobutyl)acetic acid, 0.3 g, 0.732 mmol), DMAP (8.95 mg, 0.073 mmol), DIEA (0.192 mL, 1.098 mmol), EDC (0.211 g, 1.098 mmol) and HOBt (0.148 g, 1.098 mmol) in DMF (10 mL) was added a solution of N⁷-butyl-2-(2-methoxy-4-(piperazin-1-yl)benzyl)-2H-pyrazolo[4,3-d]pyrimidine-5,7-diamine (0.391 g, 0.952 mmol) in DMF (10 mL) at 25° C. After 6 hr the mixture was diluted with H₂O then extracted with EtOAc (3×10 mL). The combined organic layers were washed with brine (3×10 mL) then dried (Na₂SO₄) then filtered then the filtrate was concentrated. The crude product was subjected to silica gel chromatography (0-20% MeOH/DCM) to give a purified mixture of title compounds. The mixture was further purified by chiral chromatography (CHIRALPAK OD, 30% EtOH in hexanes containing 0.1% TFA modifier) to give P1 and P2.

P1: (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide Compound B-10: MS m/z (M+H)⁺: calculated 774.6, observed 774.6. ¹H-NMR (400 MHz, DMSO-d6) δ 8.37 (br., 1H), 7.67 (s, 1H), 7.58 (t, J=5.6 Hz, 1H), 6.96 (d, J=8.4 Hz, 1H), 6.60 (d, J=2.4 Hz, 1H), 6.50-6.43 (m, 1H), 5.32 (s, 2H), 3.80 (s, 3H), 3.55-3.43 (m, 5H), 3.16-3.11 (m, 2H), 2.99-2.95 (m, 2H), 2.85-2.73 (m, 1H), 2.46-2.43 (m, 4H), 2.14-2.13 (m, 2H), 1.76-1.75 (m, 2H), 1.58-1.55 (m, 2H), 1.23 (s, 32H), 0.95-0.85 (m, 6H).

P2: (1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide Compound B-9: MS m/z (M+H)⁺: calculated 802.6, observed 802.6. ¹H-NMR (400 MHz, DMSO-d6) δ 8.11 (br., 1H), 7.67 (s, 1H), 7.58 (t, J=5.6 Hz, 1H), 6.96 (d, J=8.4 Hz, 1H), 6.60 (d, J=2.4 Hz, 1H), 6.50-6.43 (m, 1H), 6.18 (br., 1H), 5.32 (s, 2H), 3.80 (s, 3H), 3.55 (t, J=5.2 Hz, 4H), 3.43 (d, J=6.8 Hz, 1H), 3.16-3.11 (m, 4H), 2.99-2.95 (m, 2H), 2.85-2.73 (m, 1H), 2.46-2.43 (m, 4H), 2.14-2.13 (m, 2H), 1.76-1.75 (m, 2H), 1.58-1.55 (m, 2H), 1.23 (s, 34H), 0.93-0.80 (m, 6H).

Utilizing the procedures described for Compounds B-9 and B-10, the following compounds were prepared substituting the appropriate reagents for 2-(3-(octadecylcarbamoyl)cyclobutyl)acetic acid.

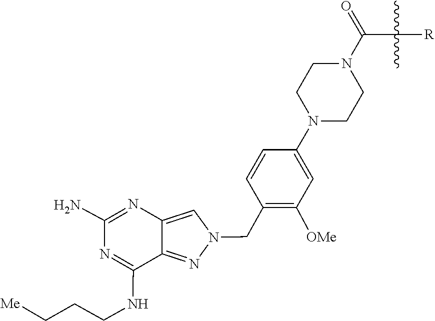

| Cmpd/ | | | Calc'd | Observed | |
|---|---|---|---|---|---|
| Ex. # | R | Name | [M + H]⁺ | [M + H]⁺ | Comments |
| B-11 | 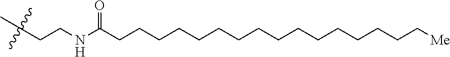 [SMILES: *CCNC(=O)CCCCCCCCCCCCCCCCC] | N-(3-(4-(4-((5- amino-7- (butylamino)- 2H- pyrazolo[4,3- d]pyrimidin-2- yl)methyl)-3- methoxyphenyl) piperazin-1-yl)- 3-oxopropyl) stearamide | 748.6 | 748.6 | |
| B-12 | 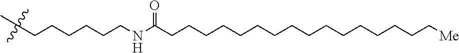 [SMILES: *CCCCCCNC(=O)CCCCCCCCCCCCCCCCC] | N-(7-(4-(4-((5- amino-7- (butylamino)- 2H- pyrazolo[4,3- d]pyrimidin-2- yl)methyl)-3- methoxyphenyl) piperazin-1-yl)- 7-oxoheptyl) stearamide | 804.6 | 804.7 | |
| B-13 | 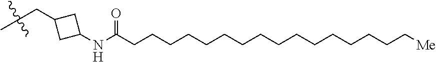 [SMILES: *CC1CC(NC(=O)CCCCCCCCCCCCCCCCC)C1] | N-(3-(2-(4-(4- ((5-amino-7- (butylamino)- 2H- pyrazolo[4,3- d]pyrimidin-2- yl)methyl)-3- methoxyphenyl) piperazin-1-yl)- 2-oxoethyl) cyclobutyl) stearamide | 788.6 | 788.8 | |
| B-14 | 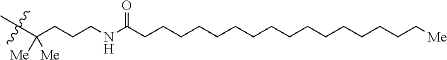 [SMILES: *C(C)(C)CCCNC(=O)CCCCCCCCCCCCCCCCC] | N-(5-(4-(4-((5- amino-7- (butylamino)- 2H- pyrazolo[4,3- d]pyrimidin-2- yl)methyl)-3- methoxyphenyl) piperazin-1-yl)- 4,4-dimethyl-5- oxopentyl) stearamide | 804.6 | 804.7 | |
| B-15 | 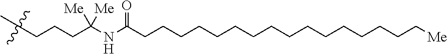 [SMILES: *CCCC(C)(C)NC(=O)CCCCCCCCCCCCCCCCC] | N-(6-(4-(4-((5- amino-7- (butylamino)- 2H- pyrazolo[4,3- d]pyrimidin-2- yl)methyl)-3- methoxyphenyl) piperazin-1-yl)- 2-methyl-6- oxohexan-2- yl)stearamide | 804.6 | 804.8 | |
| B-16 | 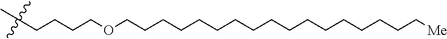 [SMILES: *CCCCOCCCCCCCCCCCCCCCCCC] | 1-(4-(4-((5- amino-7- (butylamino)- 2H- pyrazolo[4,3- d]pyrimidin-2- yl)methyl)-3- methoxyphenyl) piperazin-1-yl)- 5-(octadecyloxy) pentan-1-one | 763.6 | 763.6 | |
| B-17 | 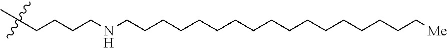 [SMILES: *CCCCNCCCCCCCCCCCCCCCCCC] | 1-(4-(4-((5- amino-7- (butylamino)- 2H- pyrazolo[4,3- d]pyrimidin-2- yl)methyl)-3- methoxyphenyl) piperazin-1-yl)- 5- (octadecylamino) pentan-1-one | 762.6 | 762.6 | from Int. 12-1 then deprotected using HCl in 1,4-dioxane |

Utilizing the procedures described for Compound B-1, the following compounds were prepared substituting the appropriate reagents for 5-azido-N-butyl-H-pyrazolo[4,3-d]pyrimidin-7-amine in step 1.

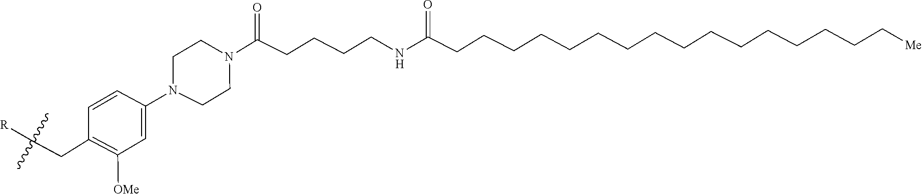

| Cmpd/ | | | Calc'd | Observed | |
|---|---|---|---|---|---|
| Ex. # | R | Name | [M + H]⁺ | [M + H]⁺ | Comments |
| B-18 | 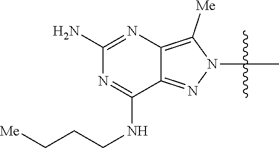 [SMILES: *n1nc2c(NCCCC)nc(N)nc2c1C] | N-(5-(4-(4-((5-amino- 7-(butylamino)-3- methyl-2H- pyrazolo[4,3- d]pyrimidin-2- yl)methyl)-3- methoxyphenyl) piperazin-1-yl)-5- oxopentyl) stearamide | 790.6 | 790.6 | from Int. 2-3 |

### Example 5

### Preparation of Compound C-1

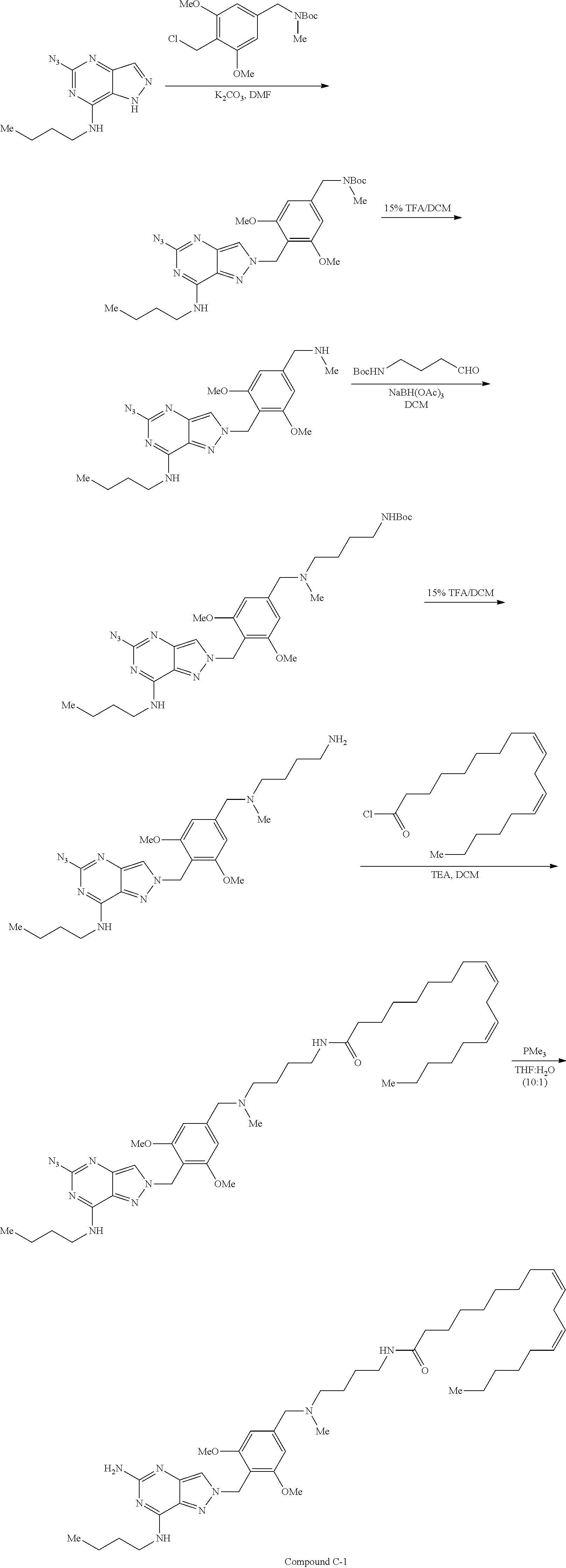

### (9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide (Compound C-1)

### Step 1: tert-butyl (4-((5-azido-7-(butylamino)-2H-pyrazolo[4,3-d]pyrimidin-2-yl)methyl)-3,5-dimethoxybenzyl)(methyl)carbamate

To a mixture of tert-butyl (4-(chloromethyl)-3,5-dimethoxybenzyl)(methyl)carbamate (Int. 2-1, 1 g, 3.03 mmol) in DMF (20 mL) was added 5-azido-N-butyl-1H-pyrazolo[4,3-d]pyrimidin-7-amine (Int. 4-1, 0.634 g, 2.73 mmol) and K₂CO₃ (0.503 g, 3.64 mmol) at ambient temperature. After 2 h, the mixture was diluted with H₂O (20 mL) then extracted with EtOAc (3×30 mL). The combined organic extracts were washed with brine (2×30 mL) then dried (Na₂SO₄) then filtered then the filtrate was concentrated. The crude product was subjected to silica gel chromatography (0-70% EtOAc/petroleum ether) to give the title compound. MS m/z (M+H)⁺: calculated 526.3, observed 526.3.

### Step 2: 5-azido-N-butyl-2-(2,6-dimethoxy-4-((methylamino)methyl)benzyl)-2H-pyrazolo[4,3-d]pyrimidin-7-amine

To a solution of tert-butyl (4-((5-azido-7-(butylamino)-2H-pyrazolo[4,3-d]pyrimidin-2-yl)methyl)-3,5-dimethoxybenzyl)(methyl)carbamate (1.2 g, 2.283 mmol) in DCM (10 mL) was added TFA (2 mL) at ambient temperature. After 3 h, the mixture was concentrated to give the TFA salt of the title compound which was used directly in the next step. MS m/z (M+H)⁺: calculated 426.2, observed 426.3.

### Step 3: tert-butyl (4-((4-((5-azido-7-(butylamino)-2H-pyrazolo[4,3-d]pyrimidin-2-yl)methyl)-3,5-dimethoxybenzyl)(methyl)amino)butyl)carbamate

To a mixture of crude 5-azido-N-butyl-2-(2,6-dimethoxy-4-((methylamino)methyl)benzyl)-2H-pyrazolo[4,3-d]pyrimidin-7-amine (TFA salt, 1.1 g, 2.163 mmol) and tert-butyl (4-oxobutyl)carbamate (Int. 5-1, 0.608 g, 3.24 mmol) in DCM (8 mL) was added NaBH(OAc)₃ (0.917 g, 4.33 mmol) at ambient temperature. After 3 h the mixture was concentrated. The crude product was subjected to silica gel chromatography (0-20% MeOH/DCM) to give the title compound. MS m/z (M+H)⁺: calculated 597.4, observed 597.5.

### Step 4: N′-(4-((5-azido-7-(butylamino)-2H-pyrazolo[4,3-d]pyrimidin-2-yl)methyl)-3,5-dimethoxybenzyl)-N′-methylbutane-1,4-diamine

To a solution of tert-butyl (4-((4-((5-azido-7-(butylamino)-2H-pyrazolo[4,3-d]pyrimidin-2-yl)methyl)-3,5-dimethoxybenzyl)(methyl)amino)butyl)carbamate (790 mg, 1.324 mmol) in DCM (10 mL) was added TFA (2 mL) at room temperature. After 3 h the mixture was concentrated to give the TFA salt of the title compound which was used directly in the next step. MS m/z (M+H)⁺: calculated 497.3, observed 497.4.

### Step 5: (9Z,12Z)—N-(4-((4-((5-azido-7-(butylamino)-2H-pyrazolo[4,3-d]pyrimidin-2-yl)methyl)-3,5-dimethoxybenzyl)(methyl)amino)butyl)octadeca-9,12-dienamide

To a mixture of crude N′-(4-((5-azido-7-(butylamino)-2H-pyrazolo[4,3-d]pyrimidin-2-yl)methyl)-3,5-dimethoxybenzyl)-Ni-methylbutane-1,4-diamine (TFA salt, 447 mg, 0.9 mmol) in DCM (6 mL) was added DIEA (0.314 mL, 1.800 mmol) and (9Z,12Z)-octadeca-9,12-dienoyl chloride (296 mg, 0.990 mmol) at 0° C. After the additions were complete the mixture was warmed to ambient temperature. After 2 h EtOAc (30 mL) was added then the mixture was washed with saturated NaHCO₃ (2×30 mL). The organic layer was dried (Na₂SO₄) then filtered then the filtrate was concentrated. The crude product was subjected to silica gel chromatography (0-15% MeOH/DCM) to give the title compound. MS m/z (M+H)⁺: calculated 759.5, observed 759.6.

### Step 6: (9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide

To a mixture of (9Z,12Z)—N-(4-((4-((5-azido-7-(butylamino)-2H-pyrazolo[4,3-d]pyrimidin-2-yl)methyl)-3,5-dimethoxybenzyl)(methyl)amino)butyl)octadeca-9,12-dienamide (430 mg, 0.566 mmol) in THF (5 mL) and water (0.5 mL) was added trimethylphosphane (1 M in THF, 1.699 mL, 1.699 mmol) then the mixture was heated to 50° C. After 16 h the mixture was cooled to ambient temperature then diluted with EtOAc (30 mL). The resulting mixture was washed with water (30 mL) then dried (Na₂SO₄) then filtered then the filtrate was concentrated. The crude product was purified by prep-TLC developed using MeOH/DCM/ammonia (1/10/0.05) to give the title compound. Compound C-1: MS m/z (M+H)⁺: calculated 733.5, observed 733.5. ¹H-NMR (400 MHz, CD₃OD): δ 7.49 (s, 1H), 6.70 (s, 2H), 5.46 (s, 2H), 5.33-5.28 (m, 4H), 3.85 (s, 6H), 3.52-3.48 (m, 4H), 3.16 (t, J=6.4 Hz, 2H), 2.74 (t, J=6.2 Hz, 2H), 2.41 (t, J=7.1 Hz, 2H), 2.22 (s, 3H), 2.14 (t, J=7.5 Hz, 2H), 2.04-2.01 (m 4H), 1.66-1.41 (m, 10H), 1.36-1.29 (m, 14H), 0.97 (t, J=7.4 Hz, 3H), 0.88 (t, J=6.8 Hz, 3H).

Utilizing the procedures described for Compound C-1, the following compounds were prepared substituting the appropriate reagents for (9Z,12Z)-octadeca-9,12-dienoyl chloride.

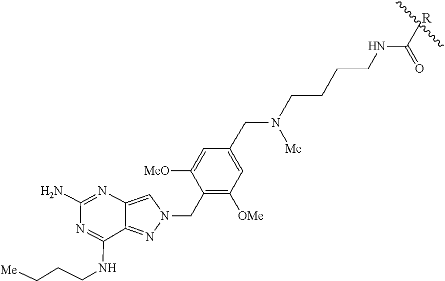

| | | | Calc'd | Observed |
|---|---|---|---|---|
| Cmpd. | R | Name | [M + H]⁺ | [M + H]⁺ |
| C-2 | 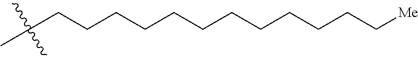 [SMILES: *CCCCCCCCCCCCC] | N-(4-((4-((5-amino- 7-(butylamino)-2H- pyrazolo[4,3- d]pyrimidin-2- yl)methyl)-3,5- dimethoxybenzyl) (methyl)amino)butyl) tetradecanamide | 681.5 | 681.7 |
| C-3 | 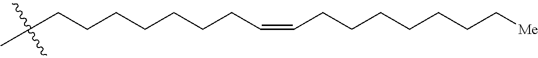 [SMILES: *CCCCCCC/C=C\CCCCCCCC] | N-(4-((4-((5-amino- 7-(butylamino)-2H- pyrazolo[4,3- d]pyrimidin-2- yl)methyl)-3,5- dimethoxybenzyl) (methyl)amino)butyl) oleamide | 735.6 | 735.6 |
| C-4 | 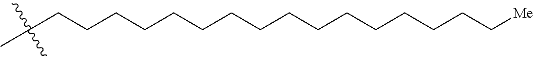 [SMILES: *CCCCCCCCCCCCCCCCC] | N-(4-((4-((5-amino- 7-(butylamino)-2H- pyrazolo[4,3- d]pyrimidin-2- yl)methyl)-3,5- dimethoxybenzyl) (methyl)amino)butyl) stearamide | 737.6 | 737.8 |

### Preparation of Compound C-5

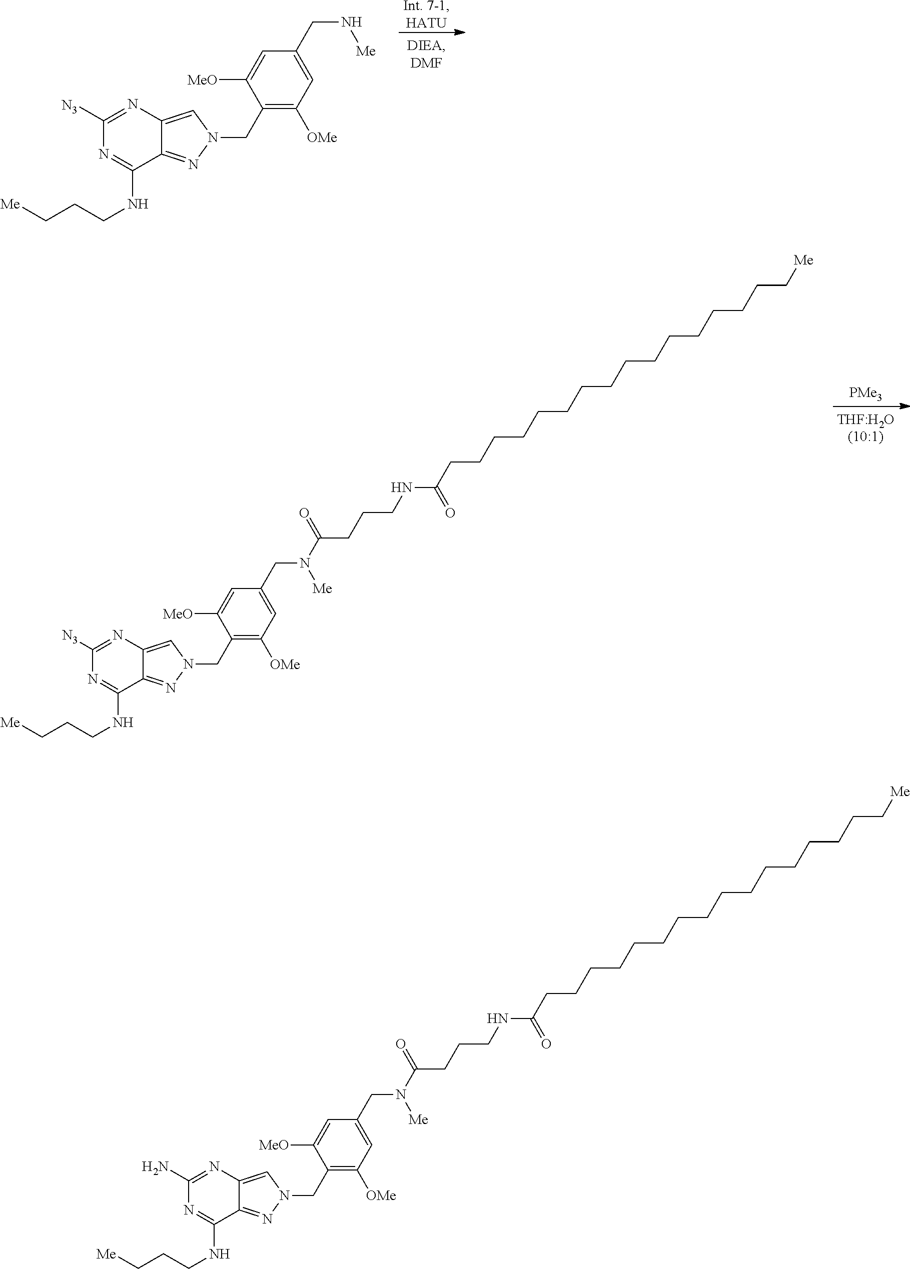 [SMILES: "Int. 7-1,HATU" ; CCCCNc1nc(N=[N+]=[N-])nc2cn(Cc3c(OC)cc(CNC)cc3OC)nc12 ; CCCCCCCCCCCCCCCCCC(=O)NCCCC(=O)N(C)Cc1cc(OC)c(Cn2cc3nc(N=[N+]=[N-])nc(NCCCC)c3n2)c(OC)c1 ; CCCCCCCCCCCCCCCCCC(=O)NCCCC(=O)N(C)Cc1cc(OC)c(Cn2cc3nc(N)nc(NCCCC)c3n2)c(OC)c1 ; "PMe3"]

### N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide

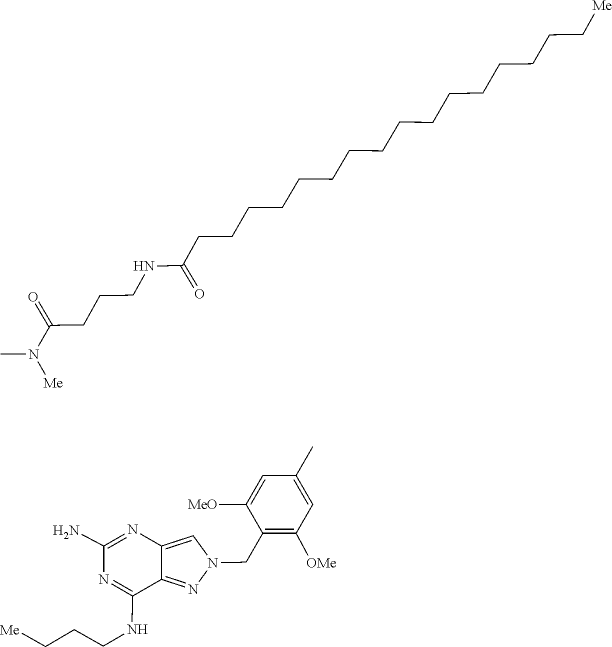 [SMILES: CCCCCCCCCCCCCCCCCC(=O)NCCCC(=O)N(C)C ; CCCCNc1nc(N)nc2cn(Cc3c(OC)cc(C)cc3OC)nc12]

### Step 1: N-(4-((4-((5-azido-7-(butylamino)-2H-pyrazolo[4,3-d]pyrimidin-2-yl)methyl)-3,5-dimethoxybenzyl)(methyl)amino)-4-oxobutyl)stearamide

A mixture of 4-stearamidobutanoic acid (Int. 7-1, 132 mg, 0.357 mmol), 5-azido-N-butyl-2-(2,6-dimethoxy-4-((methylamino)methyl)benzyl)-2H-pyrazolo[4,3-d]pyrimidin-7-amine hydrochloride (Compound C-1 step 2, 110 mg, 0.238 mmol), HATU (181 mg, 0.476 mmol) and DIEA (0.208 mL, 1.191 mmol) in DMF (3 mL) was stirred at rt. After stirring overnight the mixture was purified directly by reverse-phase chromatography (CH₃CN/water with 0.05% FA modifier) to give the title compound. MS m/z (M+H)⁺: calculated 777.5, observed 777.6.

### Step 2: N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide

To a solution of N-(4-((4-((5-azido-7-(butylamino)-2H-pyrazolo[4,3-d]pyrimidin-2-yl)methyl)-3,5-dimethoxybenzyl)(methyl)amino)-4-oxobutyl)stearamide (160 mg, 0.206 mmol) in THF (3 mL):H₂O (0.60 mL) was added PMe₃ (1M in THF, 0.618 mL, 0.618 mmol) then the mixture was heated to 50° C. After 5 hr the mixture was cooled to rt then quenched with MeOH (5 mL) then the resulting mixture was concentrated. The crude product was purified by reverse-phase chromatography (1:3 CH₃CN:MeOH/water) to give the title compound. Compound C-5: MS m/z (M+H)⁺: calculated 751.6, observed 751.6. ¹H-NMR (400 MHz, DMSO-d6) δ 7.99-7.54 (m, 2H), 7.37 (d, J=3.6 Hz, 1H), 6.54 (d, J=19.6 Hz, 2H), 5.92 (s, 2H), 5.36 (s, 2H), 4.54 (d, J=16.8 Hz, 2H), 3.79 (d, J=8.4 Hz, 6H), 3.44-3.39 (m, 2H), 3.10-3.00 (m, 2H), 2.93 (s, 2H), 2.84 (s, 1H), 2.38-2.33 (m, 2H), 2.03-1.98 (m, 2H), 1.70-1.65 (m, 2H), 1.60-1.55 (m, 2H), 1.34-1.30 (m, 2H), 1.22 (d, J=3.2 Hz, 30H), 0.90-0.82 (m, 6H).

### Example 6

### Preparation of Compound D-1

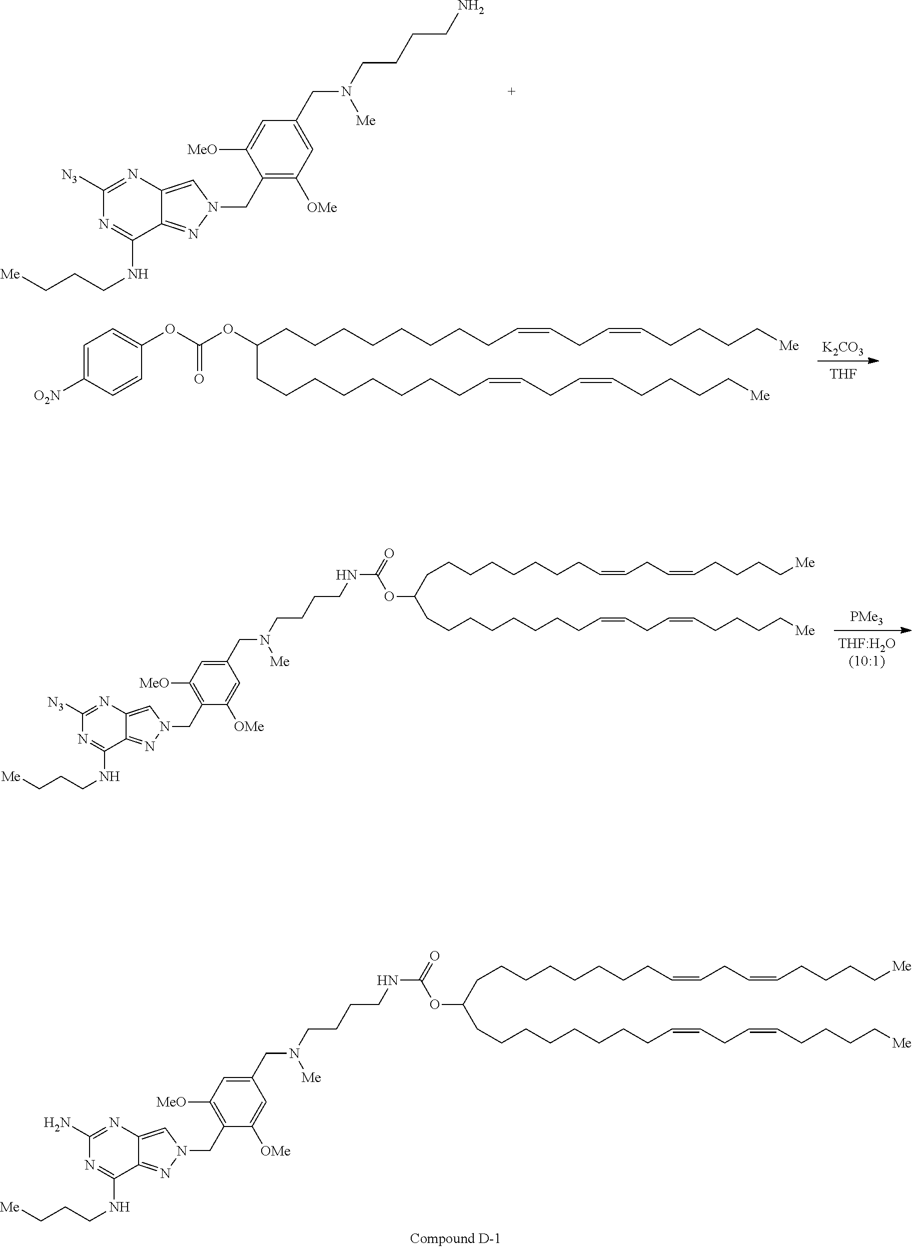

### (6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)amino)butyl)carbamate (Compound D-1)

### Step 1: (6Z,9Z,28Z,31Z)-heptatriaconta-6,9,28,31-tetraen-19-yl (4-((4-((5-azido-7-(butyl-amino)-2H-pyrazolo[4,3-d]pyrimidin-2-yl)methyl)-3,5-dimethoxybenzyl)(methyl)-amino)butyl)carbamate

To a mixture of (6Z,9Z,28Z,31Z)-heptatriaconta-6,9,28,31-tetraen-19-yl (4-nitrophenyl) carbonate (Int. 6-1, 355 mg, 0.511 mmol) and crude Ni-(4-((5-azido-7-(butylamino)-2H-pyrazolo[4,3-d]pyrimidin-2-yl)methyl)-3,5-dimethoxybenzyl)-Ni-methylbutane-1,4-diamine (TFA salt, 274 mg, 0.460 mmol) in THF (5 mL) was added K₂CO₃ (0.058 mL, 1.023 mmol) then the mixture was heated to 65° C. After 15 h, the mixture was cooled to ambient temperature then concentrated. The crude product was subjected to silica gel chromatography (0-10% MeOH/DCM) to give the title compound. MS m/z (M+H)⁺: calculated 1051.8, observed 1052.0.

### Step 2: (6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)-amino)butyl)carbamate

To a mixture of (6Z,9Z,28Z,31Z)-heptatriaconta-6,9,28,31-tetraen-19-yl (4-((4-((5-azido-7-(butylamino)-2H-pyrazolo[4,3-d]pyrimidin-2-yl)methyl)-3,5-dimethoxybenzyl)(methyl)-amino)butyl)carbamate (500 mg, 0.475 mmol) in THF (10 mL) and H₂O (1.0 mL) was added trimethylphosphane (1 M in THF, 1.426 mL, 1.426 mmol) at ambient temperature. After 15 h the mixture was quenched with MeOH (1.42 mL) then concentrated. The crude product was purified by prep-TLC (DCM:MeOH=10:1) to give the title compound. Compound D-1: MS m/z (M+H)⁺: calculated 1025.8, observed 1025.7. ¹H-NMR (300 MHz, CD₃OD): δ 7.65 (s, 1H), 6.71 (s, 2H), 5.49 (s, 2H), 5.35-5.32 (m, 8H), 4.86-4.85 (m, 1H), 3.86 (s, 6H), 3.56-3.55 (m, 4H), 3.12-3.10 (m, 2H), 2.77-2.75 (m, 4H), 2.45-2.43 (m, 2H), 2.25 (s, 3H), 2.05-2.03 (m, 8H), 1.68-1.66 (m, 6H), 1.46-1.44 (m, 6H), 1.32-1.27 (m, 36H), 0.97-0.94 (m, 9H).

### Preparation of Compound D-2

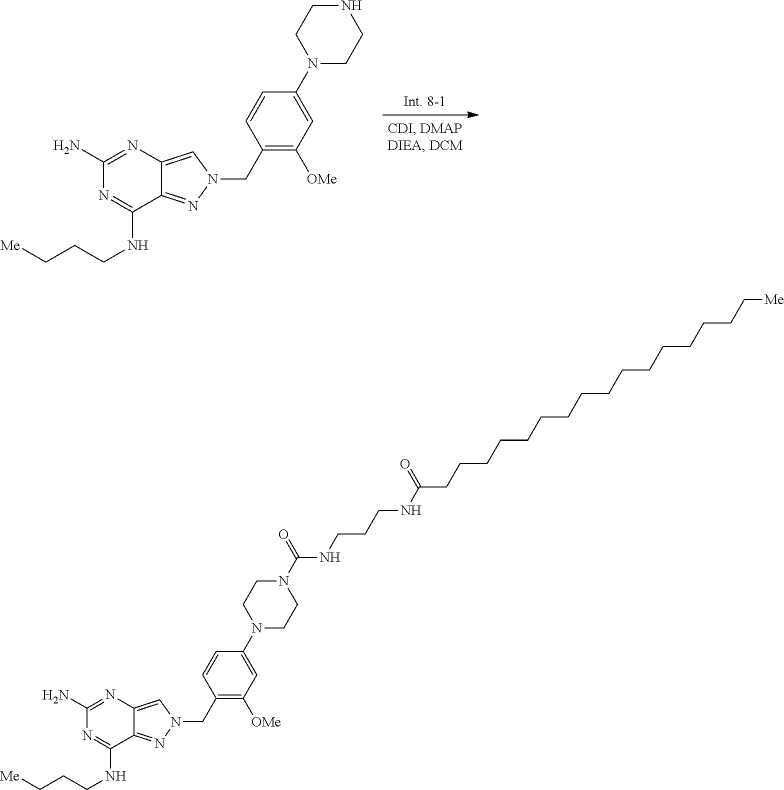 [SMILES: CCCCNc1nc(N)nc2cn(Cc3ccc(N4CCNCC4)cc3OC)nc12 ; CCCCCCCCCCCCCCCCCC(=O)NCCCNC(=O)N1CCN(c2ccc(Cn3cc4nc(N)nc(NCCCC)c4n3)c(OC)c2)CC1]

### 4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide

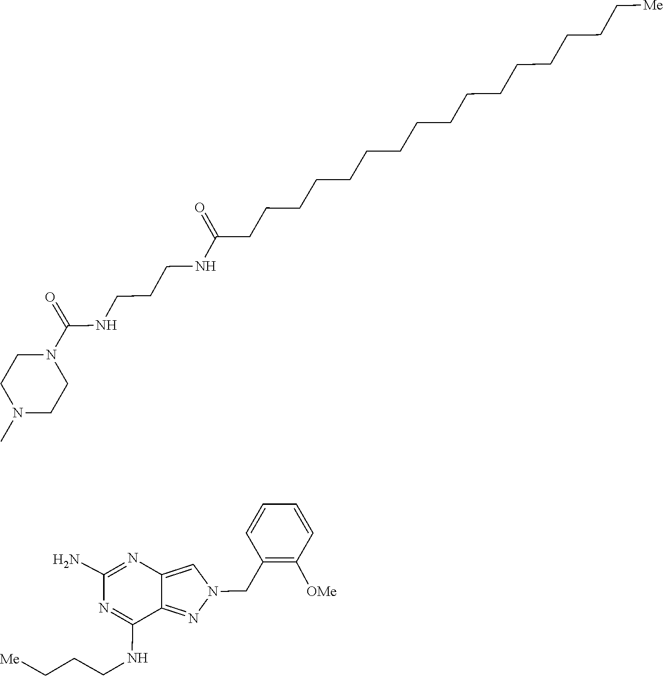 [SMILES: CCCCCCCCCCCCCCCCCC(=O)NCCCNC(=O)N1CCN(C)CC1 ; CCCCNc1nc(N)nc2cn(Cc3ccccc3OC)nc12]

### Step 1: 4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide

A mixture of N-(3-aminopropyl)stearamide (Int. 8-1, 457 mg, 1.342 mmol), CDI (136 mg, 0.839 mmol), DMAP (13.67 mg, 0.112 mmol) and DIEA (0.586 mL, 3.36 mmol) in DCM (10 mL) was stirred at 0° C. After 30 minutes N⁷-butyl-2-(2-methoxy-4-(piperazin-1-yl)benzyl)-2H-pyrazolo[4,3-d]pyrimidine-5,7-diamine hydrochloride (500 mg, 1.119 mmol) was added. The mixture was warmed to rt then heated to 50° C. After 3 hr the mixture was cooled to rt then concentrated. The crude product was purified by reverse-phase chromatography (CH₃CN/water with 0.1% TFA modifier) to give the title compound. Compound D-2: MS m/z (M+H)⁺: calculated 777.6, observed 777.6. ¹H-NMR (300 MHz, DMSO-d6) δ 8.89 (s, 1H), 7.83-7.55 (m, 2H), 7.16-6.98 (m, 2H), 6.60-6.48 (m, 2H), 5.34 (s, 2H), 3.80 (s, 3H), 3.47 (s, 6H), 3.12 (s, 4H), 3.04-3.01 (m, 4H), 2.03 (t, J=7.2 Hz, 2H), 1.60-1.48 (m, 6H), 1.22 (s, 28H), 0.96-0.77 (m, 6H).

### Preparation of Compound D-3

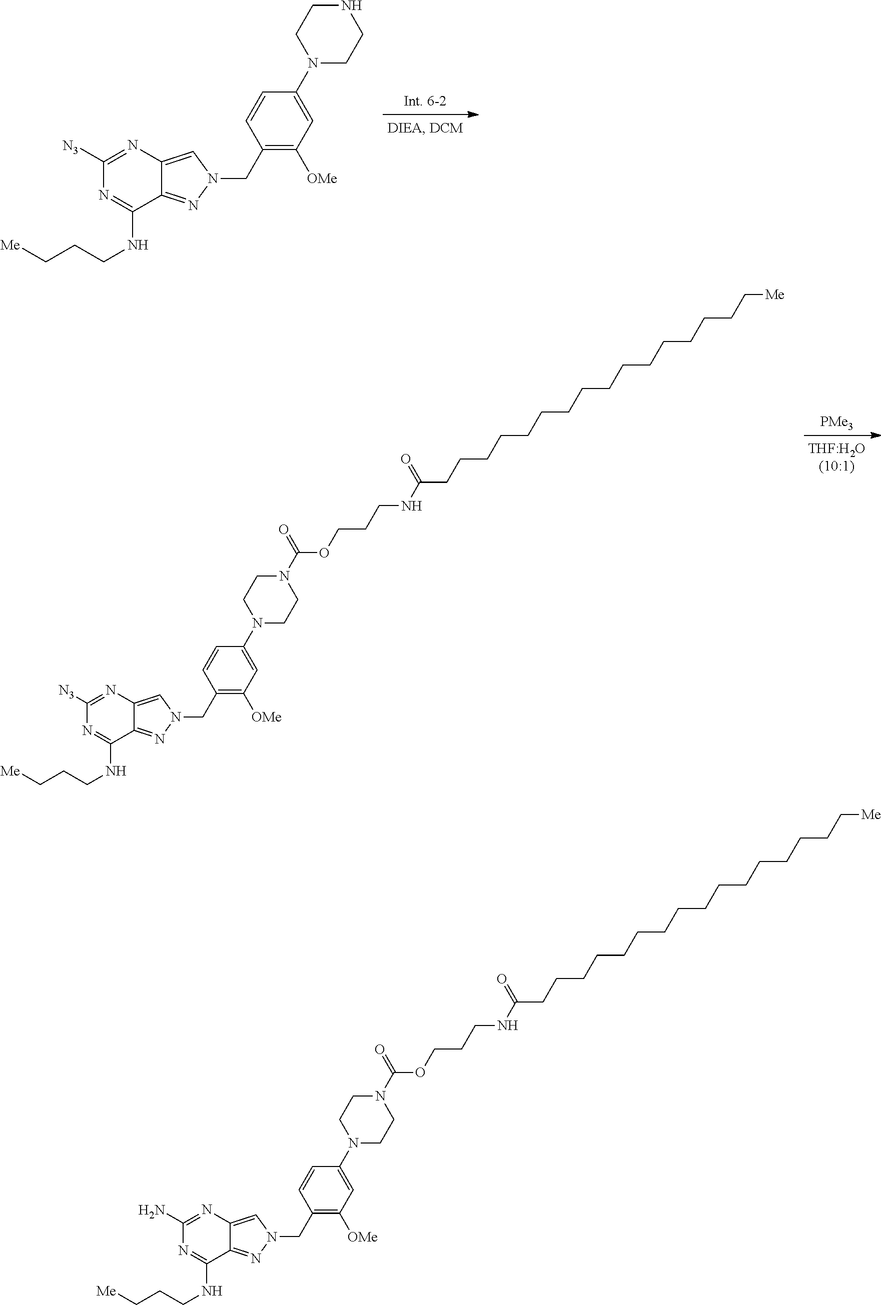 [SMILES: CCCCNc1nc(N=[N+]=[N-])nc2cn(Cc3ccc(N4CCNCC4)cc3OC)nc12 ; CCCCCCCCCCCCCCCCCC(=O)NCCCOC(=O)N1CCN(c2ccc(Cn3cc4nc(N=[N+]=[N-])nc(NCCCC)c4n3)c(OC)c2)CC1 ; CCCCCCCCCCCCCCCCCC(=O)NCCCOC(=O)N1CCN(c2ccc(Cn3cc4nc(N)nc(NCCCC)c4n3)c(OC)c2)CC1 ; "PMe3"]

### 3-stearamidopropyl 4-(4-((5-amino-7-(butylamino)-2H-pyrazolo[4,3-d]pyrimidin-2-yl)methyl)-3-methoxyphenyl)piperazine-1-carboxylate

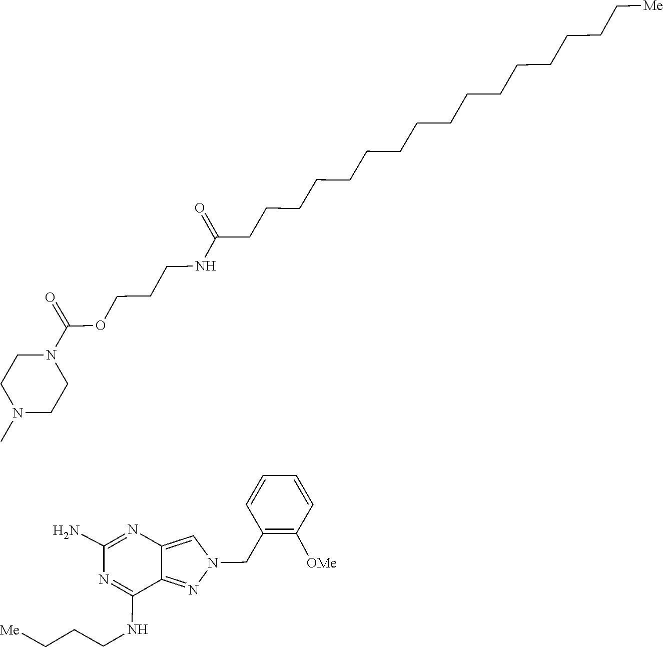 [SMILES: CCCCCCCCCCCCCCCCCC(=O)NCCCOC(=O)N1CCN(C)CC1 ; CCCCNc1nc(N)nc2cn(Cc3ccccc3OC)nc12]

### Step 1: 3-stearamidopropyl 4-(4-((5-azido-7-(butylamino)-2H-pyrazolo[4,3-d]pyrimidin-2-yl)methyl)-3-methoxyphenyl)piperazine-1-carboxylate

A mixture of 4-nitrophenyl (3-stearamidopropyl) carbonate (Int. 6-2, 130 mg, 0.257 mmol), 5-azido-N-butyl-2-(2-methoxy-4-(piperazin-1-yl)benzyl)-2H-pyrazolo[4,3-d]pyrimidin-7-amine (Compound B-1 step 3, 56 mg, 0.128 mmol) and DIEA (0.045 mL, 0.257 mmol) in DCM (0.5 mL) was stirred at rt. After 1 hr the mixture was purified directly by prep-TLC (DCM/MeOH=15:1) to give the title compound. MS m/z (M+H)⁺: calculated 804.6, observed 804.7.

### Step 2: 3-stearamidopropyl 4-(4-((5-amino-7-(butylamino)-2H-pyrazolo[4,3-d]pyrimidin-2-yl)methyl)-3-methoxyphenyl)piperazine-1-carboxylate

To a solution of 4-(4-((5-azido-7-(butylamino)-2H-pyrazolo[4,3-d]pyrimidin-2-yl)methyl)-3-methoxyphenyl)piperazine-1-carboxylate (80 mg, 0.099 mmol) in THF (2 mL):H₂O (0.5 mL) was added PMe₃ (1M in THF, 0.298 mL, 0.298 mmol) then the mixture was heated to 50° C. After 5 hr the mixture was cooled to rt then quenched with MeOH (5 mL) then the resulting mixture was concentrated. The crude product was purified by prep-TLC(DCM:MeOH=10:1) to give the title compound. Compound D-3: MS m/z (M+H)⁺: calculated 778.6, observed 778.6. ¹H-NMR (400 MHz, DMSO-d6) δ 7.80 (t, J=5.6 Hz, 1H), 7.64 (s, 1H), 7.55 (s, 1H), 6.91 (d, J=8.0 Hz, 1H), 6.60 (d, J=2.0 Hz, 1H), 6.49-6.45 (m, 1H), 5.56 (s, 2H), 5.29 (s, 2H), 4.01 (t, J=6.4 Hz, 2H), 3.80 (s, 3H), 3.49-3.38 (m, 6H), 3.14-3.11 (m, 6H), 2.03 (t, J=7.2 Hz, 2H), 1.71-1.68 (m, 2H), 1.58-1.53 (m, 2H), 1.47-1.26 (m, 4H), 1.28-1.15 (m, 28H), 0.91-0.83 (m, 6H).

### Preparation of Compound D-4

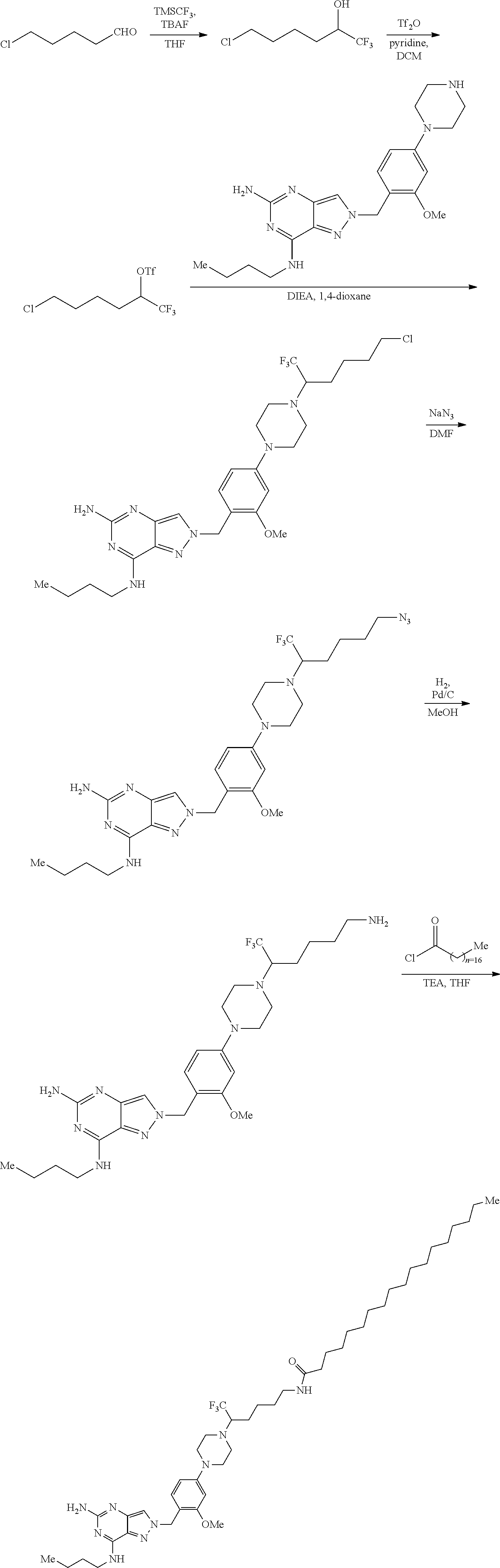

### N-(5-(4-(4-((5-amino-7-(butylamino)-2H-pyrazolo[4,3-d]pyrimidin-2-yl)methyl)-3-methoxyphenyl)piperazin-1-yl)-6,6,6-trifluorohexyl)stearamide

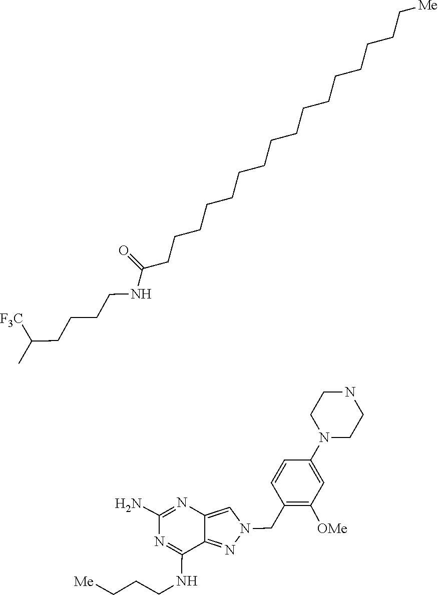 [SMILES: CCCCCCCCCCCCCCCCCC(=O)NCCCCC(C)C(F)(F)F ; CCCCNc1nc(N)nc2cn(Cc3ccc(N4CCNCC4)cc3OC)nc12]

### Step 1: 6-chloro-1,1,1-trifluorohexan-2-ol

To a solution of 5-chloropentanal (4.5 g, 37.3 mmol) and trimethyl(trifluoromethyl)silane (6.37 g, 44.8 mmol) in THF (80 mL) was added tetrabutylammonium fluoride (0.485 mL, 0.485 mmol, 1M in THF) at 0° C. After 1 hr the cooling bath was removed and the mixture warmed to rt. After 2 hr the mixture was quenched with 1N HCl (60 mL). After stirring for 2 hr the mixture was diluted with water (60 mL) then extracted with EtOAc (35 mL×2). The combined organic layers were dried (Na₂SO₄) then filtered then the filtrate was concentrated. The crude product was subjected to silica gel chromatography (0-5% EtOAc/pet. ether) to give the title compound. ¹H NMR (400 MHz, CDCl₃) δ 3.99-3.83 (m, 1H), 3.56 (t, J=6.4 Hz, 2H), 2.85-2.66 (m, 1H), 1.90-1.79 (m, 2H), 1.78-1.68 (m, 2H), 1.67-1.54 (m, 2H).

### Step 2: 6-chloro-1,1,1-trifluorohexan-2-yl trifluoromethanesulfonate

To a solution of 6-chloro-1,1,1-trifluorohexan-2-ol (1.6 g, 8.39 mmol) and pyridine (1.013 mL, 12.59 mmol) in DCM (25 mL) was added trifluoromethanesulfonic anhydride (1.702 mL, 10.07 mmol) dropwise at 0° C. After 1 hr the cooling bath was removed and the mixture warmed to rt. After 2 hr the mixture was diluted with water (10 mL) then extracted with EtOAc (15 mL×2). The combined organic layers were washed with 1 N HCl (20 mL) then saturated NaHCO₃ then dried (Na₂SO₄) then filtered then the filtrate was concentrated to give the title compound which was used in the next step without purification. ¹H NMR (400 MHz, CDCl₃) δ 5.11-4.96 (m, 1H), 3.58 (t, J=6.4 Hz, 2H), 2.06-1.96 (m, 2H), 1.94-1.82 (m, 2H), 1.79-1.62 (m, 2H).

### Step 3: N⁷-butyl-2-(4-(4-(6-chloro-1,1,1,-trifluorohexan-2-yl)piperazin-1-yl)-2-methoxybenzyl)-2H-pyrazolo[4,3-d]pyrimidine-5,7-diamine

To a solution of N⁷-butyl-2-(2-methoxy-4-(piperazin-1-yl)benzyl)-2H-pyrazolo[4,3-d]pyrimidine-5,7-diamine (1.4 g, 3.41 mmol) in 1,4-dioxane (16 mL) was added DIEA (1.787 mL, 10.23 mmol) and 6-chloro-1,1,1-trifluorohexan-2-yl trifluoromethanesulfonate (1.650 g, 5.12 mmol) then the mixture was heated to 80° C. After heating overnight the mixture was cooled to rt then concentrated. The crude product was purified by reverse-phase chromatography (CH₃CN/water with 10 mM NH₄HCO₃ modifier) to give the title compound. MS m/z (M+H)⁺: calculated 583.3, observed 583.4. ¹H NMR (400 MHz, CD₃OD) δ 7.52 (s, 1H), 7.08 (d, J=8.4 Hz, 1H), 6.57 (d, J=2.0 Hz, 1H), 6.50 (dd, J=2.0, 8.4 Hz, 1H), 5.32 (s, 2H), 3.81 (s, 3H), 3.58 (t, J=6.4 Hz, 2H), 3.52-3.45 (m, 2H), 3.23-3.03 (m, 7H), 2.82-2.75 (m, 2H), 1.84-1.70 (m, 4H), 1.66-1.55 (m, 4H), 1.47-1.38 (m, 2H), 0.96 (t, J=7.2 Hz, 3H).

### Step 4: 2-(4-(4-(6-azido-1,1,1-trifluorohexan-2-yl)piperazin-1-yl)-2-methoxybenzyl)-N⁷-butyl-2H-pyrazolo[4,3-d]pyrimidine-5,7-diamine

To a solution of N⁷-butyl-2-(4-(4-(6-chloro-1,1,1-trifluorohexan-2-yl)piperazin-1-yl)-2-methoxybenzyl)-2H-pyrazolo[4,3-d]pyrimidine-5,7-diamine (160 mg, 0.274 mmol) in DMF (2 mL) was added sodium azide (80 mg, 1.231 mmol) then the mixture was heated to 100° C. After 2 hr the mixture was cooled to rt then diluted with water (5 mL). The pH was adjusted to >9 with saturated NaHCO₃ (15 mL) then the mixture was extracted with EtOAc (15 mL×2). The combined organic layers were dried (Na₂SO₄) then filtered then the filtrate was concentrated. The crude product was purified by prep-TLC (DCM/MeOH=10:1) to give the title compound. MS m/z (M+H)⁺: calculated 590.3, observed 590.3.

### Step 5: 2-(4-(4-(6-amino-1,1,1-trifluorohexan-2-yl)piperazin-1-yl)-2-methoxybenzyl)-N⁷-butyl-2H-pyrazolo[4,3-d]pyrimidine-5,7-diamine

To a solution of 2-(4-(4-(6-azido-1,1,1-trifluorohexan-2-yl)piperazin-1-yl)-2-methoxybenzyl)-N⁷-butyl-2H-pyrazolo[4,3-d]pyrimidine-5,7-diamine (100 mg, 0.170 mmol) in MeOH (2 mL) was added Pd/C (18.05 mg). The mixture was purged with H₂ (15 psi). After 2 hr the mixture was concentrated. The crude product was purified by prep-TLC (DCM/MeOH=10:1) to give the title compound. MS m/z (M+H)⁺: calculated 564.3, observed 564.3.

### Step 6: N-(5-(4-(4-((5-amino-7-(butylamino)-2H-pyrazolo[4,3-d]pyrimidin-2-yl)methyl)-3-methoxyphenyl)piperazin-1-yl)-6,6,6-trifluorohexyl)stearamide

To a solution of 2-(4-(4-(6-amino-1,1,1-trifluorohexan-2-yl)piperazin-1-yl)-2-methoxybenzyl)-N⁷-butyl-2H-pyrazolo[4,3-d]pyrimidine-5,7-diamine (60 mg, 0.106 mmol) in DCM (1 mL) was added DIEA (0.056 mL, 0.319 mmol) and stearoyl chloride (32.2 mg, 0.106 mmol) at rt. After 2 hr the mixture was concentrated. The crude product was purified by reverse-phase chromatography (CH₃CN/water with 0.2% FA modifier) to give the title compound. Compound D-4: MS m/z (M+H)⁺: calculated 830.6, observed 830.5. ¹H NMR (400 MHz, CD₃OD) δ 7.63 (s, 1H), 7.14 (d, J=8.4 Hz, 1H), 6.58 (d, J=2.0 Hz, 1H), 6.52 (dd, J=2.0, 8.4 Hz, 1H), 5.37 (s, 2H), 3.83 (s, 3H), 3.58 (t, J=7.2 Hz, 2H), 3.25-3.12 (m, 7H), 3.10-3.03 (m, 2H), 2.84-2.75 (m, 2H), 2.19-2.11 (m, 2H), 1.78-1.51 (m, 9H), 1.40 (s, 3H), 1.32-1.20 (m, 28H), 0.98 (t, J=7.2 Hz, 3H), 0.89 (t, J=6.8 Hz, 3H).

### Preparation of Compound D-5

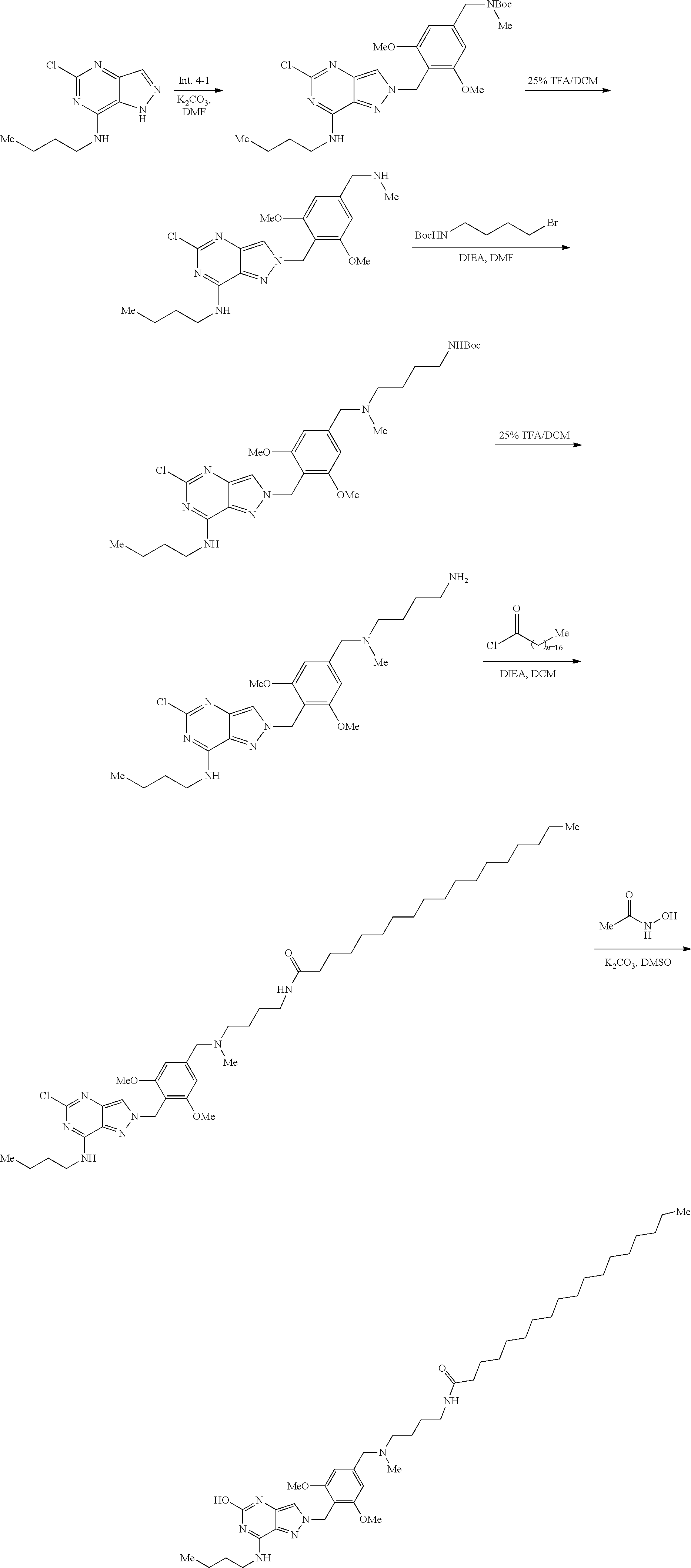

### N-(4-((4-((7-(butylamino)-5-hydroxy-2H-pyrazolo[4,3-d]pyrimidin-2-yl)methyl)-3,5-dimethoxybenzyl)(methyl)amino)butyl)stearamide

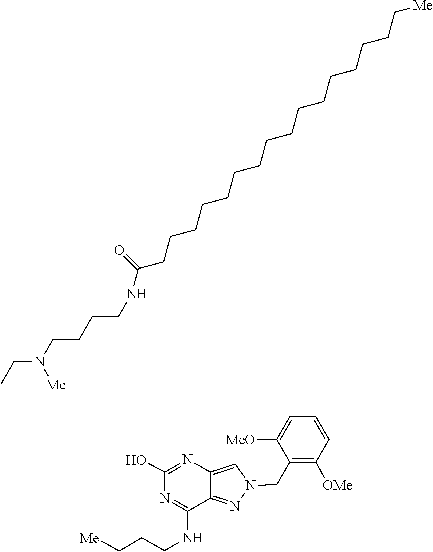 [SMILES: CCCCCCCCCCCCCCCCCC(=O)NCCCCN(C)CC ; CCCCNc1nc(O)nc2cn(Cc3c(OC)cccc3OC)nc12]

### Step 1: tert-butyl (4-((7-(butylamino)-5-chloro-2H-pyrazolo[4,3-d]pyrimidin-2-yl)methyl)-3,5-dimethoxybenzyl)(methyl)carbamate

To a solution of N-butyl-5-chloro-2H-pyrazolo[4,3-d]pyrimidin-7-amine (Int. 2-1 step 1, 800 mg, 3.54 mmol) in DMF (15 mL) was added tert-butyl (4-(chloromethyl)-3,5-dimethoxybenzyl)(methyl)carbamate (Int. 4-1, 1169 mg, 3.54 mmol) and K₂CO₃ (1470 mg, 10.63 mmol) at rt. After stirring overnight the mixture was diluted with water (80 mL) then extracted with EtOAc (15 mL×3). The combined organic layers were washed with brine (20 mL×2) then dried (Na₂SO₄) then filtered then the filtrate was concentrated. The crude product was subjected to silica gel chromatography (0-40% EtOAc/pet. ether) to give the title compound. MS m/z (M+H)⁺: calculated 519.2, observed 519.3. ¹H NMR (400 MHz, CD₃OD) δ 7.88 (s, 1H), 6.59 (s, 2H), 5.57 (s, 2H), 4.44 (s, 2H), 3.85 (s, 6H), 3.55 (t, J=7.2 Hz, 2H), 2.86 (s, 3H), 1.66 (quin, J=7.2 Hz, 2H), 1.48 (br s, 9H), 1.45-1.39 (m, 2H), 0.98 (t, J=7.6 Hz, 3H).

### Step 2: N-butyl-5-chloro-2-(2,6-dimethoxy-4-((methylamino)methyl)benzyl)-2H-pyrazolo[4,3-d]pyrimidin-7-amine

To a solution of tert-butyl (4-((7-(butylamino)-5-chloro-2H-pyrazolo[4,3-d]pyrimidin-2-yl)methyl)-3,5-dimethoxybenzyl)(methyl)carbamate (1 g, 1.927 mmol) in DCM (15 mL) was added TFA (3 mL) at rt. After 3 hr the mixture was concentrated to give the TFA salt of the title compound which was used without purification. MS m/z (M+H)⁺: calculated 419.2, observed 419.3. ¹H NMR (400 MHz, CD₃OD) δ 7.99 (s, 1H), 6.85 (s, 2H), 5.65 (s, 2H), 4.19 (s, 2H), 3.91 (s, 6H), 3.63 (t, J=7.2 Hz, 2H), 2.74 (s, 3H), 1.68 (quin, J=7.2 Hz, 2H), 1.44 (qd, J=7.6, 15.2 Hz, 2H), 0.98 (t, J=7.6 Hz, 3H).

### Step 3: tert-butyl (4-((4-((7-(butylamino)-5-chloro-2H-pyrazolo[4,3-d]pyrimidin-2-yl)methyl)-3,5-dimethoxybenzyl)(methyl)amino)butyl)carbamate

To a solution of N-butyl-5-chloro-2-(2,6-dimethoxy-4-((methylamino)methyl)benzyl)-2H-pyrazolo[4,3-d]pyrimidin-7-amine (300 mg, 0.716 mmol) in DMF (10 mL) was added DIEA (0.375 mL, 2.148 mmol) and tert-butyl (4-bromobutyl)carbamate (217 mg, 0.859 mmol) then the mixture was heated to 40° C. After heating overnight the mixture was cooled to rt then diluted with water (50 mL) then extracted with EtOAc (10 mL×3). The combined organic layers were washed with brine (30 mL) then dried (Na₂SO₄) then filtered then the filtrate was concentrated. The crude product was subjected to silica gel chromatography (0-5% MeOH/DCM) to give the title compound. MS m/z (M+H)⁺: calculated 590.3, observed 590.3.

### Step 4: N′-(4-((7-(butylamino)-5-chloro-2H-pyrazolo[4,3-d]pyrimidin-2-yl)methyl)-3,5-dimethoxybenzyl)-N1-methylbutane-1,4-diamine

To a solution of tert-butyl (4-((4-((7-(butylamino)-5-chloro-2H-pyrazolo[4,3-d]pyrimidin-2-yl)methyl)-3,5-dimethoxybenzyl)(methyl)amino)butyl)carbamate (250 mg, 0.424 mmol) in DCM (5 mL) was added TFA (1 mL) at rt. After stirring overnight the mixture was concentrated to give the TFA salt of the title compound which was used without purification. MS m/z (M+H)⁺: calculated 490.3, observed 490.3.

### Step 5: N-(4-((4-((7-(butylamino)-5-chloro-2H-pyrazolo[4,3-d]pyrimidin-2-yl)methyl)-3,5-dimethoxybenzyl)(methyl)amino)butyl)stearamide

To a solution of Ni-(4-((7-(butylamino)-5-chloro-2H-pyrazolo[4,3-d]pyrimidin-2-yl)methyl)-3,5-dimethoxybenzyl)-N¹-methylbutane-1,4-diamine (200 mg, 0.408 mmol) in DCM (5 mL) was added DIEA (0.214 mL, 1.224 mmol) then stearoyl chloride (124 mg, 0.408 mmol) at 0° C. After the addition was complete the mixture was warmed to rt. After 2 hr the mixture was diluted with water (15 mL) then extracted with DCM (3 mL×3). The combined organic layers were washed with brine (15 mL) then dried (Na₂SO₄) then filtered then the filtrate was concentrated. The crude product was subjected to silica gel chromatography (0-5% MeOH/DCM) to give the title compound. MS m/z (M+H)⁺: calculated 756.5, observed 756.4.

### Step 6: N-(4-((4-((7-(butylamino)-5-hydroxy-2H-pyrazolo[4,3-d]pyrimidin-2-yl)methyl)-3,5-dimethoxybenzyl)(methyl)amino)butyl)stearamide

To a solution of N-(4-((4-((7-(butylamino)-5-chloro-2H-pyrazolo[4,3-d]pyrimidin-2-yl)methyl)-3,5-dimethoxybenzyl)(methyl)amino)butyl)stearamide (90 mg, 0.119 mmol) in DMSO (7.5 mL) was added K₂CO₃ (49.3 mg, 0.357 mmol) and acetohydroxamic acid (17.86 mg, 0.238 mmol) then the mixture was heated to 140° C. by microwave irradiation for 2 hr. Acetohydroxamic acid (17.86 mg, 0.238 mmol) was added then the mixture was heated to 140° C. by microwave irradiation for 2 hr. The mixture was diluted with water (20 mL) then extracted with DCM (3 mL×3). The combined organic layers were washed with brine (15 mL) then dried (Na₂SO₄) then filtered then the filtrate was concentrated. The crude product was purified by reverse-phase chromatography (CH₃CN/water with 0.2% FA modifier) to give the title compound. Compound D-5: MS m/z (M+H)⁺: calculated 738.6, observed 738.4. ¹H NMR (400 MHz, CD₃OD) δ 7.41 (s, 1H), 6.76 (s, 2H), 5.46 (s, 2H), 3.88 (s, 6H), 3.77 (br s, 2H), 3.53 (t, J=7.2 Hz, 2H), 3.18 (t, J=6.8 Hz, 2H), 2.71-2.61 (m, 2H), 2.41 (s, 3H), 2.16 (t, J=7.2 Hz, 2H), 1.68-1.61 (m, 4H), 1.60-1.50 (m, 4H), 1.43 (dd, J=7.2, 15.2 Hz, 2H), 1.27 (br s, 28H), 0.97 (t, J=7.2 Hz, 3H), 0.92-0.86 (m, 3H).

### Preparation of Compound D-6

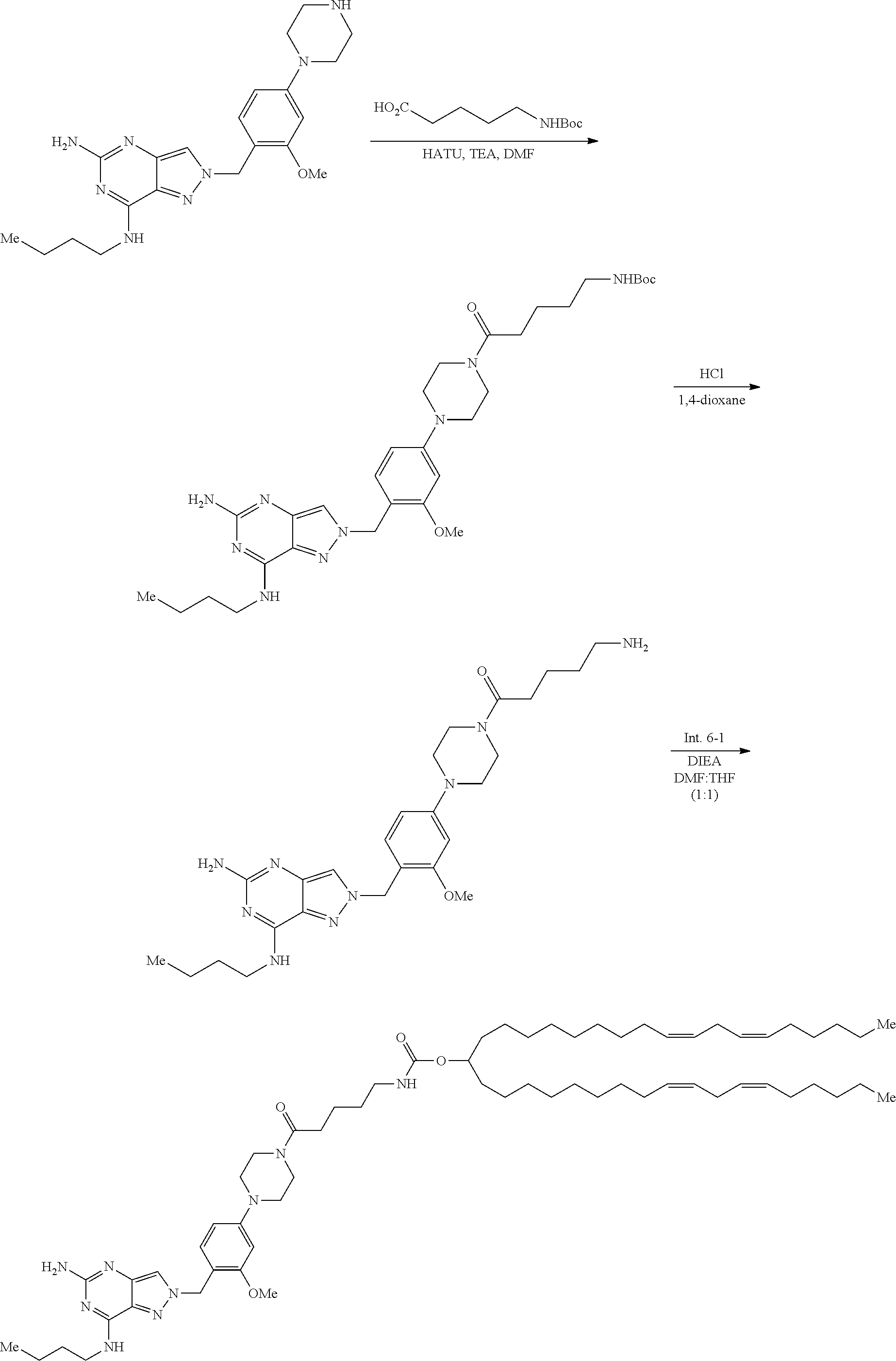

### (6Z,9Z,28Z,31Z)-heptatriaconta-6,9,28,31-tetraen-19-yl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate

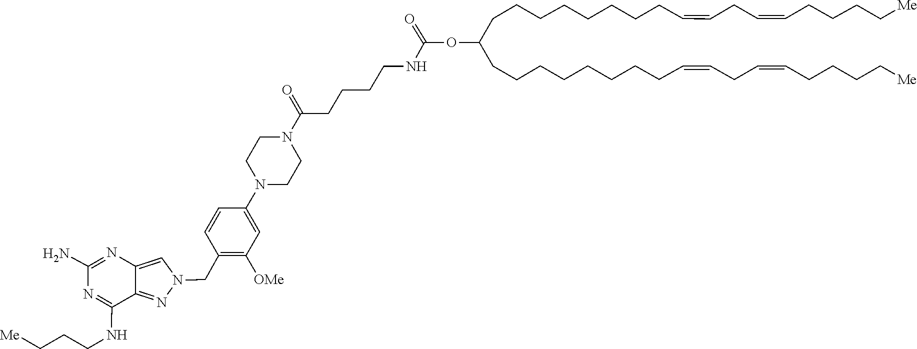 [SMILES: CCCCC/C=C\C/C=C\CCCCCCCCC(CCCCCCCC/C=C\C/C=C\CCCCC)OC(=O)NCCCCC(=O)N1CCN(c2ccc(Cn3cc4nc(N)nc(NCCCC)c4n3)c(OC)c2)CC1]

### Step 1: tert-butyl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate

To a solution of 5-((tert-butoxycarbonyl)amino)pentanoic acid (0.699 g, 3.22 mmol) in DMF (20 ml) was added TEA (0.678 g, 6.70 mmol) and HATU (1.528 g, 4.02 mmol) at rt. After 20 minutes N⁷-butyl-2-(2-methoxy-4-(piperazin-1-yl)benzyl)-2H-pyrazolo[4,3-d]pyrimidine-5,7-diamine (1.1 g, 2.68 mmol) was added. After stirring overnight the mixture was filtered then purified directly by reverse-phase chromatography (CH₃CN/water with 10 mM NH₄HCO₃ modifier) to give the title compound. MS m/z (M+H)⁺: calculated 610.4, observed 610.5. ¹H NMR (400 MHz, CD₃OD) δ 7.53 (s, 1H), 7.11 (d, J=8.4 Hz, 1H), 6.60 (d, J=2.0 Hz, 1H), 6.53 (dd, J=2.0, 8.4 Hz, 1H), 5.34 (s, 2H), 3.83 (s, 3H), 3.75-3.65 (m, 4H), 3.50 (t, J=7.2 Hz, 2H), 3.25-3.21 (m, 2H), 3.20-3.16 (m, 2H), 3.06 (t, J=6.8 Hz, 2H), 2.45 (t, J=7.6 Hz, 2H), 1.68-1.59 (m, 4H), 1.57-1.49 (m, 2H), 1.48-1.43 (m, 2H), 1.41 (s, 9H), 0.97 (t, J=7.2 Hz, 3H).

### Step 2: 5-amino-1-(4-(4-((5-amino-7-(butylamino)-2H-pyrazolo[4,3-d]pyrimidin-2-yl)methyl)-3-methoxyphenyl)piperazin-1-yl)pentan-1-one

To a solution of tert-butyl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate (800 mg, 1.312 mmol) in DCM (10 ml) was added TFA (2 ml) at rt. After 1 hr the mixture was concentrated to give the TFA salt of the title compound which was used without purification. MS m/z (M+H)⁺: calculated 510.3, observed 510.3.

### Step 3: (6Z,9Z,28Z,31Z)-heptatriaconta-6,9,28,31-tetraen-19-yl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate

To a solution of 5-amino-1-(4-(4-((5-amino-7-(butylamino)-2H-pyrazolo[4,3-d]pyrimidin-2-yl)methyl)-3-methoxyphenyl)piperazin-1-yl)pentan-1-one (660 mg, 1.295 mmol) in DMF (8 ml) and THF (8 ml) was added DIEA (4.52 ml, 25.9 mmol) and (6Z,9Z,28Z,31Z)-heptatriaconta-6,9,28,31-tetraen-19-yl (4-nitrophenyl) carbonate (Int. 6-1, 1168 mg, 1.683 mmol) at rt. After 30 minutes the mixture was diluted with water (20 mL) then extracted with EtOAc (20 mL×2). The combined organic layers were dried (Na₂SO₄) then filtered then the filtrate was concentrated. The crude product was subjected to silica gel chromatography (0-10% MeOH/DCM) to give the title compound with impurities. The compound was further purified by reverse-phase chromatography (CH₃CN/water with 0.1% TFA modifier) to give the title compound as the TFA salt. The salt was taken up in saturated NaHCO₃ (20 mL) then extracted with EtOAc (20 mL×2). The combined organic layers were dried (Na₂SO₄) then filtered then the filtrate was concentrated to give the title compound. Compound D-6: MS m/z (M+H)⁺: calculated 1064.8, observed 1064.8. ¹H NMR (400 MHz, CD₃OD) δ 7.61 (s, 1H), 7.14 (d, J=8.4 Hz, 1H), 6.60 (d, J=1.6 Hz, 1H), 6.53 (dd, J=2.0, 8.4 Hz, 1H), 5.39-5.26 (m, 10H), 4.67 (td, J=6.0, 12.0 Hz, 1H), 3.84 (s, 3H), 3.74-3.66 (m, 4H), 3.55 (t, J=7.2 Hz, 2H), 3.25-3.21 (m, 2H), 3.20-3.16 (m, 2H), 3.13 (br t, J=6.4 Hz, 2H), 2.76 (t, J=6.0 Hz, 4H), 2.46 (br t, J=7.2 Hz, 2H), 2.09-2.00 (m, 8H), 1.70-1.61 (m, 4H), 1.56-1.47 (m, 5H), 1.45-1.39 (m, 2H), 1.35-1.25 (m, 36H), 0.97 (t, J=7.2 Hz, 3H), 0.90 (t, J=6.8 Hz, 6H).

### Example 7

### Preparation of Pneumococcal Polysaccharide-Carrier Protein Conjugates

Polysaccharide(s) (as highlighted below and in the Tables and Examples) was dissolved, sized to a target molecular mass, chemically activated and buffer-exchanged by ultrafiltration. Activated polysaccharide and purified CRM197 (a carrier protein) were individually lyophilized and re-dissolved in DMSO. Re-dissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction

Purified pneumococcal capsular polysaccharide (otherwise termed “Ps”) powder was dissolved in water. With the exception of ST-19A, (serotype is otherwise termed “ST”) which is not sized reduced, dissolved polysaccharide was 0.45-micron filtered and either homogenized or acid hydrolyzed to reduce the molecular mass of the Ps. Target Ps size was achieved for homogenization by controlling the pressure and number of passes. Target Ps size was achieved for acid hydrolysis by controlling the temperature and time. Polysaccharide was then 0.2-micron filtered and concentrated and diafiltered against water using a 5 or 10 kDa NMWCO tangential flow ultrafiltration membrane.

### De-O-Acetylation (ST-15B Only)

Size reduced ST-15B Ps solution was heated to 60° C. and sodium bicarbonate pH 9.4 buffer was added to a final concentration of 50 mM. The batch was incubated at 60° C. to release O-acetyl groups. Potassium phosphate pH 6 buffer was added to neutralize pH and the solution was cooled to ambient temperature. The solution was then concentrated and diafiltered against water using a 5 or 10 kDa NMWCO tangential flow ultrafiltration membrane.

Deketalization (ST-4 only)

Sized reduced ST-4 Ps solution was adjusted to 50° C. and pH 4.1 with a sodium acetate buffer to partially deketalize the polysaccharide. The polysaccharide solution was then cooled to 22° C. prior to activation.

### Polysaccharide Oxidation

The polysaccharide solution was adjusted to 22° C. for all serotypes, except for ST-5, 7F and 19F, which were adjusted to 4° C. The solution was also adjusted to pH 4-5 with a sodium acetate buffer to minimize polysaccharide size reduction due to activation. Polysaccharide activation was initiated with the addition of a sodium metaperiodate solution. The amount of sodium metaperiodate added was controlled to achieve a target level of polysaccharide activation (moles aldehyde per mole of polysaccharide repeating unit).

The activated product for all serotypes except ST-5 was diafiltered against 10 mM potassium phosphate, pH 6.4 followed by diafiltration against water using a 5 or 10 kDa NMWCO tangential flow ultrafiltration membrane. For ST-5, the activated product was diafiltered against 10 mM sodium acetate, pH 4.1 followed by diafiltration against water using a 5 kDa NMWCO tangential flow ultrafiltration membrane. Ultrafiltration was conducted at 2-8° C. for all serotypes.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered. Activated polysaccharides were formulated for lyophilization with water and sucrose. CRM197 was formulated for lyophilization at 6 mg Pr/mL (the CRM197 protein is otherwise referred to as “Pr”) with sucrose concentration of 1% w/v. Formulated Ps and CRM197 solutions were individually lyophilized. Lyophilized Ps and CRM197 materials were re-dissolved individually in equal volumes of DMSO. Additives such as salt were spiked into the Ps-DMSO for some serotypes. The polysaccharide and CRM197 solutions were blended to achieve a target polysaccharide concentration and polysaccharide to CRM197 mass ratio. The mass ratio was selected to control the polysaccharide to CRM197 ratio in the resulting conjugate. A reducing agent such as sodium cyanoborohydride was added for most serotypes and conjugation proceeded at 22° C.

### Final Reduction

A reducing agent such as sodium borohydride was added following the conjugation reaction and incubated at 22° C. for all serotypes. The batch was diluted into 150 mM sodium chloride, with approximately 0.025% (w/v) polysorbate 20, at approximately 4° C. Potassium phosphate buffer was then added to neutralize the pH. Some lots were concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

Individual batches were then concentrated and diafiltered against 10 mM histidine in 150 mM sodium chloride, pH 7.0, with 0.015% (w/v) PS-20, at 4° C. using a 300 kDa NMWCO tangential flow ultrafiltration membrane. Specifically, for ST-5, halfway through the diafiltration step, ST-5 conjugate was harvested and incubated with 50 mM sodium bicarbonate, pH 9.3 for 3 hours. The ST-5 solution was neutralized with 1.5 M potassium phosphate, pH 6.0 prior to completing diafiltration.

The individual retentate batches were 0.2-micron filtered (with 0.5-micron prefilter) then diluted with additional 10 mM histidine in 150 mM sodium chloride, pH 7.0 with 0.015% (w/v) PS-20, dispensed into aliquots and frozen at <−60° C. Serotype specific conjugate details can be found as previously described (WO2011/100151, WO2019/139692 and WO2020/131763).

### Example 8

### Formulation of Pneumococcal Conjugate Compositions

Individual pneumococcal polysaccharide-carrier protein conjugates prepared utilizing different chemistries as described in Example 7 were used for the formulation of a 1- or 24-valent pneumococcal conjugate composition referred to as PCV1 or PCV24, respectively.

The PCV1 formulation, to be added to the Compound A-1-SNE, Compound B-1-SNE or Compound D-1-SNE, contained serotype 6B conjugated using reductive amination as described in Examples, supra, and formulated in 20 mM L-Histidine pH 5.8, 150 mM NaCl and 0.1% (w/v) PS-20 for a final concentration of 0.4 μg/mL (w/v) pneumococcal polysaccharide (PnPs) in the vaccine. The PCV1 vaccine formulation, prepared with APA and serotype 6B conjugated using reductive amination in an aprotic (DMSO) solvent, as described in Example 7, was formulated in 20 mM L-Histidine pH 5.8, 150 mM NaCl and 0.2% (w/v) PS-20 and 250 μg (Al)/mL in the form of APA for a final concentration of 0.4 μg/mL (w/v) pneumococcal polysaccharide (PnPs) in the vaccine.

The PCV24 formulation, to be added to Compound A-1-SNE for IRM study, contained serotypes 1, 3, 4, 5, 6A, 6B, 7F, 8, 9V, 10A, 11A, 12F, 14, 15A, de-O—Ac15B, 18C, 19A, 19F, 22F, 23B, 23F, 24F, 33F and 35B, all conjugated to CRM197 using reductive amination and formulated in 20 mM L-Histidine pH 5.8 150 mM NaCl and 0.1% PS-20. Each polysaccharide-carrier protein conjugate was formulated at 0.8 μg/mL (w/v) pneumococcal polysaccharide (PnPs) for a final concentration of 19.2 μg/mL PnPs in the vaccine.

The PCV24 formulation, either with or without APA for IRM study, contained serotypes 1, 3, 4, 5, 6A, 6B, 7F, 8, 9V, 10A, 11A, 12F, 14, 15A, de-O—Ac15B, 18C, 19A, 19F, 22F, 23B, 23F, 24F, 33F and 35B, all conjugated to CRM197 as described in Examples, supra using reductive amination and formulated in 20 mM L-Histidine pH 5.8 150 mM NaCl and 0.2% PS-20 and 250 μg (Al)/mL in the form of APA (if needed). Each polysaccharide-carrier protein conjugate was formulated at 4.0 μg/mL (w/v) pneumococcal polysaccharide (PnPs) for a final concentration of 96 μg/mL PnPs in the vaccine.

PCV20, which contains the following serotypes (1, 3, 4, 5, 6A, 6B, 7F, 8, 9V, 10A, 11A, 12F, 14, 15B, 18C, 19A, 19F, 22F, 23F, 33F), all conjugated to CRM197, was purchased from Myonex.

The PCV26 formulation, to be added to Compound B-1-SNE for mouse study, contained serotypes 1, 3, 4, 5, 6A, 6B, 7F, 8, 9V, 10A, 11A, 12F, 14, 15A, de-O—Ac15B, 16F, 18C, 19A, 19F, 22F, 23A, 23B, 23F, 24F, 33F and 35B, all conjugated to CRM197 using reductive amination and formulated in 20 mM L-Histidine pH 5.8 150 mM NaCl and 0.1% PS-20. Each polysaccharide-carrier protein conjugate was formulated at 0.8 μg/mL (w/v) pneumococcal polysaccharide (PnPs) for a final concentration of 19.2 μg/mL PnPs in the vaccine.

To prepare the PCV formulations, the required volumes of monovalent bulk conjugates needed to obtain the indicated final concentration of (w/v) pneumococcal polysaccharide (also referred to as PnPs) were calculated based on the batch volume or mass and the bulk polysaccharide concentration.

The formulation process consisted of a conjugate bulk blend preparation at 2× the final concentration of PnPs blends in 20 mM Histidine, 0.05 to 0.15% (w/v) PS-20, and 150 mM sodium chloride, pH 5.8.

Histidine pH 5.8, PS-20 and sodium chloride solutions were prepared and added to the formulation vessel. The individual pneumococcal polysaccharide-carrier protein conjugates, stored frozen, were thawed at 2-8° C. and then added to the formulation vessel. During the addition of polysaccharide-carrier protein conjugate to the formulation buffer (conjugate blend), the vessel was mixed to ensure homogeneity using a magnetic sir bar or magnetic impeller. After all additions were made and the solution was stirred, the conjugate blend was passed through sterilizing filters and collected in a vessel with or without APA. In some cases, the sterilizing filters were chased with 150 mM sodium chloride to adjust the batch to target concentration.

The formulations were filled into plastic syringes, glass syringes, or vials.

### Example 9

Preparation of a Stable Nanoemulsion (SNE) Adjuvant System with and without Compounds

The SNE adjuvant can be prepared with and without the compounds:
(N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide (Compound A-1);
(S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide (Compound A-2);
(S)-1-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)octadecan-1-one (Compound A-3);
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide (Compound B-1);
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)tetradecanamide (Compound B-2);
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)oleamide (Compound B-3);
(9Z,12Z)—N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)octadeca-9,12-dienamide (Compound B-4);
N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide (Compound B-5);
N-(5-(4-(4-((5-amino-7-(butylamino)-2H-pyrazolo[4,3-d]pyrimidin-2-yl)methyl)-3-methoxyphenyl)piperidin-1-yl)-5-oxopentyl)stearamide (Compound B-6);
N-(5-(3-(4-((5-amino-7-(butylamino)-2H-pyrazolo[4,3-d]pyrimidin-2-yl)methyl)-3-methoxyphenyl)azetidin-1-yl)-5-oxopentyl)stearamide (Compound B-7);
1-(4-((5-amino-7-(butylamino)-2H-pyrazolo[4,3-d]pyrimidin-2-yl)methyl)-3-methoxyphenyl)-N-(3-stearamidopropyl)piperidine-4-carboxamide (Compound B-8);
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-octadecylcyclobutane-1-carboxamide (Compound B-9);
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide (Compound B-10);
N-(3-(4-(4-((5-amino-7-(butylamino)-2H-pyrazolo[4,3-d]pyrimidin-2-yl)methyl)-3-methoxyphenyl)piperazin-1-yl)-3-oxopropyl)stearamide (Compound B-11);
N-(7-(4-(4-((5-amino-7-(butylamino)-2H-pyrazolo[4,3-d]pyrimidin-2-yl)methyl)-3-methoxyphenyl)piperazin-1-yl)-7-oxoheptyl)stearamide (Compound B-12);
N-(3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)cyclobutyl)stearamide (Compound B-13);
N-(5-(4-(4-((5-amino-7-(butylamino)-2H-pyrazolo[4,3-d]pyrimidin-2-yl)methyl)-3-methoxyphenyl)piperazin-1-yl)-4,4-dimethyl-5-oxopentyl)stearamide (Compound B-14);
N-(6-(4-(4-((5-amino-7-(butylamino)-2H-pyrazolo[4,3-d]pyrimidin-2-yl)methyl)-3-methoxyphenyl)piperazin-1-yl)-2-methyl-6-oxohexan-2-yl)stearamide (Compound B-15);
1-(4-(4-((5-amino-7-(butylamino)-2H-pyrazolo[4,3-d]pyrimidin-2-yl)methyl)-3-methoxyphenyl)piperazin-1-yl)-5-(octadecyloxy)pentan-1-one (Compound B-16);
1-(4-(4-((5-amino-7-(butylamino)-2H-pyrazolo[4,3-d]pyrimidin-2-yl)methyl)-3-methoxyphenyl)piperazin-1-yl)-5-(octadecylamino)pentan-1-one (Compound B-17);
N-(5-(4-(4-((5-amino-7-(butylamino)-3-methyl-2H-pyrazolo[4,3-d]pyrimidin-2-yl)methyl)-3-methoxyphenyl)piperazin-1-yl)-5-oxopentyl)stearamide (Compound B-18);
(9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide (Compound C-1);
N-(4-((4-((5-amino-7-(butylamino)-2H-pyrazolo[4,3-d]pyrimidin-2-yl)methyl)-3,5-dimethoxybenzyl)(methyl)amino)butyl)tetradecanamide (Compound C-2);
N-(4-((4-((5-amino-7-(butylamino)-2H-pyrazolo[4,3-d]pyrimidin-2-yl)methyl)-3,5-dimethoxybenzyl)(methyl)amino)butyl)oleamide (Compound C-3);
N-(4-((4-((5-amino-7-(butylamino)-2H-pyrazolo[4,3-d]pyrimidin-2-yl)methyl)-3,5-dimethoxybenzyl)(methyl)amino)butyl)stearamide (Compound C-4);
N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide (Compound C-5);
(6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)amino)butyl)carbamate (Compound D-1);
4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide (Compound D-2);
3-stearamidopropyl 4-(4-((5-amino-7-(butylamino)-2H-pyrazolo[4,3-d]pyrimidin-2-yl)methyl)-3-methoxyphenyl)piperazine-1-carboxylate (Compound) D-3);
N-(5-(4-(4-((5-amino-7-(butylamino)-2H-pyrazolo[4,3-d]pyrimidin-2-yl)methyl)-3-methoxyphenyl)piperazin-1-yl)-6,6,6-trifluorohexyl)stearamide (Compound D-4);
N-(4-((4-((7-(butylamino)-5-hydroxy-2H-pyrazolo[4,3-d]pyrimidin-2-yl)methyl)-3,5-dimethoxybenzyl)(methyl)amino)butyl)stearamide (Compound D-5); and
(6Z,9Z,28Z,31Z)-heptatriaconta-6,9,28,31-tetraen-19-yl (5-(4-(4-((5-amino-7-(butylamino)-2H-pyrazolo[4,3-d]pyrimidin-2-yl)methyl)-3-methoxyphenyl)piperazin-1-yl)-5-oxopentyl)carbamate (Compound D-6).

The SNE is a multi-component emulsion formulation consisting of 3 stabilizing ingredients; SPAN-85, PS-20 and squalene with a compound, for example, Compound A-1 (referred to as Compound A-1-SNE, see Table 1) and without a compound (referred to as SNE, see Table 2). This formulation is prepared by combining and mixing the compound, SPAN-85, PS-20 and squalene components after dissolving in 100% EtOH together (Table 1).

The process of making the compound-stable nanoemulsion (Compound-SNE) consists of 5 steps: 1) solution preparation of a component mixture including the 3 stabilizing ingredients; SPAN-85, PS-20 and squalene, with a compound 2) SNE formation by means of T-mixing; 3) ultra-filtration; 4) bioburden reduced filtration; and 5) sterile filtration and vial filling.

### Solution Preparation of Stabilizer/Compound Mixture

The stabilizer/compound components were weighed and combined before being dissolved in ethanol before being sterile filtered to form the component mixture. Histidine buffer (20 mM Histidine pH 5.8) was prepared.

### SNE Formation by Means of T-Mixing

The lipid mixture and Histidine buffer were then mixed together at adjacent ends of a T-tube mixer. The stream exiting the T-mix apparatus was immediately diluted 1:1 with 20 Histidine 0.05% PS-20 and 75 mM NaCl, and then collected as formed SNE.

### Ultra-Filtration

The SNE intermediate was then subjected to Dialysis or ultra-filtration with a 500 kDA NMWCO in order to both concentrate the material approximately 10-fold as well as buffer exchange the material against 20 mM Histidine, 0.05% (w/v) PS-20 and 75 mM NaCl, pH 5.8. or 20 mm Histidine pH 5.8 or 20 mM Histidine 0.05% PS-20 pH 5.8. After the dialysis or diafiltration, there was a final concentration step performed in order to achieve final target concentration.

### Bioburden Reduced Filtration

The adjuvant bulk was then pre-filtered with a 0.45 μm cellulose acetate (CA) filter followed by a 0.2 μm CA bioburden-reducing filter, and stored liquid at 4° C.

### Sterile Filtration and Vial Filling

The adjuvant bulk was passed through a 0.45 μm polyvinylidene fluoride (PVDF) bioburden reducing filter and a 0.22 μm PVDF sterilizing grad filter and received. The filtered adjuvant bulk was then diluted with 20 mM Histidine 0.05% (w/v) PS-20, and 75 mM NaCl pH 5.8 to the target SNE adjuvant concentration. This diluted final bulk adjuvant was then filled into glass vials and stored at 4° C.

TABLE 1
Composition of Compound A-1-SNE Adjuvant

| | | Content of | | Content of |
|---|---|---|---|---|
| | | Each | Molecular | Each |
| | | Component | Weight | Component |
| Component | Description | (Mole %) | (g/mol) | (Mass %) |
| Compound | N-(5-(4-(4-((5-amino-7- | 2.42-2.71 | 599.3 | 2.44-3.03 |
| A-1 | (butylamino)-2H-pyrazolo[4,3- | | | |
| | d]pyrimidin-2-yl)methyl)-3,5- | | | |
| | dimethoxyphenyl)piperazin-1- | | | |
| | yl)-5-oxopentyl)stearamide | | | |
| Squalene | Squalene | 70.62-79.16 | 410.72 | 48.780-60.61 |
| SPAN-85 | sorbitan trioleate | 10.19-15.15 | 957.5 | 18.18-24.39 |
| PS-20 | polysorbate-20 | 7.94-11.81 | 1228 | 18.18-24.39 |
| Buffer Matrix | 20 mM Histidine, 0.05% PS- | N/A | | |
| | 20, 75 mM NaCl, pH 5.8 | | | |

TABLE 2
Composition of Compound B-1-SNE Adjuvant

| | | Content of | | Content of |
|---|---|---|---|---|
| | | Each | Molecular | Each |
| | | Component | Weight | Component |
| Component | Description | (Mole %) | (g/mol) | (Mass %) |
| Compound | N-(5-(4-(4-((5-amino-7- | 1.67-3.73 | 431.3 | 1.22-3.03 |
| B-1 | (butylamino)-2H-pyrazolo[4,3- | | | |
| | d]pyrimidin-2-yl)methyl)-3- | | | |
| | methoxyphenyl)piperazin-1-yl)- | | | |
| | 5-oxopentyl)stearamide | | | |
| Squalene | Squalene | 78.33-141.1 | 410.72 | 60.61-97.6 |
| SPAN-85 | sorbitan trioleate | 2.02-15.1 | 957.5 | 4.44-24.4 |
| PS-20 | polysorbate-20 | 1.57-11.7 | 1228 | 4.44-24.4 |
| Buffer Matrix | 20 mM Histidine, 0-0.05% PS- | N/A | | |
| | 20, 0-75 mM NaCl, pH 5.8-70 | | | |

TABLE 3
Composition of Compound C-4-SNE Adjuvant

| | | Content of | | Content of |
|---|---|---|---|---|
| | | Each | Molecular | Each |
| | | Component | Weight | Component |
| Component | Description | (Mole %) | (g/mol) | (Mass %) |
| Compound | N-(4-((4-((5-amino-7- | 2.22 | 737.6 | 3.03 |
| C-4 | (butylamino)-2H-pyrazolo[4,3- | | | |
| | d]pyrimidin-2-yl)methyl)- 3,5- | | | |
| | dimethoxybenzyl) | | | |
| | (methyl)amino)butyl) stearamide | | | |
| Squalene | Squalene | 79.56 | 410.72 | 60.61 |
| SPAN-85 | sorbitan trioleate | 10.24 | 957.5 | 18.18 |
| PS-20 | polysorbate-20 | 7.98 | 1228 | 18.18 |
| Buffer | 20 mM Histidine, 0.05% | N/A | | |
| Matrix | PS-20, 75 mM NaCl, pH 5.8 | | | |

TABLE 4
Composition of SNE Adjuvant

| | | Content of | | Content of |
|---|---|---|---|---|
| | | Each | Molecular | Each |
| | | Component | Weight | Component |
| Component | Description | (Mole %) | (g/mol) | (Mass %) |
| Squalene | Squalene | 83.33 | 410.72 | 92.91 |
| SPAN-85 | sorbitan trioleate | 8.33 | 957.5 | 3.99 |
| PS-20 | polysorbate-20 | 8.33 | 1228 | 3.11 |
| Buffer | 20 mM Histidine, 0.05% PS-20, | N/A | | |
| Matrix | 75 mM NaCl, pH 5.8 | | | |

### Example 9A

### Alternate Preparation of a Stable Nanoemulsion (Compound B-1-SNE) Adjuvant System

The Compound B-1-SNE is a multi-component emulsion formulation consisting of 3 stabilizing ingredients: SPAN-85, PS-20 and squalene and the Compound B-1 (see Table 5). This formulation is prepared by combining and mixing the Compound B-1, SPAN-85, PS-20 and squalene components after dissolving in 100% EtOH together.

The process of making the compound-stable nanoemulsion (Compound B-1-SNE) consists of 5 steps: 1) solution preparation of a component mixture that includes the 3 stabilizing ingredients: SPAN-85, PS-20 and squalene and Compound B-1; 2) SNE formation by means of T-mixing or controlled precipitation process utilizing mixing systems to drive self-assembly of the nanoemulsion; 3) ultra-filtration; 4) bioburden reduced filtration; and 5) sterile filtration and vial filling.

### Solution Preparation of Stabilizer/Compound Mixture

The stabilizer/compound components were weighed and combined before being dissolved in ethanol before being heated at 40° C. for 30 min and sterile filtered to form the component mixture.

### SNE Formation by Means of T-Mixing or Controlled Precipitation Process Utilizing Mixing Systems to Drive Self-Assembly of the Nanoemulsion

The component mixture and Histidine buffer were then mixed together at adjacent ends of a T-tube mixer or fluidic assembly. The stream exiting the apparatus was immediately diluted 1:1 with 20 Histidine pH 5.8, and then collected as the formed SNE.

### Ultra-Filtration

The SNE intermediate was then subjected to Dialysis or ultra-filtration with a 500 kDA NMWCO in order to both concentrate the material approximately 10-fold as well as buffer exchange the material against 20 mM Histidine, 0.05% (w/v) PS-20 and 75 mM NaCl, pH 5.8 or 20 mM Histidine pH 5.8 or 20 mM Histidine 0.05% PS-20 pH 5.8. After the dialysis or diafiltration, there was a final concentration step performed in order to achieve final target concentration.

Histidine pH 5.8, PS-20, sodium chloride, L-met, and EDTA solutions were prepared and added to a formulation vessel. The adjuvant bulk SNE was added to the formulation vessel. During the addition of the SNE to the formulation buffer, the vessel was mixed to ensure homogeneity using a magnetic sir bar or magnetic impeller. After all additions were made and the solution was stirred the formulations were filled into plastic syringes, glass syringes, or vials.

TABLE 5
Composition of Compound B-1-SNE Adjuvant

| | | Content of | | Content of |
|---|---|---|---|---|
| | | Each | Molecular | Each |
| | | Component | Weight | Component |
| Component | Description | (Mole %) | (g/mol) | (Mass %) |
| Compound | N-(5-(4-(4-((5-amino-7- | 0.01-0.1 | 431.3 | 0.30-3.03 |
| B-1 | (butylamino)-2H-pyrazolo[4,3- | | | |
| | d]pyrimidin-2-yl)methyl)-3- | | | |
| | methoxyphenyl)piperazin-1-yl)- | | | |
| | 5-oxopentyl)stearamide | | | |
| Squalene | Squalene | 80.0-98.0 | 410.72 | 61.5-97.0 |
| SPAN-85 | sorbitan trioleate | 1.5-15.0 | 957.5 | 3.1-25.0 |
| PS-20 | polysorbate-20 | 1.0-10.0 | 1228 | 1.2-24.0 |
| Buffer Matrix | 5-25 mM Histidine, pH 5.8, | N/A | | |
| | 0.05% PS-20, 0-75 mM NaCl, | | | |
| | 5-12 mM L-met, 5-10 mM | | | |
| | EDTA | | | |

### Example 10

### Cell-Based TLR 7/8 Assay to Evaluate Compound-SNE Activity

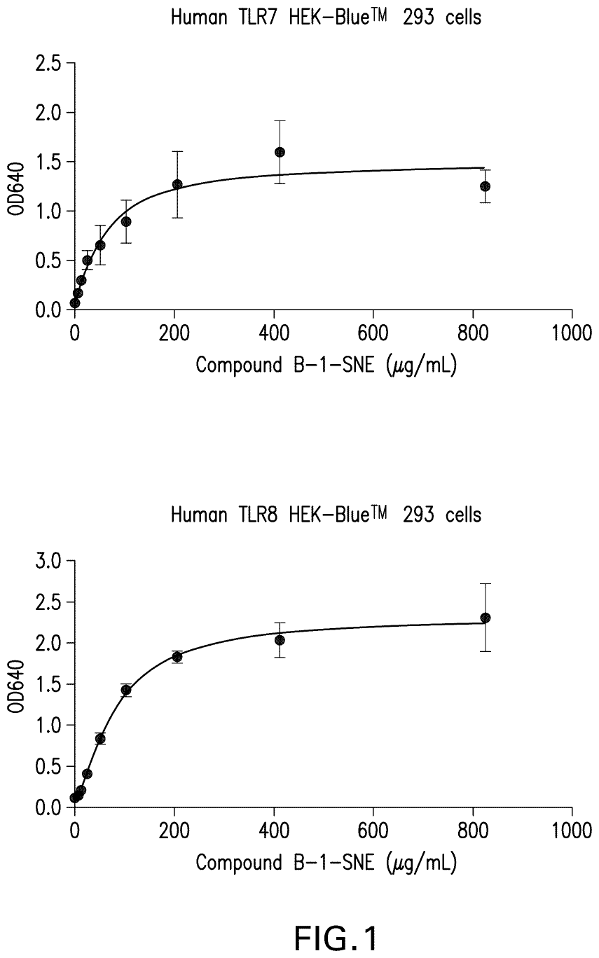

As shown in FIG. 1, a cell-based activation system was used to assess activity of the Compound-formulated nanoemulsion systems (Compound B-1-SNE), prepared as described in examples, supra. HEK-Blue™ TLR7 and TLR8 cells (InvivoGen) were designed to assess the stimulation of TLR7 and TLR8 by monitoring the activation of NF-κB and AP-1. Compound B-1-SNE samples were diluted 1:2 across an 8-point titration in diluent buffer histidine/PS-20 in a sterile V-bottom plate. 20 μL of samples were transferred to corresponding wells of two flat bottom assay plates. Media was aspirated from HEK-Blue™ human TLR 7 and TLR 8 cells (T-75 flasks) and gently rinsed with pre-warmed (37° C.) DPBS for a total volume of 5 mL/flask. The DPBS was gently removed from the cells and cells were gently manually dislodged and resuspended in 2 mL of DPBS. Cell assay suspensions were then prepared at approximately 2.2×105 cells per mL of HEK-Blue™ detection medium (InvivoGen). 180 μL of cell suspension in detection medium was added to each well of the two flat bottom assay plates containing the diluted Compound B-1-SNE samples for a final dilution of sample equaling 1:10 (180 μL of cell suspension in detection medium and 20 μL of sample). The cells were then incubated at 37° C. in 5% CO2 for 20 hrs. The reporter gene, NF-Kb, induces the production of secreted embryonic alkaline phosphatase (SEAP) when stimulated and can be monitored using the vendor supplied SEAP detection kit. SEAP colorimetric changes were read at an absorbance of 640 nm using a SpectroMax Stakmax plate reader. As shown in FIG. 1, Compound B-1-SNE activates both TLR7 and TLR8 receptors with an EC50 of 65.4 μg/mL for TLR7 and 82.3 μg/mL for TLR8. EC50 values were calculated based on total Compound B-1-SNE concentration using GraphPad Prism Software and analyzed using agonist vs. response variable slope (four parameters) least squares fit.

### Example 10A

### Further Cell-Based TLR 7/8 Assay Information to Evaluate Compound-SNE Activity

Further to the description in Example 10, a cell-based activation system was used to assess activity of the Compound-formulated nanoemulsion systems, prepared as described in examples, supra. HEK-Blue™ TLR7 and TLR8 cells (InvivoGen) were designed to assess the stimulation of TLR7 and TLR8 by monitoring the activation of NF-κB and AP-1. Compound-SNE samples were diluted 1:2 across an 8-point titration in diluent buffer histidine/PS-20 in a sterile V-bottom plate. 20 μL of samples were transferred to corresponding wells of two flat bottom assay plates. Media was aspirated from HEK-Blue™ human TLR 7 and TLR 8 cells (T-75 flasks) and gently rinsed with pre-warmed (37° C.) DPBS for a total volume of 5 mL/flask. The DPBS was gently removed from the cells and cells were gently manually dislodged and resuspended in 2 mL of DPBS. Cell assay suspensions were then prepared at approximately 2.2×105 cells per mL of HEK-Blue™ detection medium (InvivoGen). 180 μL of cell suspension in detection medium was added to each well of the two flat bottom assay plates containing the diluted Compound-SNE samples for a final dilution of sample equaling 1:10 (180 μL of cell suspension in detection medium and 20 μL of sample). The cells were then incubated at 37° C. in 5% CO₂ for 20 hrs. The reporter gene, NF-Kb, induces the production of secreted embryonic alkaline phosphatase (SLAP) when stimulated and can be monitored using the vendor supplied SLAP detection kit. SLAP colorimetric changes were read at an absorbance of 640 nm using a SpectroMax Stakmax plate reader. EC50s were calculated based on Compound concentration only, not total Compound-SNE concentration (Table 6) using GraphPad Prism Software and analyzed using agonist vs. response variable slope (four parameters) least squares fit. As shown in Table 6, Compounds formulated as an SNE activate both TLR7 and TLR8 receptors.

TABLE 6
EC50 values, based on Compound concentration,
for Compounds formulated as SNEs

| | huTLR7 | huTLR8 | | huTLR7 | huTLR8 |
|---|---|---|---|---|---|
| Compound | EC₅₀ | EC₅₀ | Compound | EC₅₀ | EC₅₀ |
| Number | (μg/mL) | (μg/mL) | Number | (μg/mL) | (μg/mL) |
| B-1 | 0.65 | 0.43 | B-13 | 67.68 | 2.38 |
| B-2 | 0.15 | 0.22 | C-5 | 0.48 | 0.07 |
| B-3 | 0.12 | 0.1 | B-11 | 2.3 | 0.3 |
| B-4 | 0.3 | 0.3 | B-6 | 3.25 | 2.01 |
| A-1 | 12.01 | 4.33 | B-10 | 90.94 | 56.99 |
| C-4 | 3.65 | 0.86 | D-2 | 13.76 | 4.52 |
| C-2 | 0.23 | 0.12 | B-8 | 50.18 | 41.9 |
| C-3 | 0.37 | 0.12 | B-5 | 10.59 | 3.33 |
| C-1 | 0.19 | 0.07 | B-7 | 6.3 | 2.53 |
| D-1 | 17.63 | 159.2 | B-14 | 108.3 | 9.53 |
| B-12 | 18.8 | 20.53 | A-2 | 6.3 | 0.38 |
| D-3 | 18.46 | 10.77 | B-18 | 0.25 | 0.17 |

### Example 11

### PCV1 Immunogenicity in Mice: Evaluation of Adjuvant Systems

Young female Balb/C mice (6-8 weeks old, n=10/group) were intramuscularly (IM) immunized with 0.2 mL of PCV1 formulated with different adjuvants (Table 7) on day 0, day 28, and day 56. PCV1 was dosed at 0.08, μg PnPs (6B conjugated to CRM197) per immunization. Mice were observed at least daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc. (Rahway, NJ, USA).

TABLE 7
Formulations Assessed in PCV1 Immunogenicity Study in Mice
Formulation

ST-6B-CRM197; 0.08 μg PnPs/mL 20 mM L-histidine,
150 mM NaCl, 0.1% w/v PS-20, pH 5.8
[25 μg/dose APA]
ST-6B-CRM197; 0.08 μg PnPs/mL; 20 mM L-histidine,
150 mM NaCl, 0.1% w/v PS-20, pH 5.8
[15 μg/dose Compound A-1-SNE]
ST-6B-CRM197; 0.08 μg PnPs/mL; 20 mM L-histidine,
150 mM NaCl, 0.1% w/v PS-20, pH 5.8
[1.5 μg/dose Compound A-1-SNE]
ST-6B-CRM197; 0.08 μg PnPs/mL; 20 mM L-histidine,
150 mM NaCl, 0.1% w/v PS-20, pH 5.8
[0.15 μg/dose Compound A-1-SNE]
ST-6B-CRM197; 0.8 μg PnPs/mL; 20 mM L-histidine,
150 mM NaCl, 0.1% w/v PS-20, pH 5.8
[30 μg/dose Compound B-1-SNE]
ST-6B-CRM197; 0.08 μg PnPs/mL; 20 mM L-histidine,
150 mM NaCl, 0.1% w/v PS-20, pH 5.8
[3 μg/dose Compound B-1-SNE]
ST-6B-CRM197; 0.08 μg PnPs/mL; 20 mM L-histidine,
150 mM NaCl, 0.1% w/v PS-20, pH 5.8
[0.3 μg/dose Compound B-1-SNE]
ST-6B-CRM197; 0.08 μg PnPs/mL; 20 mM L-histidine,
150 mM NaCl, 0.1% w/v PS-20, pH 5.8
[30 μg/dose Compound C-4-SNE]
ST-6B-CRM197; 0.08 μg PnPs/mL; 20 mM L-histidine,
150 mM NaCl, 0.1% w/v PS-20, pH 5.8
[3 μg/dose Compound C-4-SNE]
ST-6B-CRM197; 0.08 μg PnPs/mL; 20 mM L-histidine,
150 mM NaCl, 0.1% w/v PS-20, pH 5.8
[0.3 μg/dose Compound C-4-SNE]

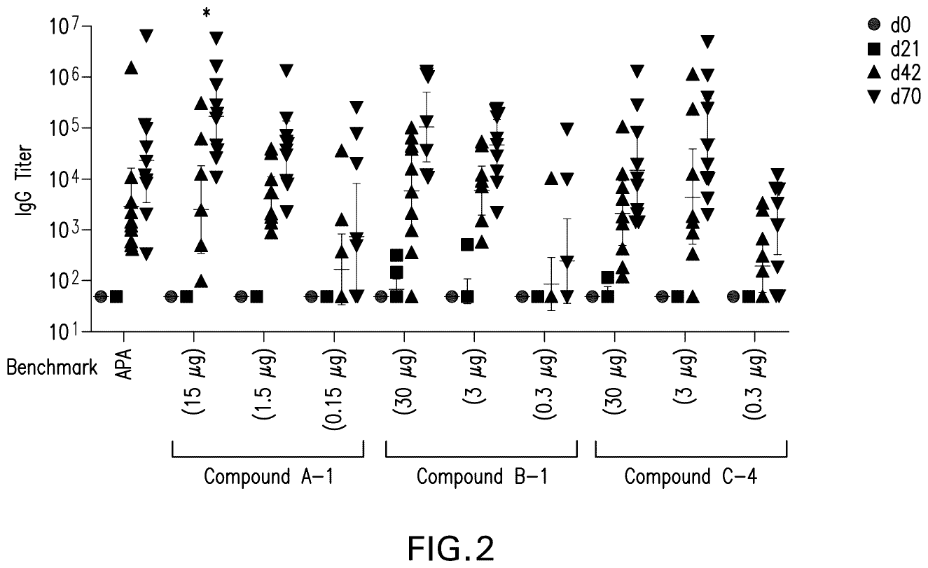

Mouse sera were evaluated for serotype 6B (ST-6B)-specific IgG antibody titers using an ELISA immunoassay (FIG. 2). PCV1 immunization of Balb/C mice generated ST-6B-specific IgG antibody titers when formulated with APA, Compound A-1-SNE (at either 0.15, 1.5 or 15 μg Compound A-1 per dose), Compound B-1-SNE (at either 0.3, 3 or 30 μg Compound B-1 per dose), or Compound C-4-SNE (0.3, 3 or 30 μg Compound C-4 per dose). All formulations were found to be immunogenic, resulted in increased antibody titers after boosting, and, in general, demonstrated a dose-dependent response profile.

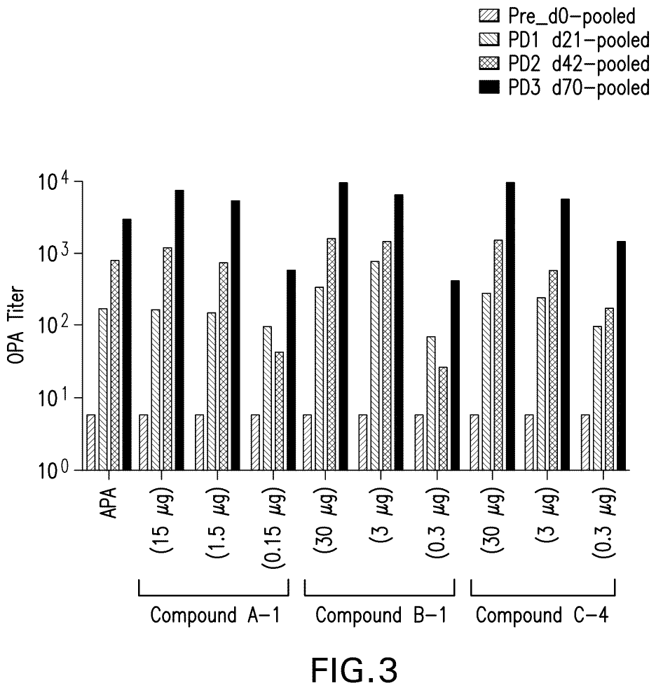

Functional antibody titers were determined through opsonophagocytic assays (OPA, FIG. 3) based on previously described protocols at www.vaccine.uab.edu and Opsotiter® 3 software owned by and licensed from University of Alabama (UAB) Research Foundation (See Caro-Aguilar I. et al., Vaccine (2017) 35(6):865-72 and Burton R. L. and Nahm M. H. Clin. Vaccine Immunol. (2006) 13(9):1004-9). ST-6B-specific functional antibody titers were generated in Balb/C mice, further confirming all formulations were immunogenic.

### Example 12

### PCV24 Immunogenicity Study in Infant Rhesus Monkey (IRM)

PCV24 (serotypes-1, 3, 4, 5, 6A, 6B, 7F, 8, 9V, 10A, 11A, 12F, 14, 15A, 15C, 18C, 19A, 19F, 22F, 23B, 23F, 24F, 33F and 35B each individually conjugated to CRM197) and adjuvant formulations were prepared or obtained (PCV20) as described in Examples, supra. Serotype “15C” is a de-O-acetylated 15B serotype. The PCV24 serotypes can also be defined as (serotypes-1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B). IRMs (Infant Rhesus Monkeys, n=5/group) were intramuscularly immunized with either 0.1 mL (No SNE Adjuvant or Aluminum Adjuvant) or 0.5 mL (Compound B-1-SNE) vaccine, as described in Table 8, below, on days 0, 28 and 56. Sera were collected prior to study start (pre) and on days 14 (PD1), 42 (PD2), 70 (PD3). IRMs were observed at least once daily by trained animal care staff for any signs of illness or distress. The vaccine formulations in IRMs were deemed to be safe and well tolerated, and no vaccine-related adverse events were noted.

TABLE 8
PCV24 Formulations Evaluated in an Infant
Rhesus Macaque Immunogenicity Model
Formulation

PCV24 in 20 mM L-histidine, 150 mM NaCl, 0.2% w/v PS-20, pH 5.8
[No Adjuvant]
PCV24 in 20 mM L-histidine, 150 mM NaCl, 0.2% w/v
PS-20, pH 5.8, 250 μg/mL APA
[Aluminum Phosphate Adjuvant]
PCV24 in 20 mM L-histidine, 75 mM NaCl, 0.05% w/v PS-20, pH 5.8
[300 μg/mL Compound B-1-SNE (6.0 mg/mL of squalene;
1.8 mg/mL of PS-20; 1.8 mg/mL of SPAN-85)]
PCV24 in 20 mM L-histidine, 75 mM NaCl, 0.05% w/v PS-20, pH 5.8
[100 μg/mL Compound B-1-SNE (2.0 mg/mL of squalene;
0.6 mg/mL of PS-20; 0.6 mg/mL of SPAN-85)]
PCV24 in 20 mM L-histidine, 75 mM NaCl, 0.05% w/v PS-20, pH 5.8
[30 μg/mL Compound B-1-SNE (0.6 mg/mL of squalene;
0.18 mg/mL of PS-20; 0.18 mg/mL of SPAN-85)]

Serotype-specific IgG responses were evaluated using a multiplexed electrochemiluminescence (ECL) assay developed for use with rhesus monkey serum and based on the human assay described in Marchese et al., Clin Vaccine Immunol (2009) 16(3):387-96.

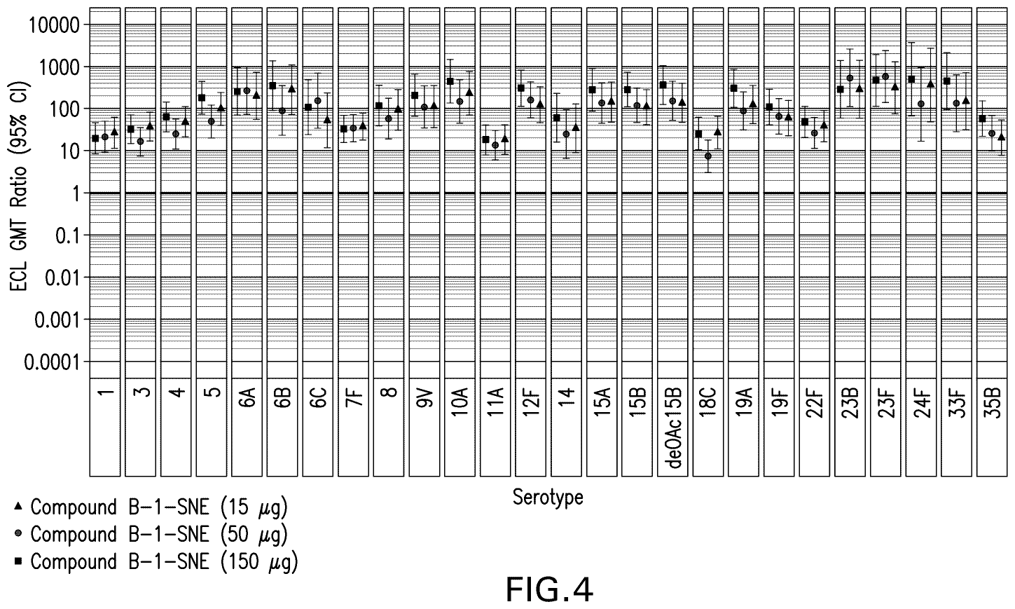

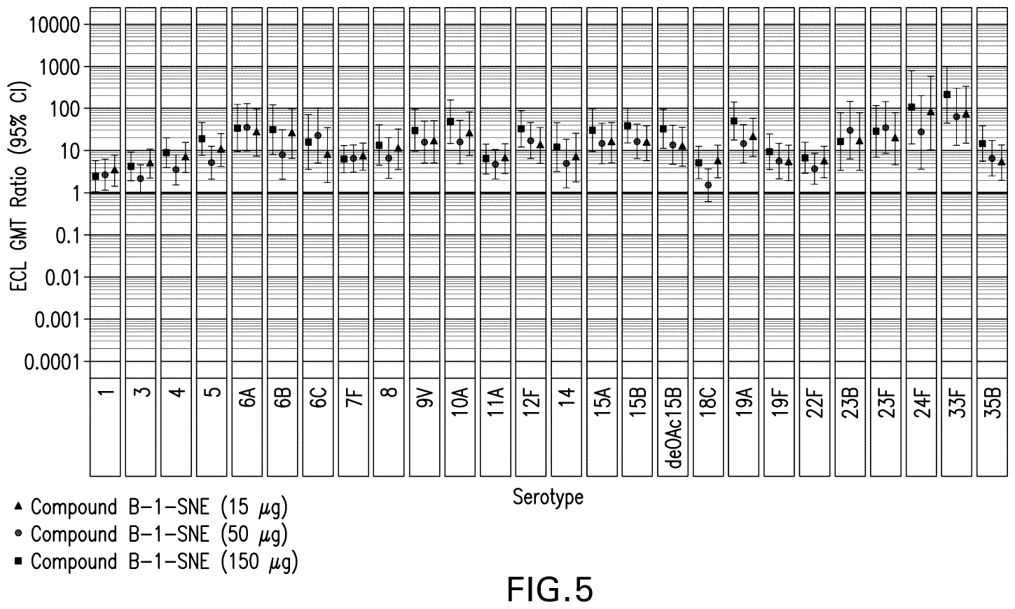

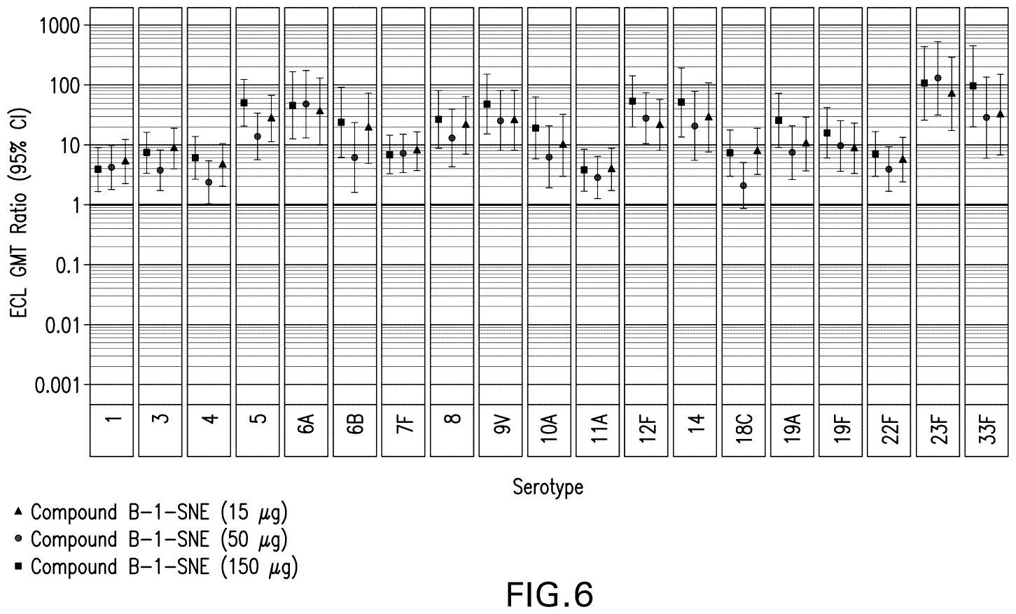

At post-dose 3 (day 70), PCV24 formulated with Compound B-1-SNE at all dose levels showed significantly higher antibody titers when compared to PCV24 without adjuvant (FIG. 4). Statistical significance was defined as having a GMT ratio with lower 95% CI exceeding 1.0. As shown in FIG. 5, PCV24 formulated with Compound B-1-SNE, at all dose levels, showed significantly higher antibody titers when compared to PCV24 formulated with APA for the majority of serotypes at post-dose 3. PCV24 formulated with Compound B-1-SNE, at all dose levels, showed significantly higher antibody titers when compared to PCV20 for the majority of shared serotypes at post-dose 3 (FIG. 6).

### Example 13

### PCV24 Immunogenicity Study in Infant Rhesus Monkey (IRM)

PCV24 (serotypes-1, 3, 4, 5, 6A, 6B, 7F, 8, 9V, 10A, 11A, 12F, 14, 15A, 15C, 18C, 19A, 19F, 22F, 23B, 23F, 24F, 33F and 35B each individually conjugated to CRM197) and adjuvant formulations were prepared or obtained (PCV20) as described in Examples, supra. Serotype “15C” is a de-O-acetylated 15B serotype. The PCV24 serotypes can also be defined as (serotypes-1, 3, 4, 5, 6A, 6B, 7F, 8, 9V, 10A, 11A, 12F, 14, 15A, de-O-acetylated 15B, 18C, 19A, 19F, 22F, 23B, 23F, 24F, 33F and 35B). IRMs (Infant Rhesus Monkeys, n=5/group) were intramuscularly immunized with 0.1 mL PCV20 (Aluminum Adjuvant) or 0.5 mL PCV24 adjuvanted with Compound B-1-SNE, as described in Table 9, below, on days 0, 28 and 56. Sera were collected prior to study start (pre) and on days 14 (PD1), 42 (PD2), 70 (PD3). IRMs were observed at least once daily by trained animal care staff for any signs of illness or distress. The vaccine formulations in IRMs were deemed to be safe and well tolerated, and no vaccine-related adverse events were noted.

TABLE 9
PCV24 Formulations Evaluated in an Infant
Rhesus Macaque Immunogenicity Model
Formulation

PCV20 [Aluminum Phosphate Adjuvant], PREVNAR 20 ™
PCV24 in 20 mM L-histidine, 150 mM NaCl, 0.1% w/v PS-20, pH 5.8
[200 μg/mL Compound B-1-SNE (8.0 mg/mL of squalene;
2 mg/mL of PS-20; 2 mg/mL of SPAN-85)]
PCV24 in 20 mM L-histidine, 150 mM NaCl, 0.1% w/v PS-20, pH 5.8
[20 μg/mL Compound B-1-SNE (0.8 mg/mL of squalene;
0.2 mg/ml of PS-20; 0.2 mg/mL of SPAN-85)]
PCV24 in 20 mM L-histidine, 150 mM NaCl, 0.1% w/v PS-20, pH 5.8
[2 μg/mL Compound B-1-SNE (0.08 mg/mL of squalene;
0.02 mg/mL of PS-20; 0.02 mg/mL of SPAN-85)]
PCV24 in 20 mM L-histidine, 150 mM NaCl, 0.1% w/v PS-20, pH 5.8
[0.2 μg/mL Compound B-1-SNE (0.008 mg/mL of squalene;
0.002 mg/mL of PS-20; 0.002 mg/mL of SPAN-85)]

Serotype-specific IgG responses were evaluated using a multiplexed electrochemiluminescence (ECL) assay developed for use with rhesus monkey serum and based on the human assay described in Marchese et al., Clin Vaccine Immunol (2009) 16(3):387-96.

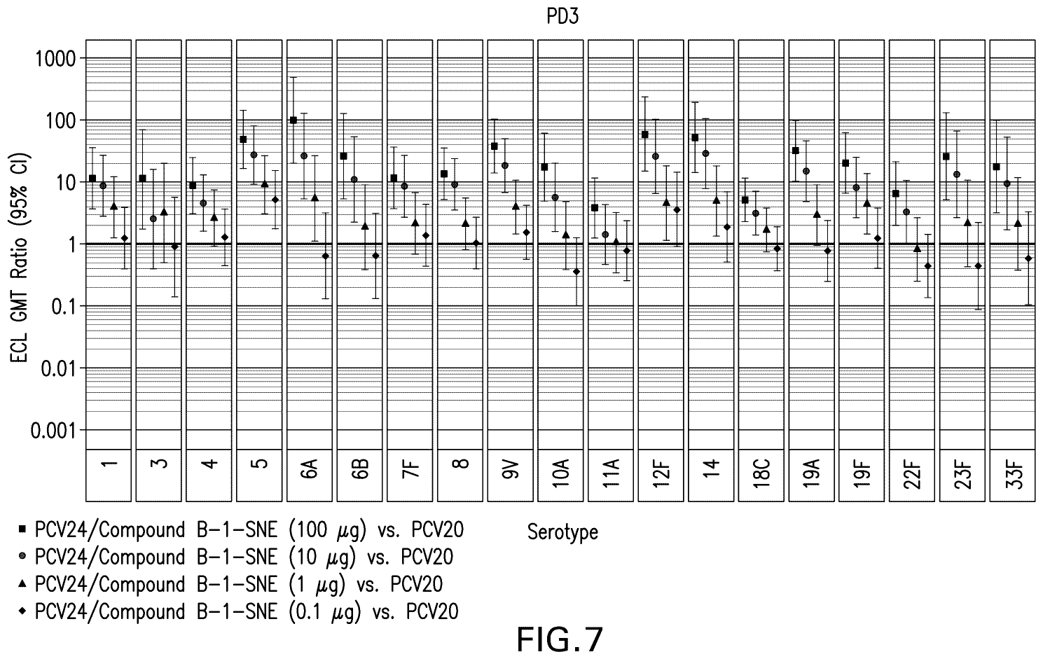

At post-dose 3 (day 70), PCV24 formulated with Compound B-1-SNE at the 100 μg dose level showed significantly higher antibody titers for all shared serotypes when compared to PCV20 (FIG. 7). Statistically higher antibody titers were observed for 17 of 19 shared serotypes at the 10 μg dose level, 7 of 19 shared serotypes at the 1 μg dose level, 1 of 19 shared serotypes at the 0.1 μg dose level when compared to PCV20. All other titers were statistically comparable between the two groups. Statistical significance was defined as having a GMT ratio with lower 95% CI exceeding 1.0.

### Example 14

### PCV26 Immunogenicity and Functional Antibody Responses in Mice

Female Balb/c mice (6-8 weeks old, n=15/group) were intramuscularly immunized with 0.1 mL of 26-valent pneumococcal conjugate vaccine (PCV) on day 0, day 28, and day 56. PCV26 was administered at 0.4 μg of each pneumococcal polysaccharide (1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F, 35B) all individually conjugated to CRM197 and adjuvanted with aluminum phosphate adjuvant (APA) or Compound B-1-SNE. Adjuvants were utilized at concentrations as described in Table 10. Mice were observed at least daily by trained animal care staff for any signs of illness or distress.

TABLE 10
PCV26 Formulations
Formulation

PCV26 (104 mg (PnPs)/mL) in 18.1 mM L-histidine,
150 mM NaCl, 0.09% w/v PS-20, pH 5.8 250 mg/mL APA
PCV26 (104 mg (PnPs)/mL) in 13.3 mM L-histidine,
75 mM NaCl, 0.09% w/v PS-20, pH 5.8 6.5 mg/mL Compound
B-1-SNE (0.1 mg/mL of Compound B-1, 4 mg/mL of squalene;
1.2 mg/mL of SPAN-85; and 1.2 mg/mL of PS-20)

Mouse sera were collected prior to study start (pre-immune, day 0) and on days 14 (PD1), 42 (PD2), and 70 (PD3). Sera were evaluated for IgG immunogenicity using a multiplexed electrochemiluminescence (ECL) assay. This assay was developed for use with mouse serum based on the human assay described by Marchese et al. (Marchese R. D., et al., Clin. Vaccine Immunol. (2009) 16(3):387-396) using technology developed by MesoScale Discovery (a division of MesoScale Diagnostics, LLC, Gaithersburg, MD) which utilizes a SULFO-TAG™ label that emits light upon electrochemical stimulation. SULFO-TAG™-labeled anti-mouse IgG was used as the secondary antibody for testing mouse serum samples. Functional antibody titers were determined through multiplexed opsonophagocytic assays (MOPA) based on previously described protocols at www.vaccine.uab.edu and Opsotiter® 3 software owned by and licensed from University of Alabama (UAB) Research Foundation (Caro-Aguilar, I. et al., Vaccine (2017) 35(6):865-872; and Burton R. L., and Nahm M. H., Clin. Vaccine Immunol. (2006) 19(9):1004-1009).

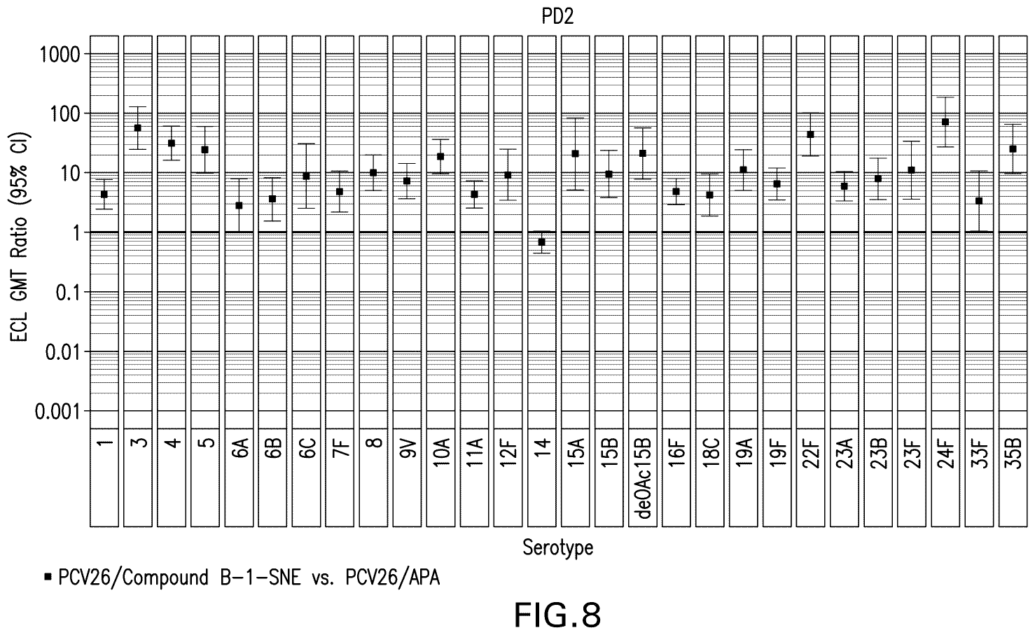

Mouse sera were tested individually in the ECL assay to determine antibody titers and generated antibody titers for all serotypes in the vaccine. Day 0 sera was pooled by group prior to testing in the ECL assay. Antibody titers in mice immunized with PCV26/Compound B-1-SNE were comparable to or higher than titers in mice immunized with PCV26/APA and the maximal difference was observed by day 42 (FIG. 8).

It is also of note that PCV26, which contains polysaccharide conjugates 15A-CRM197, deOAc15B-CRM197, 6A-CRM197, and 6B-CRM197 also provided cross-reactivity to 15B and 6C and these titers in mice immunized with PCV26/Compound B-1-SNE were comparable to or higher than those in mice immunized with PCV26/APA (FIG. 8).

For all timepoints, mouse sera was pooled by group prior to testing in the MOPA to determine functional antibody titers and PCV26 generated functional antibody titers in mice which killed vaccine-type bacterial serotypes. Similar to results from the ECL assay, functional antibody titers in mice immunized with PCV26/Compound B-1-SNE were comparable to or trended higher than titers in mice immunized with PCV26/APA and the maximal difference was observed by day 42 (PD2).

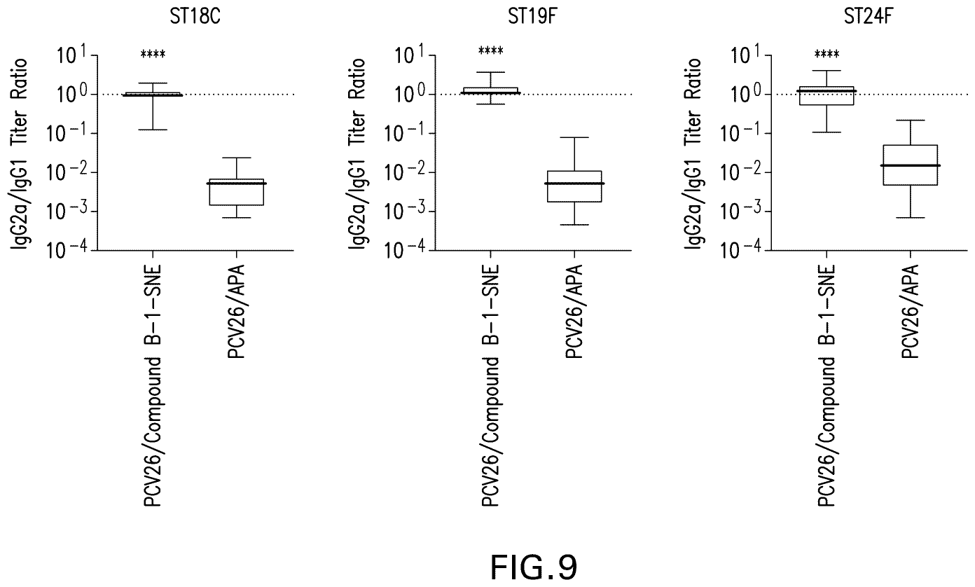

Antibody subclassing was performed to determine if the immune response was driven towards Th1 or Th2. Mouse sera from day 70 (PD3) were tested individually in an IgG subclassing assay to quantify IgG2a and IgG1 antibody titers and the ratio of IgG2a/IgG1 was calculated. A higher ratio indicates immunity driven more towards a Th1 response while a lower ratio indicates immunity driven more towards a Th2 response. Data from three representative serotypes (18C, 19F, and 24F) indicate PCV26/Compound B-1-SNE immunized mice have a more Th1-like response while PCV26/APA immunized mice have a more Th2-like response (FIG. 9). Unpaired T test showed a significant difference in the IgG2a/IgG1 ratio between the PCV26/APA and PCV26/Compound B-1-SNE immunized groups across all three representative serotypes (P<0.0001). The addition of Compound B-1-SNE to a pneumococcal antigen composition is beneficial to not only the response but also to the quality of the response. This data indicates there is a difference in the quality of the immune response induced by Compound B-1-SNE adjuvant and APA adjuvant.

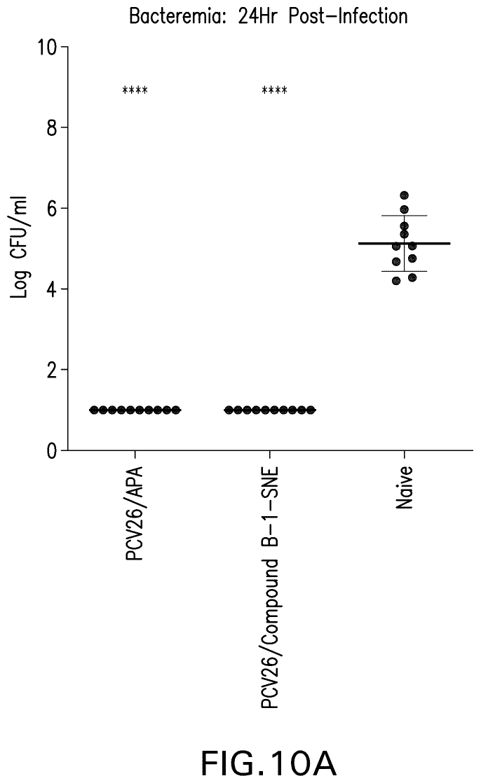

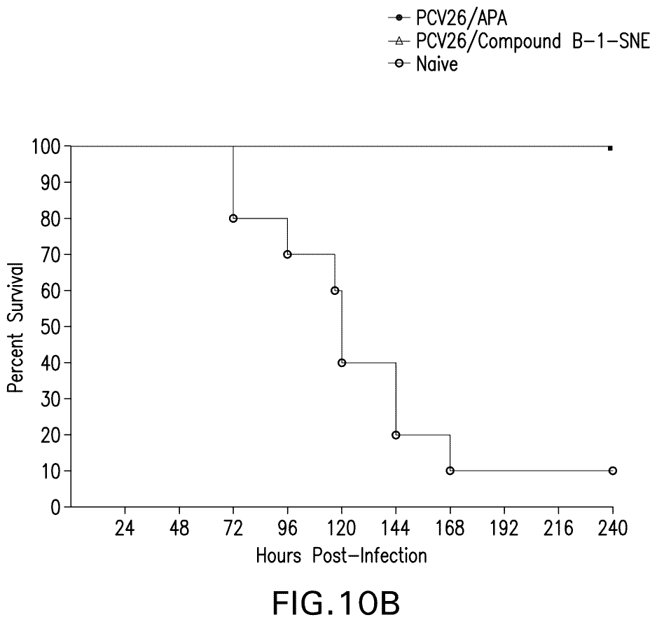

On day 77, 10 mice per group were intratracheally challenged with 10⁵ cfu of S. pneumoniae serotype 24F bacteria in 0.1 mL of PBS. After challenge, mice were weighed daily and blood was collected at 24 hours, 48 hours and 72 hours to assess bacteremia. Mice were monitored by trained animal care staff for any sign of illness or distress in accordance with a schedule approved by the Institutional Animal Care and Use Committee and euthanized if defined criterion are met. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc. All PCV26 immunized mice were protected from bacteremia and showed 100% survival after intratracheal challenge with S. pneumoniae serotype 24F. One-Way ANOVA with Dunnett Multiple comparisons test indicated that PCV26/APA and PCV26/Compound B-1-SNE immunized groups were significantly protected from bacteremia when compared to the naïve control group (P<0.0001) (FIG. 10A). Mantel-Cox log-rank test of survival curves indicated that PCV26/APA and PCV26/Compound B-1-SNE immunized groups were significantly protected from challenge when compared to the naïve control group (P<0.0001) (FIG. 10B).

What is claimed is:

1. An immunogenic composition comprising:
(i) at least one Streptococcus pneumoniae polysaccharide-carrier protein conjugate; and
(ii) a compound having the structure set forth in Formula I: 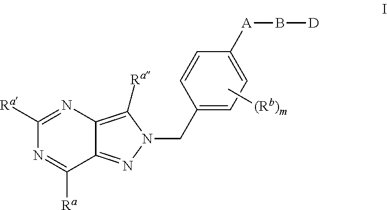
wherein:
R^{a} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;
R^{a′} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and —NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;
R^{a″} is selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and —NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;
R′ and R″ are independently selected from H, (C₁-C₆)alkyl, (C₁-C₆)alkenyl, and (C₁-C₆)alkynyl, wherein said (C₁-C₆)alkyl, (C₁-C₆)alkenyl and (C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine, or R′ and R″, together with the nitrogen to which they are attached, join together to form a (C₃—C₆)heterocycloalkyl, wherein said (C₃-C₆)heterocycloalkyl is optionally substituted with one to four substituents, independently selected from the group consisting of —OH, —O(C₁-C₄)alkyl, —O(C₁-C₄)alkenyl, —O(C₁-C₄)alkynyl, chlorine and fluorine;
each occurrence of R^{b} is independently selected from H, —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, or NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine;
A is a carbon- or nitrogen-linked spacer selected from (C₁-C₆)alkyl, heterocycloalkyl, heterocycloalkyl-C(O)—R^{z}—, (C₁-C₄)alkyl-N(R^{z})—R^{z}—, aryl, and heteroaryl, wherein said (C₁-C₆)alkyl, heterocycloalkyl, aryl and heteroaryl are optionally substituted with one to six substituents, independently selected from the group consisting of —OH, (C₁-C₆)alkyl, (C₁-C₆)alkenyl, (C₁-C₆)alkynyl, —O(C₁-C₆)alkyl, —O(C₁-C₆)alkenyl, —O(C₁-C₆)alkynyl, chlorine, fluorine, and NR′R″, wherein said (C₁-C₆)alkyl, (C₁-C₆)alkenyl, (C₁-C₆)alkynyl, —O(C₁-C₆)alkyl, —O(C₁-C₆)alkenyl, and —O(C₁-C₆)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine;
each occurrence of R^{z} is independently H or (C₁-C₆)alkyl;
B is a functional group selected from 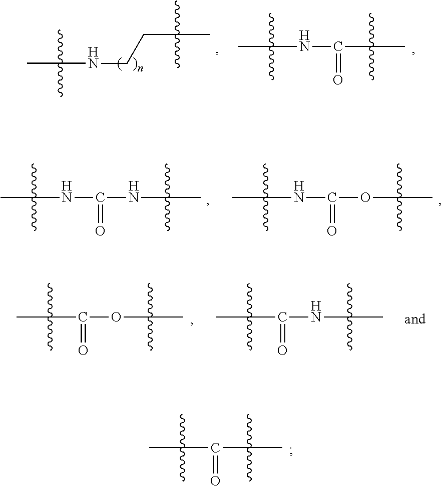
D is a lipid selected from (C₆-C₂₀)alkyl, (C₆-C₂₀)alkenyl and (C₆-C₂₀)alkynyl, wherein said (C₆-C₂₀)alkyl, (C₆-C₂₀)alkenyl, and (C₆-C₂₀)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine; or D is 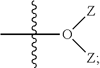
each occurrence of Z is independently selected from (C₆-C₂₀)alkyl, (C₆-C₂₀)alkenyl, and (C₆-C₂₀)alkynyl, wherein said (C₆-C₂₀)alkyl, (C₆-C₂₀)alkenyl, and (C₆-C₂₀)alkynyl are optionally substituted with one to six substituents, independently selected from the group consisting of —OH, —O(C₁-C₄)alkyl, —O(C₁-C₄)alkenyl, —O(C₁-C₄)alkynyl, chlorine and fluorine;
m is 0, 1, 2, 3, 4 or 5; and
n is 0, 1, 2, 3, 4 or 5;
or a pharmaceutically acceptable salt thereof.

2. The immunogenic composition of claim 1, wherein the compound has the structure set forth in Formula Ia: 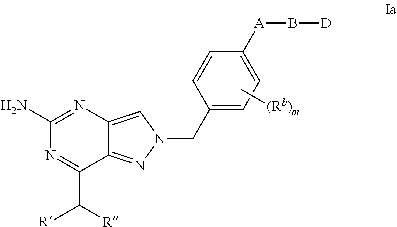
wherein:
R′ and R″ are independently selected from H, (C₁-C₆)alkyl, (C₁-C₆)alkenyl, and (C₁-C₆)alkynyl, wherein said (C₁-C₆)alkyl, (C₁-C₆)alkenyl and (C₁-C₆)alkynyl are optionally substituted with one to four substituents, independently selected from the group consisting of —OH, —O(C₁-C₄)alkyl, —O(C₁-C₄)alkenyl, —O(C₁-C₄)alkynyl, chlorine and fluorine, or R′ and R″, together with the nitrogen to which they are attached, join together to form a (C₃-C₆)heterocycloalkyl, wherein said (C₃-C₆)heterocycloalkyl is optionally substituted with one to four substituents, independently selected from the group consisting of —OH, —O(C₁-C₄)alkyl, —O(C₁-C₄)alkenyl, —O(C₁-C₄)alkynyl, chlorine and fluorine;
each occurrence of R^{b} is —O(C₁-C₄)alkyl, wherein said —O(C₁-C₄)alkyl is optionally substituted with one or two substituents, independently selected from the group consisting of —OH, —O(C₁-C₄)alkyl, —O(C₁-C₄)alkenyl, —O(C₁-C₄)alkynyl, chlorine and fluorine;
A is selected from 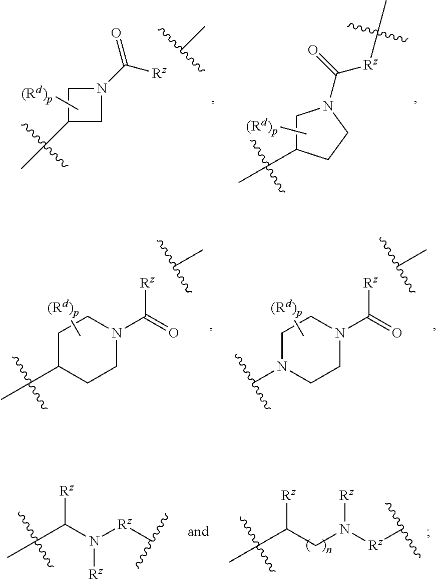
each occurrence of R^{z} is independently H or (C₁-C₆)alkyl;
each occurrence of R^{d} is independently selected from —OH, (C₁-C₄)alkyl, —O(C₁-C₄)alkyl, chlorine and fluorine;
B is 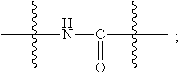 [SMILES: *NC(*)=O]
D is a lipid chain selected from: 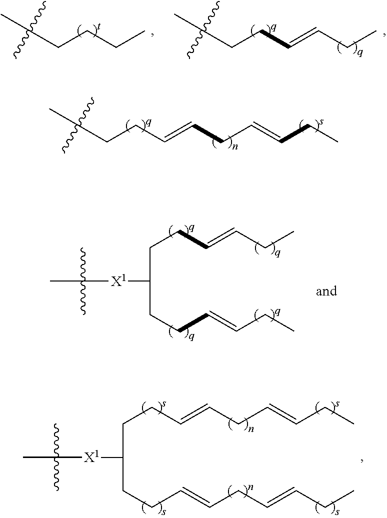
wherein any carbon on the lipid chain is optionally substituted with —OH, —O(C₁-C₄)alkyl, —O(C₁-C₄)alkenyl, —O(C₁-C₄)alkynyl, chlorine or fluorine,
wherein [symbol] is cis or trans stereochemistry,
X¹ is —O—, —C(R)²—, or —NR—, and
each occurrence of R is independently selected from H, (C₁-C₄)alkyl, (C₁-C₄)alkenyl, (C₁-C₄)alkynyl, —OH, —O(C₁-C₄)alkyl, —O(C₁-C₄)alkenyl, —O(C₁-C₄)alkynyl, chlorine and fluorine;
m is 0, 1 or 2;
n is 0, 1, 2 or 3;
p is 0, 1 or 2;
q is 0, 1, 2, 3, 4, 5, 6, 7, 8 or 9;
s is, 1, 2, 3, 4, 5, 6, 7 or 8; and
t is 6, 7, 8, 9, 10, 11, 12, 13, 14, 15, 16, 17 or 18;
or a pharmaceutically acceptable salt thereof.

3. The immunogenic composition of claim 1, wherein the composition comprises more than twenty Streptococcus pneumoniae polysaccharide-carrier protein conjugates, each comprising polysaccharides of a particular Streptococcus pneumoniae serotype.

4. The immunogenic composition of claim 1, wherein the composition comprises more than twenty-five Streptococcus pneumoniae polysaccharide-carrier protein conjugates, each comprising polysaccharides of a particular S. pneumoniae serotype.

5. The immunogenic composition of claim 1, wherein the composition comprises 26 Streptococcus pneumoniae polysaccharide-carrier protein conjugates, each comprising polysaccharide of a particular Streptococcus pneumoniae serotype conjugated to a carrier protein, wherein the Streptococcus pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

6. The immunogenic composition of claim 1, wherein the compound has the structure set forth in Formula II: 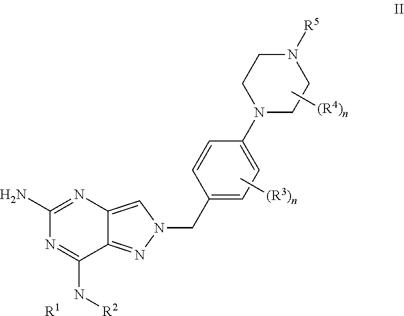
wherein:
R¹ is (C₁-C₆)alkyl, wherein said (C₁-C₆)alkyl is optionally substituted with one to four substituents independently selected from —OH and —O(CH₃);
R² is H, methyl or —O(CH₃);
each occurrence of R³ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);
each occurrence of R⁴ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);
R⁵ is 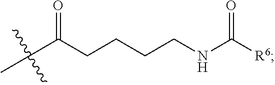
R⁶ is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl, and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine and fluorine; and
each occurrence of n is 4;
or a pharmaceutically acceptable salt thereof.

7. The immunogenic composition of claim 6, wherein the compound has the structure set forth in Formula IIa: 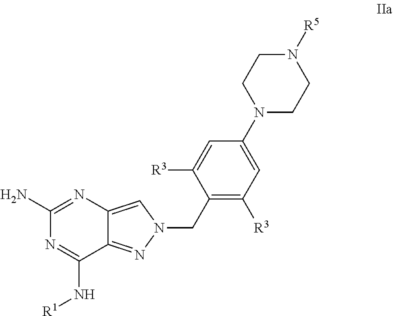
wherein:
R¹ is butyl, wherein said butyl is optionally substituted with one or two —OH;
each occurrence of R³ is independently H or —O(CH₃); and
R⁵ is 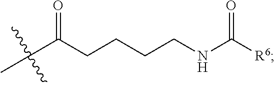
and
R⁶ is selected from (C₁₀-C₂₀)alkyl, (C₁₀-C₂₀)alkenyl and (C₁₀-C₂₀)alkynyl;
or a pharmaceutically acceptable salt thereof.

8. The immunogenic composition of claim 6, wherein the composition comprises more than twenty Streptococcus pneumoniae polysaccharide-carrier protein conjugates, each comprising polysaccharides of a particular Streptococcus pneumoniae serotype.

9. The immunogenic composition of claim 6, wherein the composition comprises more than twenty-five Streptococcus pneumoniae polysaccharide-carrier protein conjugates, each comprising polysaccharides of a particular S. pneumoniae serotype.

10. The immunogenic composition of claim 6, wherein the composition comprises 26 Streptococcus pneumoniae polysaccharide-carrier protein conjugates, each comprising polysaccharides of a particular Streptococcus pneumoniae serotype conjugated to a carrier protein, wherein the Streptococcus pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

11. The immunogenic composition of claim 1, wherein the compound has the structure set forth in Formula III: 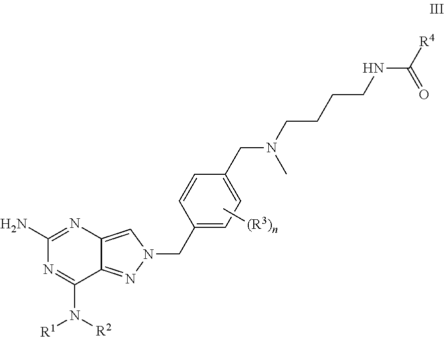
wherein:
R¹ is (C₁-C₆)alkyl, wherein said (C₁-C₆)alkyl is optionally substituted with one to four substituents independently selected from —OH and —O(CH₃);
R² is H, methyl or —O(CH₃);
each occurrence of R³ is independently H, (C₁-C₄)alkyl, (C₁-C₄)alkenyl, (C₁-C₄)alkynyl or —O(C₁-C₄)alkyl wherein said (C₁-C₄)alkyl, (C₁-C₄)alkenyl, (C₁-C₄)alkynyl or —O(C₁-C₄)alkyl are optionally substituted with one or two substituents independently selected from —OH and —O(CH₃);
R⁴ is selected from (C₆-C₂₀)alkyl, (C₆-C₂₀)alkenyl and (C₆-C₂₀)alkynyl, wherein said (C₆-C₂₀)alkyl, (C₆-C₂₀)alkenyl and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine and fluorine; and
n is 4;
or a pharmaceutically acceptable salt thereof.

12. The immunogenic composition of claim 11, wherein the compound has the structure set forth in Formula IIIa: 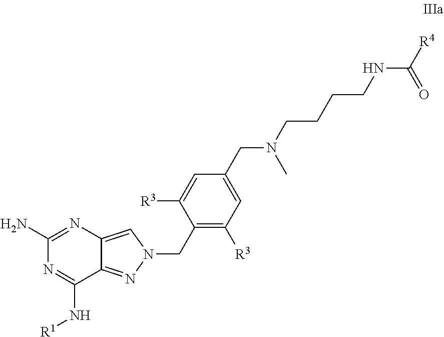
wherein:
R¹ is butyl, wherein said butyl is optionally substituted with one or two —OH;
each occurrence of R³ is independently H or —O(CH₃); and
R⁴ is selected from (C₁₀-C₂₀)alkyl, (C₁₀-C₂₀)alkenyl and (C₁₀-C₂₀)alkynyl;
or a pharmaceutically acceptable salt thereof.

13. The immunogenic composition of claim 11, wherein the composition comprises more than twenty Streptococcus pneumoniae polysaccharide-carrier protein conjugates, each comprising polysaccharides of a particular Streptococcus pneumoniae serotype.

14. The immunogenic composition of claim 11, wherein the composition comprises more than twenty-five Streptococcus pneumoniae polysaccharide-carrier protein conjugates, each comprising polysaccharides of a particular Streptococcus pneumoniae serotype.

15. The immunogenic composition of claim 11, wherein the composition comprises 26 Streptococcus pneumoniae polysaccharide-carrier protein conjugates, each comprising polysaccharides of a particular Streptococcus pneumoniae serotype conjugated to a carrier protein, wherein the Streptococcus pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

16. The immunogenic composition of claim 1, wherein the compound has the structure set forth in Formula IV: 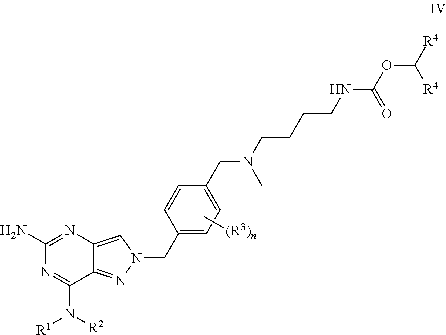
wherein:
R¹ is (C₁-C₆)alkyl, wherein said (C₁-C₆)alkyl is optionally substituted with one to four substituents independently selected from —OH and —O(CH₃);
R² is H, methyl or —O(CH₃);
each occurrence of R³ is independently H, (C₁-C₄)alkyl, (C₁-C₄)alkenyl, (C₁-C₄)alkynyl, or —O(C₁-C₄)alkyl wherein said (C₁-C₄)alkyl, (C₁-C₄)alkenyl, (C₁-C₄)alkynyl, and —O(C₁-C₄)alkyl are optionally substituted with one or two substituents independently selected from —OH and —O(CH₃);
each occurrence of R⁴ is independently selected from (C₆-C₂₀)alkyl, (C₆-C₂₀)alkenyl and (C₆-C₂₀)alkynyl, wherein said (C₆-C₂₀)alkyl, (C₆-C₂₀)alkenyl and (C₆-C₂₀)alkynyl are optionally substituted with one to six substituents independently selected from —OH, —O(C₁-C₄)alkyl, —O(C₁-C₄)alkenyl, —O(C₁-C₄)alkynyl, chlorine or fluorine; and
n is 4;
or a pharmaceutically acceptable salt thereof.

17. The immunogenic composition of claim 16, wherein the compound has the structure set forth in Formula IVa: 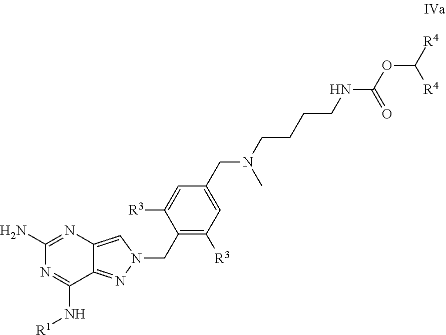
wherein:
R¹ is butyl, wherein said butyl is optionally substituted with one or two —OH;
each occurrence of R³ is independently H or —O(CH₃); and
each occurrence of R⁴ is independently selected from (C₁₀-C₂₀)alkyl, (C₁₀-C₂₀)alkenyl and (C₁₀-C₂₀)alkynyl;
or a pharmaceutically acceptable salt thereof.

18. The immunogenic composition of claim 16, wherein the composition comprises more than twenty Streptococcus pneumoniae polysaccharide-carrier protein conjugates, each comprising polysaccharides of a particular Streptococcus pneumoniae serotype.

19. The immunogenic composition of claim 16, wherein the composition comprises more than twenty-five Streptococcus pneumoniae polysaccharide-carrier protein conjugates, each comprising polysaccharides of a particular Streptococcus pneumoniae serotype.

20. The immunogenic composition of claim 16, wherein the composition comprises 26 Streptococcus pneumoniae polysaccharide-carrier protein conjugates each comprising polysaccharide of a particular Streptococcus pneumoniae serotype conjugated to a carrier protein, wherein the Streptococcus pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

21. The immunogenic composition of claim 1, wherein the compound is selected from:
(N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide;
(S)—N-(5-(4-(4-((5-amino-7-((1-hydroxypentan-2-yl)amino)-2H-pyrazolo[4,3-d]pyrimidin-2-yl)methyl)-3,5-dimethoxyphenyl)piperazin-1-yl)-5-oxopentyl)stearamide;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)tetradecanamide;
N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)oleamide;
(9Z,12Z)—N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)octadeca-9,12-dienamide;
N-(5-(4-(4-((5-amino-7-(butylamino)-2H-pyrazolo[4,3-d]pyrimidin-2-yl)methyl)-3-methoxyphenyl)-1,4-diazepan-1-yl)-5-oxopentyl)stearamide;
N-(5-(4-(4-((5-amino-7-(butylamino)-2H-pyrazolo[4,3-d]pyrimidin-2-yl)methyl)-3-methoxyphenyl)piperidin-1-yl)-5-oxopentyl)stearamide;
N-(5-(3-(4-((5-amino-7-(butylamino)-2H-pyrazolo[4,3-d]pyrimidin-2-yl)methyl)-3-methoxyphenyl)azetidin-1-yl)-5-oxopentyl)stearamide;
1-(4-((5-amino-7-(butylamino)-2H-pyrazolo[4,3-d]pyrimidin-2-yl)methyl)-3-methoxyphenyl)-N-(3-stearamidopropyl)piperidine-4-carboxamide;
(1s,3s)-3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)-N-hexadecylcyclobutane-1-carboxamide;
N-(3-(4-(4-((5-amino-7-(butylamino)-2H-pyrazolo[4,3-d]pyrimidin-2-yl)methyl)-3-methoxyphenyl)piperazin-1-yl)-3-oxopropyl)stearamide;
N-(7-(4-(4-((5-amino-7-(butylamino)-2H-pyrazolo[4,3-d]pyrimidin-2-yl)methyl)-3-methoxyphenyl)piperazin-1-yl)-7-oxoheptyl)stearamide;
N-(3-(2-(4-(4-((5-amino-7-(butylamino)-2H-pyrazolo[4,3-d]pyrimidin-2-yl)methyl)-3-methoxyphenyl)piperazin-1-yl)-2-oxoethyl)cyclobutyl)stearamide;
N-(5-(4-(4-((5-amino-7-(butylamino)-2H-pyrazolo[4,3-d]pyrimidin-2-yl)methyl)-3-methoxyphenyl)piperazin-1-yl)-4,4-dimethyl-5-oxopentyl)stearamide;
N-(5-(4-(4-((5-amino-7-(butylamino)-3-methyl-2H-pyrazolo[4,3-d]pyrimidin-2-yl)methyl)-3-methoxyphenyl)piperazin-1-yl)-5-oxopentyl)stearamide;
(9Z,12Z)—N-(4-((4-((5-amino-7-(butylamino)-2H-pyrazolo[4,3-d]pyrimidin-2-yl)methyl)-3,5-dimethoxybenzyl)(methyl)amino)butyl)octadeca-9,12-dienamide;
N-(4-((4-((5-amino-7-(butylamino)-2H-pyrazolo[4,3-d]pyrimidin-2-yl)methyl)-3,5-dimethoxybenzyl)(methyl)amino)butyl)tetradecanamide;
N-(4-((4-((5-amino-7-(butylamino)-2H-pyrazolo[4,3-d]pyrimidin-2-yl)methyl)-3,5-dimethoxybenzyl)(methyl)amino)butyl)oleamide;
N-(4-((4-((5-amino-7-(butylamino)-2H-pyrazolo[4,3-d]pyrimidin-2-yl)methyl)-3,5-dimethoxybenzyl)(methyl)amino)butyl)stearamide;
N-(4-((4-((5-amino-7-(butylamino)-2H-pyrazolo[4,3-d]pyrimidin-2-yl)methyl)-3,5-dimethoxybenzyl)(methyl)amino)-4-oxobutyl)stearamide;
(6Z,9Z,28Z,31Z)-heptatriaconta-6,9,28,31-tetraen-19-yl (4-((4-((5-amino-7-(butylamino)-2H-pyrazolo[4,3-d]pyrimidin-2-yl)methyl)-3,5-dimethoxybenzyl)(methyl)amino)butyl)carbamate;
4-(4-((5-amino-7-(butylamino)-2H-pyrazolo[4,3-d]pyrimidin-2-yl)methyl)-3-methoxyphenyl)-N-(3-stearamidopropyl)piperazine-1-carboxamide; and
3-stearamidopropyl 4-(4-((5-amino-7-(butylamino)-2H-pyrazolo[4,3-d]pyrimidin-2-yl)methyl)-3-methoxyphenyl)piperazine-1-carboxylate;
or a pharmaceutically acceptable salt thereof.

22. The immunogenic composition of claim 1, wherein the compound is: N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide;
or a pharmaceutically acceptable salt thereof.

23. The immunogenic composition of claim 1, further comprising (iii) sorbitan trioleate (SPAN-85); (iv) polysorbate-20 (PS-20) or polysorbate-80 (PS-80); and (v) squalene.

24. The immunogenic composition of claim 23, wherein the compound is N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide, or a pharmaceutically acceptable salt thereof.

25. The immunogenic composition of claim 24, wherein the composition comprises PS-20.

26. The immunogenic composition of claim 23, wherein the composition comprises more than twenty Streptococcus pneumoniae polysaccharide-carrier protein conjugates, each comprising polysaccharides of a particular Streptococcus pneumoniae serotype.

27. The immunogenic composition of claim 23, wherein the composition comprises more than twenty-five Streptococcus pneumoniae polysaccharide-carrier protein conjugates, each comprising polysaccharides of a particular Streptococcus pneumoniae serotype.

28. The immunogenic composition of claim 23, wherein the composition comprises 26 Streptococcus pneumoniae polysaccharide-carrier protein conjugates, each comprising polysaccharides of a particular Streptococcus pneumoniae serotype conjugated to a carrier protein, wherein the Streptococcus pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

29. The immunogenic composition of claim 25, wherein the composition comprises 26 Streptococcus pneumoniae polysaccharide-carrier protein conjugates, each comprising polysaccharides of a particular Streptococcus pneumoniae serotype conjugated to a carrier protein, wherein the Streptococcus pneumoniae serotypes consist of 1, 3, 4, 5, 6A, 6B, 7F, 8, 9V, 10A, 11A, 12F, 14, 15A, de-O-acetylated 15B, 16F, 18C, 19A, 19F, 22F, 23A, 23B, 23F, 24F, 33F and 35B.

30. The immunogenic composition of claim 23, further comprising (vi) a pharmaceutically acceptable carrier.

31. The immunogenic composition of claim 25, further comprising (vi) a pharmaceutically acceptable carrier.

32. The immunogenic composition of claim 1, wherein the carrier protein is CRM197.

33. The immunogenic composition of claim 23, wherein the carrier protein is CRM197.

34. The immunogenic composition of claim 25, wherein the carrier protein is CRM197.

35. The immunogenic composition of claim 23, wherein the concentration of compound is 0.1 μg/mL to 100 μg/mL, the concentration of SPAN-85 is 0.01 mg/mL to 50 mg/mL, the concentration of PS-20 or PS-80 is 0.01 mg/mL to 50 mg/mL, and the concentration of squalene is 0.02 mg/mL to 20 mg/mL.

36. A method of treating or preventing pneumococcal disease in a human patient comprising administrating to the patient the immunogenic composition of claim 1.

37. A method of treating or preventing pneumococcal disease in a human patient comprising administrating to the patient the immunogenic composition of claim 23.

38. A method of treating or preventing pneumococcal disease in a human patient comprising administrating to the patient the immunogenic composition of claim 25.
